(19) United States
(12) Patent Application Publication
(10) Pub. No.: US 2007/0043057 A1
(43) Pub. Date: Feb. 22, 2007

(54) Lonidamine analogs

(75) Inventors: Mark Matteucci, Portola Valley, CA (US); Photon Rao, Foster City, CA (US); Jian-Xin Duan, So. San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Threshold Pharmaceuticals, Inc., Redwood City, CA (US)

(21) Appl. No.: 11/346,632

(22) Filed: Feb. 1, 2006

Related U.S. Application Data
(63) Continuation-in-part of application No. PCT/US05/27092, filed on Jul. 29, 2005.
(60) Provisional application No. 60/683,087, filed on May 19, 2005.
(60) Provisional application No. 60/764,427, filed on Feb. 1, 2006.
(60) Provisional application No. 60/661,067, filed on Mar. 11, 2005.
(60) Provisional application No. 60/651,705, filed on Feb. 9, 2005.

(51) Int. Cl. A61K 31/506; A61K 31/501; A61K 31/497; A61K 31/4745; A61K 31/4439; A61K 31/416; A61K 31/403

(52) U.S. Cl. 514/252.04; 514/406; 548/113; 548/361.1; 514/306; 514/412; 514/303; 546/117; 546/118; 514/338; 514/275; 544/333; 544/238; 544/405; 548/452; 514/255.05; 544/331; 546/275.7; 546/277.1

(57) ABSTRACT

Lonidamine analogs are useful in the treatment and prevention of cancer, benign prostatic hyperplasia, macular degeneration and prostatic intraepithelial neoplasia, or for use as an antispermatigenic agent.

### CROSS-REFERENCES TO RELATED APPLICATIONS

This application is a Continuation-in-Part of PCT Application No. PCT/US2005/027092, filed 29 Jul. 2005; U.S. Patent Application No. 60/683,087, filed May 19, 2005; and claims the benefit of U.S. Patent Application No. 60/______, filed Jan. 31, 2006 (Attorney Docket No. 021305-007200US); U.S. Patent Application No. 60/661,067, filed Mar. 11, 2005; U.S. Patent Application No. 60/651,705, filed Feb. 9, 2005; U.S. Patent Application No. 60/646,188, filed Jan. 21, 2005; U.S. Patent Application No. 60/599,666, filed Aug. 5, 2004; U.S. Patent Application No. 60/592,833, filed Jul. 29, 2004; and U.S. Patent Application No. 60/592,723, filed Jul. 29, 2004, the contents of each of which is incorporated herein by reference.

### BACKGROUND OF THE INVENTION

Lonidamine (LND), also known as 1-(2,4-dichlorobenzyl)-1H-indazole-3-carboxylic acid, is an anti-cancer drug approved for the treatment of lung, breast, prostate, and brain cancer. The mechanism of action of lonidamine may involve interference with the energy metabolism of neoplastic cells by disruption of the mitochondrial membrane and by inhibition of hexokinase. Lonidamine also has anti-spermatogenic activity and has been shown to inhibit germ cell respiration. Lonidamine has perhaps been most extensively been studied for use in the treatment of advanced breast cancer. For example, the reference Mansi et al., September 1991, Br. J. Cancer 64(3): 593-7, reports a phase II study in which lonidamine was administered in a daily divided oral dose of 600 mg. Of the 28 patients evaluable for response, three (11%) achieved a partial response (4-24+ months); three (11%) a minor response; two had stable disease (greater than 3 months); and 20 progressed. The investigators reported no clear relationship between lonidamine levels and clinical response or toxicity and concluded that lonidamine appeared to be active against advanced breast cancer; and that lonidamine's low toxicity would allow combination studies.

Combination studies of lonidamine in advanced breast cancer followed this report, particularly studies in combination with epirubicin or doxorubicin. For examples, see Iaffaioli et al., September 1995, Breast Cancer Res. Treat 35(3): 243-8 (phase II trial of high-dose epirubicin, lonidamine, and alpha 2b interferon); Gardin et al., January 1996, Eur J Cancer 32A(1): 176-7 (phase II trial of lonidamine plus epirubicin and cyclophosphamide); Dogliotti et al., April 1996, J Clin Oncol 14(4): 1165-72 (multicenter prospective randomized trial—reports that lonidamine significantly increases the activity of epirubicin); Gebbia et al., November 1997, Anticancer Drugs 8(10): 943-8 (phase II trial of cisplatin and epirubicin plus oral lonidamine as first-line treatment for metastatic breast cancer); Amadori et al., June 1998, Breast Cancer Res. Treat 49(3): 209-17 (multicenter prospective randomized trial—reports modulating effect of lonidamine on response to doxorubicin in metastatic breast cancer); Dogliotti et al., 1998, Cancer Chemother Pharmacol 41(4): 333-8 (pilot study of cisplatin, epirubicin, and lonidamine combination regimen as first-line chemotherapy for metastatic breast cancer); Nistico et al., August 1999, Breast Cancer Res. Treat 56(3): 233-7 (study of weekly dosed epirubicin plus lonidamine in advanced breast carcinoma); and Pacini et al., May 2000, Eur J Cancer 36(8): 966-75 (multicentric randomised study of FEC (5-fluorouracil, epidoxorubicin and cyclophosphamide) versus EM (epidoxorubicin and mitomycin-C) with or without lonidamine as first-line treatment). Surprisingly, however, a more recent reference, Berruti et al., 15 Oct. 2002, J. Clin. Oncol. 20(20): 4150-9, reports that, in a phase III study with a factorial design, time to progression in metastatic breast cancer patients treated with epirubicin was not improved by the addition of either cisplatin or lonidamine (see also Berruti et al., July-August 1997, Anticancer Res. 17(4A): 2763-8).

Lonidamine has also been studied in lung cancer, particularly non-small cell lung cancer (see Joss et al., September 1984, Cancer Treat Rev 11(3): 205-36) in combination with radiation or other anti-cancer agents. For examples, see Privitera et al., December 1987, Radiother Oncol 10(4): 285-90 (phase II double-blind randomized study of lonidamine and radiotherapy in epidermoid carcinoma of the lung); Gallo-Curcio et al., December 1988, Semin Oncol 15(6 Suppl 7): 26-31 (chemotherapy or radiation therapy plus and minus lonidamine); Giaccone et al., 28 Feb. 1989, Tumori 75(1): 43-6 (preliminary analysis of lonidamine versus polychemotherapy); Ianniello et al., 1 Jul. 1996, Cancer 78(1): 63-9 (multicenter randomized clinical trial of cisplatin, epirubicin, and vindesine with or without lonidamine); Gridelli et al., March-April 1997, Anticancer Res. 17(2B): 1277-9 (phase II trial of VM-26 plus lonidamine in pretreated small cell lung cancer); Comella et al., May 1999, J Clin Oncol 17(5): 1526-34 (phase II randomized trial of cisplatin, gemcitabine, and vinorelbine); DeMarinis et al., May-June 1999, Tumori 85(3): 177-82 (phase III randomized trial of vindesine and lonidamine in elderly patients); and Portalone et al., July-August 1999, Tumori 85 (4): 239-42 (phase II study with cisplatin, epidoxorubicin, vindesine and lonidamine).

Lonidamine has been studied as a treatment for other cancers (see Robustelli et al., April 1991, Semin. Oncol. 18(2 Suppl 4):18-22; and Pacilio et al., 1984, Oncology 41 Suppl 1:108-12), including: favorable B-cell neoplasms (see Robins et al., April 1990, Int J Radiat Oncol Biol Phys. 18(4):909-20, which describes two pilot clinical trials and laboratory investigations of adjunctive therapy (whole body hyperthermia versus lonidamine) to total body irradiation); advanced colorectal cancer (see the references Passalacqua et al., Jun. 30 1989, Tumori 75(3):277-9, and Zaniboni et al., November-December 1995, Tumori 81(6):435-7, which describes a phase II study of mitomycin C and lonidamine as second-line therapy); advanced gastric carcinoma (see Barone et al., 15 Apr. 1998, Cancer 82(8):1460-7, which describes two parallel randomized phase II studies with a 5-fluorouracil-based or a cisplatin-based regimen); malignant glioma (see Carapella et al., May 1989, J Neurooncol 7(1):103-8, and July-December 1990, J Neurosurg Sci. 34(3-4):261-4); metastatic cancers (see the references Weinerman, 1990, Cancer Invest. 8(5):505-8, which describes a phase I study of lonidamine and human lymphoblastoid alpha interferon; DeAngelis et al., September 1989, J Neurooncol 7(3):241-7, and U.S. Pat. No. 5,260,327, which describe the combined use of radiation therapy and lonidamine in the treatment of brain metastases; and Weinerman et al., June 1986, Cancer Treat Rep 70(6):751-4, which reports a phase II study of lonidamine in patients with metastatic renal cell carcinoma); advanced ovarian cancer (see the references Bottalico et al., November-December 1996, Anticancer Res 16(6B):3865-9; DeLena et al., October 1997, J Clin Oncol 15(10):3208-13, which reports the revertant and potentiating activity of lonidamine in patients with ovarian cancer previously treated with platinum; and DeLena et al., February 2001, Eur J Cancer 37(3):364-8, which describes a phase II study of paclitaxel, cisplatin and lonidamine); and recurrent papillary carcinomas of the urinary bladder (see the reference Giannotti et al., 1984, Oncology 41 Suppl 1:104-7, which describes treatment results after administration of lonidamine plus adriamycin versus adriamycin alone in adjuvant treatment).

Lonidamine has been studied as a treatment of Benign Prostatic Hypertrophy or Benign Prostatic Hyperplasia (BPH) (see U.S. Pat. No. 6,989,400, incorporated herein by reference). BPH is a disease in which prostate epithelial cells grow abnormally and block urine flow, and currently afflicts more than 10 million adult males in the United States alone and many millions more throughout the rest of the world.

There remains a need for compounds in addition to lonidamine that are efficacious in the treatment of cancer, either alone or in combination with other anti-cancer agents, and for the treatment of BPH. The present invention meets this need.

### BRIEF SUMMARY OF THE INVENTION

The present invention provides lonidamine analogs and pharmaceutical formulations of those compounds suitable for use as drugs in the methods of the invention for treating cancer and/or BPH. The drugs can have high aqueous solubility and extended pharmacokinetics in vivo.

In one aspect, the present invention provides compounds which are analogs of lonidamine. The compounds of the present invention have the formula (I): 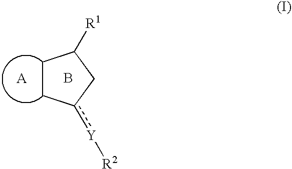
wherein A-B is a 7,5, 6,5 or a 5,5 cyclic ring system, optionally substituted with from one to five V⁶ substituents, each independently selected from the group consisting of hydrogen, amino, halo, oxo, cyano, nitro, (C₁-C₈) alkyl, (C₁-C₆) alkoxy, nitro, acetamido, L¹-CO₂H, L¹-dialkylamino, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CUR³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₂R³, SOR³, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, cyano, nitrileoxide, and —NO, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

R¹ is selected from the group consisting of CO₂R³, COR⁴, COCOR³, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH—V⁵, COCOR⁴, CON(R³)N═CR³R⁷, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;

L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₆) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl;

R² is an aryl or heteroaryl group, optionally substituted with from one to five R⁶ substituents independently selected from the group consisting of halo, nitro, cyano, nitrileoxide, —NO, R³, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, nitrileoxide, and —NO;

each R³ is a member independently selected from the group consisting of H, (C₁-C₈) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl and heteroaryl;

each R⁴ is a member independently selected from the group consisting of NR³R⁷, NR³OR⁷, NR⁷NR³R⁷ or NR³CN;

R⁵ is H, OH or halogen;

R⁷ is selected from the group consisting of H, (C₁-C₈) alkyl, (C₂-C₆) alkenyl, (C₂-C₈) alkynyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;

R⁸ is H, halo, nitro, cyano, nitrileoxide, —NO, R³, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₃R³¹, SO₂NR³R⁷, SO₂NR³CUR³, SO₂N(CUR³), SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(R³CUNR³R⁷)₂, (NR³)₂, or 2R⁸ taken together form a (C₃-C₈) cycloalkyl, (C₃-C₈) heterocyclyl or heteroaryl ring;

R³¹ is aryl or heteroaryl;

each V⁵ is a member independently selected from the group consisting of COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂R³, CONHSO₂R³, and C(═NCN)NH₂;

Y is CR⁸₂, CR⁸, NR⁸, S or O;

U is O, S, NR³, NCOR³, or NCONR³R⁷;

U¹ is O or S;

[symbol] represents a single or double bond; and

pharmaceutically acceptable salts, solvates, hydrates, and prodrugs thereof.

In a second aspect, the present invention provides lonidamine analogs that have improved aqueous solubility and extended pharmacokinetics in vivo.

In a third aspect, the present invention provides methods for treating cancer in a subject, comprising administering to the subject an effective amount of a compound of the invention.

In a fourth aspect, the present invention provides methods for treating BPH in a subject, comprising administering to the subject an effective amount of a compound of the invention.

In a fifth aspect, the present invention provides methods for synthesizing the compounds of the invention and compounds useful as intermediates in such synthetic methods.

In a sixth aspect, the present invention provides pharmaceutical formulations of the compounds of the invention.

These and other aspects and embodiments of the invention are described in more detail in the detailed description and claims that follow.

### BRIEF DESCRIPTION OF THE DRAWINGS

FIG. 1 shows the morphology of prostate in a normal mouse.

FIG. 2 shows the morphology of prostate in a mouse treated with 5 mg/kg Compound 1.

FIG. 3 shows the morphology of prostate in a mouse treated with 20 mg/kg Compound 1.

FIG. 4 illustrates a dose dependent reduction in relative right testis weight upon administration of Compound 1.

FIG. 5 illustrates a dose dependent reduction in relative left testis weight upon administration of Compound 1.

FIG. 6 illustrates a dose dependent reduction in relative whole prostate weight upon administration of Compound 1.

FIG. 7 illustrates a dose dependent reduction in relative dorsal prostate weight upon administration of Compound 1.

FIG. 8 illustrates a dose dependent reduction in relative ventral prostate weight upon administration of Compound 1.

FIG. 9 illustrates a dose dependent reduction in absolute ventral prostate weight upon administration of Compound 1.

FIG. 10 illustrates a dose dependent reduction in absolute dorsal prostate weight upon administration of Compound 1.

FIG. 11 illustrates a dose dependent reduction in absolute whole prostate weight upon administration of Compound 1

FIG. 12 illustrates a dose dependent reduction in absolute right testis weight upon administration of Compound 1.

FIG. 13 illustrates a dose dependent reduction in absolute left testis weight upon administration of Compound 1.

FIG. 14 illustrates a reduction in absolute ventral prostate weight upon administration of Compound 3.

FIG. 15 illustrates a reduction in absolute dorsal prostate weight upon administration of Compound 3.

FIG. 16 illustrates a reduction in absolute anterior prostate weight upon administration of Compound 3.

FIG. 17 illustrates a reduction in absolute right testis weight upon administration of Compound 3.

FIG. 18 illustrates a reduction in absolute left testis weight upon administration of Compound 3.

### DETAILED DESCRIPTION OF THE INVENTION

The description below is organized into sections for convenience only, and disclosure found in any organizational section is applicable to any aspect of the invention.

Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the terms “a” or “an” means “at least one” or “one or more.”

As used herein, the term “Alkyl” refer to a linear saturated monovalent hydrocarbon radical or a branched saturated monovalent hydrocarbon radical having the number of carbon atoms indicated in the prefix. For example, (C₁-C₈) alkyl is meant to include methyl, ethyl, n-propyl, 2-propyl, n-butyl, 2-butyl, tert-butyl, pentyl, and the like. For each of the definitions herein (e.g., alkyl, alkenyl, alkoxy, araalkyloxy), when a prefix is not included to indicate the number of main chain carbon atoms in an alkyl portion, the radical or portion thereof will have six or fewer main chain carbon atoms. (C₁-C₈) Alkyl may be further substituted with substituents, including for example, hydroxyl, amino, mono or di(C₁-C₆) alkyl amino, halo, (C₂-C₆) alkenyl ether, cyano, nitro, ethenyl, ethynyl, (C₁-C₆) alkoxy, (C₁-C₆) alkylthio, acyl, —COOH, —CONH₂, mono- or di-(C₁-C₆) alkyl-carboxamido, —SO₂NH₂, —OSO₂—(C₁-C₆) alkyl, mono or di(C₁-C₆) alkylsulfonamido, cyclohexyl, heterocyclyl, aryl and heteroaryl.

As used herein, the terms “acyl” or “alkanoyl” means the group —C(O)R′, where R′ is hydrogen, alkyl, cycloalkyl, cycloalkyl-alkyl, aryl, arylalkyl, and variations of these groups in which one or more carbon atoms have been replaced with heteroatoms.

As used herein, the term “Alkylene” refer to a linear saturated divalent hydrocarbon radical or a branched saturated divalent hydrocarbon radical having the number of carbon atoms indicated in the prefix. For example, (C₁-C₆) alkylene is meant to include methylene, ethylene, propylene, 2-methylpropylene, pentylene, and the like.

As used herein, the term “Alkenyl” refers to a linear monovalent hydrocarbon radical or a branched monovalent hydrocarbon radical having the number of carbon atoms indicated in the prefix and containing at least one double bond, but no more than three double bonds. For example, (C₂-C₆) alkenyl is meant to include, ethenyl, propenyl, 1,3-butadienyl and the like.

As used herein, the term “Alkynyl” means a linear monovalent hydrocarbon radical or a branched monovalent hydrocarbon radical containing at least one triple bond and having the number of carbon atoms indicated in the prefix. The term “alkynyl” is also meant to include those alkyl groups having one triple bond and one double bond. For example, (C₂-C₆) alkynyl is meant to include ethynyl, propynyl, and the like.

As used herein, the terms “Alkoxy”, “aryloxy” or “araalkyloxy” refer to a radical —OR wherein R is an alkyl, aryl or arylalkyl, respectively, as defined herein, e.g., methoxy, phenoxy, benzyloxy, and the like.

As used herein, the terms “Aryl” or “arylene” or “arene” refer to a monovalent monocyclic or bicyclic aromatic hydrocarbon radical of 6 to 10 ring atoms which is substituted independently with one to four substituents, preferably one, two, or three substituents selected from alkyl, cycloalkyl, cycloalkylalkyl, halo, nitro, cyano, hydroxy, alkoxy, amino, acylamino, mono-alkylamino, di-alkylamino, haloalkyl, haloalkoxy, heteroalkyl, COR (where R is hydrogen, alkyl, cycloalkyl, cycloalkyl-alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl) or —(CR′R″)ₙ—CONR^{x}R^{y} (where n is an integer from 0 to 5, R′ and R″ are independently hydrogen or alkyl, and R^{x} and R^{y} are independently selected from hydrogen, alkyl, cycloalkyl, cycloalkylalkyl, phenyl or phenylalkyl). In one embodiment, R^{x} and R^{y} together is heterocyclyl. More specifically the term aryl includes, but is not limited to, phenyl, biphenyl, 1-naphthyl, and 2-naphthyl, and the substituted forms thereof.

As used herein, the terms “Araalkyl” or “Aryl (C₁-Cₓ) alkyl” refer to the radical —R^{x}R^{y} where R^{x} is an alkylene group (having eight or fewer main chain carbon atoms) and R^{y} is an aryl group as defined above. Thus, “araalkyl” refers to groups such as, for example, benzyl, phenylethyl, 3-(4-nitrophenyl)-2-methylbutyl, and the like. Similarly, “Araalkenyl” means a radical —R^{x}R^{y} where R^{x} is an alkenylene group (an alkylene group having one or two double bonds) and R^{y} is an aryl group as defined above, e.g., styryl, 3-phenyl-2-propenyl, and the like.

As used herein, the term “cyclic ring system” means a single heterocyclyl, cycloalkyl, aryl, or heteroaryl ring or combination of heterocyclyl, cycloalkyl, aryl, or heteroaryl rings as defined herein.

As used herein, the term “Cycloalkyl” refers to a monovalent cyclic hydrocarbon radical of three to seven ring carbons. The cycloalkyl group may have double bonds which may but not necessarily be referred to as “cycloalkene” or “cycloalkenyl”. The cycloalkyl ring may be optionally substituted independently with one, two, or three substituents selected from alkyl, aryl, arylalkyl, heteroaryl, heteroarylalkyl, cycloalkylalkyl, halo, nitro, cyano, hydroxy, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl), —(CR′R″)ₙ—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically, the term cycloalkyl includes, for example, cyclopropyl, cyclohexyl, cyclohexenyl, phenylcyclohexyl, 4-carboxycyclohexyl, 2-carboxamidocyclohexenyl, 2-dimethylaminocarbonyl-cyclohexyl, and the like.

As used herein, the term “Cycloalkyl-alkyl” means a radical —R^{x}R^{y} wherein R is an alkylene group and R^{y} is a cycloalkyl group as defined herein, e.g., cyclopropylmethyl, cyclohexenylpropyl, 3-cyclohexyl-2-methylpropyl, and the like. The prefix indicating the number of carbon atoms (e.g., C₄-C₁₀) refers to the total number of carbon atoms from both the cycloalkyl portion and the alkyl portion.

As used herein, the term “halo” and the term “halogen” when used to describe a substituent, refer to —F, —Cl, —Br and —I.

As used herein, the term “Heteroalkyl” means an alkyl radical as defined herein with one, two or three substituents independently selected from cyano, —OR^{w}, —NR^{x}R^{y}, and —S(O)ₚR^{z} (where p is an integer from 0 to 2), with the understanding that the point of attachment of the heteroalkyl radical is through a carbon atom of the heteroalkyl radical. R^{w} is hydrogen, alkyl, cycloalkyl, cycloalkyl-alkyl, aryl, araalkyl, alkoxycarbonyl, aryloxycarbonyl, carboxamido, or mono- or di-alkylcarbamoyl. R^{x} is hydrogen, alkyl, cycloalkyl, cycloalkyl-alkyl, aryl or araalkyl. R^{y} is hydrogen, alkyl, cycloalkyl, cycloalkyl-alkyl, aryl, araalkyl, alkoxycarbonyl, aryloxycarbonyl, carboxamido, mono- or di-alkylcarbamoyl or alkylsulfonyl. R^{z} is hydrogen (provided that p is 0), alkyl, cycloalkyl, cycloalkyl-alkyl, aryl, araalkyl, amino, mono-alkylamino, di-alkylamino, or hydroxyalkyl. Representative examples include, for example, 2-hydroxyethyl, 2,3-dihydroxypropyl, 2-methoxyethyl, benzyloxymethyl, 2-cyanoethyl, and 2-methylsulfonyl-ethyl. For each of the above, R^{w}, R^{x}, R^{y}, and R^{z} can be further substituted by amino, fluorine, alkylamino, di-alkylamino, OH or alkoxy. Additionally, the prefix indicating the number of carbon atoms (e.g., C₁-C₁₀) refers to the total number of carbon atoms in the portion of the heteroalkyl group exclusive of the cyano, —OR^{w}, —NR^{x}R^{y}, or —S(O)ₚR^{z} portions. The term “heteroalkyl,” by itself or in combination with another term, also refers to a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂₋.

As used herein, the term “heteroaryl” or “heteroaryl ring” means a monovalent monocyclic or bicyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring is optionally substituted independently with one to four substituents, preferably one or two substituents, selected from alkyl, cycloalkyl, cycloalkyl-alkyl, halo, nitro, cyano, hydroxy, alkoxy, amino, acylamino, mono-alkylamino, di-alkylamino, haloalkyl, haloalkoxy, heteroalkyl, —COR (where R is hydrogen, alkyl, phenyl or phenylalkyl, —(CR′R″)ₙ—COOR (where n is an integer from 0 to 5, R′ and R″ are independently hydrogen or alkyl, and R is hydrogen, alkyl, cycloalkyl, cycloalkyl-alkyl, phenyl or phenylalkyl), or —(CR′R″)ₙ—CONR^{x}R^{y} (where n is an integer from 0 to 5, R′ and R″ are independently hydrogen or alkyl, and R^{x} and R^{y} are, independently of each other, hydrogen, alkyl, cycloalkyl, cycloalkyl-alkyl, phenyl or phenylalkyl). In one embodiment, R^{x} and R^{y} together is heterocyclyl. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, indazolyl, pyrrolopyrymidinyl, indolizinyl, pyrazolopyridinyl, triazolopyridinyl, pyrazolopyrimidinyl, triazolopyrimidinyl, pyrrolotriazinyl, pyrazolotriazinyl, triazolotriazinyl, pyrazolotetrazinyl, hexaaza-indenyl, and heptaaza-indenyl and the derivatives thereof. Unless indicated otherwise, the arrangement of the hetero atoms within the ring may be any arrangement allowed by the bonding characteristics of the constituent ring atoms.

As used herein, the terms “heterocycle”, “heterocyclyl”, “heterocycloalkyl” or “cycloheteroalkyl” means a saturated or unsaturated non-aromatic cyclic radical of 3 to 8 ring atoms in which one to four ring atoms are heteroatoms selected from O, NR (where R is independently hydrogen or alkyl) or S(O)ₚ (where p is an integer from 0 to 2), the remaining ring atoms being C, where one or two C atoms may optionally be replaced by a carbonyl group. The heterocyclyl ring may be optionally substituted independently with one, two, or three substituents selected from alkyl, aryl, arylalkyl, heteroaryl, heteroarylalkyl, cycloalkyl, cycloalkylalkyl, halo, nitro, cyano, hydroxy, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl), —(CR′R″)ₙ—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically the term heterocyclyl includes, but is not limited to, pyridyl, tetrahydropyranyl, N-methylpiperidin-3-yl, N-methylpyrrolidin-3-yl, 2-pyrrolidon-1-yl, furyl, quinolyl, thienyl, benzothienyl, pyrrolidinyl, piperidinyl, morpholinyl, pyrrolidinyl, tetrahydrofuranyl, tetrahydrothiofuranyl, 1,1-dioxo-hexahydro-1λ⁶-thiopyran-4-yl, tetrahydroimidazo[4,5-c]pyridinyl, imidazolinyl, piperazinyl, and piperidin-2-onyl. and the derivatives thereof. The prefix indicating the number of carbon atoms (e.g., C₃-C₁₀) refers to the total number of carbon atoms in the portion of the cycloheteroalkyl or heterocyclyl group exclusive of the number of heteroatoms. In one embodiment, R^{x} and R^{y} together is heterocyclyl. More specifically the term aryl includes, but is not limited to, phenyl, biphenyl, 1-naphthyl, and 2-naphthyl, and the substituted forms thereof.

As used herein, the terms “Heterocyclylalkyl” or “Cycloheteroalkyl-alkyl” means a radical —R^{x}R^{y} where R^{x} is an alkylene group and R^{y} is a heterocyclyl group as defined herein, e.g., tetrahydropyran-2-ylmethyl, 4-(4-substituted-phenyl)piperazin-1-ylmethyl, 3-piperidinylethyl, and the like.

As used herein, the terms “halo” and “halogen” are used interchangeably; the terms “hydroxy” and “hydroxyl” are used interchangeably; and the terms “COOR³” and “CO₂R³” are used interchangeably.

As used herein, the terms “optional” or “optionally” as used throughout the specification mean that the subsequently described event or circumstance may, but need not, occur, and that the description includes instances where the event or circumstance occurs and instances in which it does not. For example, “heterocyclyl group optionally mono- or di-substituted with an alkyl group means that the alkyl may, but need not be, present, and the description includes situations where the heterocyclyl group is mono- or disubstituted with an alkyl group and situations where the heterocyclo group is not substituted with an alkyl group.

As used herein, the term “Optionally substituted” means a ring which is optionally substituted independently with substituents.

The above terms (e.g., “alkyl,” “aryl” and “heteroaryl”), in some embodiments, will include both substituted and unsubstituted forms of the indicated radical. Preferred substituents for each type of radical are provided below. For brevity, the terms (e.g., “alkyl,” “aryl” and “heteroaryl”) will refer to substituted or unsubstituted versions as provided below.

Substituents for the radicals can be a variety of groups and are generally selected from: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —CN and —NO₂, —R′, —N₃, perfluoro(C₁-C₄) alkoxy, and perfluoro(C₁-C₄) alkyl, in a number ranging from zero to the total number of open valences on the radical; and where R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl, aryl substituted with 1-3 halogens, unsubstituted C₁₋₈ alkyl, C₁₋₈ alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. Other suitable substituents include each of the above aryl substituents attached to a ring atom by an alkylene tether of from 1-4 carbon atoms.

Two of the substituents on adjacent atoms of the aryl or heteroaryl ring may optionally be replaced with a substituent of the formula -T-C(O)—(CH₂)_{q}—U³—, wherein T and U³ are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

For each of the definitions above, the term “di-alkylamino” refers to an amino moiety bearing two alkyl groups that can be the same, or different.

A combination of substituents or variables is permissible only if such a combination results in a stable or chemically feasible compound. A stable compound or chemically feasible compound is one in which the chemical structure is not substantially altered when kept at a temperature of 4° C. or less, in the absence of moisture or other chemically reactive conditions, for at least a week. For example compounds of Formula I would exclude compounds which contain an N—CO₂H, NSO₂H or NSO₃H moiety.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 4th edition J. March, John Wiley and Sons, New York, 1992).

“Pharmaceutically acceptable salt” of a compound means a salt that is pharmaceutically acceptable and that possesses the desired pharmacological activity of the parent compound. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethylamine, N-methylglucamine, and the like.

“Protecting group” refers to a grouping of atoms that when attached to a reactive group in a molecule masks, reduces or prevents that reactivity. Examples of protecting groups can be found in T. W. Greene and P. G. Wuts, PROTECTIVE GROUPS IN ORGANIC CHEMISTRY, (Wiley, 2nd ed. 1991) and Harrison and Harrison et al., COMPENDIUM OF SYNTHETIC ORGANIC METHODS, Vols. 1-8 (John Wiley and Sons. 1971-1996). Representative amino protecting groups include formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC) and the like. Representative hydroxy protecting groups include those where the hydroxy group is either acylated or alkylated such as benzyl and trityl ethers as well as alkyl ethers, tetrahydropyranyl ethers, trialkylsilyl ethers and allyl ethers.

Turning next to the compositions of the invention, the term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of cancer or BPH, diminishment of extent of disease, delay or slowing of disease progression, amelioration, palliation or stabilization of the disease state, and other beneficial results described below.

As used herein, “reduction” of a symptom or symptoms (and grammatical equivalents of this phrase) means decreasing of the severity or frequency of the symptom(s), or elimination of the symptom(s).

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “therapeutically effective amount” of a drug is an amount of a drug that, when administered to a subject with cancer or BPH, will have the intended therapeutic effect, e.g., alleviation, amelioration, palliation or elimination of one or more manifestations of cancer or BPH in the subject. The full therapeutic effect does not necessarily occur by administration of one dose, and may occur only after administration of a series of doses. Thus, a therapeutically effective amount may be administered in one or more administrations.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose, and may occur only after administration of a series of doses. Thus, a prophylactically effective amount may be administered in one or more administrations.

Lonidamine Analogs

In another embodiment (GROUP 22), the compounds of the present invention have the formula (I): 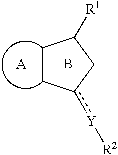
wherein A-B is a 7,5, 6,5 or a 5,5 cyclic ring system, optionally substituted with from one to five V⁶ substituents, each independently selected from the group consisting of hydrogen, amino, halo, oxo, cyano, nitro, (C₁-C₈) alkyl, (C₁-C₆) alkoxy, nitro, acetamido, L¹-CO₂H, L¹-dialkylamino, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CUR³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₂R³, SOR³, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, cyano, nitrileoxide, and —NO, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

R¹ is selected from the group consisting of CO₂R³, COR⁴, COCOR³, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH—V⁵, COCOR⁴, CON(R³)N═CR³R⁷, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;

L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₆) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl;

R² is an aryl or heteroaryl group, optionally substituted with from one to five R⁶ substituents independently selected from the group consisting of halo, nitro, cyano, nitrileoxide, —NO, R³, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³), NR³—SOR³, N—(SOR³)₂, NR³—PU₂R³, N—(PU₂R³)₂, NR³—P(═U)(UR³)R³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, nitrileoxide, and —NO;

each R³ is a member independently selected from the group consisting of H, (C₁-C₈) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl and heteroaryl;

each R⁴ is a member independently selected from the group consisting of NR³R⁷, NR³OR⁷, NR⁷NR³R⁷ or NR³CN;

R⁵ is H, OH or halogen;

R⁷ is selected from the group consisting of H, (C₁-C₈) alkyl, (C₂-C₆) alkenyl, (C₂-C₈) alkynyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;

R⁸ is H, halo, nitro, cyano, nitrileoxide, —NO, R³, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, or 2R⁸ taken together form a (C₃-C₈) cycloalkyl, (C₃-C₈) heterocyclyl or heteroaryl ring;

R³¹ is aryl or heteroaryl;

each V⁵ is a member independently selected from the group consisting of COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂R³, CONHSO₂R³, and C(═NCN)NH₂,

Y is CR⁸₂, CR⁸, NR⁸, S or O;

U is O, S, NR³, NCOR³, or NCONR³R⁷; and

U¹ is O or S;

[symbol] represents a single or double bond; and

pharmaceutically acceptable salts, solvates, hydrates, and prodrugs thereof.

In one embodiment, the present invention provides a compound of formula (I) wherein V⁶ is other than —COOR³.

In addition to compounds having formula (I) above, the present invention further includes all salts thereof, and particularly, pharmaceutically acceptable salts thereof. Still further, the invention includes compounds that are single isomers of the above formula (e.g., single enantiomers of compounds having a single chiral center), as well as solvate, hydrate and tautomeric forms thereof. In other embodiments isomers include single geometric isomers such as cis, trans, E and Z forms of compounds with geometric isomers, or single tautomers of compounds having two or more tautomers.

In one embodiment, an amino or alkylamino functionality present in a compound of formula (I) can be further substituted with one or more acyl, alkoxycarbonyl, aminocarbonyl, alkylaminocarbonyl, dialkylaminocarbonyl, alkylsulfonyl, or arylsulfonyl groups. In another embodiment, an acyl, alkoxycarbonyl, aminocarbonyl, alkylaminocarbonyl, dialkylaminocarbonyl, alkylsulfonyl or arylsulfonyl group is part of a cyclic structure. For ease of reference, certain sets of compounds of the invention are referred to as “groups,” e.g., “Group 1”. It will be appreciated, however, that no method or composition of the invention is limited to groups to which numbers have been assigned.

In one group of embodiments, (GROUP 1) the compounds of the present invention have the formula (I) with the proviso that the compound is not lonidamine, tolnidamine,

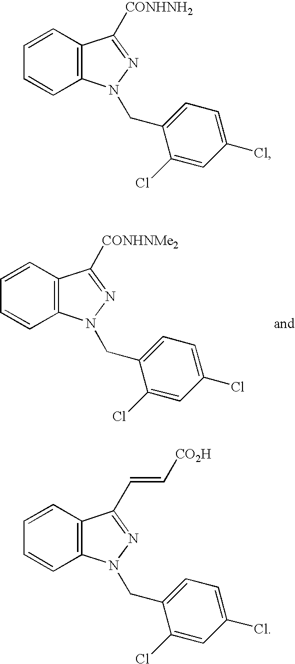

For convenience, the five analogs above can be called Group A analogs, and the set of compounds defined by formula (I) and not including the aforementioned Group A analogs can be referred to as GROUP 1 compounds.

In another embodiment, the present invention provides compounds of formula (III)

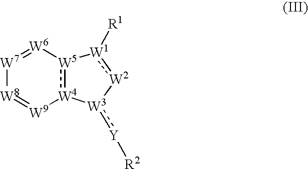

wherein

R¹ is selected from the group consisting of COOR³, COR⁴, CONR³COR³, CH═CHCO₂R³, B(OR³)₂, SO₂R⁴, NHSO₂CR⁵₃, NHSO₂CR³₃, CONHSO₂CR³₃, NHSO₂Ar, C(═NCN)NH₂, COCOR⁴ and L¹-V⁵ wherein L¹ is selected from the group consisting of, —C≡C—, —C(V¹)═C(V³)—, —C(V¹V²)C(V³V⁴)—, 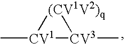
—NHCO— and —NHNH— wherein each V¹, V², V³, and V⁴ is independently selected from the group consisting of hydrogen, (C₁-C₄) alkyl or (C₁-C₈) heteroalkyl, halogen, hydroxy, (C₁-C₆) alkoxy, cyano, nitro, amino, (C₁-C₄) alkylamino and (C₁-C₄) dialkylamino or V¹ and V³ together form a (C₃-C₈) cycloalkyl, a (C₁-C₈) heterocycloalkyl, a (C₃-C₈) cycloalkenyl, an aryl or a heteroaryl ring; with the proviso that if one of V¹ and V² is hydroxyl, amino, (C₁-C₄) alkylamino or (C₁-C₄) dialkylamino, then the other is hydrogen or alkyl; and if one of V³ and V⁴ is hydroxyl, amino, (C₁-C₄) alkylamino, and (C₁-C₄) dialkylamino, then the other is hydrogen or alkyl; q is 1-6; V⁵ is selected from COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂Ar and C(═NCN)NH₂; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar or C(═NCN)NH₂; and when L¹ is —NHNH— then V⁵ is COOR³, COR⁴, COCOR⁴, B(OR³)₂, SO₂R⁴, or C(═NCN)NH₂;

R² is an aryl or heteroaryl group, optionally substituted with from one to three R⁶ substituents that are independently selected from the group consisting of halo and (C₁-C₈) alkyl;

R³ is H, (C₁-C₈) alkyl or (C₁-C₈) heteroalkyl, (C₃-C₈) cycloalkyl or (C₁-C₈) heterocyclyl, or aryl or heteroaryl;

each R⁴ is a member independently selected from the group consisting of NR³R⁷, NR³OR⁷, NR⁷NR³R⁷ and NR³CN;

R¹ is H, OH or halogen;

R⁷ is H, (C₁-C₈) alkyl or (C₁-C₈) heteroalkyl, (C₃-C₈) cycloalkyl or (C₁-C₈) heterocyclyl, or aryl or heteroaryl;

R³ and R⁷ together are (C₁-C₈) heteroalkyl or heteroaryl;

Ar is aryl or heteroaryl;

each W¹, W³, W⁴ or W⁵ is independently N or C;

W² is a member selected from the group consisting of N, CR⁵, C—R⁸, CU, O, NR⁷ and S;

each W⁶, W⁷ W⁸ or W⁹ is independently N or CV⁶ wherein V⁶ is selected from the group consisting of hydrogen, (C₁-C₄) alkyl or (C₁-C₈) heteroalkyl, halogen, hydroxy, (C₁-C₆) alkoxy, amino, cyano, nitro, (C₁-C₄) alkylamino and (C₁-C₄) dialkylamino;

Y is CHR, CR⁸₂, NR, S or O;

R⁸ is H, (C₁-C₈) alkyl or (C₁-C₈) heteroalkyl group;

[symbol] represents a single, double or normalized bond; and

pharmaceutically acceptable salts, solvates, hydrates, and prodrugs thereof;

with the proviso that when W¹, W⁴, and W⁵ are C; W² and W³ are N; W⁶ and W⁷ are CH, Y is CH₂; R⁶ is Cl; and R¹ is not COOR³ or COR⁴.

In another group of embodiments, the compounds of the present invention have the formula (I) with the proviso that the compound is not one of the following compounds (a)-(i) as defined below. The sets of compounds having the formulas (a), (b), (c), (d), (e), (f), (g), (h), and (i) can be referred to as GROUPS A, B, C, D, E, F, G, H, and I, respectively. For convenience, the set of compounds defined by formula (I) and not including compounds (a)-(i) as defined below can be referred to as GROUP 2 compounds.

In an embodiment the present invention excludes the following compounds:

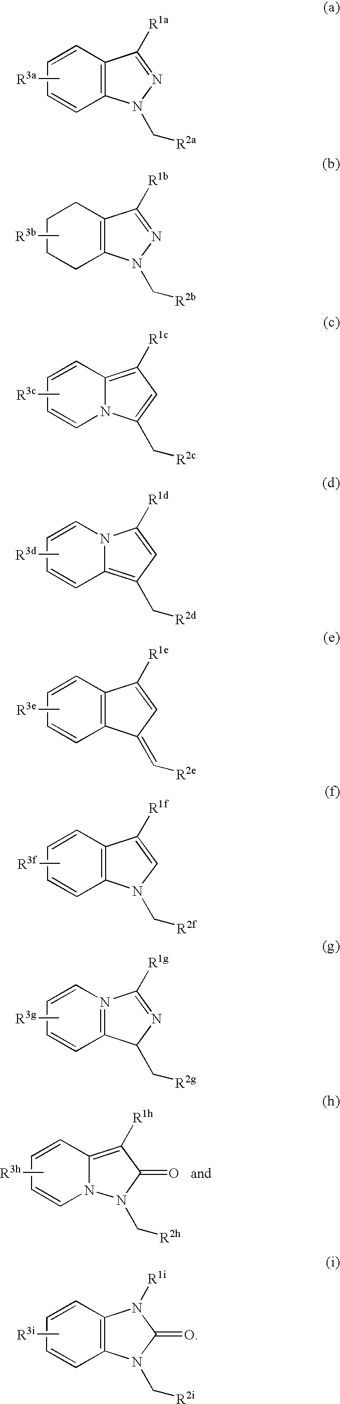

Within this embodiment; referring to formula (a)

(i) R^{1a} is selected from the group consisting of CONHNH₂, CONHN(CH₃)₂, and —CH═CHCO₂H;

R^{2a} is a group having the formula: 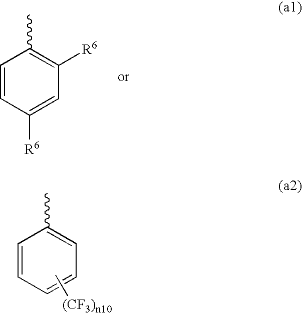
wherein each R⁶ independently is a halogen, and n10 is 1 or 2; and

R^{3a} is hydrogen;

(ii) R^{1a} is CO₂H;

R^{2a} is selected from the group consisting of 4-chlorophenyl, 3-chlorophenyl, 2-chlorophenyl, 4-fluorophenyl, 4-bromophenyl, 4-iodophenyl, 3-trifluoromethylphenyl, 4-cyanophenyl, 4-phenylsulfonyl-phenyl, 3,4-dichlorophenyl, 2,4-dichlorophenyl, 2,6-dichlorophenyl, 2,4-dibromophenyl, 2,4,5-trichlorophenyl, 4-chlorophenyl, 4-methylphenyl, 3-methylphenyl, 2-methylphenyl, 4-chlorophenyl, 3-benzoylphenyl, 4-methylsulfonylphenyl, 4-chloronaphthylmethyl, 2,4-dimethylphenyl and 2-methyl-4-chlorophenyl; and

R^{3a} is hydrogen;

iii) R^{1a} is CO₂H

R^{2a} is 4-chlorophenyl; and

R^{3a} is chloro, OH, methyl, or OMe;

iv) R^{1a} is selected from the group consisting of CO₂Me, CO₂Et, —CO-glyceryl, COCH₃, CONH₂, CH₂CO₂H, CH₂CH₂CO₂H and

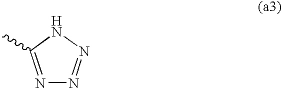

R^{2a} is 4-chlorophenyl, and

R^{3a} is H;

(v) R^{1a} is CO₂H,

R^{2a} is 2,4-dichlorophenyl,

R^{3a} is selected from the group consisting of —(OCH3)ₙ₁₀ wherein ₙ₁₀ is 1 or 2, chloro, bromo, fluoro, CO₂H, and CH₂CO₂H;

(vi) R^{1a} is —O—PO₃H, —O—SO₃H, —O—CH₂CO₂H, O—CH(CO₂H)₂, NHCH(CO₂H)₂, CH₂CH(NH₂)CO₂H, CONHCH(CO₂H)₂, and CONH(CH₂)ₙ₁₁-cyclopropyl wherein n11 is 0 or 1,

R^{2a} is 2,4-dichlorophenyl,

R^{3a} is H; and

(vii) R^{1a} is selected from the group consisting of —COCH₃, —SH, -tetrahydrofurfuryl, —CH₂CO₂H, —CH₂CH₂CO₂H, —H, —CH₃, —CH₂OH, —NH₂, —CN, -tetrazin-2-yl, O—(CH₂)₁₋₂CO₂H, O—CH₂CO₂C₁-C₄alkyl, —O—PO₃H, —O—SO₃H, O—CH(CO₂H)₂, NHCH(CO₂H)₂ and CH₂CHNH₂CO₂H;

R^{2a} is selected from the group consisting of phenyl, 2-chlorophenyl, 2-methylphenyl, 3-fluorophenyl, 3-chlorophenyl, 3-bromophenyl, 3-methylphenyl, trifluoromethylphenyl, 3-benzoyl, 4-halophenyl, 4-methylsulfonylphenyl, 4-methylphenyl, 4-cyanophenyl, 4-phenylsulfonylphenyl, 4-methoxyphenyl, 4-chloronapth-1-yl, 2,3-dimethylphenyl, 2,4-dihalophenyl, 2,4-dimethylphenyl, 2,6-dichlorophenyl, 2,6-dimethylphenyl, 3,4-dichlorophenyl, bis-trifluoromethylphenyl, 4-chloro-2-methylphenyl, 5-chloro-2-methoxyphenyl, 2,4,5-trichlorophenyl, 2,6-dimethyl-3-dimethylsulfamoylphenyl, 4-imidazoyl;

R^{3a} is selected from the group consisting of H, 2-dimethylaminoethyl, 5-amino, chloro, bromo, 5-hydroxy, 5-methyl, methoxy, dimethoxy, fluoro, CO₂H, CH₂CO₂H, 5-nitro, 5-acetamido and 7-chloro;

(viii) compounds having the formulae:

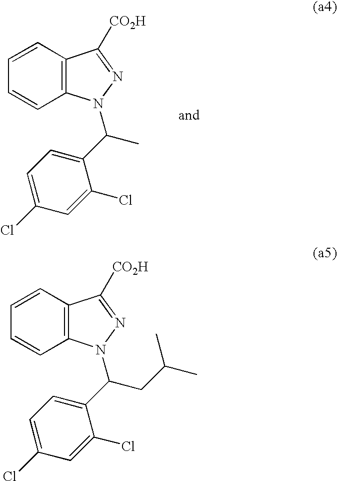

(ix) compounds having the formula:

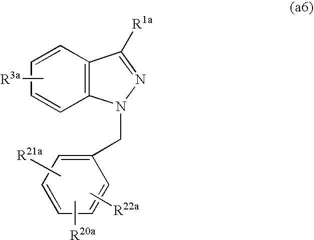

wherein R^{1a} is COOH, CONH₂, COO CH₂CH₂OH, COOCH₂CHOHCH₂OH, or COOCH(CH₂OH)₂;

R^{22a} is H or halo,

R^{20a} is halo, Me, methoxy, trifluoromethyl, CONH₂, or methanesulfonyl, and

R^{21a} is H, Me, halo, or a group forming with the benzene ring to which it is attached a naphthyl ring; and

R^{3a} is H, Me, methoxy and halogen.

Within this embodiment, referring to formula (b):

R^{1b} is CO₂H;

R^{2b} is phenyl;

R^{3b} is H;

Within this embodiment, referring to formula (c):

(i) R^{1c} is CH₂CONH₂;

R^{2c} is phenyl, 2-phenyl-phenyl, 2-benzyl-phenyl, 3-chlorophenyl, 3-trifluoromethylphenyl, 4-phenyl-phenyl, naphthyl, 3,5-di-t-butylphenyl, benzyl, 2-thienyl, 3-(thien-2-yl)thienyl, cyclohexylmethyl, 3-methoxyphenyl, 3-nitrophenyl, cyclopentylmethyl, cycloheptylmethyl, pentyl, 4-heptyl, 1-adamantyl, trans-4-pentyl-cyclohexyl, 2-phenylethenyl, 2-phenylethyl,

R^{3c} is selected from the group consisting of H, methyl, ethyl, t-butyl, cyclopropyl, —O(CH₂CH₂CH₂)₁₋₄CO₂H, —OCH₂-tetraazo-2-yl and —SCH₃;

(ii) compounds having the formula:

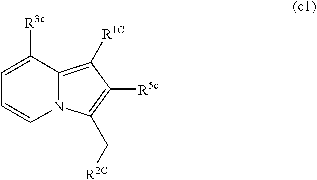

when R C is COCONH₂; R^{5c} and R^{2c} are defined as set forth in Table 2A below; and

R^{3c} is benzyl, then compounds i-xxv, xxvii, xxix, xxxvii, and xxxix are excluded;

R^{3c} is Me, then compound xxvi is excluded;

R^{3c} is H, then compounds i-xxix, xxxviii, and xxxix are excluded;

R^{3c} is —CH₂—CO₂Me, then compounds i, ii, iv, vi, viii-xxiii, xxiv, xxx-xxxviii, and xxxix are excluded;

R^{3c} is —CH₂—CO₂Et, then compounds iii, v, and vii are excluded; and

R^{3c} is —H₂—CO₂H, then compounds i-xxix, xxx-xxxvii, and xxxix are excluded.

TABLE 2A

| Comp | R^{5c} | R^{2c} |
|---|---|---|
| I | Et | Ph |
| Ii | Et | o-Ph—C6H4 |
| iii | Et | m-Cl—C6H4 |
| Iv | Et | m-CF₃—C₆H₄ |
| V | Et | 1-naphthyl |
| vi | cycloPr | o-Ph—C₆H₄ |
| vii | Me | Ph |
| viii | Et | p-Ph—C₆H₄ |
| Ix | Et | cyclohexyl |
| X | Et | cyclopentyl |
| xi | Et | cycloheptyl |
| xii | Et | n-Bu |
| xiii | Et | Pent-4-yl |
| xiv | Et | 2-naphthyl |
| xv | Et | 3,5-(t-Bu)₂—C₆H₃ |
| xvi | Et | Bn |
| Xvii | Et | o-Bn—C₆H₄ |
| xviii | Et | 2-thienyl |
| xix | Et | 3-(thienyl-2-yl)thienyl-2-yl |
| xx | Et | m-MeO—C₆H₄ |
| xxi | Et | o-NO₂—C₆H₄ |
| xxii | Et | trans-4-(m-n-pentyl)cyclohexyl |
| xxiii | Me | 1-adamantyl |
| xxiv | Me | o-Ph—C₆H₄ |
| xxv | cycloPr | Ph |
| xxvi | Et | p-n-Bu—C₆H₄ |
| xxvii | Me | cyclohexyl |
| xxviii | cycloPr | cyclopentyl |
| xxix | Me | cyclopentyl |
| xxx | cycloPr | cyclohexyl |
| xxxi | iPr | o-Ph—C₆H₄ |
| xxxii | tBu | o-Ph—C₆H₄ |
| xxxiii | cyclopentyl | o-Ph—C₆H₄ |
| xxxiv | Et | m-Ph—C₆H₄ |
| xxxv | Et | cinnamyl |
| xxxvi | Et | phenethyl |
| xxxvii | cycloPr | 1-naphthyl |
| xxxviii | OMe | o-Ph—C₆H₄ |
| xxxix | SMe | o-Ph—C₆H₄ |
| xl | Me | Ph |
| xli | Me | cyclohexyl |

(iii) compounds having the following structure

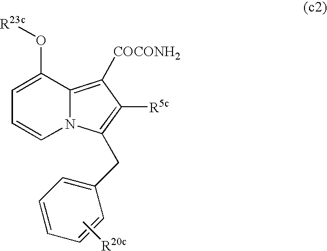

(a) wherein R^{23c} is CH₂CN or tetrazolyl,

R^{5c} is ethyl

R^{20c} is 3-chloro; and

(b) R^{23c} is CH₂-tetrazolyl, CH₂-2-pyridyl, CH₂-4-pyridyl, CH₂-2-quinolinyl, —(CH₂)₃—CO₂Et, —(CH₂)₃—CO₂H, —(CH₂)₂—CO₂H,

R^{5c} is ethyl;

R^{20c} is 2-phenyl; and

(c) R^{23c} is OCH₂CO₂H,

R^{5c} is ethyl and

R^{20c} is H;

(d) R^{23c} is Me or H, and
R^{5c} is ethyl when R^{20c} is hydrogen,
R^{5c} is cyclopropyl when R^{20c} is 2-phenyl, and
R^{5c} is ethyl when R^{20c} is 2-phenyl;

(e) wherein R^{23c} is —(CH₂)₃—CO₂Et or —(CH₂)₃—CO₂H, and
R^{5c} is ethyl when R^{20c} is hydrogen,
R^{5c} is cyclopropyl when R^{20c} is 2-phenyl, and
R^{5c} is ethyl when R^{20c} is 2-phenyl;

(f) wherein R^{23c} is —(CH₂)₂—CO₂Et, —(CH₂)₂—CO₂H, —CH₂—CO₂Et or —CH₂—CO₂H,

R^{5c} is ethyl and R^{20r} is 2-phenyl;

(iii) compounds having the following structure

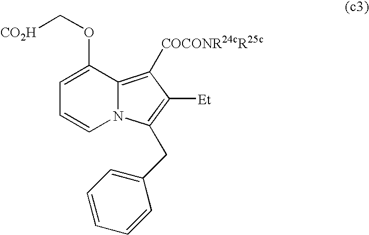

wherein R^{24c} is H or Me and R^{25c} is Me.

Within this embodiment, referring to formula (d):

R^{1d} is CH₂CONH₂;

R^{2d} is selected from the group consisting of phenyl, 2-phenyl-phenyl, 2-benzyl-phenyl, 3-chlorophenyl, 3-trifluoromethylphenyl, 4-phenyl-phenyl, naphthyl, 3,5-di-t-butylphenyl, benzyl, 2-thienyl, 3-(thien-2-yl)thienyl, cyclohexylmethyl, 3-methoxyphenyl, 3-nitrophenyl, cyclopentylmethyl, cycloheptylmethyl, pentyl, 4-heptyl, 1-adamantyl, trans-4-pentyl-cyclohexyl, 2-phenylethenyl and 2-phenylethyl;

R^{3d} is selected from the group consisting of H, methyl, ethyl, t-butyl, cyclopropyl, —O(CH₂CH₂CH₂)₁₋₄CO₂H, —OCH₂-tetraazo-2-yl and —SCH₃;

Within this embodiment referring to formula (e):

(i) R^{1e} is CH₂CONH₂,

R^{2e} is selected from the group consisting of phenyl, 2-phenyl-phenyl, 2-benzyl-phenyl, 3-chlorophenyl, 3-trifluoromethylphenyl, 4-phenyl-phenyl, naphthyl, 3,5-di-t-butylphenyl, benzyl, 2-thienyl, 3-(thien-2-yl)thienyl, cyclohexylmethyl, 3-methoxyphenyl, 3-nitrophenyl, cyclopentylmethyl, cycloheptylmethyl, pentyl, 4-heptyl, 1-adamantyl, trans-4-pentyl-cyclohexyl, 2-phenylethenyl and 2-phenylethyl;

R^{3e} is selected from the group consisting of H, methyl, ethyl, t-butyl, cyclopropyl, —O(CH₂CH₂CH₂)₁₄CO₂H, —OCH₂-tetraazo-2-yl and —SCH₃;

Within this embodiment, referring to formula (f):

R^{1f} is CO₂H;

R^{2f} is selected from the group consisting of phenyl, 2-chlorophenyl, 3-chlorophenyl, 4-chlorophenyl, 2,4-dichlorophenyl, 2,5-dichlorophenyl, 2,6-dichlorophenyl, 3,4-dichlorophenyl and 3,5-dichlorophenyl;

R^{3f} is H;

Within this embodiment, referring to formula (g):

R^{1g} is CO₂Et;

R^{2g} is phenyl;

R^{3g} is H;

Within this embodiment, referring to formula (h):

R^{1h} is CO₂Et or C(═NH)OEt;

R^{2h} is phenyl;

R^{3h} is H, 5-methyl or 7-methyl;

Within this embodiment (Group J), referring to formula (i):

R¹ⁱ is CONHCH₂CH₂Cl or CONHCH₂CH₂-piperazin-4-yl;

R²ⁱ is benzyl; and

R³ⁱ is H.

In one embodiment the present invention excludes compounds specifically disclosed in the following references: Corsi et al., 1976, J Med Chem 19:778-83; Cheng et al., 2001, Biol Reprod. 65:449-61; Silvestrini, 1981, Chemotherapy 27:9-20; Andreani et al., Arch. Pharm., Weinheim, 1984, 317: 847-51, Besner et al., Drug. Metab. Rev., 1997, 29(1 and 2): 219-34, Palacios et al., Tetrahedron 1995, 51(12):3683-90, Kakehi et al., Bull. Chem. Soc. Japan, 1978, 51(1) and :251-6, Caputo, Chemotherapy, 1981, 27(suppl. 2): 107-20, Silvestrini et al., Prog. Med. Chem., 1984, 21, 111-35, Milanesio et al., J. Org. Chem. 2000, 65, 3416-25, Hagishita et al., J. Med. Chem. 1996, 39, 3636-58, Tapia et al., J. Med. Chem. 1999, 42, 2870-80, U.S. Pat. Nos. 4,002,759, 3,895,026, 6,001,865, 5,034,398, 3,625,971, 3,470,194, 5,621,002, PCT Appl. titled Prodrugs of Lonidamine and Lonidamine Analogs, Att. Docket No. 021305-002210PC, PCT Appl. No. US2004/0167196, and PCT Pub. No. WO96/03383.

In another group of embodiments, the compounds of the present invention have the formula (I) with the proviso that the compound does not have the formula: 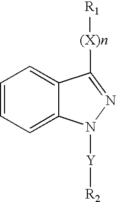
wherein R¹ is —COOH; —CONR³R⁴, —CONHNR⁶R⁷; —COOR⁵ or —COO-Z+; Z+ is a pharmaceutically acceptable cation; R² represents a aryl or heteroaryl group, optionally substituted by one, two, or three substituents independently selected from the group consisting of halo, alkyl and CF₃; R³ and R⁴ may be independently alkyl or hydrogen; R⁶ and R⁷ are usually —H or —CH₃; X represents a straight chain or branched chain, saturated or unsaturated hydrocarbon linkage group; Y is —CHR⁷—; and n is 0 or 1.

A number of other groups of embodiments are set forth below.

In any of the above embodiments, Y is NR⁸. In other embodiments Y is NH. In other embodiments Y is O. In other embodiments Y is S. In other embodiments Y is CR⁸. Y is CR⁸₂. In other embodiments Y is CH₂.

In one embodiment, the present invention provides V⁶ substituents, each independently selected from the group consisting of hydrogen, amino, halo, oxo, cyano, nitro, (C₁-C₈) alkyl, (C₁-C₆) alkoxy, nitro, acetamido, L¹-CO₂H, L¹-dialkylamino, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CUR³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₂R³, SOR³, SO₂R³, SOR³, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, cyano, nitrileoxide, and —NO, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring. In another embodiment, the present invention provides V⁶ substituents, each independently selected from the group consisting of hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; U¹—R³, R⁴, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—PU₂R³, N—(PU₂R³)₂, NR³P(═R)(UR³)R³, CUR³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₂R³, SOR³, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring. In another embodiment, the present invention provides V⁶ substituents, each independently selected from the group consisting of hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring. Within these embodiments, in one embodiment V⁶ is other than —COOR³. In another embodiment, the present invention provides V⁶ substituents, each independently selected from the group consisting of hydrogen, halo, oxo, cyano, nitro, CH₃, CH₂CH₃, CH(Me)₂, methoxymethyl, ethoxymethyl, cyclopropyl, cyclobutyl, 2-furanyl, 3-furanyl, ethynyl, 1-propynyl, 3-propynyl, O-Me, O-Et, O-cyclopropyl, O-Aryl, S— Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)—C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, PO(NMe₂)₂. In another embodiment, the present invention provides V⁶ selected from the group consisting of hydrogen, (C₁-C₄) alkyl or (C₁-C₄) heteroalkyl, halogen, hydroxy, (C₁-C₆) alkoxy, amino, cyano, nitro, (C₁-C₄) alkylamino, and (C₁-C₄) dialkylamino. In another embodiment, the present invention provides V⁶ selected from the group consisting of methyl, ethyl, propyl, isopropyl, fluoro, chloro, bromo, iodo, cyano, nitro, amino, methylamino, dimethylamino, ethylamino, methoxy, and hydroxyl.

In other embodiments the present invention provides compounds of Formula I, wherein A-B is a 7,5-fused (C₁-C₈) cyclic ring system. In one embodiment the present invention provides compounds of Formula I, wherein A-B is a 6,5-fused (C₁-C₈) cyclic ring system. In other embodiments the present invention provides compounds of Formula I, wherein A-B is a 5,5-fused (C₁-C₈) cyclic ring system.

In one embodiment (GROUP 4) the present invention provides compounds of formula I, wherein the cyclic ring system A-B has the formula IIA:

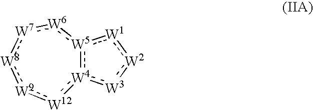

wherein each W¹, W³, W⁴ or W⁵ is independently N or C;

W² is a member selected from the group consisting of N, CR⁵, CO, O, NR⁷ and S;

each W⁶, W⁷, W⁸ W⁹ or W¹² is independently N, NV⁶, CO, CS, SO, SO₂ or CV⁶

[symbol] represents a single or double bond;

R¹, Y, R² and V⁶ are as defined above in formula (I); and

pharmaceutically acceptable salts, solvates, hydrates, and prodrugs thereof.

Returning to formula IIA those of skill in the art will appreciate, upon considering the entirety of this disclosure, that the total number of nitrogens in W¹ and W³-W⁹ and W¹² will typically not exceed 5, and the substitution pattern of the 5-membered ring is such that none of W¹, W³, W⁴, and W⁵ is CH or CV⁶. In one embodiment, all of W⁶-W⁹ and W¹² are independently CV⁶. In another embodiment, three of W⁶-W⁹ and W¹² are independently CV⁶ and the other is CH or N. In another embodiment, two of W⁶-W⁹ and W¹² are independently CV⁶ and the rest are CH or N. In another embodiment, one of W⁶-W⁹ and W¹² is CV⁶ and the rest are CH or N.

In one embodiment, the present invention provides V⁶ substituents, each independently selected from the group consisting of hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; U¹—R³, R⁴, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—PU₂R³, N—(PU₂R³)₂, NR³—P(═U)(UR³)R³, CUR³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₂R³, SOR³, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring. In another embodiment, the present invention provides V⁶ substituents, each independently selected from the group consisting of hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring. In another embodiment, V⁶ is other than —COOR³. In another embodiment, the present invention provides V⁶ substituents, each independently selected from the group consisting of hydrogen, halo, oxo, cyano, nitro, CH₃, CH₂CH₃, CH(Me)₂, methoxymethyl, ethoxymethyl, cyclopropyl, cyclobutyl, 2-furanyl, 3-furanyl, ethynyl, 1-propynyl, 3-propynyl, O-Me, O-Et, O-cyclopropyl, O-Aryl, S— Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)—C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, PO(NMe₂)₂. In another embodiment, the present invention provides V⁶ selected from the group consisting of hydrogen, (C₁-C₄) alkyl or (C₁-C₄) heteroalkyl, halogen, hydroxy, (C₁-C₆) alkoxy, amino, cyano, nitro, (C₁-C₄) alkylamino, and (C₁-C₄) dialkylamino. In another embodiment, the present invention provides V⁶ selected from the group consisting of methyl, ethyl, propyl, isopropyl, fluoro, chloro, bromo, iodo, cyano, nitro, amino, methylamino, dimethylamino, ethylamino, methoxy, and hydroxyl.

In one embodiment (GROUP 5) the present invention provides compounds of formula I, wherein the cyclic ring system A-B has the formula IIIA:

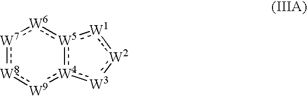

wherein each W¹, W³ W⁴ or W⁵ is independently N or C;

W² is a member selected from the group consisting of N, CR⁵, CO, O, NR⁷ and S;

each W⁶, W⁷ W⁸ or W⁹ is independently N, NV⁶, CO, CS, SO, SO₂ or CV⁶;

[symbol] represents a single or double bond;

R¹, Y, R² and V⁶ are as defined above in formula (I); and

pharmaceutically acceptable salts, solvates, hydrates, and prodrugs thereof.

Returning to formulas IIIA, those of skill in the art will appreciate, upon considering the entirety of this disclosure, that the total number of nitrogens in W¹ and W³-W⁹ will typically not exceed 5, and the substitution pattern of the 5-membered ring is such that none of W¹, W³, W⁴, and W¹ is CH or CV⁶. In one embodiment, all of W⁶-W⁹ are independently CV⁶. In another embodiment, three of W⁶-W⁹ are independently CV⁶ and the other is CH or N. In another embodiment, two of W⁶-W⁹ are independently CV⁶ and the rest are CH or N. In another embodiment, one of W⁶-W⁹ is CV⁶ and the rest are CH or N. In one embodiment, V⁶ is selected from the group consisting of methyl, ethyl, propyl, isopropyl, fluoro, chloro, bromo, iodo, amino, methylamino, dimethylamino, ethylamino, methoxy, and hydroxyl.

In another embodiment (GROUP 6), the compounds of the present invention have the formulas IIIB, 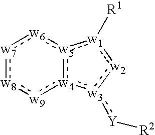
(IIIB)
wherein

R¹ is selected from the group consisting of COOR³, COR⁴, CONR³COR³, CH═CHCO₂R³, B(OR³)₂, SO₂R⁴, NHSO₂CR⁵₃, NHSO₂CR³₃, CONHSO₂CR³₃, NHSO₂Ar, C(═NCN)NH₂, COCOR⁴CON(R³)N═CR³R⁷, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl; and L¹-V⁵ wherein L¹ is selected from the group consisting of —C≡C—, —C(V¹)═C(V³)—, —C(V¹V²)C(V³V⁴)—, 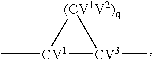
—NHCO— and —NHNH— wherein each V¹, V², V³, and V⁴ is independently selected from the group consisting of hydrogen, (C₁-C₄) alkyl or (C₁-C₈) heteroalkyl, halogen, hydroxy, (C₁-C₆) alkoxy, cyano, nitro, amino, (C₁-C₄) alkylamino and (C₁-C₄) dialkylamino or V¹ and V³ together form a (C₃-C₈) cycloalkyl, a (C₁-C₈) heterocycloalkyl, a (C₃-C₈) cycloalkenyl, an aryl or a heteroaryl ring; with the proviso that if one of V¹ and V² is hydroxyl, amino, (C₁-C₄) alkylamino or (C₁-C₄) dialkylamino, then the other is hydrogen or alkyl; and if one of V³ and V⁴ is hydroxyl, amino, (C₁-C₄) alkylamino, and (C₁-C₄) dialkylamino, then the other is hydrogen or alkyl; q is 1-6; V⁵ is selected from COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂Ar and C(═NCN)NH₂; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar or C(═NCN)NH₂; and when L¹ is —NHNH— then V¹ is COOR³, COR⁴, COCOR⁴, B(OR³)₂, SO₂R⁴, or C(═NCN)NH₂;

R² is an aryl or heteroaryl group, optionally substituted with from one to three R⁶ substituents that are independently selected from the group consisting of halo and (C₁-C₈) alkyl;

R³ is H, (C₁-C₈) alkyl, (C₁-C₈) heteroalkyl, (C₃-C₈) cycloalkyl, (C₁-C₈) heterocyclyl, aryl or heteroaryl;

each R⁴ is a member independently selected from the group consisting of NR³R⁷, NR³OR⁷, NR⁷NR³R⁷ and NR³CN;

R⁵ is H, OH or halogen;

R⁷ is H, (C₁-C₈) alkyl, (C₁-C₈) heteroalkyl, (C₃-C₈) cycloalkyl, (C₁-C₈) heterocyclyl, aryl or heteroaryl;

R³ and R⁷ together are (C₁-C₈) heteroalkyl or heteroaryl;

Ar is aryl or -heteroaryl;

each W¹, W³, W⁴ or W¹ is independently N or C;

W² is a member selected from the group consisting of N, CR⁵, CO, O, NR⁷ and S; each W⁶, W⁷, W⁸ or W⁹ is independently N or CV⁶ wherein V⁶ is selected from the group consisting of hydrogen, (C₁-C₄) alkyl, (C₁-C₈) heteroalkyl, halogen, hydroxy, (C₁-C₆) alkoxy, amino, cyano, nitro, oxo, U¹—R³, UL-COR³, (C₁-C₄) alkylamino and (C₁-C₄) dialkylamino;

Y is CHR⁸, CR⁸₂, NR⁸, S or O;

R⁸ is H, (C₁-C₈) alkyl or (C₁-C₈) heteroalkyl;

[symbol] represents a single, double or normalized bond; and

pharmaceutically acceptable salts, solvates, hydrates, and prodrugs thereof.

In another embodiment (GROUP 7), the present invention provides compounds of formula IIIA wherein the A-B ring moiety has the following structure

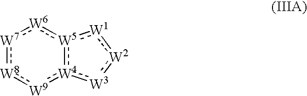

wherein W¹-W⁵ is defined as follows in Table 1A:

TABLE 1A

| Ring B | W¹ | W² | W³ | W⁴ | W⁵ |
|---|---|---|---|---|---|
| 1 | C | N | N | C | C |
| 2 | N | N | C | C | C |
| 3 | N | C═O | N | C | C |
| 4 | N | SO₂ | N | C | C |
| 5 | N | SO | N | C | C |
| 6 | N | C═O | C | C | N |
| 7 | N | SO₂ | C | C | N |
| 8 | N | SO | C | C | N |
| 9 | C | C═O | N | N | C |
| 10 | C | SO₂ | N | N | C |
| 11 | C | SO | N | N | C |
| 12 | C | N | C | N | C |
| 13 | C | N | C | C | N |
| 14 | C | CR⁵ | C | N | C |
| 15 | C | CR⁵ | C | C | N |
| 16 | C | O | C | C | C |
| 17 | C | S | C | C | C |
| 18 | C | SO | C | C | C |
| 19 | C | SO₂ | C | C | C |
| 20 | C | NR⁷ | C | C | C |
| 21 | C | CR⁵ | C | C | C |

and for each ring B 1-21 as defined above, W⁶-W⁹ is defined as follows in Table 1B:

TABLE 1B

| Ring A | W⁶ | W⁷ | W⁸ | W⁹ |
|---|---|---|---|---|
| 1 | CV⁶ | CV⁶ | CV⁶ | CV⁶ |
| 2 | CV⁶ | CV⁶ | CV⁶ | N |
| 3 | CV⁶ | CV⁶ | N | CV⁶ |
| 4 | CV⁶ | N | CV⁶ | CV⁶ |
| 5 | N | CV⁶ | CV⁶ | CV⁶ |
| 6 | CV⁶ | CV⁶ | N | N |
| 7 | CV⁶ | N | N | CV⁶ |
| 8 | N | N | CV⁶ | CV⁶ |
| 9 | CV⁶ | N | CV⁶ | N |
| 10 | N | CV⁶ | N | CV⁶ |
| 11 | N | CV⁶ | CV⁶ | N |
| 12 | N | N | N | CV⁶ |
| 13 | N | N | CV⁶ | N |
| 14 | N | CV⁶ | N | N |
| 15 | CV⁶ | N | N | N |

In one embodiment (GROUP 3), the present invention provides compounds of formulae IIIA and for each ring B 1-21, W⁶-W⁹ is defined as follows in Table 1C:

Table 1C 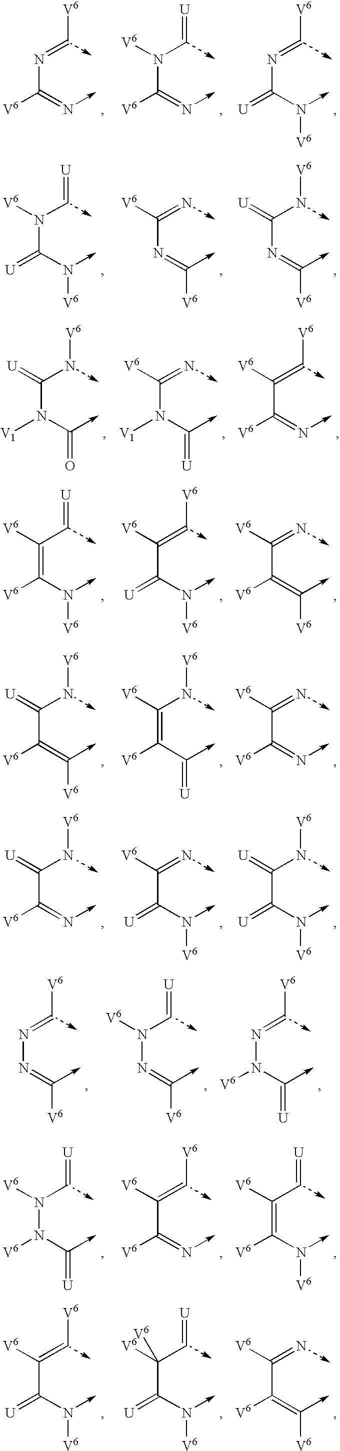 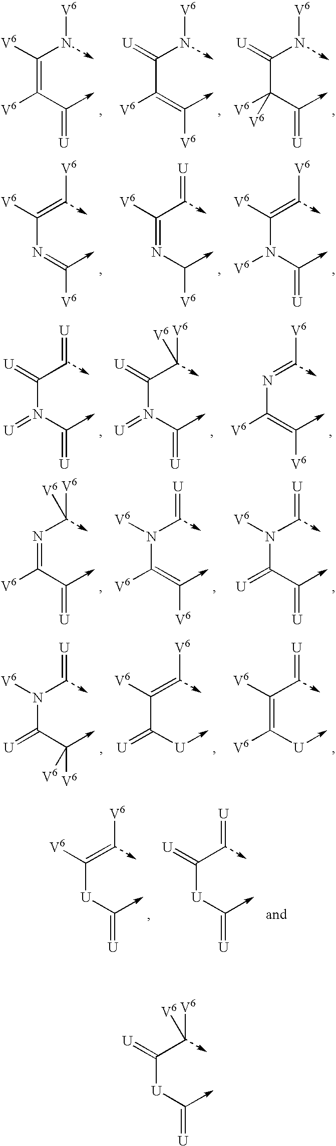
wherein → indicates a single bond to W and ---------→ indicates a single bond to W⁵ and V⁶ and U are as defined above.

In another embodiment (GROUP 9), the present invention provides compounds wherein the C═U bond in the structural formulas in Table 1C is independently replaced with an SO or an SO₂ moiety, such as, for example, providing a compound containing the moiety

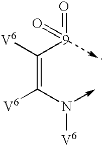

In another embodiment (GROUP 10), the compounds of the present invention have the formula IVA

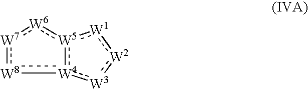

In one embodiment (GROUP 11), W¹-W⁵ of formula (IV) are as defined in Table 1A above; and for each W¹-W⁵ as defined above, W⁶-W⁸ are defined as follows in Table 1D:

TABLE 1D

| Ring A | W⁶ | W⁷ | W⁸ |
|---|---|---|---|
| 16 | CV⁶ | CV⁶ | CV⁶ |
| 17 | NV⁶ | CV⁶ | CV⁶ |
| 18 | N | CV⁶ | NV⁶ |
| 19 | NV⁶ | CV⁶ | N |
| 20 | NV⁶ | C═O | NV⁶ |
| 21 | C═O | NV⁶ | C═O |
| 22 | C═O | NV⁶ | SO₂ |
| 23 | SO₂ | NV⁶ | C═O |
| 24 | SO₂ | NV⁶ | SO₂ |
| 25 | NV⁶ | N | N |
| 26 | N | N | NV⁶ |
| 27 | NV⁶ | N | CV⁶ |
| 28 | CV⁶ | N | NV⁶ |
| 29 | CV⁶ | CV⁶ | U¹ |
| 30 | U¹ | CV⁶ | CV⁶ |

In one embodiment (GROUP 12), A-B in formula IVA is selected from the group consisting of: 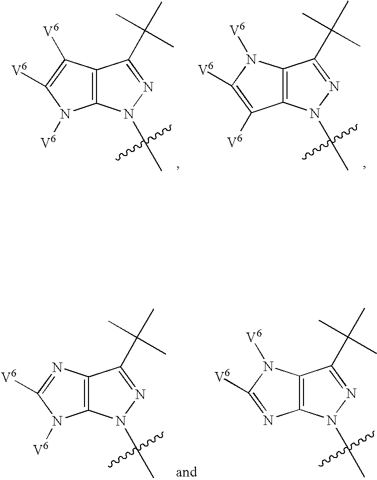
wherein the solid line indicates the point of attachment to R¹ and the wavy line indicates the point of attachment to Y.

In another group of embodiments, (GROUP 13), the compounds of the present invention have the formula (IIID):

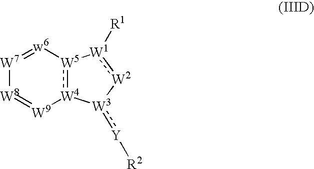

wherein

R¹ is selected from the group consisting of COOR³, COR⁴, CONR³COR³, CH═CHCO₂R³, B(OR³)₂, SO₂R⁴, NHSO₂CR⁵₃, NHSO₂CR³₃, CONHSO₂CR³₃, NHSO₂Ar, C(═NCN)NH₂, COCOR⁴ and L¹-V⁵ wherein L¹ is selected from the group consisting of —C≡C—, —C(V¹)═C(V³)—, —C(V¹V²)C(V³V⁴)—, 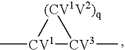
—NHCO— and —NHNH— wherein each V¹, V², V³, and V⁴ is independently selected from the group consisting of hydrogen, (C₁-C₄) alkyl or (C₁-C₈) heteroalkyl, halogen, hydroxy, (C₁-C₆) alkoxy, cyano, nitro, amino, (C₁-C₄) alkylamino and (C₁-C₄) dialkylamino or V¹ and V³ together form a (C₃-C₈) cycloalkyl, a (C₁-C₈) heterocycloalkyl, a (C₃-C₈) cycloalkenyl, an aryl or a heteroaryl ring; with the proviso that if one of V¹ and V² is hydroxyl, amino, (C₁-C₄) alkylamino or (C₁-C₄) dialkylamino, then the other is hydrogen or alkyl; and if one of V³ and V⁴ is hydroxyl, amino, (C₁-C₄) alkylamino, and (C₁-C₄) dialkylamino, then the other is hydrogen or alkyl; q is 1-6; V⁵ is selected from COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂Ar and C(═NCN)NH₂; with the proviso that in NHSO₂CR⁵₃, R⁵ is not OH; when L¹ is —NHCO— then V¹ is COR⁴, NHSO₂CR⁵₃, NHSO₂CR³₃, NHSO₂Ar or C(═NCN)NH₂; and when L¹ is —NHNH— then V⁵ is COOR³, COR⁴, COCOR⁴, B(OR³)₂, SO₂R⁴, or C(═NCN)NH₂;

R² is an aryl or heteroaryl group, optionally substituted with from one to three R⁶ substituents that are independently selected from the group consisting of halo and (C₁-C₈) alkyl;

R³ is H, (C₁-C₈) alkyl or (C₁-C₈) heteroalkyl, (C₃-C₈) cycloalkyl or (C₁-C₈) heterocyclyl, or aryl or heteroaryl;

each R⁴ is a member independently selected from the group consisting of NR³R⁷, NR³OR⁷, NR⁷NR³R⁷ and NR³CN;

R⁵ is H, OH or halogen;

R⁷ is H, (C₁-C₈) alkyl or (C₁-C₈) heteroalkyl, (C₃-C₈) cycloalkyl or (C₁-C₈) heterocyclyl, or aryl or heteroaryl;

R³ and R⁷ together are (C₁-C₈) heteroalkyl or heteroaryl;

Ar is aryl or heteroaryl;

each W¹, W³, W⁴ or W⁵ is independently N or C;

W² is a member selected from the group consisting of N, CR⁵, CO, O, NR⁷ and S;

each W⁶, W⁷, W⁸ or W⁹ is independently N or CV⁶ wherein V⁶ is selected from the group consisting of hydrogen, (C₁-C₄) alkyl or (C₁-C₈) heteroalkyl, halogen, hydroxy, (C₁-C₆) alkoxy, amino, cyano, nitro, (C₁-C₄) alkylamino and (C₁-C₄) dialkylamino;

Y is CHR⁸, CR⁸₂, NR⁸, S or O;

R⁸ is H, (C₁-C₈) alkyl or (C₁-C₈) heteroalkyl group;

[symbol] represents a single, double or normalized bond; and

pharmaceutically acceptable salts, solvates, hydrates, and prodrugs thereof.

In another group of embodiments (GROUP 14), the compounds of the present invention have the formula (IIID) of embodiment:

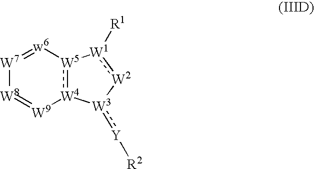

when R¹ is selected from the group consisting of COOR³, COR⁴, CH═CHCO₂R³, B(OR³)₂, SO₂R⁴, NHSO₂CR⁵₃, NHSO₂CR³₃, NHSO₂Ar, C(═NCN)NH₂, COCOR⁴ and L¹-V⁵ when L¹ is selected from the group consisting of —C≡C—, —C(V¹)═C(V³)—, —C(V¹V²)C(V³V⁴)—, 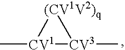
—NHCO— and —NHNH— wherein each V¹, V², V³, and V⁴ is independently selected from the group consisting of hydrogen, (C₁-C₄) alkyl or (C₁-C₈) heteroalkyl, halogen, hydroxy, (C₁-C₄) alkoxy, cyano, nitro, amino, (C₁-C₄) alkylamino and (C₁-C₄) dialkylamino or V¹ and V³ together form a (C₃-C₈) cycloalkyl, a (C₁-C₈) heterocycloalkyl, a (C₃-C₈) cycloalkenyl, an aryl or a heteroaryl ring; with the proviso that if one of V¹ and V² is hydroxyl, amino, (C₁-C₄) alkylamino or (C₁-C₄) dialkylamino, then the other is hydrogen or alkyl; and if one of V³ and V⁴ is hydroxyl, amino, (C₁-C₄) alkylamino, and (C₁-C₄) dialkylamino, then the other is hydrogen or alkyl; q is 1-6; V⁵ is selected from COOR³, COR⁴, COCOR⁴, B(OR³)₂, SO₂R⁴, NHSO₂CR⁵₃, NHSO₂CR³₃, NHSO₂Ar and C(═NCN)NH₂; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar or C(═NCN)NH₂; and when L¹ is —NHNH— then V⁵ is COOR³, COR⁴, COCOR⁴, B(OR³)₂, SO₂R⁴, or C(═NCN)NH₂;

R² is an aryl or heteroaryl group, optionally substituted with from one to three R⁶ substituents that are independently selected from the group consisting of halo and (C₁-C₈) alkyl;

R³ is H, (C₁-C₈) alkyl or (C₁-C₈) heteroalkyl;

each R⁴ is a member independently selected from the group consisting of NR³R⁷, NR³OR⁷, NR⁷NR³R⁷ and NR³CN;

R⁵ is H, OH or halogen;

R⁷ is H, (C₁-C₈) alkyl or (C₁-C₈) heteroalkyl;

R³ and R⁷ together are (C₁-C₈) heteroalkyl or heteroaryl;

Ar is aryl or heteroaryl;

each W¹, W³, W⁴ or W⁵ is independently N or C;

W² is a member selected from the group consisting of N, CR⁵, CO, O, NR⁷ and S;

each W⁶, W⁷ W⁸ or W⁹ is independently N or CV⁶ wherein V⁶ is selected from the group consisting of hydrogen, (C₁-C₄) alkyl or (C₁-C₈) heteroalkyl, halogen, hydroxy, (C₁-C₄) alkoxy, amino, cyano, nitro, (C₁-C₄) alkylamino and (C₁-C₄) dialkylamino;

Y is CHR⁸, CR⁸₂, NR⁸, S or O;

R⁸ is H, (C₁-C₈) alkyl or (C₁-C₈) heteroalkyl group;

[symbol]

represents a single, double or normalized bond.

In another group of embodiments, (GROUP 15), the compounds of the present invention have the formula (IIID), (IID) or (IIE): 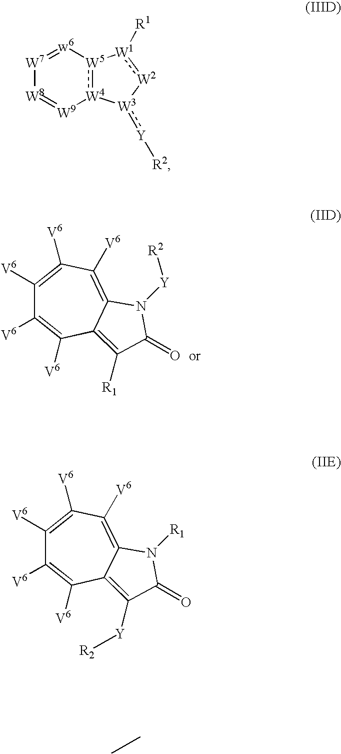
wherein in formula (IIID):

R¹ is selected from the group consisting of COOR³, COR⁴, CH═CHCO₂R³, B(OR³)₂, SO₂R⁴, NHSO₂CR⁵₃, NHSO₂CR³₃, NHSO₂Ar, C(═NCN)NH₂, COCOR⁴ and L¹-V¹ wherein L¹ is selected from the group consisting of —C≡C—, —C(V¹)═C(V³)—, —C(V¹V²)C(V³V⁴)—, 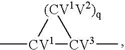
—NHCO— and —NHNH— wherein each V¹, V², V³, and V⁴ is independently selected from the group consisting of hydrogen, (C₁-C₄) alkyl or (C₁-C₈) heteroalkyl, halogen, hydroxy, (C₁-C₄) alkoxy, cyano, nitro, amino, (C₁-C₄) alkylamino and (C₁-C₄) dialkylamino or V¹ and V³ together form a (C₃-C₈) cycloalkyl, a (C₁-C₈) heterocycloalkyl, a (C₃-C₈) cycloalkenyl, an aryl or a heteroaryl ring; with the proviso that if one of V¹ and V² is hydroxyl, amino, (C₁-C₄) alkylamino or (C₁-C₄) dialkylamino, then the other is hydrogen or alkyl; and if one of V³ and V⁴ is hydroxyl, amino, (C₁-C₄) alkylamino, and (C₁-C₄) dialkylamino, then the other is hydrogen or alkyl; q is 1-6; V⁵ is selected from COOR³, COR⁴, COCOR⁴, B(OR³)₂, SO₂R⁴, NHSO₂CR⁵₃, NHSO₂CR³₃, NHSO₂Ar and C(═NCN)NH₂; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar or C(═NCN)NH₂; and when L¹ is —NHNH— then V⁵ is COOR³, COR⁴, COCOR⁴, B(OR³)₂, SO₂R⁴, or C(═NCN)NH₂;

R² is an aryl or heteroaryl group, optionally substituted with from one to three R⁶ substituents that are independently selected from the group consisting of halo and (C₁-C₈) alkyl;

R³ is H, (C₁-C₈) alkyl or (C₁-C₈) heteroalkyl;

each R⁴ is a member independently selected from the group consisting of NR³R⁷, NR³OR⁷, NR⁷NR³R⁷ and NR³CN;

R⁵ is H, OH or halogen;

R⁷ is H, (C₁-C₈) alkyl or (C₁-C₈) heteroalkyl;

R³ and R⁷ together are (C₁-C₈) heteroalkyl or heteroaryl;

Ar is aryl or heteroaryl;

each W¹, W³, W⁴ or W⁵ is independently N or C;

W² is a member selected from the group consisting of N, CR⁵, CO, O, NR⁷ and S;

each W⁶, W⁷, W⁸ or W⁹ is independently N or CV⁶ wherein V⁶ is selected from the group consisting of hydrogen, (C₁-C₄) alkyl or (C₁-C₈) heteroalkyl, halogen, hydroxy, (C₁-C₄) alkoxy, amino, cyano, nitro, (C₁-C₄) alkylamino and (C₁-C₄) dialkylamino;

Y is CHR⁸, CR⁸₂, NR⁸, S or O;

R⁸ is H, (C₁-C₈) alkyl or (C₁-C₈) heteroalkyl group;

[symbol] represents a single, double or normalized bond; and

pharmaceutically acceptable salts, solvates, hydrates, and prodrugs thereof; and in formula (IIE) or (IIE):

R¹ is selected from the group consisting of COOR³, CH═CHCO₂R³, COR⁴, B(OR³)₂, SO₂R⁴, NHSO₂CR⁵₃, NHSO₂CR³₃ and, C(═NCN)NH₂, and L¹-V₅ wherein L¹ is selected from the group consisting of —C(V¹)═C(V³)—, —C≡C—, —C(V¹V²)C(V³V⁴)—, 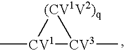
—NHCO—, and —NHNH— wherein each V¹, V², V³, and V⁴ is independently selected from the group consisting of hydrogen, C₁-C₄ alkyl or (C₁-C₈) heteroalkyl, halogen, hydroxy, (C₁-C₄) alkoxy, cyano, nitro, amino, C₁-C₄ alkylamino, and C₁-C₄ dialkylamino or V¹ and V³ together form a (C₃-C₈) cycloalkyl, a (C₁-C₈) heterocycloalkyl, a (C₃-C₈) cycloalkenyl, an aryl, or a heteroaryl ring; with the proviso that if one of V¹ and V² is hydroxyl, amino, C₁-C₄ alkylamino, or C₁-C₄ dialkylamino, then the other is hydrogen or alkyl; and if one of V³ and V⁴ is hydroxyl, amino, C₁-C₄ alkylamino, and C₁-C₄ dialkylamino, then the other is hydrogen or alkyl; V⁵ is selected from COOR³, COR⁴, B(OR³)₂, SO₂R⁴, NHSO₂CR⁵₃, NHSO₂CR³₃, NHSO₂Ar, C(═NCN)NH₂; and q is 1-6; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar and C(═NCN)NH₂; and when L¹ is —NHNH— then V⁵ is COOR³, COR⁴, COCOR⁴, B(OR³)₂, SO₂R⁴ and C(═NCN)NH₂; with the proviso that in NHSO₂CR⁵₃, R⁵ is not OH; when L¹ is —NHCO— then V⁵ is COR⁴, NHSO₂CR⁵₃ NHSO₂CR³₃, NHSO₂Ar and C(═NCN)NH₂; and when L¹ is —NHNH— then V⁵ is COOR³, COR⁴, COCOR⁴, B(OR³)₂, SO₂R⁴ and C(═NCN)NH₂;
R³ is H, (C₁-C₈) alkyl or (C₁-C₈) heteroalkyl;

each R⁴ is a member independently selected from the group consisting of NR³R⁷, NR³OR⁷, NR⁷NR³R⁷ and NR³CN;

R⁷ is H, (C₁-C₈) alkyl or (C₁-C₈) heteroalkyl;

R³ and R⁷ together are (C₁-C₈) heteroalkyl or heteroaryl;

R⁵ is H, OH or halogen;

R⁶ is halo or (C₁-C₈) alkyl;

Ar is aryl or heteroaryl;

Y is CH₂, CHR⁸₂, CR⁸, NR⁸, S or O; and

R⁸ is H or (C₁-C₈) alkyl group; and

R² is an aryl or heteroaryl group, optionally substituted with from one to three R⁶ substituents independently selected from the group consisting of halo and (C₁-C₈) alkyl.

In another group of embodiments (GROUP 16), the compounds of the present invention have the formula (IIID), (IIIE), (IID) or (IIE):

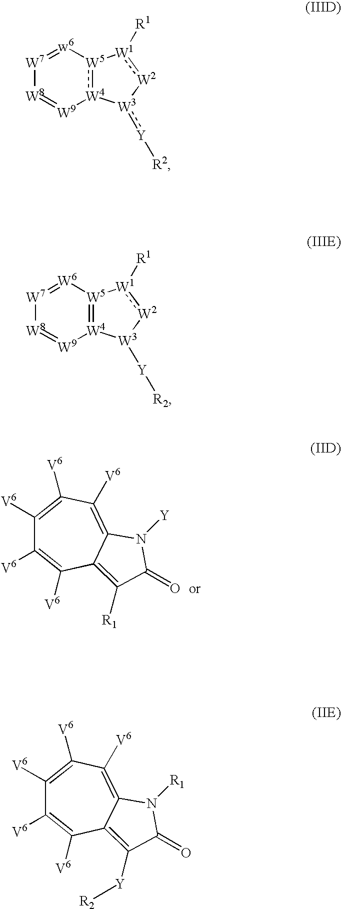

wherein in formula (IIID)

R¹ is selected from the group consisting of NHSO₂Ar, C(═NCN)NH₂, and COCOR⁴;

R² is an aryl or heteroaryl group, optionally substituted with from one to three R⁶ substituents that are independently selected from the group consisting of halo and (C₁-C₈) alkyl;

R³ is H, (C₁-C₈) alkyl or (C₁-C₈) heteroalkyl;

each R⁴ is a member independently selected from the group consisting of NR³R⁷, NR³OR⁷, NR⁷NR³R⁷ and NR³CN;

R⁵ is H, OH or halogen;

R⁷ is H, (C₁-C₈) alkyl or (C₁-C₈) heteroalkyl;

R³ and R⁷ together are (C₁-C₈) heteroalkyl or heteroaryl;

Ar is aryl or heteroaryl;

each W¹, W³, W⁴ or W⁵ is independently N or C;

W² is a member selected from the group consisting of N, CR⁵, CO, O, NR⁷ and S;

each W⁶, W⁷, W⁸ or W⁹ is independently N or CV⁶ wherein V⁶ is selected from the group consisting of hydrogen, (C₁-C₄) alkyl or (C₁-C₈) heteroalkyl, halogen, hydroxy, (C₁-C₄) alkoxy, amino, cyano, nitro, (C₁-C₄) alkylamino and (C₁-C₄) dialkylamino;

Y is CHR⁸, CR⁸₂, NR⁸, S or O;

R⁸ is H, (C₁-C₈) alkyl or (C₁-C₈) heteroalkyl group;

[symbol]

represents a single, double or normalized bond; and in formula (V):

wherein in formula (IIIE) the variables are defined as in embodiment except

R¹ is selected from the group consisting of COOR³, COR⁴, CH═CHCO₂R³, B(OR³)₂, SO₂R⁴, NHSO₂CR⁵₃ and CONH₂(═NHCN);

R² is an aryl or heteroaryl group, optionally substituted with from one to three R⁶ substituents independently selected from the group consisting of halo and (C₁-C₈) alkyl;

R³ is H, (C₁-C₈) alkyl or (C₁-C₈) heteroalkyl;

each R⁴ is a member independently selected from the group consisting of NR³R⁷, NHOR³, NHNR³R⁷ and NHCN;

R⁵ is H, OH or halogen;

R⁷ is H or (C₁-C₈) alkyl;

each W¹, W³, W⁴ or W⁵ is independently N or C;

W² is a member selected from the group consisting of N, CR⁵, CO, O, and S;

each W⁶, W⁷, W⁸ or W⁹ is independently N, C or CH;

Y is CHR⁸, NH, or O;

R⁸ is H or (C₁-C₈) alkyl group;

[symbol] represents a single, double or normalized bond; and

pharmaceutically acceptable salts, solvates, hydrates, and prodrugs thereof; and in formula (IIID) or (IIIE):

R¹ is selected from the group consisting of COOR³, CH═CHCO₂R³, COR¹, B(OR³)₂, SO₂R⁴, NHSO₂CR⁵₃, NHSO₂CR³₃ and, C(═NCN)NH₂, and L¹-V₅ wherein L¹ is selected from the group consisting of —C(V¹)═C(V³)—, —C≡C—, —C(V¹V²)C(V³V⁴)—,

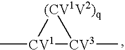

—NHCO—, and —NHNH— wherein each V¹, V², V³, and V⁴ is independently selected from the group consisting of hydrogen, C₁-C₄ alkyl or (C₁-C₈) heteroalkyl, halogen, hydroxy, (C₁-C₄) alkoxy, cyano, nitro, amino, C₁-C₄ alkylamino, and C₁-C₄ dialkylamino or V¹ and V³ together form a (C₃-C₈) cycloalkyl, a (C₁-C₈) heterocycloalkyl, a (C₃-C₈) cycloalkenyl, an aryl, or a heteroaryl ring; with the proviso that if one of V¹ and V² is hydroxyl, amino, C₁-C₄ alkylamino, or C₁-C₄ dialkylamino, then the other is hydrogen or alkyl; and if one of V³ and V⁴ is hydroxyl, amino, C₁-C₄ alkylamino, and C₁-C₄ dialkylamino, then the other is hydrogen or alkyl; V⁵ is selected from COOR³, COR⁴, B(OR³)₂, SO₂R⁴, NHSO₂CR⁵₃, NHSO₂CR³₃, NHSO₂Ar, C(═NCN)NH₂; and q is 1-6; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar and C(═NCN)NH₂; and when L¹ is —NHNH— then V⁵ is COOR³, COR⁴, COCOR⁴, B(OR³)₂, SO₂R⁴ and C(═NCN)NH₂; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar and C(═NCN)NH₂; and when L¹ is —NHNH— then V⁵ is COOR³, COR⁴, COCOR⁴, B(OR³)₂, SO₂R⁴ and C(═NCN)NH₂;

R³ is H, (C₁-C₈) alkyl or (C₁-C₈) heteroalkyl;

each R⁴ is a member independently selected from the group consisting of NR³R⁷, NR³OR⁷, NR⁷NR³R′ and NR³CN;

R⁷ is H, (C₁-C₈) alkyl or (C₁-C₈) heteroalkyl;

R³ and R⁷ together are (C₁-C₈) heteroalkyl or heteroaryl;

R⁵ is H, OH or halogen;

R⁶ is halo or (C₁-C₈) alkyl;

Ar is aryl or heteroaryl;

Y is CH₂, CHR⁸₂, CR⁸, NR⁸, S or O; and

R⁸ is H or (C₁-C₈) alkyl group; and
R² is an aryl or heteroaryl group, optionally substituted with from one to three R⁶ substituents independently selected from the group consisting of halo and (C₁-C₈) alkyl.

In another group of embodiments, (GROUP 17), the compounds of the present invention have the formula (IIID): 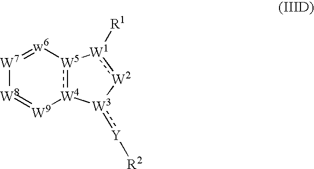
wherein R¹ is L¹-V⁵ wherein L¹ selected from the group consisting of —C≡C—, —CV¹═C(V³)—, —CV¹V²C(V³V⁴)—, 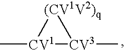
—NHCO—, and —NHNH— wherein each V¹, V², V³, and V⁴ is independently selected from the group consisting of hydrogen, (C₁-C₄) alkyl or (C₁-C₈) heteroalkyl, halogen, hydroxy, (C₁-C₄) alkoxy, cyano, nitro, amino, (C₁-C₄) alkylamino, and (C₁-C₄) dialkylamino or V¹ and V³ together form a (C₃-C₈) cycloalkyl, a (C₁-C₈) heterocycloalkyl, a (C₃-C₈) cycloalkenyl, an aryl, or a heteroaryl ring; with the proviso that if one of V¹ and V² is hydroxyl, amino, (C₁-C₄) alkylamino, or (C₁-C₄) dialkylamino, then the other is hydrogen or alkyl; and if one of V³ and V⁴ is hydroxyl, amino, (C₁-C₄) alkylamino, and (C₁-C₄) dialkylamino, then the other is hydrogen or alkyl; q is 1-6; V⁵ is selected from COOR³, COR⁴, COCOR⁴, B(OR³)₂, SO₂R⁴, NHSO₂CR⁵₃ wherein R⁵ is not OH, NHSO₂CR³₃, NHSO₂Ar, C(═NHCN)NH₂;

R² is an aryl or heteroaryl group, optionally substituted with from one to three R⁶ substituents that are independently selected from the group consisting of halo and (C₁-C₈) alkyl;

R³ is H, (C₁-C₈) alkyl or (C₁-C₈) heteroalkyl;

each R⁴ is a member independently selected from the group consisting of NR³R⁷, NR³OR⁷, NR⁷NR³R⁷ and NR³CN;

R⁵ is H, OH or halogen;

Ar is aryl or heteroaryl;

R⁷ is H, (C₁-C₈) alkyl or (C₁-C₈) heteroalkyl;

R³ and R⁷ together are (C₁-C₈) heteroalkyl or heteroaryl;

each W¹, W³, W⁴, or W⁵ is independently N or C;

W² is a member selected from the group consisting of N, NR⁷, CR⁵, CO, O, and S;

each W⁶, W⁷, W⁸ or W⁹ is independently N or CV⁶ wherein V⁶ is selected from the group consisting of hydrogen, (C₁-C₄) alkyl or (C₁-C₈) heteroalkyl, halogen, hydroxy, (C₁-C₄) alkoxy, amino, cyano, nitro, (C₁-C₄) alkylamino, and (C₁-C₄) dialkylamino;

Y is CHR⁸, CR⁸₂, NR⁸, S, or O;

R⁸ is H or (C₁-C₈) alkyl group; and

[symbol] represents a single, double or normalized bond.

In another group of embodiments, (GROUP 18), the compounds of the present invention have the formula (IIIE) of embodiment:

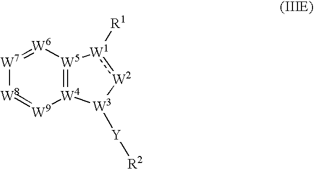

wherein R¹ is CH═CHCO₂R³ when

R² is an aryl or heteroaryl group, optionally substituted with from one to three R⁶ substituents independently selected from the group consisting of halo and (C₁-C₈) alkyl;

R³ is H, (C₁-C₈) alkyl or (C₁-C₈) heteroalkyl;

each R⁴ is a member independently selected from the group consisting of NR³R⁷, NHOR³, NHNR³R⁷ and NHCN;

R⁵ is H, OH or halogen;

R⁷ is H or (C₁-C₈) alkyl;

each W¹, W³, W⁴ or W¹ is independently N or C;

W² is a member selected from the group consisting of N, CR⁵, CO, O, and S;

each W⁶, W⁷, W⁸ or W⁹ is independently N, C or CH;

Y is CHR⁸, NH, or O; and

R⁸ is H or (C₁-C₈) alkyl group;

[symbol]

represents a single, double or normalized bond.

In another group of embodiments, (GROUP 19), the compounds of the present invention have the formula (IIIE), (IIF) or (IIG):

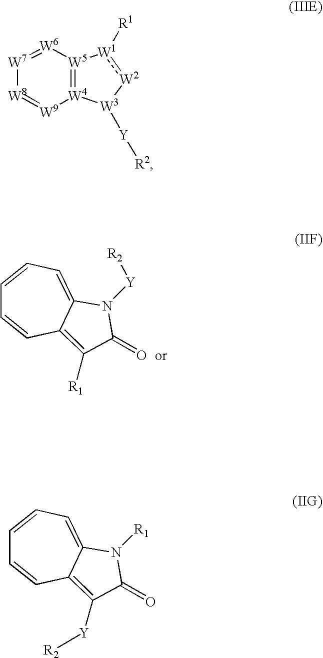

wherein in formula (IIIE): R¹ is selected from the group consisting of COOR³, COR⁴, CH═CHCO₂R³, B(OR³)₂, SO₂R⁴, NHSO₂CR⁵₃ and CONH₂(═NHCN);

R² is an aryl or heteroaryl group, optionally substituted with from one to three R⁶ substituents independently selected from the group consisting of halo and (C₁-C₈) alkyl;

R³ is H, (C₁-C₈) alkyl or (C₁-C₈) heteroalkyl;

each R⁴ is a member independently selected from the group consisting of NR³R⁷, NHOR³, NHNR³R⁷ and NHCN;

R⁵ is H, OH or halogen;

R⁷ is H or (C₁-C₈) alkyl;

each W¹, W³, W⁴ or W⁵ is independently N or C;

W² is a member selected from the group consisting of N, CR⁵, CO, O, and S;

each W⁶, W W⁸ or W⁹ is independently N, C or CH;

Y is CHR⁸, NH, or O;

R⁸ is H or (C₁-C₈) alkyl group;

[symbol] represents a single, double or normalized bond; with the proviso that when W¹, W⁴, and W⁵ are C; W² and W³ are N; W⁶ and W⁷ are CH, Y is CH₂; R⁶ is Cl; R¹ is not COOR³ or COR⁴; and the compound does not have the formula selected from the group consisting of:

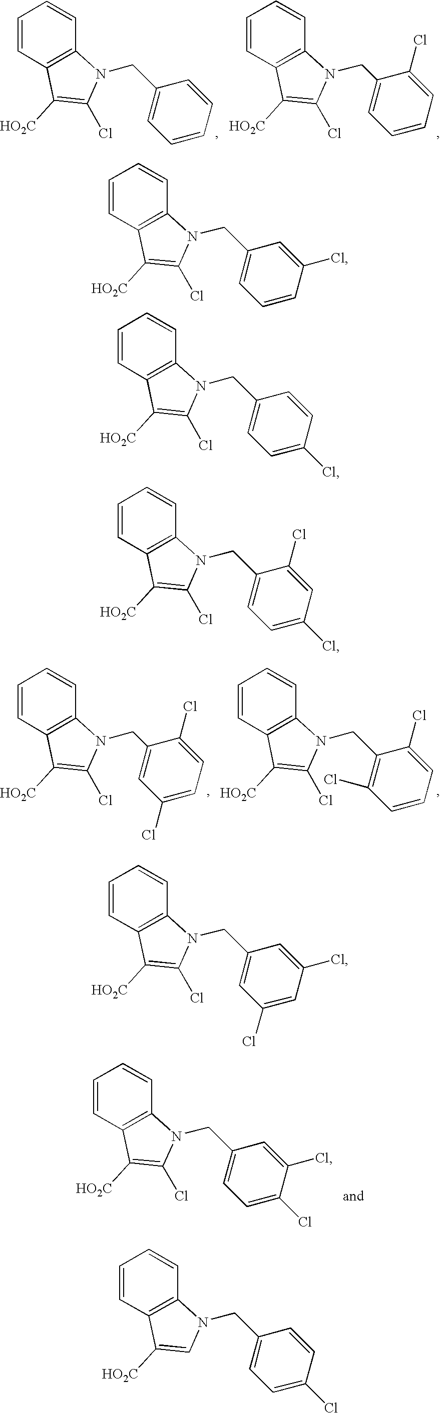

pharmaceutically acceptable salts, solvates, hydrates, and prodrugs thereof; and wherein in formula (IIF) and (IIG):

R¹ is selected from the group consisting of COOR³, CH═CHCO₂R³, COR⁴, B(OR³)₂, SO₂R⁴, NHSO₂CR⁵₃ and CONH₂(═NHCN);

R² is an aryl or heteroaryl group, optionally substituted with from one to three R⁶ substituents independently selected from the group consisting of halo and (C₁-C₈) alkyl; and

Y is CH₂, O, NH, or S.

In another group of embodiments, (GROUP 20), the compounds of the present invention have the formula (IIE), (IIF) or (IIG):

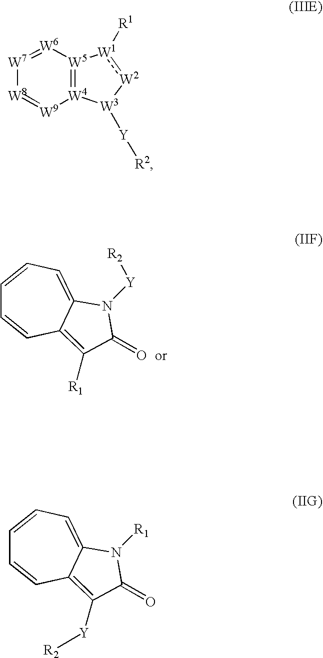

wherein in formula (IIIE) R¹ is CONH₂(═NHCN);

R² is an aryl or heteroaryl group, optionally substituted with from one to three R⁶ substituents independently selected from the group consisting of halo and (C₁-C₈) alkyl;

R³ is H, (C₁-C₈) alkyl or (C₁-C₈) heteroalkyl;

each R⁴ is a member independently selected from the group consisting of NR³R⁷, NHOR³, NHNR³R⁷ and NHCN;

R⁵ is H, OH or halogen;

R⁷ is H or (C₁-C₈) alkyl;

each W¹, W³, W⁴ or Ws is independently N or C;

W² is a member selected from the group consisting of N, CR⁵, CO, O, and S;

each W⁶, W W⁸ or W⁹ is independently N, C or CH;

Y is CHR⁸, NH, or O;

R⁸ is H or (C₁-C₈) alkyl group;

[symbol] represents a single, double or normalized bond; with the proviso that when W¹, W⁴, and W⁵ are C; W² and W³ are N; W⁶ and W⁷ are CH, Y is CH₂; R⁶ is Cl; R¹ is not COOR³ or COR⁴; and pharmaceutically acceptable salts, solvates, hydrates, and prodrugs thereof; and in formula (IIF) and (IIG):

R¹ is selected from the group consisting of COOR³, CH═CHCO₂R³, COR⁴, B(OR³)₂, SO₂R⁴, NHSO₂CR⁵₃ and CONH₂(═NHCN);

R² is an aryl or heteroaryl group, optionally substituted with from one to three R⁶ substituents independently selected from the group consisting of halo and (C₁-C₈) alkyl; and

Y is CH₂, O, NH, or S.

Within this group of embodiments, the compounds of the present invention also have the formula (IIIE) wherein R¹ is selected from the group consisting of COOR³, COR⁴, CH═CHCO₂R³, B(OR³)₂, SO₂R⁴, NHSO₂CR⁵₃ and CONH₂(═NHCN);

R² is an aryl or heteroaryl group, optionally substituted with from one to three R⁶ substituents independently selected from the group consisting of halo and (C₁-C₈) alkyl;

R³ is H, (C₁-C₈) alkyl or (C₁-C₈) heteroalkyl;

each R⁴ is NHCN;

R⁵ is H, OH or halogen;

R⁷ is H or (C₁-C₈) alkyl;

each W¹, W³, W⁴ or W⁵ is independently N or C;

W² is a member selected from the group consisting of N, CR⁵, CO, O, and S;

each W⁶, W⁷, W⁸ or W⁹ is independently N, C or CH;

Y is CHR⁸, NH, or O;

R⁸ is H or (C₁-C₈) alkyl group;

[symbol] represents a single, double or normalized bond; with the proviso that when W¹, W⁴, and W⁵ are C; W² and W³ are N; W⁶ and W⁷ are CH, Y is CH₂; R⁶ is Cl; R¹ is not COOR³ or COR⁴; pharmaceutically acceptable salts, solvates, hydrates, and prodrugs thereof.

In another group of embodiments, (GROUP 21), the compounds of the present invention have the formula (IIIE):

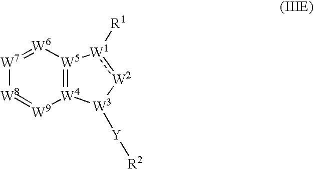

wherein R¹ is selected from the group consisting of COOR³, COR⁴, CH═CHCO₂R³, B(OR³)₂, SO₂R⁴, and NHSO₂CR⁵₃;

R² is an aryl or heteroaryl group, optionally substituted with from one to three R⁶ substituents independently selected from the group consisting of halo and (C₁-C₈) alkyl;

R³ is H, (C₁-C₈) alkyl or (C₁-C₈) heteroalkyl;

each R⁴ is a member independently selected from the group consisting of NR³R⁷, NHOR³ and NHNR³R⁷;

R⁵ is H, OH or halogen;

R⁷ is H or (C₁-C₈) alkyl;

each W¹, W³, W⁴, W⁵, W⁶, W⁷, W⁸ or W⁹ is independently N, C or CH;

W² is a member selected from the group consisting of N, CR⁵, CO, O, and S;

Y is CHR⁸, NH, or O;

R⁸ is H or (C₁-C₈) alkyl group;

[symbol] represents a single, double or normalized bond; with the proviso that when W¹, W⁴, and W⁵ are C; W² and W³ are N; W⁶ and W⁷ are CH, Y is CH₂; R⁶ is Cl; R¹ is not COOR³ or COR⁴; and

pharmaceutically acceptable salts, solvates, hydrates, tautomer and prodrugs thereof.

In another group of embodiments (GROUP 8), the formula: 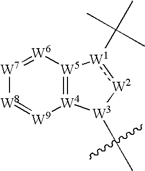
is a member selected from the group consisting of: 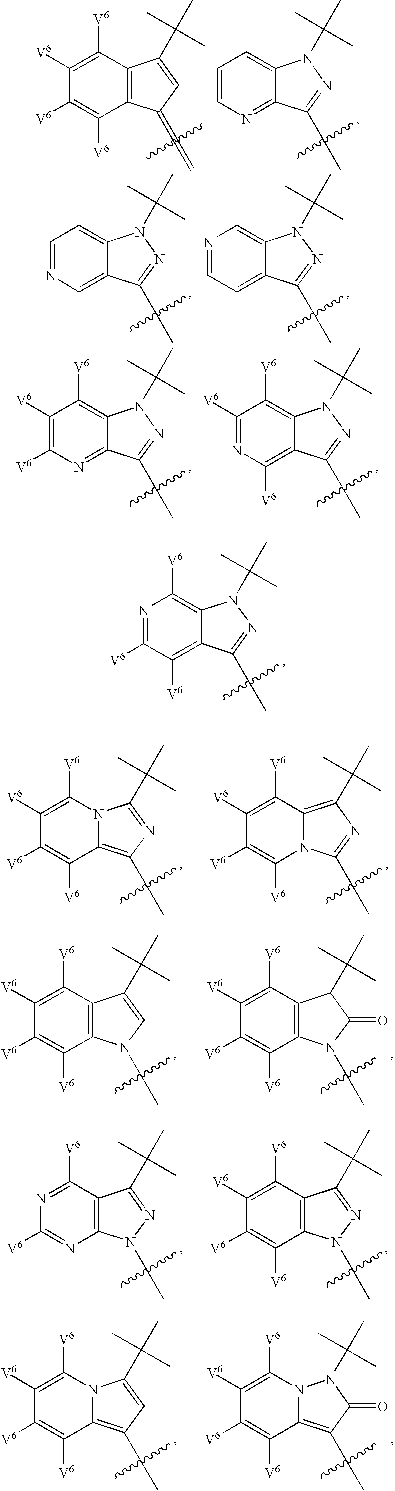 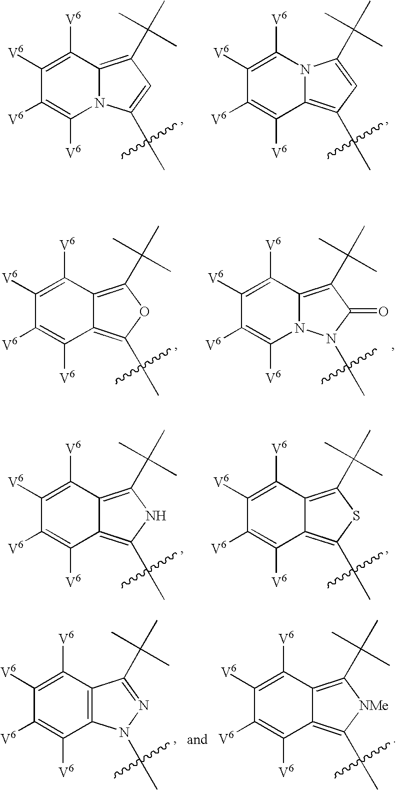
wherein the solid line indicates the point of attachment to R¹ and the wavy line indicates the point of attachment to Y and V⁶ is defined as above.

In another group of embodiments (GROUP 23), the formula: 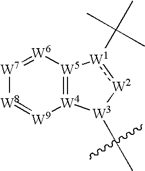
is a member selected from the group consisting of: 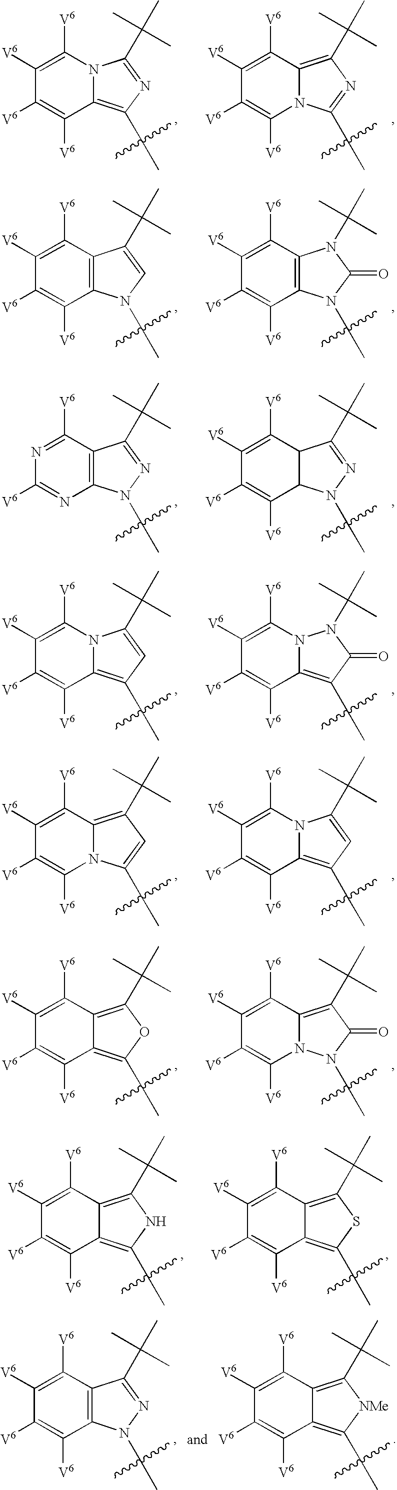
wherein the solid line indicates the point of attachment to R¹ and the wavy line indicates the point of attachment to Y and V⁶ is defined as above. Within these embodiments, W⁶-W⁹ are independently CV⁶. In another embodiment, three of W⁶-W⁹ are independently CV⁶ and the other is CH or N. In another embodiment, two of W⁶-W⁹ are independently CV⁶ and the rest are CH or N. In another embodiment, one of W⁶-W⁹ is CV⁶ and the rest are CH or N. In one embodiment, V⁶ is selected from the group consisting of methyl, ethyl, propyl, isopropyl, fluoro, chloro, bromo, iodo, amino, methylamino, dimethylamino, ethylamino, methoxy, and hydroxyl.

In another group of embodiments (GROUP 24), the formula: 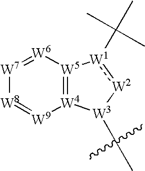
is a member selected from the group consisting of: 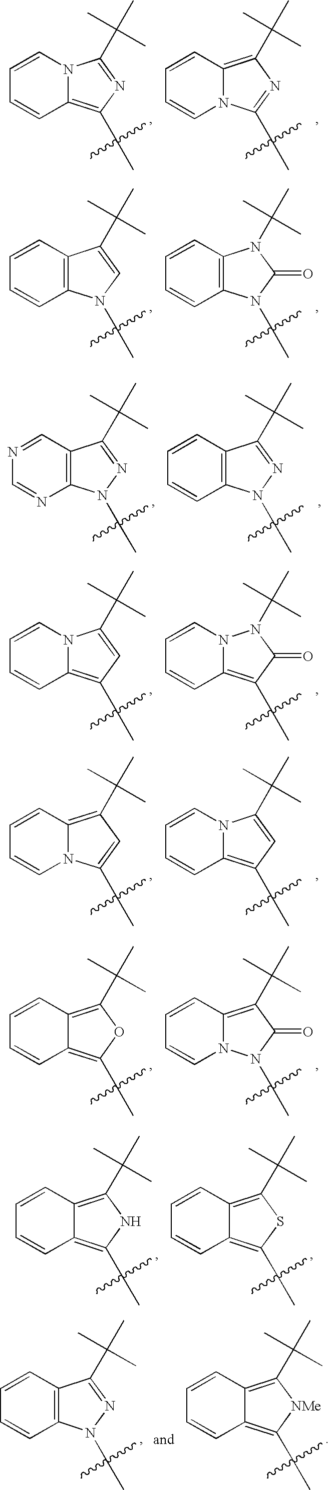
wherein the solid line indicates the point of attachment to R¹ and the wavy line indicates the point of attachment to Y.

In another group of embodiments, (GROUP 25) the formula: 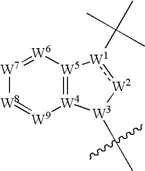
is a member selected from the group consisting of: 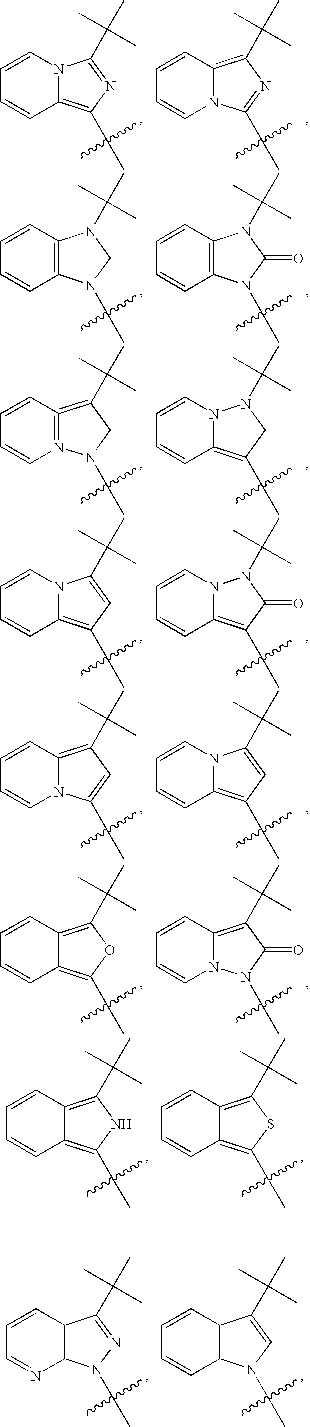 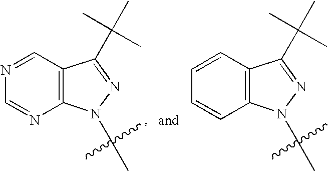
wherein the solid line indicates the point of attachment to R¹ and the wavy line indicates the point of attachment to Y.

In another embodiment, (GROUP 26) R¹ is selected from the group consisting of: CONHNH₂, CONH₂, CONHNMe₂, CONMe₂

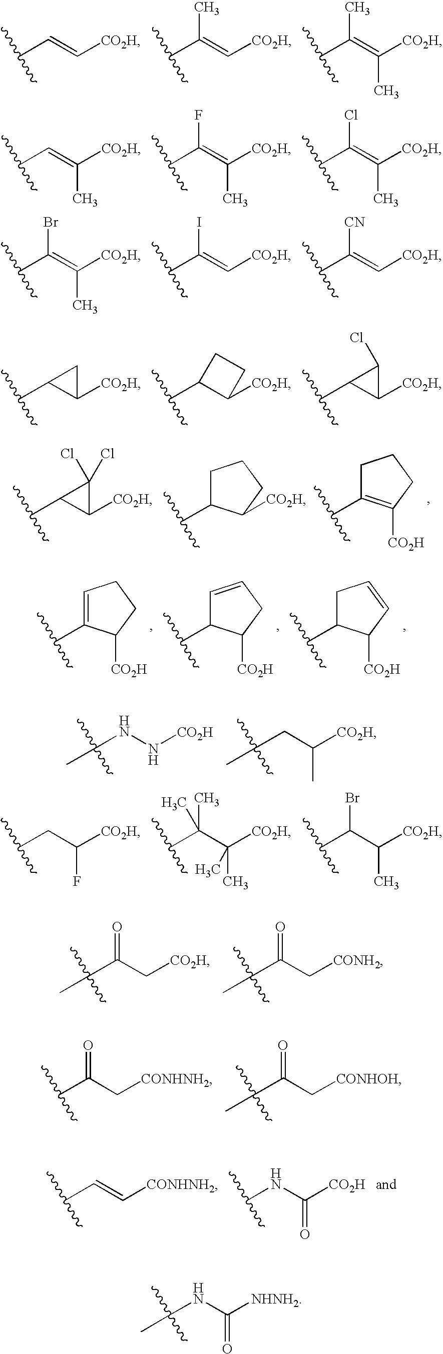

In another embodiment, (GROUP 27) R¹ is selected from the group consisting of:

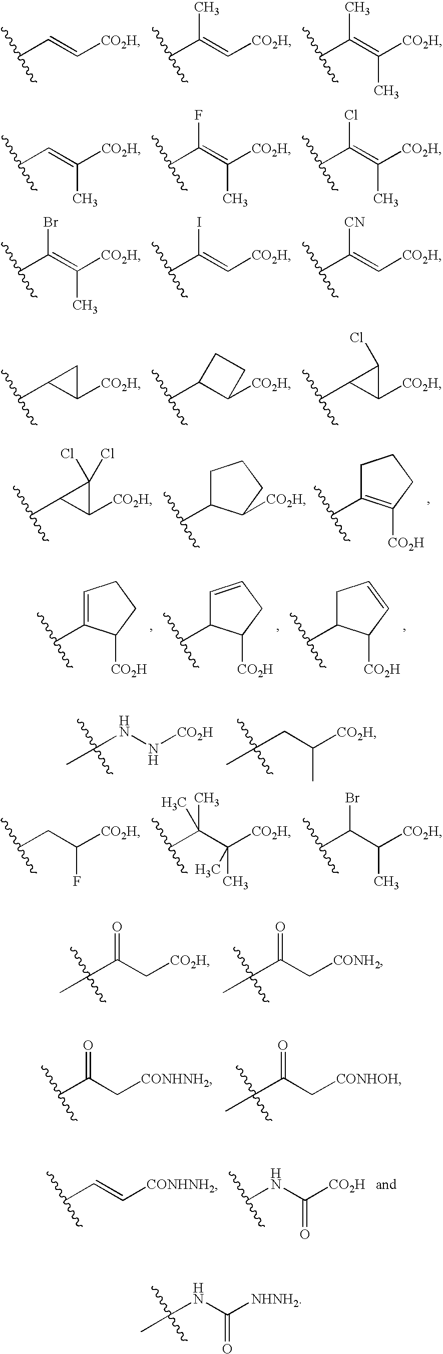

Within these embodiments, R¹ is a COOR³ or L¹-CO₂R³, wherein L¹ is defined as above in formula (I); and R³ is H or (CH₂)_{q}NR⁹R¹⁰ and each R⁹ and R¹⁰ is (C₁-C₈) alkyl, or optionally, if both are present on the same substituent, joined together to form a three- to eight-membered heterocyclyl ring system; and the subscript q is an integer of from 1 to 4.

In one embodiment, L¹ is —CV¹═CV³— wherein V¹ and V³ together form a (C₁-C₈) heterocycloalkyl, a (C₃-C₈) cycloalkenyl, an aryl, or a heteroaryl ring. In another embodiment the (C₁-C₈) heterocycloalkyl, (C₃-C₈) cycloalkenyl, aryl, or heteroaryl ring is a five-membered ring. In another embodiment, the heteroaryl ring contains one or more nitrogen atoms.

In another embodiment, (GROUP 28) R¹ is preferably a COOR³ moiety, and R³ is preferably H or (CH₂)ₙNR⁹R¹⁰ wherein each R⁹ and R¹⁰ is (C₁-C₈) alkyl, or optionally, if both are present on the same substituent, may be joined together to form a three- to eight-membered heterocyclyl ring system; and the subscript n is an integer of from 1 to 4.

In one embodiment, any R¹ and V⁶ or any two V⁶ attached to the same, adjacent or within two atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring. Within this embodiment, the (C₃-C₈) cycloalkyl moiety is selected from the group consisting of cyclopentane, cyclobutane, cyclohexane, and cycloheptane. In a related embodiment, (C₃-C₈) cycloalkenyl moiety is selected from the group consisting of cyclobutene, cyclopentene, cyclohexene, cycloheptene, and cyclooctene. In a related embodiment, the aryl moiety is selected from benzene or naphthalene. In another related embodiment, the heteraryl moiety selected from the group consisting of pyridine, furane, thiophene, thiazole, isothiazole, triazole, imidazole, isoxazole, pyrrole, pyrazole, pyridazine, pyrimidine, benzofurane, tetrahydrobenzofurane, isobenzofurane, benzothiazole, benzoisothiazole, benzotriazole, indole, isoindole, benzoxazole, quinoline, tetrahydroquinoline, isoquinoline, benzimidazole, benzisoxazole benzothiophene, indazole, pyrrolopyrymindine, indolizine, pyrazolopyridine, triazolopyridine, pyrazolopyrimidine, triazolopyrimidine, pyrrolotriazine, pyrazolotriazine, triazolotriazine, pyrazolotetrazine, hexaaza-indene, and heptaaza-indene and the derivatives thereof. In another related embodiment, the (C₁-C₈) heterocyclyl moiety is selected from the group consisting of piperidine, tetrahydropyran, N-methylpiperidine, N-methylpyrrolidine, pyrrolidone, tetrahydrofurane, morpholine, pyrrolidine, tetrahydrothiophene, 1,1-dioxo-hexahydro-1λ⁶-thiopyran, tetrahydroimidazo[4,5-c]pyridine, imidazoline, and piperazine. In another related embodiment, two V⁶ groups together forms a (C₁-C₈) heterocycle moiety selected from the group consisting of: 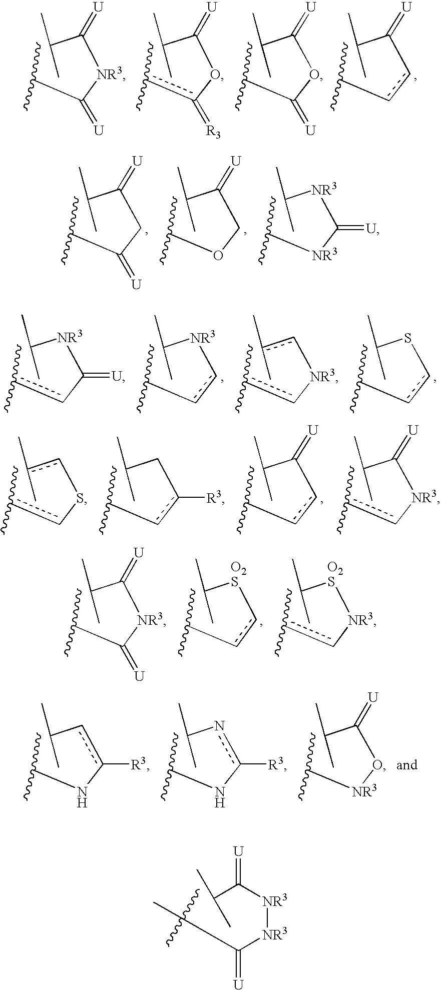
wherein the straight and wavy lines indicate the point of attachment to the rest of the molecule. In one embodiment the compound is selected from the group consisting of:

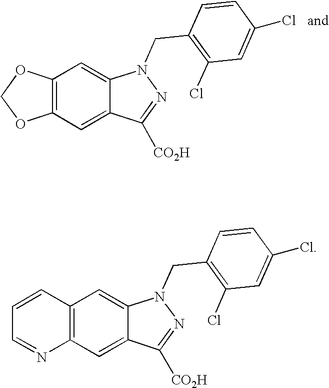

In other embodiments, the present invention provides a compound wherein the R¹ group is attached to the A-B ring system such that it is rotationally restricted. In one embodiment W¹ (or a substituent thereon) taken together with W² or W⁶ (or a substituent thereon) form a (C₃-C₈) cycloalkyl, a (C₁-C₈) heterocycloalkyl, a (C₃-C₈) cycloalkenyl, an aryl or a heteroaryl ring, such as for example

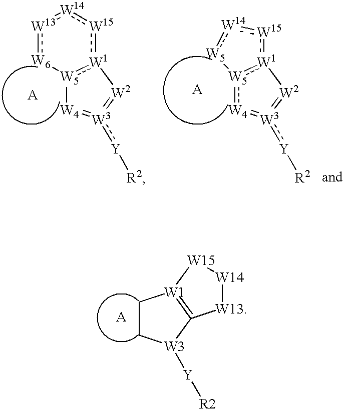

In one embodiment, W¹ taken together with W² or W⁶ form a (C₁-C₈) heterocycle moiety selected from the group consisting of 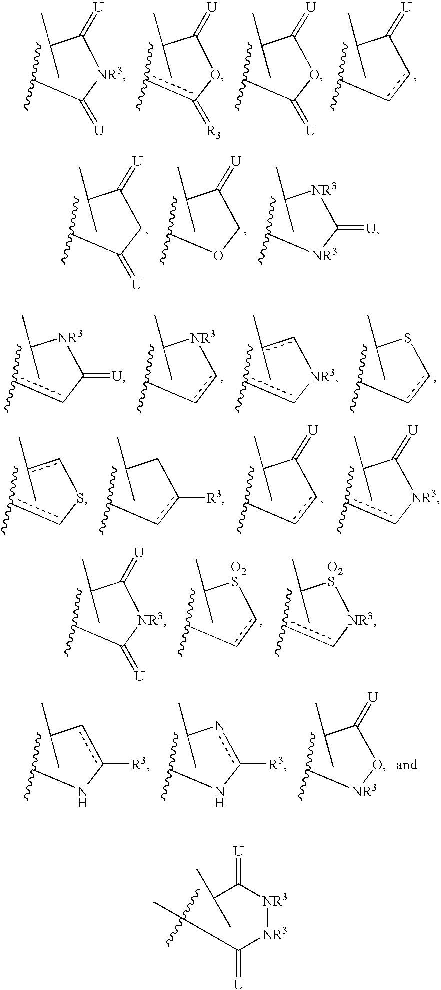
wherein the straight line indicates the point of attachment to W¹ and the wavy line indicates the points of attachment within two atoms of W¹ on the rest of the molecule. In one embodiment the straight line indicates the point of attachment to W². In another embodiment the straight line indicates the point of attachment to W⁶. 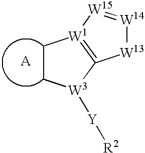
wherein ring A, W¹, W³, Y, and R² are defined as in formula (II), and each W¹³, W¹⁴ and W¹⁵ is independently selected from the group consisting of N, NV⁶, CO, CS, SO, SO₂ and CV⁶ wherein V⁶ is as defined above in formula (I). Within this embodiment, compounds of the present invention have the formulae: 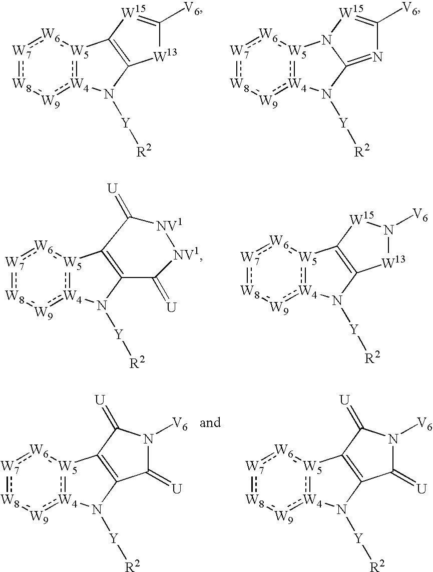
wherein the variables are as defined herein. Within the embodiment, compounds of the present invention have the formulae: 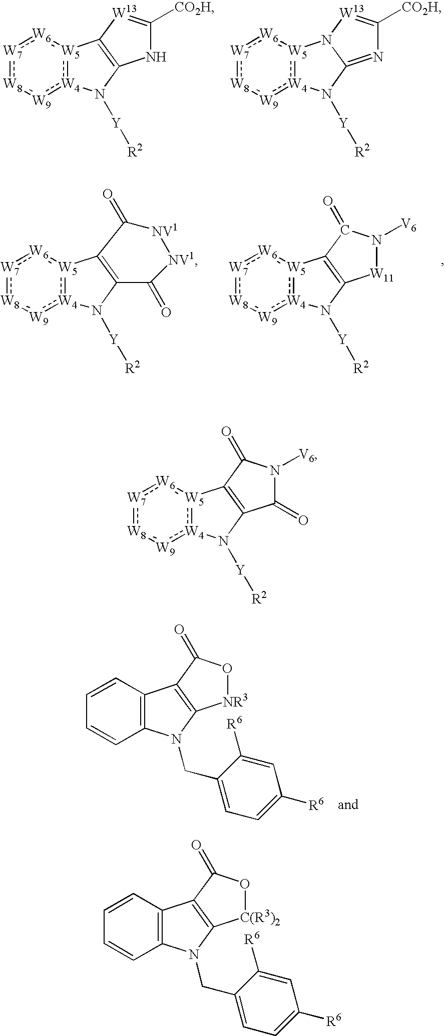
wherein the variables are as defined herein. Within the embodiment, compounds of the present invention have the formulae: 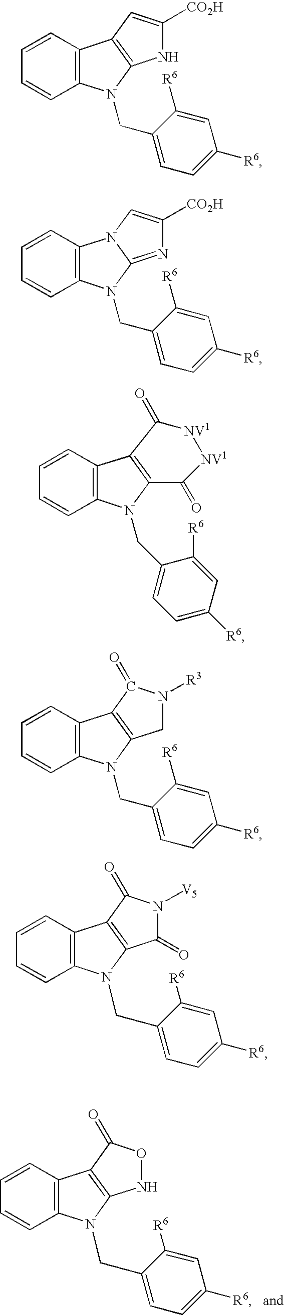 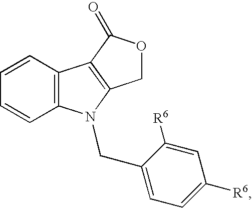
wherein R³, R⁶ and V⁶ is as defined above.

In another embodiment (GROUP 29), compounds of the present invention have the formulae: 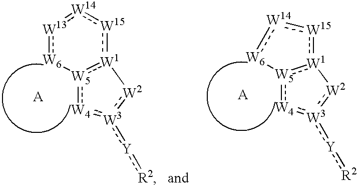
wherein W¹-W⁶ and W¹³-W¹⁵ is as defined above.

Within this embodiment (GROUP 9, compounds of the present invention have the formulae: 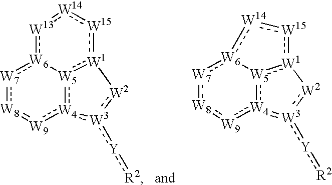
wherein W¹-W⁶ and W¹³-W¹⁵ is as defined herein.

Within this embodiment, compounds of the present invention have the formulae: 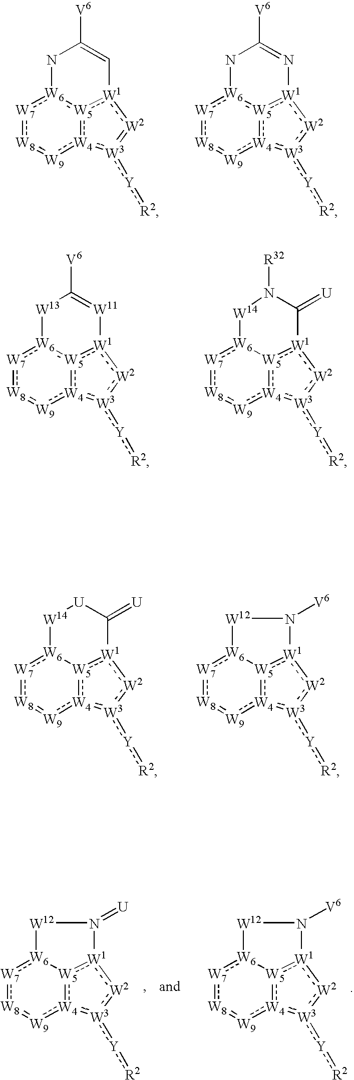
wherein the variables are as defined herein.

In another embodiment (GROUP 30), compounds of the present invention have the formulae: 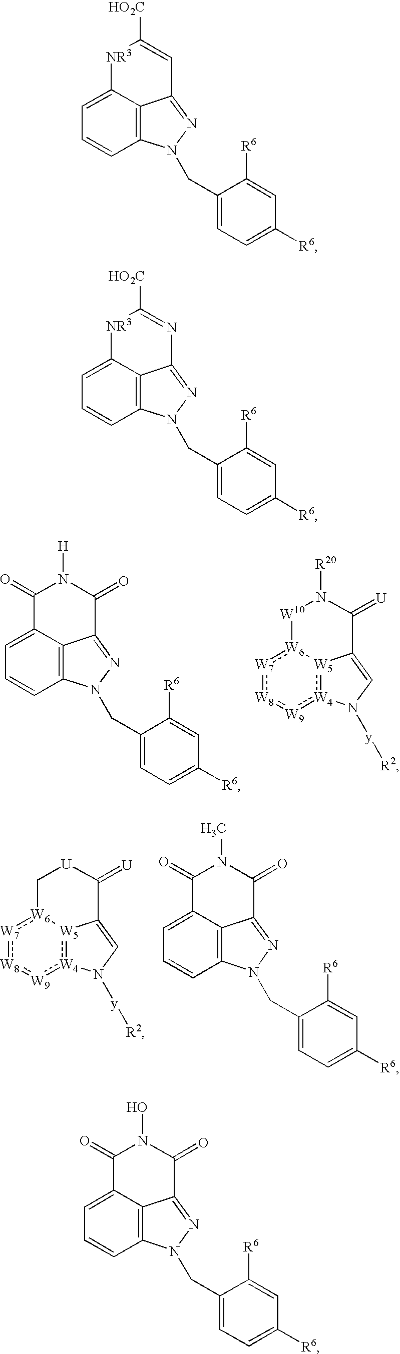 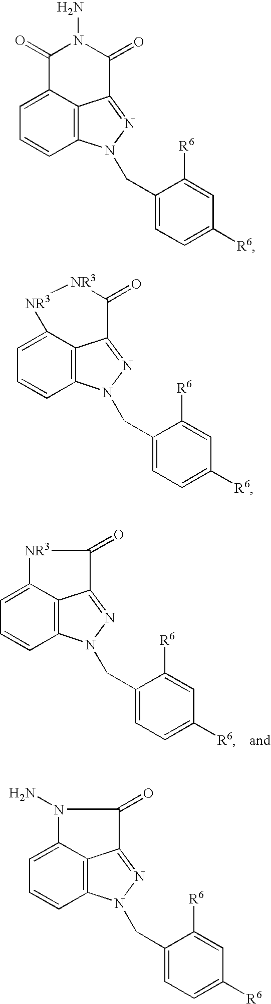
wherein the variables are as defined herein.

In one embodiment, the present invention provides R⁶ substituents, each independently selected from the group consisting of halo, nitro, cyano, nitrileoxide, —NO, R³, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₂R³, SOR³, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, nitrileoxide, and —NO, or any two R⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring. In another embodiment, the present invention provides R⁶ substituents, each independently selected from the group consisting of halo, nitro, cyano, U¹—R³, R³, R⁴, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—PU₂R³, N—(PU₂R³)₂, NR³—P(═U)(UR³)R³, CUR³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₂R³, SOR³, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, or any two R⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring. In another embodiment, the present invention provides R⁶ substituents, each independently selected from the group consisting of halo, cyano, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂, or any two R⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring. In another embodiment, the present invention provides R⁶ substituents, each independently selected from the group consisting of halo, cyano, CH₃, CH₂CH₃, CH(Me)₂, methoxymethyl, ethoxymethyl, cyclopropyl, cyclobutyl, 2-furanyl, 3-furanyl, ethynyl, 1-propynyl, 3-propynyl, O-Me, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)-C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, PO(NMe₂)₂. In another embodiment, the present invention provides R⁶ each independently selected from the group consisting of hydrogen, (C₁-C₄) alkyl or (C₁-C₄) heteroalkyl, halogen, hydroxy, (C₁-C₆) alkoxy, amino, cyano, nitro, (C₁-C₄) alkylamino, and (C₁-C₄) dialkylamino. In another embodiment, the present invention provides R⁶ each independently selected from the group consisting of methyl, ethyl, propyl, isopropyl, fluoro, chloro, bromo, iodo, amino, methylamino, dimethylamino, ethylamino, methoxy, and hydroxyl. In one embodiment, R⁶ the present invention provides each independently selected from the group consisting of hydrogen, F, Cl, Br, CN, CF₃, CH₃, CHMe₂, —C≡CH, and —C≡C—CH₃.

In one embodiment, R² has 1, 2 or 3 substituents. In another embodiment, R² has two R⁶ substituents. R⁶ substituents are independently selected from the group consisting of halo, (C₁-C₈) alkyl, C₁-C₈ heteroalkyl, aryl, heteroaryl, NR³COR³, hydroxy, alkoxy and CO₂R³.

In one embodiment, R² is selected from the group consisting of pyrroyl, pyrazoyl, imidazoyl, pyridinyl, dihydropyridinyl, pyrazinyl, pyridazinyl, pyrimidinyl and phenyl, optionally substituted with from one to two substituents selected from the group consisting of halo or (C₁-C₈) alkyl.

In another embodiment, R² is selected from the group consisting of 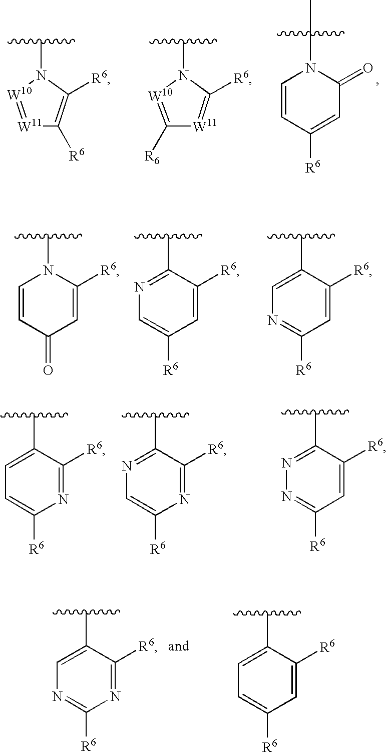
wherein each W¹⁰ or W¹¹ is preferably, independently selected from the group consisting of N, C and CH. In this embodiment R⁶ is preferably halo or (C₁-C₈) alkyl; and the wavy line indicates the point of attachment to the rest of the molecule. Within this embodiment R² is more preferably phenyl and R⁶ is preferably independently selected from the group consisting of Cl, Br, or CH₃, and more preferably is Cl.

In another embodiment R² is selected from the group consisting of: 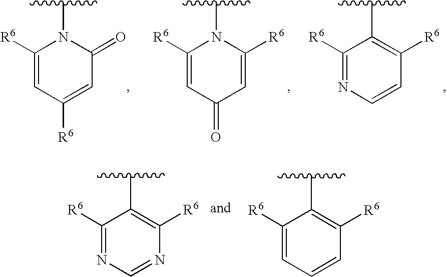
wherein the variables are as defined herein.

In another embodiment, R³, R⁷, and R⁸ are independently selected from the group consisting of: H, —CH₃, —CH₂CH₃,

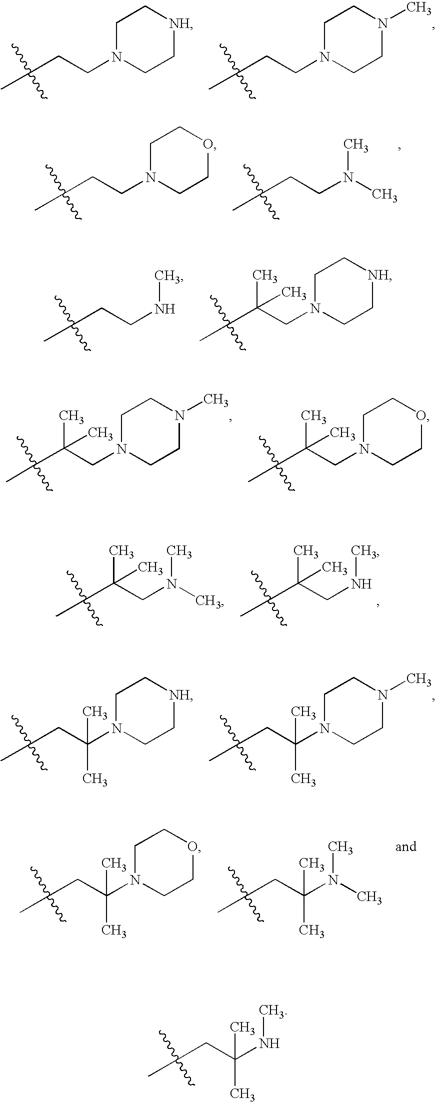

In another embodiment, Y is preferably NH, O, CR⁸₂ or CHR⁸. In another embodiment, Y is preferably NH, O, or CHR⁸. R⁸ is preferably H.

In another embodiment, (GROUP 31) compounds preferably have the formula: 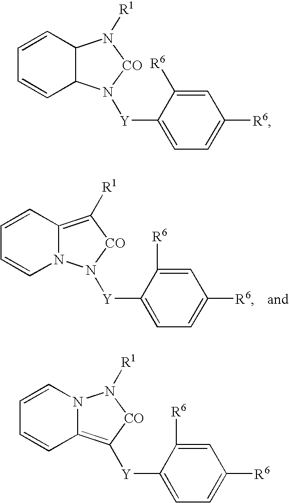
wherein R¹, R⁶ and Y are defined as in formula (IIID). Within this embodiment, compounds preferably have the formula: 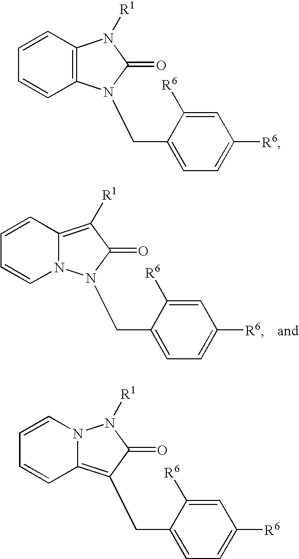
wherein R¹ is selected from the group consisting of COOR³, COR⁴, CH═CHCO₂R³, B(OR³)₂, SO₂R⁴, NHSO₂CR⁵₃ and CONH₂(═NHCN); R³ is H, (C₁-C₈) alkyl or (C₁-C₈) heteroalkyl; each R⁴ is a member independently selected from the group consisting of NR³R⁷, NHOR³, NHNR³R⁷ and NHCN; R⁵ is H, OH or halogen; R⁷ is H or (C₁-C₈) alkyl; and R⁶ is halo or (C₁-C₈) alkyl. Within this embodiment, R¹ is selected from the group consisting of COOR³, COR⁴, CH═CHCO₂R³, B(OR³)₂, SO₂R⁴, and NHSO₂CR⁵₃; each R⁴ is a member independently selected from the group consisting of NR³R⁷, NHOR³ and —NHNR³R⁷. Within these embodiments, R⁶ is preferably independently selected from the group consisting of Cl, Br, or CH₃, and more preferably is Cl.

In another embodiment (GROUP 39), compounds of the present invention have the structure:

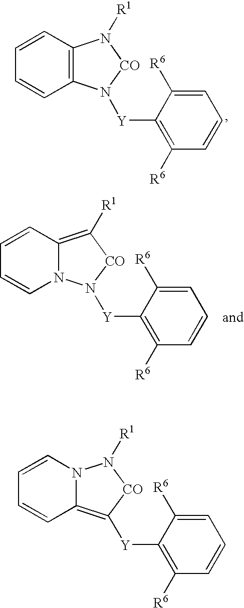

wherein R¹, R⁶ and Y are defined as in formulae (I) and (II). Within this embodiment, compounds preferably have the formula: 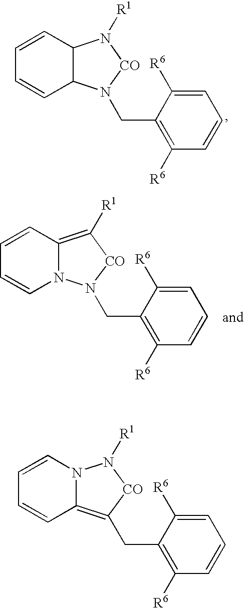
wherein R¹ is selected from the group consisting of COOR³, COR⁴, CH═CHCO₂R³, B(OR³)₂, SO₂R⁴, NHSO₂CR⁵₃ and CONH₂(═NHCN); R³ is H, (C₁-C₈) alkyl or (C₁-C₈) heteroalkyl; each R⁴ is a member independently selected from the group consisting of NR³R⁷, NHOR³, NHNR³R⁷ and NHCN; R⁵ is H, OH or halogen; R⁷ is H or (C₁-C₈) alkyl; and R⁶ is hydrogen, halo, (C₁-C₈) alkyl, C₁-C₈ heteroalkyl, aryl, heteroaryl, NR³COR³, hydroxy, alkoxy or CO₂R³. Within this embodiment, R¹ is selected from the group consisting of COOR³, COR⁴, CH═CHCO₂R³, B(OR³)₂, SO₂R⁴, and NHSO₂CR⁵₃; each R⁴ is a member independently selected from the group consisting of NR³R⁷, NHOR³ and NHNR³R⁷. Within these embodiments, R⁶ is preferably independently selected from the group consisting of Cl, Br, or CH₃, and more preferably is Cl.

In another embodiment, (GROUP 32) compounds have the formula: 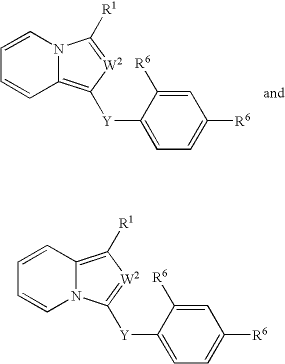
wherein R¹, W², Y, and R⁶ are defined as in formula (IIID). Within this embodiment, R¹ is selected from the group consisting of COOR³, COR⁴, CH═CHCO₂R³, B(OR³)₂, SO₂R⁴, NHSO₂CR⁵₃, NHSO₂CR³₃, C(═NCN)NH₂ and L¹-V⁵; L¹ and V⁵ are defined as above in formula (IIID); W² is N or CR⁹, R⁹ is H or halo; Y is CH₂, O, NH, or S; and R⁶ is halo or (C₁-C₈) alkyl. Within this embodiment, R¹ is COOR³ or —CV¹═CV³—R³. R³ is H or (CH₂)ₙNR¹⁰R¹¹, wherein each R¹⁰ and R¹¹ is (C₁-C₈) alkyl, or optionally, if both are present on the same substituent, may be joined together to form a three- to eight-membered (C₁-C₈) heterocyclyl ring system; and the subscript n is an integer of from 1 to 4. In another embodiment, R¹ is selected from the group consisting of COOR³, COR⁴, CH CHCO₂R³, B(OR³)₂, SO₂R⁴, NHSO₂CR⁵₃ and CONH₂(═NHCN); W² is N or CR⁹, R⁹ is H or halo; Y is CH₂, O, NH, or S; and R⁶ is halo or (C₁-C₈) alkyl. Within these embodiments, R¹ is preferably selected from the group consisting of COOR³, COR⁴, CH═CHCO₂R³, B(OR³)₂, SO₂R⁴ and NHSO₂CR⁵₃. Within this embodiment, R¹ is preferably COOR³. R³ is preferably H or (CH₂), NR¹⁰R¹¹, wherein each R¹⁰ and R¹¹ is (C₁-C₈) alkyl, or optionally, if both are present on the same substituent, may be joined together to form a three- to eight-membered heterocyclyl ring system; and the subscript n is an integer of from 1 to 4. R⁶ is preferably independently selected from the group consisting of Cl, Br, or CH₃, and more preferably is Cl.

In another embodiment, (GROUP 40) the compounds have the formula:

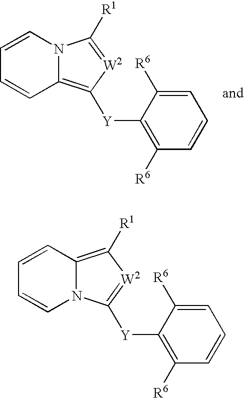

wherein R¹, W², Y, and R⁶ are defined as in formulae (I) and (II).

In another embodiment (GROUP 33), the compound has the formula:

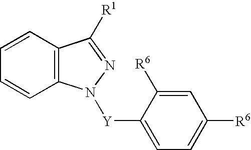

wherein

R¹ is selected from the group consisting of CO₂R³, COR⁴, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH— V⁵, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;

L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₄) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl; each R³ is a member independently selected from the group consisting of H, (C₁-C₈) alkyl, (C₁-C₈) heteroalkyl, (C₃-C₈) cycloalkyl, (C₁-C₈) heterocyclyl, aryl and heteroaryl;

each R⁴ is selected from the group consisting of NR³R⁷, NR³OR⁷, NR⁷NR³R⁷ and NR³CN;

R⁵ is H, OH or halogen;

each R⁶ independently is hydrogen, Cl, F, Br, OH, OCH₃, OCF₃, CN, CF₃, CH₃, CH₂F, CHF₂, CHMe₂, —C≡CH, and —C≡C—CH₃, or NHCOCH₃;

each V⁵ is a member independently selected from the group consisting of COOR³, COR⁴, CONR³COR³, COCOR⁴, B(OR³)₂, SO₂R⁴, NHSO₂CR⁵₃, NHSO₂R³₃, CONHSO₂CR³₃ and C(═NCN)NH₂;

R⁷ is selected from the group consisting of H, (C₁-C₈) alkyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;

Y is CHR⁸, CR⁸₂, NR⁸, S or O; and

R⁸ is H, (C₁-C₈) alkyl or (C₁-C₈) heteroalkyl; or two R⁸ taken together form a (C₁-C₈) heterocyclyl or heteroaryl ring.

In another embodiment (GROUP 41), the compound has the formula:

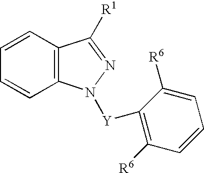

wherein

R¹ is selected from the group consisting of CO₂R³, COR⁴, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH— V⁵, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;

L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₄) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl;

each R³ is a member independently selected from the group consisting of H, (C₁-C₈) alkyl, (C₁-C₈) heteroalkyl, (C₃-C₈) cycloalkyl, (C₁-C₈) heterocyclyl, aryl and heteroaryl;

each R⁴ is selected from the group consisting of NR³R⁷, NR³OR⁷, NR⁷NR³R⁷ and NR³CN;

R⁵ is H, OH or halogen;

each R⁶ independently is hydrogen, Cl, F, Br, OH, OCH₃, OCF₃, CN, CF₃, CH₃, CH₂F, CHF₂, CHMe₂, —C≡CH and —C≡C—CH₃, or NHCOMe;

each V⁵ is a member independently selected from the group consisting of COOR³, COR⁴, CONR³COR³, COCOR⁴, B(OR³)₂, SO₂R⁴, NHSO₂CR⁵₃, NHSO₂R³₃, CONHSO₂CR³₃ and C(═NCN)NH₂;

R⁷ is selected from the group consisting of H, (C₁-C₈) alkyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;

Y is CHR⁸, CR⁸₂, NR⁸, S or O; and

R⁸ is H, (C₁-C₈) alkyl or (C₁-C₈) heteroalkyl; or two R⁸ taken together form a (C₁-C₈) heterocyclyl or heteroaryl ring.

In another embodiment, (GROUP 34) compounds have the formula: 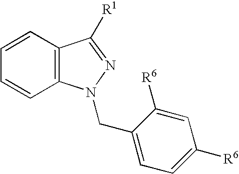
wherein each R⁶ independently is selected from the group consisting of hydrogen, F, Cl, Br, OH, OCH₃, OCF₃, CN, CF₃, CH₃, CH₂F, CHF₂, CHMe₂, —C≡CH, —C≡C—CH₃, and NHCOMe; and

R¹ is COOH, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂; —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂; with the proviso that when R¹ is CH═CHCO₂H, CONHNH₂ or CONHNMe₂, then at least one of R⁶ is CN, CF₃, CH₃, CHMe₂, —C≡CH, and —C≡C—CH₃. Within this embodiment, the compounds have the formula 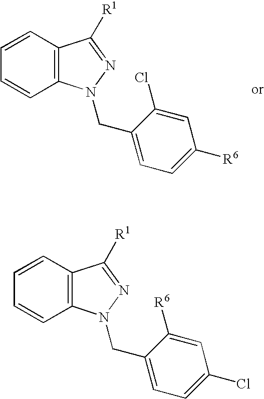
wherein each R⁶ independently is hydrogen, Cl, F, Br, OH, OCH₃, OCF₃, CN, CF₃, CH₃, CH₂F, CHF₂, CHMe₂, —C≡CH, —C≡C—CH₃, and NHCOMe and

R¹ is CO₂H, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂, —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂. Within this embodiment, the compounds have the formula: 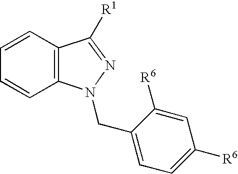
wherein each R⁶ independently is hydrogen, F, Cl, Br, OH, OCH₃, OCF₃, CN, CF₃, CH₂F, CHF₂, CH₃, CHMe₂, —C≡CH, —C≡C—CH₃, NHCOMe; R¹ is COOH, CONH₂, CONHOH, CONHR³, CONHAr, CONH(Py) wherein Py is 2-, 3-, or 4-pyridyl, CONHSO₂R³, and CONHN═CR³R⁷. Within this embodiment, the compound has the formula: 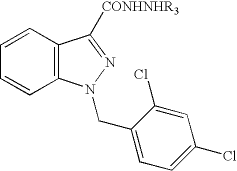
wherein R³ is

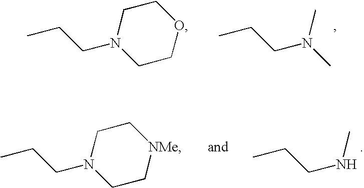

In another embodiment (GROUP 42), compounds have the formula: 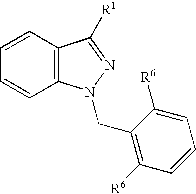
wherein each R⁶ independently is selected from the group consisting of hydrogen, F, Cl, Br, OH, OCH₃, OCF₃, CN, CF₃, CH₂F, CHF₂, CH₃, CHMe₂, —C≡CH, —C≡C—CH₃, NHCOMe; and

R¹ is COOH, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂; —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂; with the proviso that when R¹ is CH═CHCO₂H, CONHNH₂, or CONHNMe₂, then at least one of R⁶ is F, Cl, Br, OH, OCH₃, OCF₃, CN, CF₃, CH₂F, CHF₂, CH₃, CHMe₂, —C≡CH, and —C≡C—CH₃. Within this embodiment, the compounds have the formula 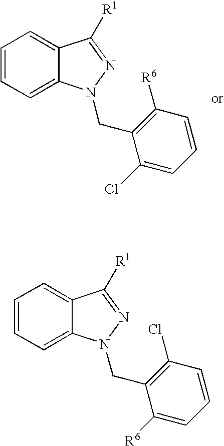
wherein each R⁶ independently is hydrogen, F, Cl, Br, OH, OCH₃, OCF₃, CN, CF₃, CH₂F, CHF₂, CH₃, CHMe₂, —C≡CH, —C≡C—CH₃, NHCOMe; R¹ is CO₂H, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂, —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂. Within this embodiment, the compounds have the formula: 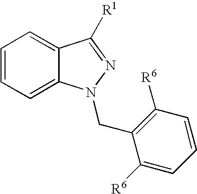
wherein each R⁶ independently is hydrogen, F, Cl, Br, OH, OCH₃, OCF₃, CN, CF₃, CH₂F, CHF₂, CH₃, CHMe₂, —C≡CH, —C≡C—CH₃, NHCOMe; R¹ is COOH, CONH₂, CONHOH, CONHR³, CONHAr, CONH(Py) wherein Py is 2-, 3-, or 4-pyridyl, CONHSO₂R³, and CONHN═CR³R⁷.
Within this embodiment, the compounds has the formula: 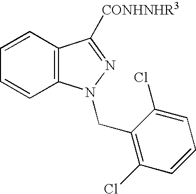
wherein R³ is

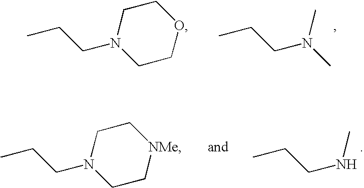

In another embodiment, (GROUP 35), the compounds have the formulas 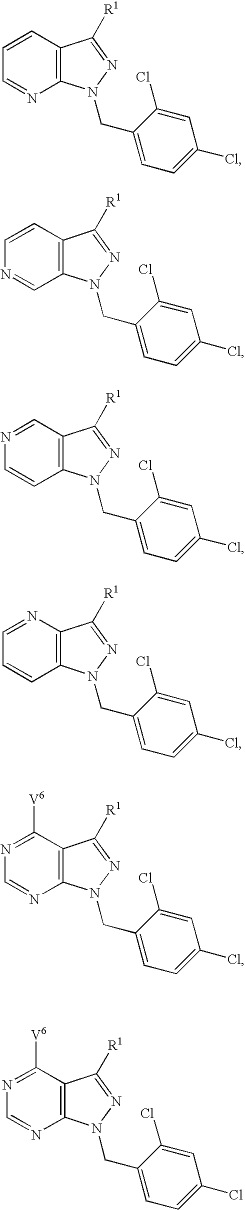
wherein R¹ is CO₂H, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂, —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂;
and V⁶ is hydrogen, amino, or alkylamino.

In another embodiment, (GROUP 43), the compounds have the formulas 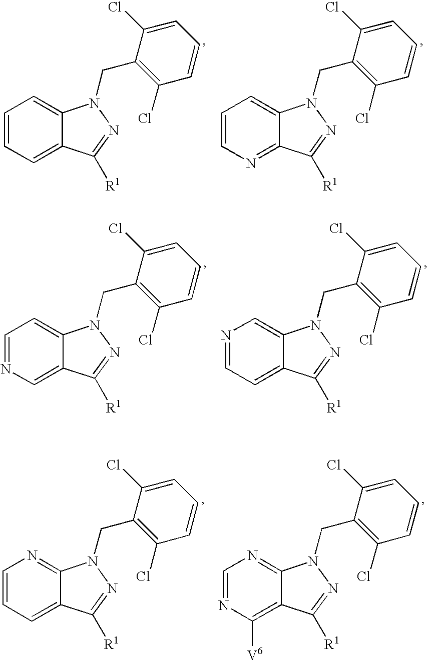
wherein R¹ is CO₂H, CH═CHCO₂, CONHOH, CONHNH₂CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂, —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂;
and V⁶ is hydrogen, amino, or alkylamino.

In another embodiment (GROUP 36), compounds have the formula:

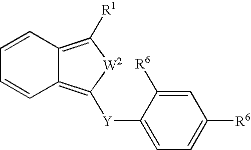

wherein R¹, W², Y, and R⁶ are defined as in formula (IIID).

In another embodiment (GROUP 44), compounds have the formula:

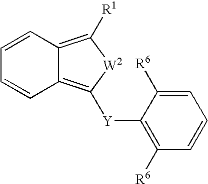

wherein R¹, W², Y, and R⁶ are defined as in formula (IIID).

In another embodiment, (GROUP 37) the compounds have the formula: 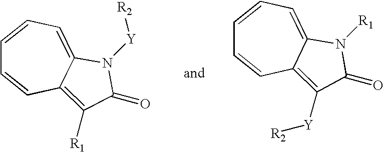
wherein R¹, R² and Y are as defined above in formula (I).

In one embodiment (GROUP 38), the present invention provides the compounds having the formula selected from the group consisting of: 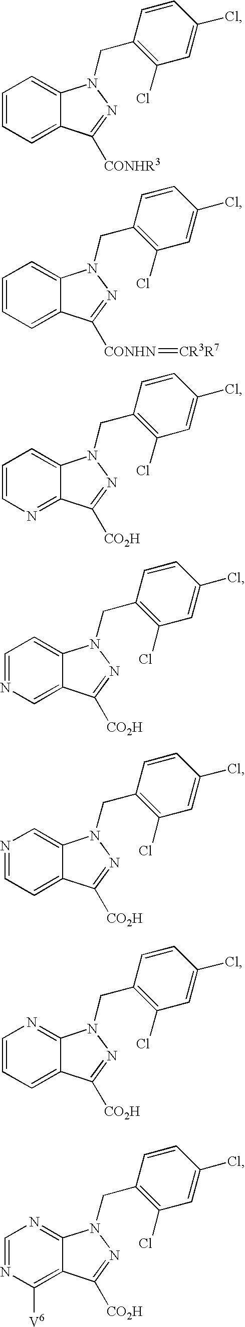 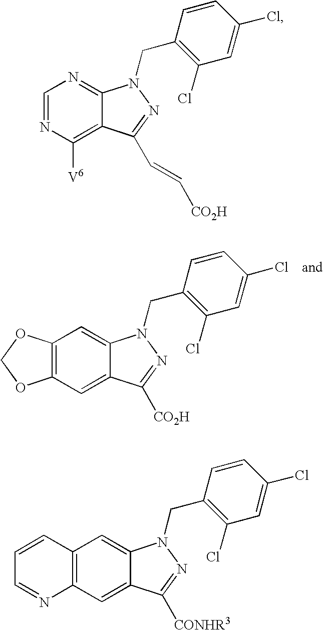
wherein R³ is phenyl, pyridyl or SO₂R⁷; and V⁶ is H or NHR³.

In one embodiment (GROUP 45), the present invention provides the compounds having the formula selected from the group consisting of: 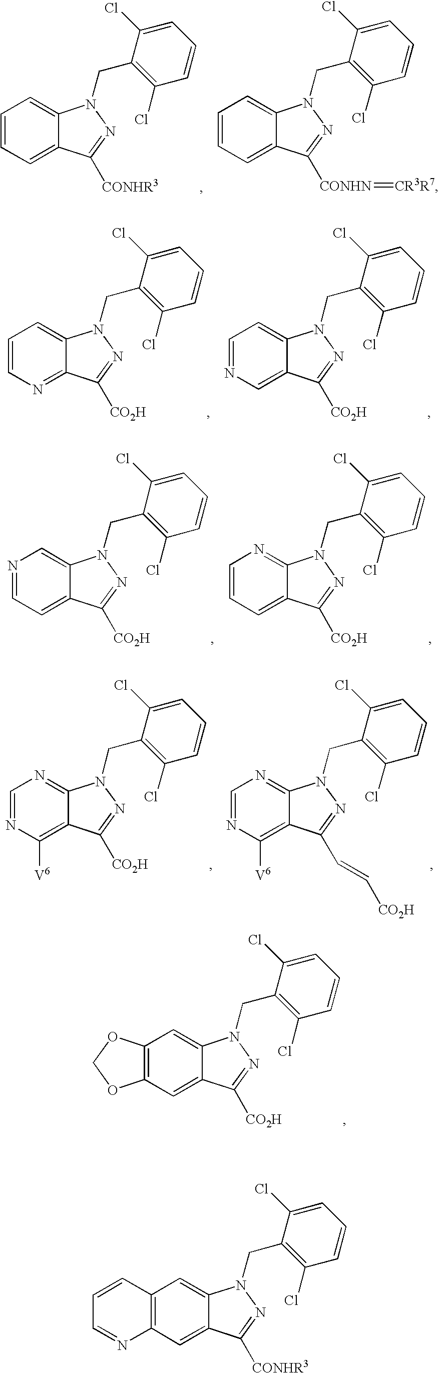
wherein R³ is phenyl, pyridyl or SO₂R⁷; and V⁶ is H or NHR³.

Within any of the above embodiments, R¹ is L¹-V¹ or CO₂R³. In another embodiment R¹ is C₂alkenyl-CO₂R³. In another embodiment, R³ is H or (CH₂)_{q}NR³₂; each R¹³ is independently (C₁-C₈) alkyl, or, if both present on the same substituent may be joined together to form a three- to eight-membered heterocyclyl ring system; and the subscript n is an integer of from 1 to 4.

In another embodiment (GROUP 46), the compound has the formula: 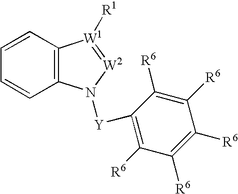
wherein

R¹ is selected from the group consisting of CO₂R³, COR⁴, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH— V⁵, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;

L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₄) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then a V¹ attached to the same atom is hydrogen or alkyl;

each R³ is a member independently selected from the group consisting of H, (C₁-C₈) alkyl, (C₁-C₈) heteroalkyl, (C₃-C₈) cycloalkyl, (C₁-C₈) heterocyclyl, aryl and heteroaryl;

each R⁴ is selected from the group consisting of NR³R⁷, NR³OR⁷, NR⁷NR³R⁷ and NR³CN;

R⁵ is H, OH or halogen;

each R⁶ is a member independently selected from the group consisting of H, halo, (C₁C₈) alkyl, and (C₁-C₈) heteroalkyl, aryl, heteroaryl, NR³COR³, hydroxy, alkoxy and CO₂R³,

R⁷ is selected from the group consisting of H, (C₁-C₈) alkyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;

each V⁵ is a member independently selected from the group consisting of COOR³, COR⁴, CONR³COR³, COCOR⁴, B(OR³)₂, SO₂R⁴, NHSO₂CR⁵₃, NHSO₂R³₃, CONHSO₂CR³₃ and C(═NCN)NH₂;

each V⁶ is independently a member selected from the group consisting of hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂ and PO(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

the subscript p10 is an integer of from 0 to 4;

W¹ independently C or N;

W² is N, CR⁵ or CO;

Y is CHR⁸, CR⁸₂, NR⁸, S or O; and

R⁸ is H, (C₁-C₈) alkyl or (C₁-C₈) heteroalkyl.

Within this embodiment, R¹ is selected from the group consisting of: CO₂R³, COR⁴, CH═CHCO₂R³ and CONHSO₂CR³₃;

each R³ is a member independently selected from the group consisting of H, (C₁-C₈) alkyl, aryl, (C₁-C₈) heteroalkyl and (C₁-C₈) heterocyclyl;

each R⁴ is a member independently selected from the group consisting of NR³R⁷, NR³OR⁷ and NR⁷NR³R⁷;

each R⁶ is a member independently selected from the group consisting of H, halo, (C₁C₈) alkyl, (C₁-C₈) heteroalkyl, C₁-C₈ heteroalkyl, aryl, heteroaryl, NR³COR³, hydroxy, alkoxy, CO₂R³, haloalkyl, and haloalkoxy;

R⁷ is selected from the group consisting of H, (C₁-C₈) alkyl, (C₁-C₈) heteroalkyl, aryl and (C₁-C₈) heterocyclyl;

each V⁶ is independently a member selected from the group consisting of hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, and PO(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

p10 is an integer of from 0 to 4;

Y is CH₂, NH, S or O; and

W¹ and W² is each C or N.

Within this embodiment, V⁶ is H. In another embodiment, W¹ and W² are CR⁵. In another embodiment, W¹ is CR⁵ and W² is N. In another embodiment, W¹ is N and W² is CR⁵. In another embodiment, Y is O. In another embodiment, Y is S. In another embodiment, Y is NH. In another embodiment, Y is CH₂ Within these embodiments, the compound has the formula:

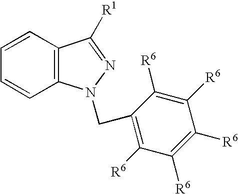

wherein R¹ is selected from the group consisting of: CO₂R³, COR⁴, CONHSO₂CR³₃;

each R³ is a member independently selected from the group consisting of H, (C, C₈) alkyl, aryl, (C₁-C₈) heteroalkyl, (C₁-C₈) heterocyclyl;

each R⁴ is a member independently selected from the group consisting of NR³R⁷, NR³OR⁷, NR⁷NR³R⁷;

R⁶ is independently selected from the group consisting of H, halo, (C₁-C₈) alkyl, (C₁-C₈) heteroalkyl, C₁-C₈ heteroalkyl, aryl, heteroaryl, NR³COR³, hydroxy, alkoxy, CO₂R³, haloalkyl, and haloalkoxy;

R⁷ is H, (C, C₈) alkyl, (C₁-C₈) heteroalkyl, aryl, (C₁-C₈) heterocyclyl; and
Y is CH₂

In another embodiment (GROUP 47), the compound has the formula:

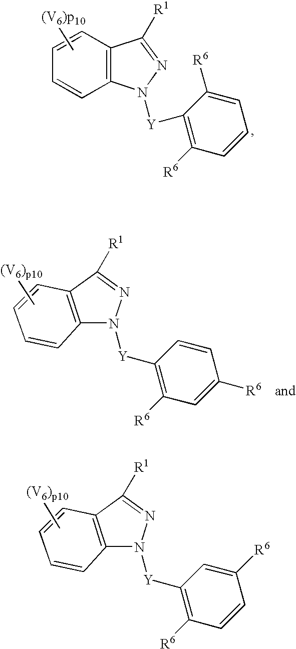

wherein

R¹ is selected from the group consisting of CO₂R³, COR⁴, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH—V⁵, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;

L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₄) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl;

p₁₀ is 1-4;

each V⁶ is, independently hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³), PO(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

R² is an aryl or heteroaryl group, optionally substituted with from one to three R⁶ substituents independently selected from the group consisting of halo, cyano, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂, or any two R⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

each R³ is a member independently selected from the group consisting of H, (C₁-C₈) alkyl, (C₁-C₈) heteroalkyl, (C₃-C₈) cycloalkyl, (C₁-C₈) heterocyclyl, aryl and heteroaryl;

each R⁴ is selected from the group consisting of NR³R⁷, NR³OR⁷, NR⁷NR³R⁷ and NR³CN;

R⁵ is H, OH or halogen;

each V⁵ is a member independently selected from the group consisting of COOR³, COR⁴, CONR³COR³, COCOR⁴, B(OR³)₂, SO₂R⁴, NHSO₂CR⁵₃, NHSO₂R³₃, CONHSO₂CR³₃ and C(═NCN)NH₂;

R⁷ is selected from the group consisting of H, (C₁-C₈) alkyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;

Y is CHR⁸, C(R⁸)₂, NR⁸, S or O; and

R⁸ is H, (C₁-C₈) alkyl, (C₁-C₈) heteroalkyl, alkenyl, alkynyl, aryl, heteroaryl, cycloalkyl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂; or two R⁸ taken together form a (C₁-C₈) heterocyclyl or heteroaryl ring.

In another embodiment, (GROUP 48) compounds have the formula: 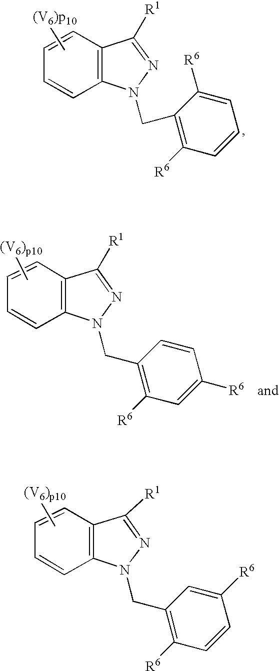
wherein each R⁶ independently is selected from the group consisting of hydrogen, F, Cl, Br, OH, OCH₃, OCF₃, CN, CF₃, CH₂F, CHF₂, CH₃, CHMe₂, —C≡CH, and —C≡C—CH₃;
p₁₀ is 1-4;

each V⁶ independently is hydrogen, halo, oxo, cyano, nitro, COOH, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF₃, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)-C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and

R¹ is COOH, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂; —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂; with the proviso that when each V⁶ is H and R¹ is CH═CHCO₂H, CONHNH₂, or CONHNMe₂, then at least one of R⁶ is hydrogen, F, Cl, Br, OH, OCH₃, OCF₃, CN, CF₃, CH₂F, CHF₂, CH₃, CHMe₂, —C≡CH and —C≡C—CH₃. Within this embodiment, the compounds have the formula 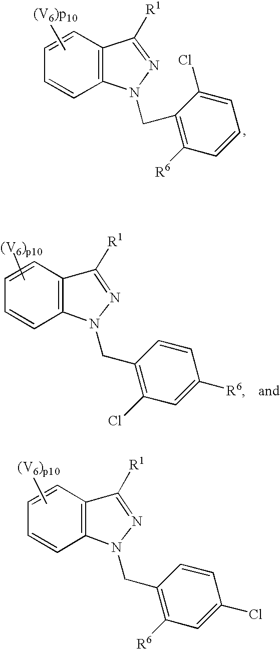
wherein R⁶ is hydrogen, Cl, F, Br, OH, OCH₃, OCF₃, CN, CF₃, CH₃, CH₂F, CHF₂, CHMe₂, —C≡CH, and —C≡C—CH₃;

p10 is 1-4;

each V⁶ independently is hydrogen, halo, oxo, cyano, nitro, COOH, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF₃, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)—C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and R¹ is CO₂H, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂, —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂, —C≡C—CONMeNHNe and —C≡C—CONMeNMe₂.

Within this embodiment, the compounds have the formula: 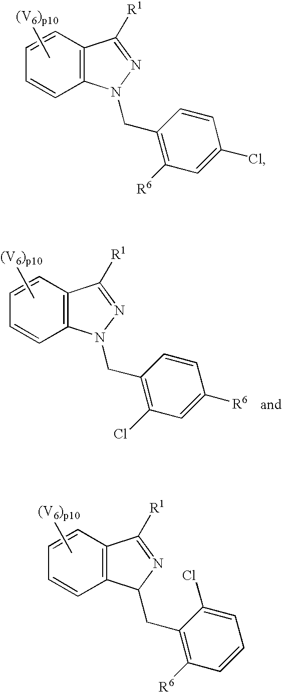
wherein each R⁶ independently is hydrogen, F, Cl, Br, OH, OCH₃, OCF₃, CN, CF₃, CH₂F, CHF₂, CH₃, CHMe₂, —C≡CH and —C≡C—CH₃; p10 is 1-4, each V⁶ independently is hydrogen, halo, oxo, cyano, nitro, COOH, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF₃, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)-C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and R¹ is COOH, CONH₂, CONHOH, CONHR³, CONHAr, CONH(Py) wherein Py is 2-, 3-, or 4-pyridyl, CONHSO₂R³, and CONHN═CR³R⁷. Within this embodiment, the compound has the formula: 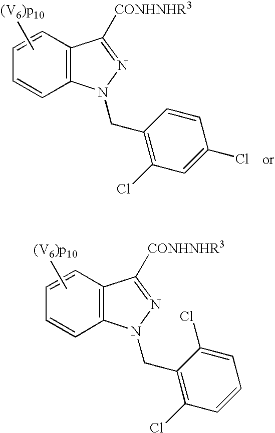
wherein each V⁶ independently is hydrogen, halo, oxo, COOH, cyano, nitro, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF³, O-Et, O-cyclopropyl, O-Aryl, S-Me, S-Et, N H₂, NHMe, NMe₂, NHAc, NHOH, NHNH₂, NHNHAc, NH—CONH₂, NMe-CONMe₂, NH—CSNH₂, NH—C(═NH)NH₂, N(Me)-C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and R³ is

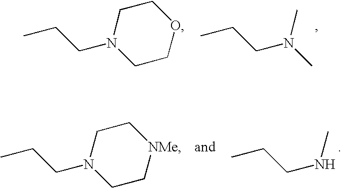

In another embodiment, (GROUP 49) compounds preferably have the formula:

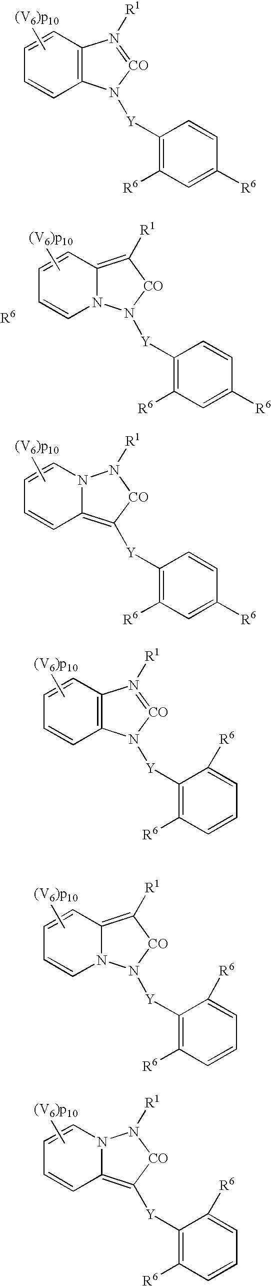

wherein

R¹ is selected from the group consisting of CO₂R³, COR⁴, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH—V⁵, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;

L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₄) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl;

p₁₀ is 1-4;

each V⁶ is, independently hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SOR³, SOR³, SO₃R³¹, SO₂NR³R⁷, PO(OR³)₂, PS(OR³)₂, PO(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

R² is an aryl or heteroaryl group, optionally substituted with from one to three R⁶ substituents independently selected from the group consisting of halo, cyano, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂, or any two R⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

each R³ is a member independently selected from the group consisting of H, (C₁-C₈) alkyl, (C₁-C₈) heteroalkyl, (C₃-C₈) cycloalkyl, (C₁-C₈) heterocyclyl, aryl and heteroaryl;

each R⁴ is selected from the group consisting of NR³R⁷, NR³OR⁷, NR⁷NR³R⁷ and NR³CN;

R⁵ is H, OH or halogen;

each V⁵ is a member independently selected from the group consisting of COOR³, COR⁴, CONR³COR³, COCOR⁴, B(OR³)₂, SO₂R⁴, NHSO₂CR⁵₃, NHSO₂R³₃, CONHSO₂CR³₃ and C(═NCN)NH₂;

R⁷ is selected from the group consisting of H, (C₁-C₈) alkyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;

Y is CHR⁸, C(R⁸)₂, NR⁸, S or O; and

R⁸ is H, (C₁-C₈) alkyl, (C₁-C₈) heteroalkyl, alkenyl, alkynyl, aryl, heteroaryl, cycloalkyl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂; or two R⁸ taken together form a (C₁-C₈) heterocyclyl or heteroaryl ring.

Within this embodiment, Y is CH²; each R⁶ independently is selected from the group consisting of hydrogen, F, Cl, Br, OH, OCH₃, OCF₃, CN, CF₃, CH₂F, CHF₂, CH₃, CHMe₂, —C≡CH and —C≡C—CH₃; p₁₀ is 1-4;

each V⁶ independently is hydrogen, halo, oxo, COOH, cyano, nitro, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF₃, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)—C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and

R¹ is COOH, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂; —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂; with the proviso that when each V⁶ is H and R¹ is CH═CHCO₂H, CONHNH₂, or CONHNMe₂, then at least one of R⁶ is CN, CF₃, CH₃, CHMe₂, —C≡CH, and —C≡C—CH₃.

Within this embodiment, Y is CH²; each R⁶ independently is hydrogen, F, Cl, Br, OH, OCH₃, OCF₃, CN, CF₃, CH₂F, CHF₂, CH₃, CHMe₂, —C≡CH and —C≡C—CH₃; p10 is 1-4, each V⁶ independently is hydrogen, halo, oxo, cyano, nitro, COOH, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF₃, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)-C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and R¹ is COOH, CONH₂, CONHOH, CONHR³, CONHAr, CONH(Py) wherein Py is 2-, 3-, or 4-pyridyl, CONHSO₂R³, and CONHN═CR³R⁷.

Within this embodiment, R¹ is CONHNHR³; each V⁶ independently is hydrogen, halo, oxo, cyano, nitro, CH₃, CH₂CH₃, CH(Me)₂, methoxymethyl, ethoxymethyl, cyclopropyl, cyclobutyl, 2-furanyl, 3-furanyl, ethynyl, 1-propynyl, 3-propynyl, O-Me, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)—C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and R³ is

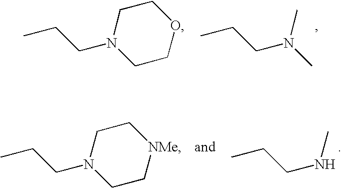

In another embodiment, (GROUP 50) compounds have the formula:

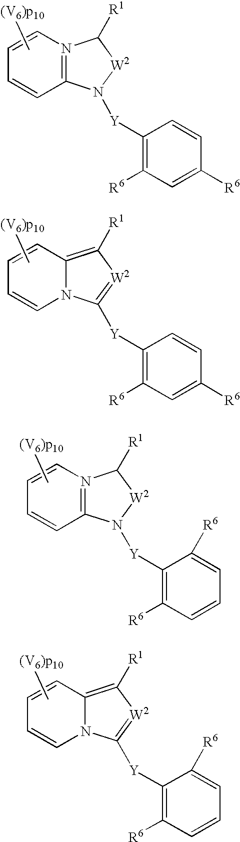

wherein

R¹ is selected from the group consisting of CO₂R³, COR⁴, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH—V⁵, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;

L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₄) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl;

p₁₀ is 1-4;

each V⁶ is, independently hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

R² is an aryl or heteroaryl group, optionally substituted with from one to three R⁶ substituents independently selected from the group consisting of halo, cyano, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SOR³¹, SO₂NR³R⁷, PO(OR³)₂, PS(OR³)₂, PO(NR³R⁷)₂, or any two R⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

each R³ is a member independently selected from the group consisting of H, (C₁-C₈) alkyl, (C₁-C₈) heteroalkyl, (C₃-C₈) cycloalkyl, (C₁-C₈) heterocyclyl, aryl and heteroaryl;

each R⁴ is selected from the group consisting of NR³R⁷, NR³OR⁷, NR⁷NR³R⁷ and NR³CN;

R⁵ is H, OH or halogen;

each V⁵ is a member independently selected from the group consisting of COOR³, COR⁴, CONR³COR³, COCOR⁴, B(OR³)₂, SO₂R⁴, NHSO₂CR⁵₃, NHSO₂R³₃, CONHSO₂CR³₃ and C(═NCN)NH₂;

R⁷ is selected from the group consisting of H, (C₁-C₈) alkyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;

Y is CHR⁸, C(R⁸)₂, NR⁸, S or O; and

R⁸ is H, (C₁-C₈) alkyl, (C₁-C₈) heteroalkyl, alkenyl, alkynyl, aryl, heteroaryl, cycloalkyl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂; or two R⁸ taken together form a (C₁-C₈) heterocyclyl or heteroaryl ring. Within this embodiment, Y is CH²; each R⁶ independently is selected from the group consisting of hydrogen, F, Cl, Br, OH, OCH₃, OCF₃, CN, CF₃, CH₂F, CHF₂, CH₃, CHMe₂, —C≡CH, and —C≡C—CH₃; p₁₀ is 1-4;

each V⁶ independently is hydrogen, halo, oxo, COOH, cyano, nitro, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF₃, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)—C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and

R¹ is COOH, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂; —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂; with the proviso that when each V⁶ is H and R′ is CH═CHCO₂H, CONHNH₂, or CONHNMe₂, then at least one of R⁶ is CN, CF₃, CH₃, CHMe₂, —C≡CH, and —C≡C—CH₃.

Within this embodiment, Y is CH₂; each R⁶ independently is hydrogen, F, Cl, Br, OH, OCH₃, OCF₃, CN, CF₃, CH₂F, CHF₂, CH₃, CHMe₂, —C≡CH and —C≡C—CH₃; p10 is 1-4, each V⁶ independently is hydrogen, halo, oxo, COOH, cyano, nitro, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF₃, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)-C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and R¹ is COOH, CONH₂, CONHOH, CONHR³, CONHAr, CONH(Py) wherein Py is 2-, 3-, or 4-pyridyl, CONHSO₂R³, and CONHN═CR³R⁷.

Within this embodiment, R¹ is CONHNHR³; each V⁶ independently is hydrogen, halo, oxo, COOH, cyano, nitro, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF³, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)-C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and R³ is

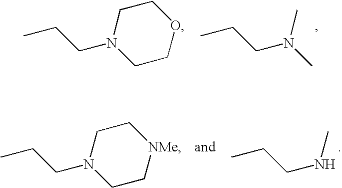

In another embodiment (GROUP 51), compounds have the formula:

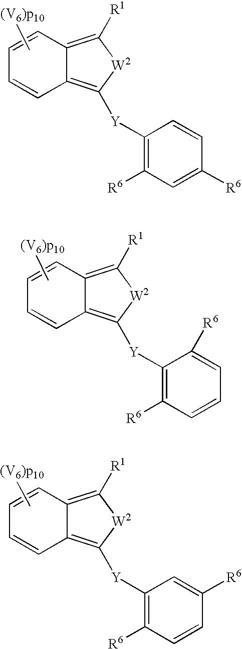

wherein

R¹ is selected from the group consisting of CO₂R³, COR⁴, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH—V⁵, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;

L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₄) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl;

p₁₀ is 1-4;

each V⁶ is, independently hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

R² is an aryl or heteroaryl group, optionally substituted with from one to three R⁶ substituents independently selected from the group consisting of halo, cyano, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SOR³¹, SO₂NR³R⁷, PO(OR³)₂, PS(OR³)₂, PO(NR³R⁷)₂, or any two R⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;

each R³ is a member independently selected from the group consisting of H, (C₁-C₈) alkyl, (C₁-C₈) heteroalkyl, (C₃-C₈) cycloalkyl, (C₁-C₈) heterocyclyl, aryl and heteroaryl;

each R⁴ is selected from the group consisting of NR³R⁷, NR³OR⁷, NR⁷NR³R⁷ and NR³CN;

R⁵ is H, OH or halogen;

each V⁵ is a member independently selected from the group consisting of COOR³, COR⁴, CONR³COR³, COCOR⁴, B(OR³)₂, SO₂R⁴, NHSO₂CR⁵₃, NHSO₂R³₃, CONHSO₂CR³₃ and C(═NCN)NH₂;

R⁷ is selected from the group consisting of H, (C₁-C₈) alkyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;

Y is CHR⁸, C(R⁸)₂, NR⁸, S or O; and

R⁸ is H, (C₁-C₈) alkyl, (C₁-C₈) heteroalkyl, alkenyl, alkynyl, aryl, heteroaryl, cycloalkyl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂, PO(NR³R⁷)₂; or two R⁸ taken together form a (C₁-C₈) heterocyclyl or heteroaryl ring.

Within this embodiment, Y is CH₂; each R⁶ independently is selected from the group consisting of hydrogen, F, Cl, Br, OH, OCH₃, OCF₃, CN, CF₃, CH₂F, CHF₂, CH₃, CHMe₂, —C≡CH, and —C≡C—CH₃; p₁₀ is 1-4;

each V⁶ independently is hydrogen, halo, oxo, COOH, cyano, nitro, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, OH, O-Me, OCF₃, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)—C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and

R¹ is COOH, CH═CHCO₂H, CONHOH, CONHNH₂, CON(Me)NH₂, CON(Me)NHMe, CON(Me)NMe₂, CONHNHMe, CONHNMe₂, CH═CHCONHOH, CH═CHCONHNH₂, CH═CHCON(Me)NH₂, CH═CHCON(Me)NHMe, CH═CHCON(Me)NMe₂, CH═CHCONHNHMe, CH═CHCONHNMe₂; —C≡C—CO₂H, —C≡C—CONHOH, —C≡C—CONHNH₂, —C≡C—CONHNMe, —C≡C—CONHNMe₂, —C≡C—CONMeNH₂, —C≡C—CONMeNHNe and —C≡C—CONMeNMe₂; with the proviso that when each V⁶ is H and R¹ is CH═CHCO₂H, CONHNH₂, or CONHNMe₂, then at least one of R⁶ is CN, CF₃, CH₃, CHMe₂, —C≡CH, and —C≡C—CH₃.

Within this embodiment, Y is CH₂; each R⁶ independently is hydrogen, F, Cl, Br, OH, OCH₃, OCF₃, CN, CF₃, CH₂F, CHF₂, CH₃, CHMe₂, —C≡CH, and —C≡C—CH₃; p10 is 1-4, each V⁶ independently is hydrogen, halo, oxo, COOH, cyano, nitro, CH₃, CH₂CH₃, CH(Me)₂, CF₃, CH₂F, CHF₂, hydroxymethyl, methoxymethyl, ethoxymethyl, cyclopropyl, cyclobutyl, 2-furanyl, 3-furanyl, ethynyl, 1-propynyl, 3-propynyl, O-Me, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)-C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and R¹ is COOH, CONH₂, CONHOH, CONHR³, CONHAr, CONH(Py) wherein Py is 2-, 3-, or 4-pyridyl, CONHSO₂R³, and CONHN═CR³R⁷.

Within this embodiment, R¹ is CONHNHR³; each V⁶ independently is hydrogen, halo, oxo, cyano, nitro, CH₃, CH₂CH₃, CH(Me)₂, methoxymethyl, ethoxymethyl, cyclopropyl, cyclobutyl, 2-furanyl, 3-furanyl, ethynyl, 1-propynyl, 3-propynyl, O-Me, O-Et, O-cyclopropyl, O-Aryl, S-Me, S-Et, NH₂, NHMe, NMe₂, NHAc, NHOH, NHNH₂, NHNHAc, NH—CONH₂, NMe-CONMe₂, NH—CSNH₂, NH—C(═NH)NH₂, N(Me)—C(═NMe)NMe₂, NH—CO₂Me, NH—SO₂Me, NH—SO₂-Aryl, COMe, COEt, COpropyl, CO-cyclopropyl, CSNMe₂, C(═NMe)NMe₂, CONHC(═NH)H₂, SO₂Me, SO₂Et, SOMe, SOEt, SO₃-Aryl, SO₂NH₂, or PO(NMe₂)₂; and R³ is

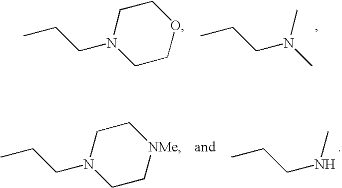

Within any of the above embodiments, R¹ is L¹-V⁵ or CO₂R³. In another embodiment R¹ is C₂alkenyl-CO₂R³. In another embodiment, R³ is H or (CH₂)_{q}NR³₂; each R¹³ is independently (C₁-C₈) alkyl, or, if both present on the same substituent may be joined together to form a three- to eight-membered heterocyclyl ring system; and the subscript n is an integer of from 1 to 4.

Within any of the above embodiments, R⁶ is independently selected from the group consisting of Cl, Br, or CH₃. In another embodiment, each R⁶ is Cl.

In another embodiment, (GROUP 52) the present invention provides compounds selected from the group consisting of formulae (V-A), (V-B), (V-C), (V-D), (V-E), (V-F), (V-G), (V-H), (V-I) and (V-J): 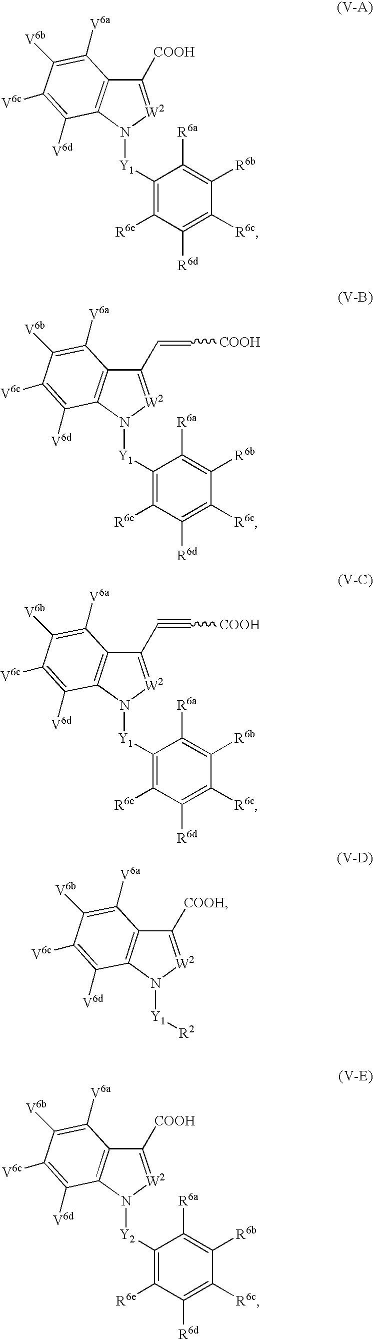 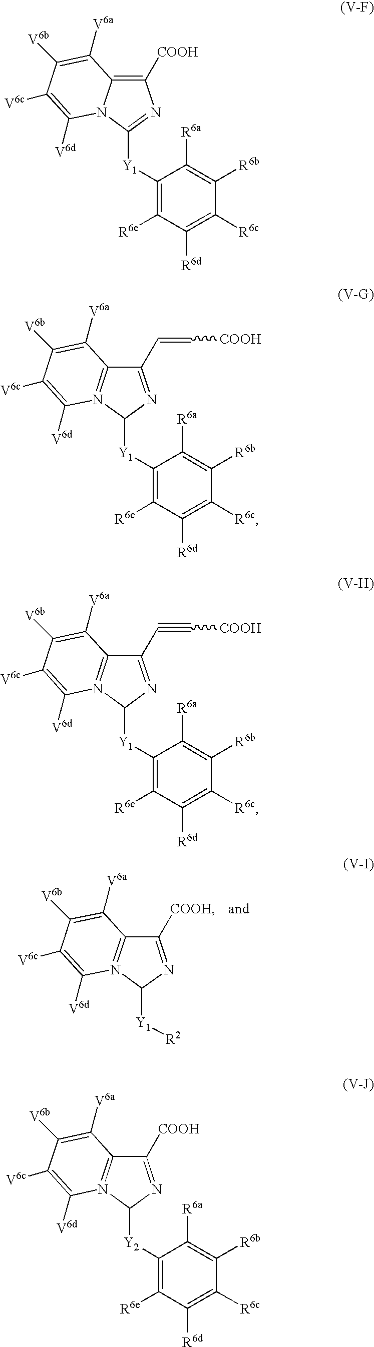
wherein

each V^{6a}, V^{6b}, V^{6c} and V^{6d} are independently a member selected from the group consisting of hydrogen, halogen, C₁-C₈ alkyl, C₁-C₈ heteroalkyl, aryl, heteroaryl, hydroxyl, amino, alkylamino, dialkylamino, nitro, cyano, C₂-C₆ alkenyl, C₂-C₆ alkynyl, C₁-C₆ alkoxy, and CO₂R³;

each R^{6a}, R^{6b}, R^{6d}, Rd and R^{6e} are independently a member selected from the group consisting of H, halogen, C₁-C₈alkyl, C₁-C₈ heteroalkyl, aryl, heteroaryl, NR³COR³, hydroxy, alkoxy and CO₂R³, or R^{6c} and R^{6d} may be taken together to form a dioxomethylene bridge;

R² is a defined above;

W² is N, CH or CO;

Y₁ is C(R⁸)₂ wherein R⁸ is hydrogen, alkyl, heteroalkyl, aryl or heteroaryl;

Y₂ is CO or SO₂;

and pharmaceutically acceptable salts thereof.

Within this embodiment, R² is selected from the group consisting of: 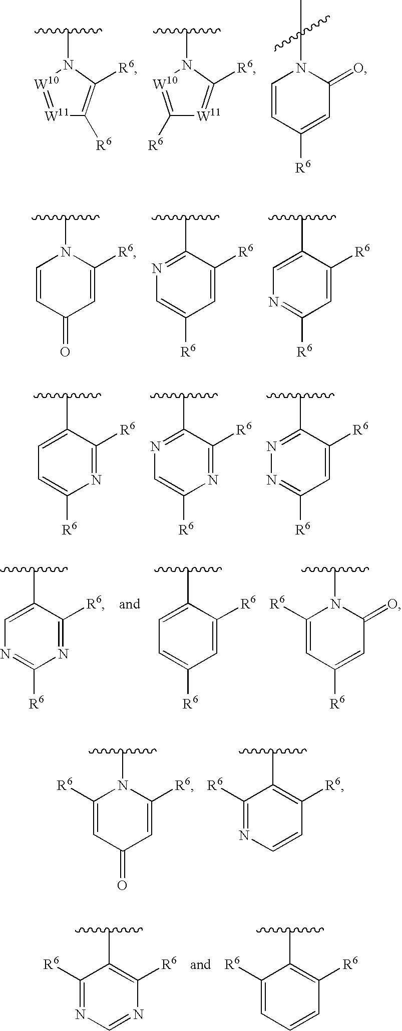
wherein each W¹⁰ or W¹¹ is preferably, independently selected from the group consisting of N, C and CH; and the wavy line indicates the point of attachment to the rest of the molecule. In one embodiment, each R⁶ independently is hydrogen, F, Cl, Br, OH, OCH₃, OCF₃, CN, CF₃, CH₃, CH₂F, CHF₂, CHMe₂, —C≡CH and —C≡C—CH₃; each V⁶ independently is hydrogen, F, Cl, Br, COOH, CH₃, CH₂CH₃, CH(Me)₂, CF₃, CH₂F, CHF₂, hydroxymethyl, methoxymethyl, ethoxymethyl, OH, O-Me, OCF₃, O-Et, O-cyclopropyl and O-Aryl; and R⁸ is hydrogen, Me, ethyl, propyl, i-propyl or MeOCH₂. In one embodiment, W² is N; R^{6a} and R^{6c} is Cl, and each V⁶ is hydrogen. In one embodiment, W² is CH; each V⁶ independently is hydrogen, F, Cl, Br, CH₃, CH(Me)₂, CF₃, hydroxymethyl, OH, O-Me and OCF₃, R^{6a} is Cl, and each R^{6c} and R^{6c} is Cl or hydrogen. In one embodiment, R² is selected from the group consisting of: 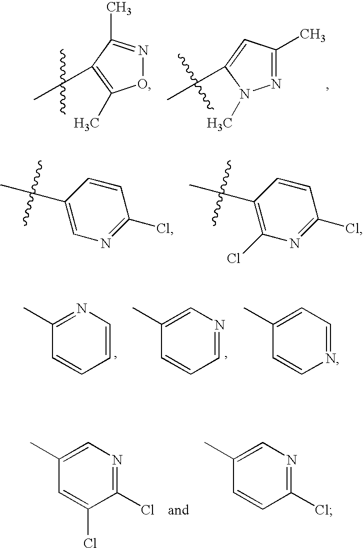
the wavy line indicates the point of attachment to the rest of the molecule. In another embodiment, (GROUP 53), compounds have the formula selected from the group consisting of formulae (V-A), (V-B), (V-C), (V-D), (V-E) and (V-F) provided that the compound does not have a formula selected from the group consisting of the following compounds (GROUPS K1-K6):

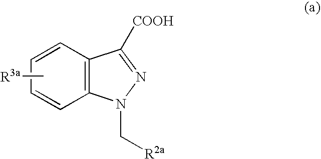

(i) wherein referring to formula (a)

(a) GROUP K1
R^{3a} is hydrogen;
R^{2a} is selected from the group consisting of 4-chlorophenyl, 3-chlorophenyl, 2-chlorophenyl, 4-fluorophenyl, 4-bromophenyl, 4-iodophenyl, 3-trifluoromethylphenyl, 4-cyanophenyl, 4-phenylsulfonyl-phenyl, 3,4-dichlorophenyl, 2,4-dichlorophenyl, 2,6-dichlorophenyl, 2,4-dibromophenyl, 2,4,5-trichlorophenyl, 4-chlorophenyl, 4-methylphenyl, 3-methylphenyl, 2-methylphenyl, 4-chlorophenyl, 3-benzoylphenyl, 4-methylsulfonylphenyl, 4-chloronaphthylmethyl, 2,4-dimethylphenyl and 2-methyl-4-chlorophenyl;

(b) GROUP K2
R^{2a} is 4-chlorophenyl; and
R^{3a} is chloro, OH, methyl, or OMe;

(c) GROUP K3
R^{2a} is 2,4-dichlorophenyl,
R^{3a} is selected from the group consisting of —(OCH₃)ₙ₁₀ wherein ₙ₁₀ is 1 or 2, chloro, bromo, fluoro, CO₂H, and CH₂CO₂H;

(ii) compounds having the formulae (a4) and (a5) (GROUP K4):

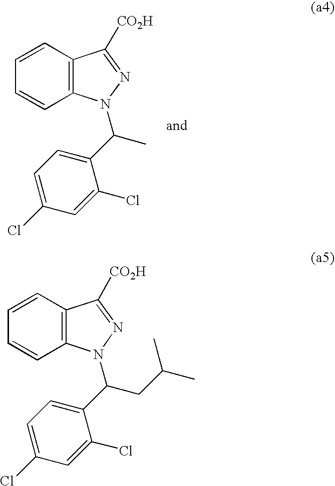

(iii) compounds having the formula (a6) (GROUP K5):

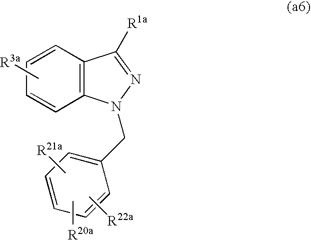

wherein R^{1a} is COOH,
R^{22a} is H or halo,
R^{20a} is halo, Me, methoxy, trifluoromethyl, CONH2, or methanesulfonyl, and
R^{21a} is H, Me, halo, or a group forming with the benzene ring to which it is attached a naphthyl ring; and
R^{3a} is H, Me, methoxy and halogen; and

(iv) compounds having the formula (GROUP K6): 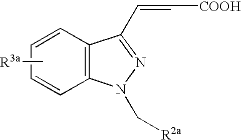
R^{2a} is a group having the formula: 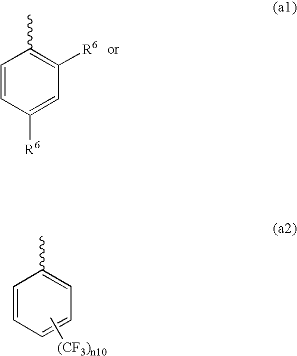
wherein each R⁶ independently is a halogen, and n₁₀ is 1 or 2; and
R^{3a} is hydrogen.

In another embodiment, (GROUP 54), compounds have the formula selected from the group consisting of formulae (V-A), (V-B), (V-C), (V-D), (V-E) and (V-F) provided that the compound does not have a formula selected from the group consisting of the following compounds (GROUPS L¹ and L²):

(a) GROUP L¹—referring to formula (V-A),

(i) wherein V^{6b} is methyl, dimethylamino, CF₃, CHF₂, CH₂F, Cl, F, OCF₃ or CH₂OH; and each R^{6a} and R^{6c} is Cl or F;

(ii) wherein V^{6c} is methyl, dimethylamino, CF₃, CHF₂, CH₂F, Cl, F, OCH₃, OCF₃, CH₂OH or COOH; and each R^{6a} and R^{6c} is Cl or F; and

(iii) wherein V^{6c} is OCH₃; R^{6a} is methyl and R^{6c} is Cl; and

(b) GROUP L² referring to formula (V-B),

(i) wherein V^{6b} is CF₃ or Cl; and each R^{6a} and R^{6c} is Cl or F;

(ii) wherein V^{6c} is methyl, dimethylamino, CF₃, CHF₂, CH₂F, Cl, F, OCH₃, OCF₃, CH₂OH or COOH; and each R^{6a} and R^{6c} is Cl or F; and

(iii) wherein V^{6c} is OCF₃; and each R^{6a} and R^{6c} is F, Cl or methyl; and

(iv) wherein V^{6c} is CF₃; R^{6a} is methyl and R^{6c} is Cl;

In another embodiment, (GROUP 55) the present invention provides compounds having a formula selected from the group consisting of formulae (VI-A), (VI-B), (VI-C), (VI-D), (VI-E) and (VI-F): 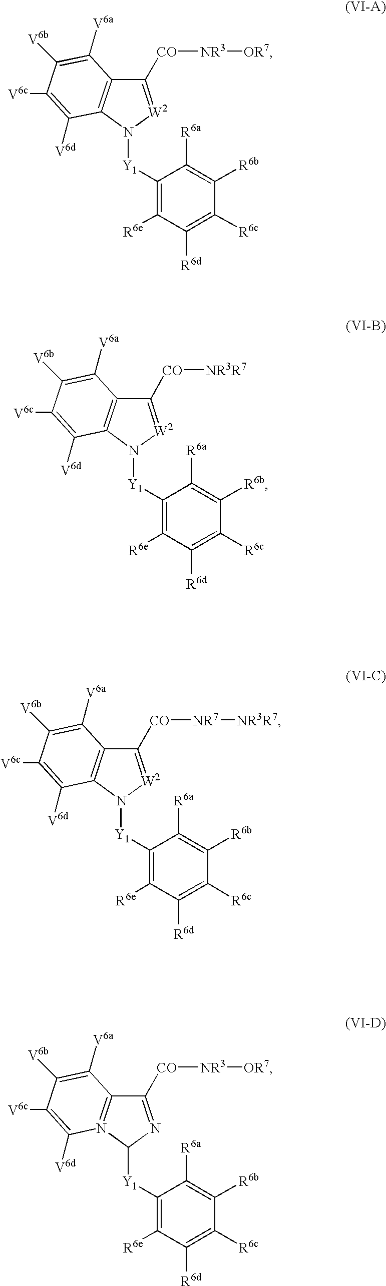 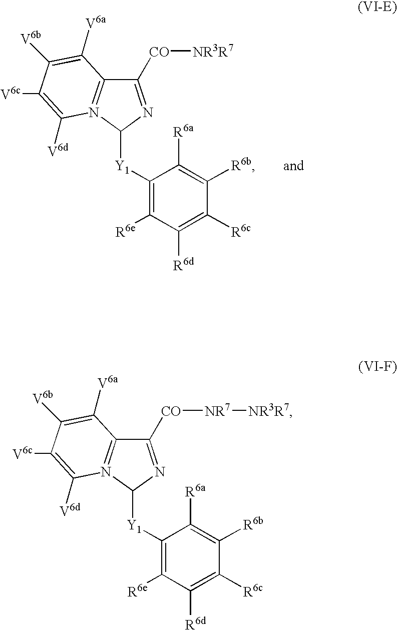
wherein

each V^{6a}, V^{6b}, V^{6c} and V^{6d} are independently a member selected from the group consisting of hydrogen, halogen, C₁-C₈ alkyl, C₁-C₈ heteroalkyl, aryl, heteroaryl, hydroxyl, amino, alkylamino, dialkylamino, nitro, cyano, C₂-C₆ alkenyl, C₂-C₆ alkynyl, C₁-C₆ alkoxy, and CO₂R³,

each R^{6a}, R^{6b}, R^{6c}, R^{6d} and R^{6e} are independently a member selected from the group consisting of H, halogen, C₁-C₈ alkyl, C₁-C₈ heteroalkyl, aryl, heteroaryl, NR³COR³, hydroxy, alkoxy and CO₂R³ or R^{6c} and R^{6d} may be taken together to form a dioxomethylene bridge;

W² is N, CH or CO;

Y₁ is C(R⁸)₂ wherein R⁸ is hydrogen, alkyl heteroalkyl, aryl or heteroaryl;

each R³ and R⁷ is a member independently selected from the group consisting of hydrogen, C₁-C₈ alkyl, C₁-C₈ heteroalkyl, C₃-C₈ cycloalkyl, C₃-C₈ heterocyclyl, aryl, heteroaryl; R³ and R⁷ taken together form a C₃-C₈ heterocyclyl or heteroaryl ring;

and pharmaceutically acceptable salts thereof.

In one embodiment, each R⁶ independently is hydrogen, F, Cl, Br, OH, OCH₃, OCF₃, CN, CF₃, CH₃, CH₂F, CHF₂, CHMe₂, —C≡CH, and —C≡C—CH₃; each V⁶ independently is hydrogen, F, Cl, Br, COOH, CH₃, CH₂CH₃, CH(Me)₂, CF₃, CH₂F, CHF₂, hydroxymethyl, methoxymethyl, ethoxymethyl, OH, O-Me, OCF₃, O-Et, O-cyclopropyl and O-Aryl; and R⁸ is hydrogen, Me, ethyl, propyl, i-propyl or MeOCH₂. In one embodiment, W² is N; R^{6a} and R^{6c} is Cl, and each V⁶ is hydrogen. In one embodiment, W² is CH; each V⁶ independently is hydrogen, F, Cl, Br, CH₃, CH(Me)₂, CF₃, hydroxymethyl, OH, O-Me and OCF₃, R^{6a} is Cl, and each R^{6c} and R^{6c} is Cl or hydrogen.

In another embodiment, (GROUP 56), compounds have the formula selected from the group consisting of formulae (VI-A), (VI-B) and (VI-C) provided that the compound does not have a formula selected from the group consisting of the following compounds (GROUPS M1-M6):

(i) GROUP M1, wherein each R³ and R⁷ is hydrogen; each V^{6a}, V^{6b}, V^{6c} and V^{6d} are hydrogen; and R^{6a} and R^{6c} are H or Cl;

(ii) GROUP M2—compound having the formula (VI-C) wherein each R³ and R⁷ is hydrogen; each V^{6a}, V^{6b}, V^{6c} and V^{6d} are hydrogen; R^{6a} is methyl and R^{6c} is Cl;

(iii) wherein referring to formula (a)

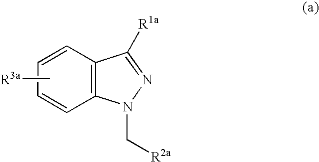

(a) GROUP M3
R^{1a} is selected from the group consisting of CONHNH₂ and CONHN(CH₃)₂;
R^{2a} is a group having the formula: 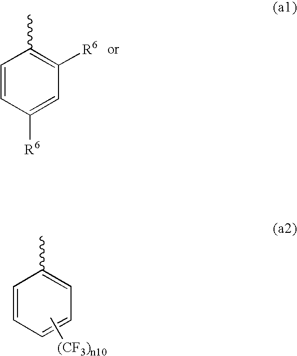
wherein each R⁶ independently is a halogen, and n10 is 1 or 2; and
R^{3a} is hydrogen;

(b) GROUP M4
R^{1a} is CONH₂,
R^{2a} is 4-chlorophenyl, and
R^{3a} is H; and

(c) GROUP M5
R^{1a} is CONHCH(CO₂H)₂ or CONH(CH₂)ₙ₁₁-cyclopropyl wherein n₁₁ is 0 or 1,
R^{2a} is 2,4-dichlorophenyl,
R^{3a} is H; and

(iv) GROUP M6—compounds having the formula (a6):

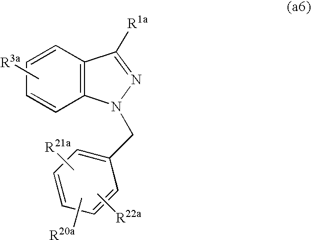

wherein R^{1a} is CONH₂;
R^{22a} is H or halo,
R^{20a} is halo, Me, methoxy, trifluoromethyl, CONH2, or methanesulfonyl, and
R^{21a} is H, Me, halo, or a group forming with the benzene ring to which it is attached a naphthyl ring; and
R^{3a} is H, Me, methoxy and halogen.

In another embodiment, (GROUP 57), compounds have the formula selected from the group consisting of formulae (VI-A), (VI-B) and (VI-C) provided that the compound does not have a formula selected from the group consisting of the following compounds (GROUP N1-N3):

(a) referring to formula (VI-C),

(i) GROUP N1, wherein R³ is H; R⁷ is methyl; each V^{6a}, V^{6b}, V^{6c} and V^{6d} are hydrogen; and R^{6a} and R^{6c} are Cl;

(ii) GROUP N2, wherein each R³ and R⁷ is hydrogen; V^{6b} is CF₃, or Cl; and each R^{6a} and R^{6c} is Cl or F; and

(iii) GROUP N3, wherein each R³ and R⁷ is hydrogen; V^{6c} is methyl, dimethylamino, CF₃, CHF₂, CH₂F, Cl, F, OCH₃, OCF₃ or CH₂OH; and each R^{6a} and R^{6c} is Cl or F.

In another embodiment, (GROUP 58) the present invention provides compounds having a formula selected from the group consisting of formulae (VII-A) and (VII-B): 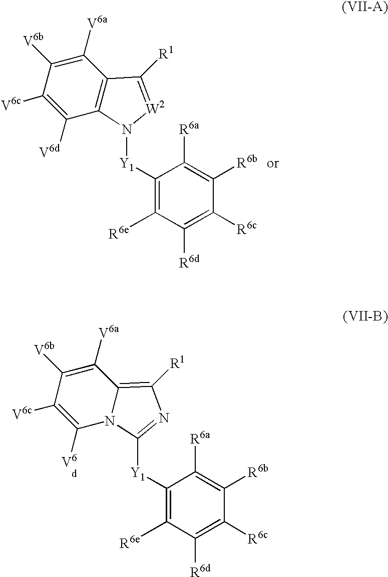
wherein

R¹ is selected from CHO, CR³R⁷OR⁷, CONR³SO₂R⁷, SO₂NR³R⁷, and tetrazole;

each V^{6a}, V^{6b}, V^{6c} and V^{6d} are independently a member selected from the group consisting of hydrogen, halogen, C₁-C₈ alkyl, C₁-C₈ heteroalkyl, aryl, heteroaryl, hydroxyl, amino, alkylamino, dialkylamino, nitro, cyano, C₂-C₆ alkenyl, C₂-C₆ alkynyl, C₁-C₆ alkoxy, and CO₂R³;

each R^{6a}, R^{6b}, R^{6c}, R^{6d} and R^{6e} are independently a member selected from the group consisting of H, halogen, C₁-C₈ alkyl, C₁-C₈ heteroalkyl, aryl, heteroaryl, NR³COR³, hydroxy, and alkoxy or R^{6c} and R^{6d} may be taken together to form a dioxomethylene bridge;

each R³ and R⁷ is a member independently selected from the group consisting of hydrogen, C₁-C₈ alkyl, C₁-C₈ heteroalkyl, C₃-C₈ cycloalkyl, C₃-C₈ heterocyclyl, aryl, heteroaryl;

W² is N, CH or CO;

Y₁ is C(R⁸)₂ wherein R⁸ is hydrogen, alkyl heteroalkyl, aryl or heteroaryl;

and pharmaceutically acceptable salts thereof.

In one embodiment, each R⁶ independently is hydrogen, F, Cl, Br, OH, OCH₃, OCF₃, CN, CF₃, CH₃, CH₂F, CHF₂, CHMe₂, —C≡CH, and —C≡C—CH₃; each V⁶ independently is hydrogen, F, Cl, Br, COOH, CH₃, CH₂CH₃, CH(Me)₂, CF₃, CH₂F, CHF₂, hydroxymethyl, methoxymethyl, ethoxymethyl, OH, O-Me, OCF₃, O-Et, O-cyclopropyl and O-Aryl; and R⁸ is hydrogen, Me, ethyl, propyl, i-propyl or MeOCH₂. In one embodiment, W² is N; R^{6a} and R^{6c} is Cl, and each V⁶ is hydrogen. In one embodiment, W² is CH; each V⁶ independently is hydrogen, F, Cl, Br, CH₃, CH(Me)₂, CF₃, hydroxymethyl, OH, O-Me and OCF₃, R^{6a} is Cl, and each R^{6c} and R^{6c} is Cl or hydrogen.

In another embodiment, (GROUP 59), compounds have the formula selected from the group consisting of formulae (VII-A) and (VII-B) provided that the compound does not have a formula selected from the group consisting of the following compounds (GROUP 0):

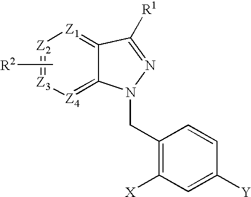

wherein R¹ is CH₂OH or CHO; R² is hydrogen, halogen, alcohol, alkyl, alkoxy, aralkyl, cycloalkyl, haloalkyl, haloalkyl, amino, or carboxyl; each X and Y are halogen or lower alkyl; and Z₁, Z₂, Z₃ and Z₄ are independently N or C.

In another embodiment, (GROUP 60), compounds have the formula selected from the group consisting of formulae (VII-A) and (VII-B) provided that the compound does not have a formula selected from the group consisting of the following compounds (GROUPS P1 and P2):

(i) GROUP P1—referring to formula (VII-A) wherein each of V^{6a}, V^{6b}, V^{6c}, V^{6d} is hydrogen, halogen, alcohol, alkyl, alkoxy, aralkyl, cycloalkyl, haloalkyl, haloalkoxy, amino or carboxyl; and each R^{6a} and R^{6c} is halogen or lower alkyl; and

(ii) GROUP P2—referring to formula (a)

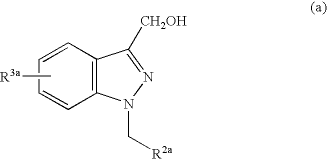

R^{2a} is selected from the group consisting of phenyl, 2-chlorophenyl, 2-methylphenyl, 3-fluorophenyl, 3-chlorophenyl, 3-bromophenyl, 3-methylphenyl, trifluoromethylphenyl, 3-benzoyl, 4-halophenyl, 4-methylsulfonylphenyl, 4-methylphenyl, 4-cyanophenyl, 4-phenylsulfonylphenyl, 4-methoxyphenyl, 4-chloronapth-1-yl, 2,3-dimethylphenyl, 2,4-dihalophenyl, 2,4-dimethylphenyl, 2,6-dichlorophenyl, 2,6-dimethylphenyl, 3,4-dichlorophenyl, bis-trifluoromethylphenyl, 4-chloro-2-methylphenyl, 5-chloro-2-methoxyphenyl, 2,4,5-trichlorophenyl, 2,6-dimethyl-3-dimethylsulfamoylphenyl, 4-imidazoyl; and

R^{3a} is selected from the group consisting of H, 2-dimethylaminoethyl, 5-amino, chloro, bromo, 5-hydroxy, 5-methyl, methoxy, dimethoxy, fluoro, CO₂H, CH₂CO₂H, 5-nitro, 5-acetamido and 7-chloro.

Still other groups of embodiments are provided in the Examples below.

Examples of compounds of Formula 1 include:
1-(2,4-Dichloro-benzyl)-imidazo[1,5-a]pyridine-3-acrylic acid; 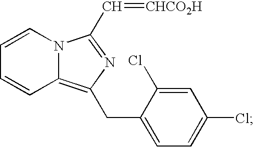
3-(2,4-Dichloro-benzyl)-imidazo[1,5-a]pyridine-3-acrylic acid: 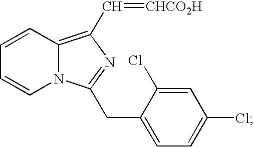
3-(2,4-Dichloro-benzyl)-benzo[c]thiophene-1-acrylic acid: 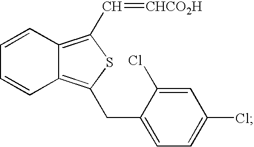
3-(2,4-Dichloro-benzyl)-isobenzofuran-1-acrylic acid: 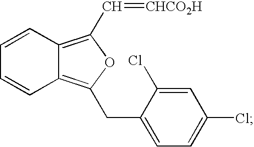
3-(2,4-Dichloro-benzyl)-2H-isoindole-1-carboxylic acid: 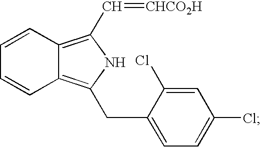
1-(2,4-Dichloro-benzyl)-imidazo[1,5-a]pyridine-3-carboxylic acid: 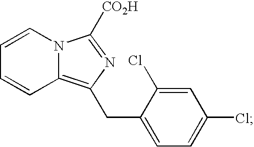
3-(2,4-Dichloro-benzyl)-imidazo[1,5-a]pyridine-1-carboxylic acid: 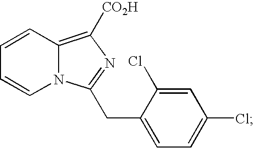
3-(2,4-Dichloro-benzyl)-benzo[c]thiophene-1-carboxylic acid: 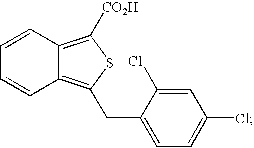
3-(2,4-Dichloro-benzyl)-isobenzofuran-1-carboxylic acid: 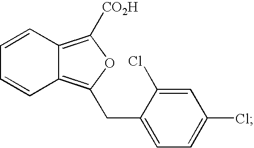
3-(2,4-Dichloro-benzyl)-2H-isoindole-1-carboxylic acid: 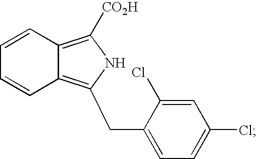
3-(2,4-Dichloro-benzyl)-2H-isoindole-1-carboxylic acid: 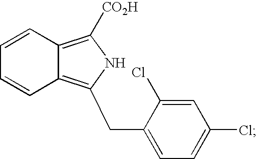
3-(2,4-Dichloro-benzyl)-benzo[c]thiophene-1-carboxylic acid: 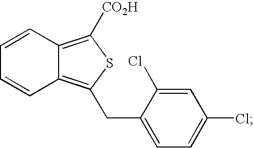
1-(4-Chloro-2-methyl-benzyl)-2-oxo-1,2-dihydro-cyclohepta[b]pyrrole-3-acrylic acid: 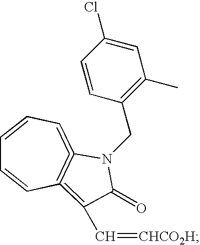
1-(4-Chloro-2-methyl-phenylamino)-2-oxo-1,2-dihydro-cyclohepta[b]pyrrole-3-acrylic acid: 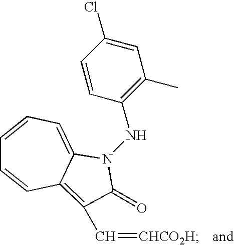
and 1-(4-Chloro-2-methyl-phenoxy)-2-oxo-1,2-dihydro-cyclohepta[b]pyrrole-3-acrylic acid: 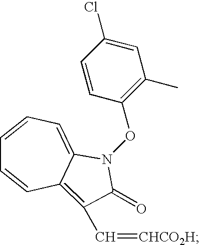
1-(4-Chloro-2-methyl-benzyl)-2-oxo-1,2-dihydro-cyclohepta[b]pyrrole-3-carboxylic acid: 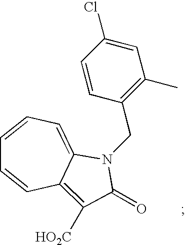
1-(4-Chloro-2-methyl-phenylamino)-2-oxo-1,2-dihydro-cyclohepta[b]pyrrole-3-carboxylic acid: 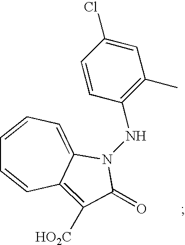
and 1-(4-Chloro-2-methyl-phenoxy)-2-oxo-1,2-dihydro-cyclohepta[b]pyrrole-3-carboxylic acid: 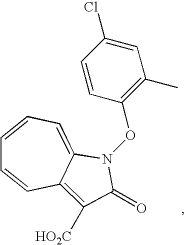 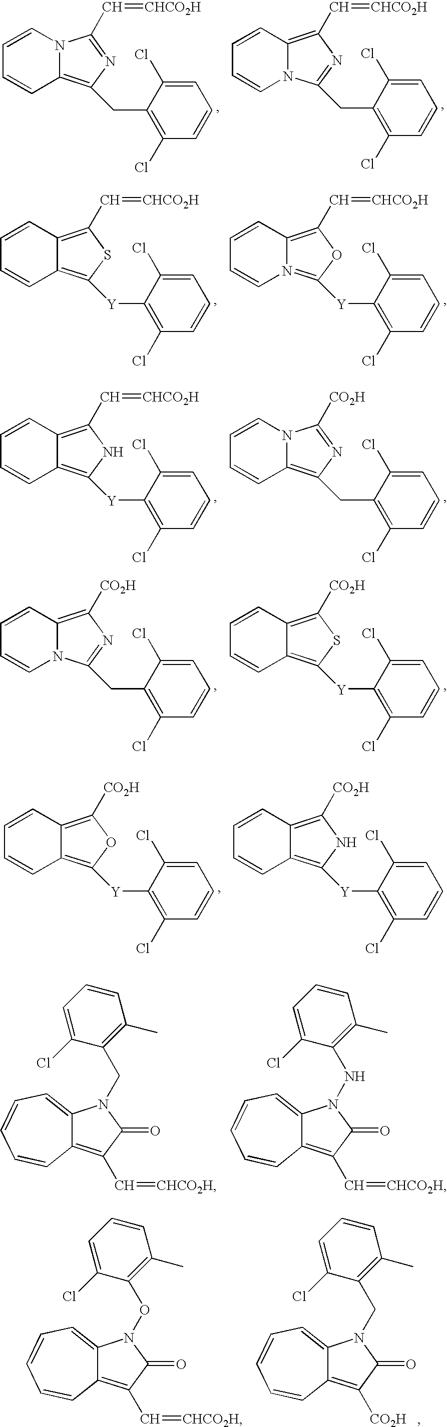 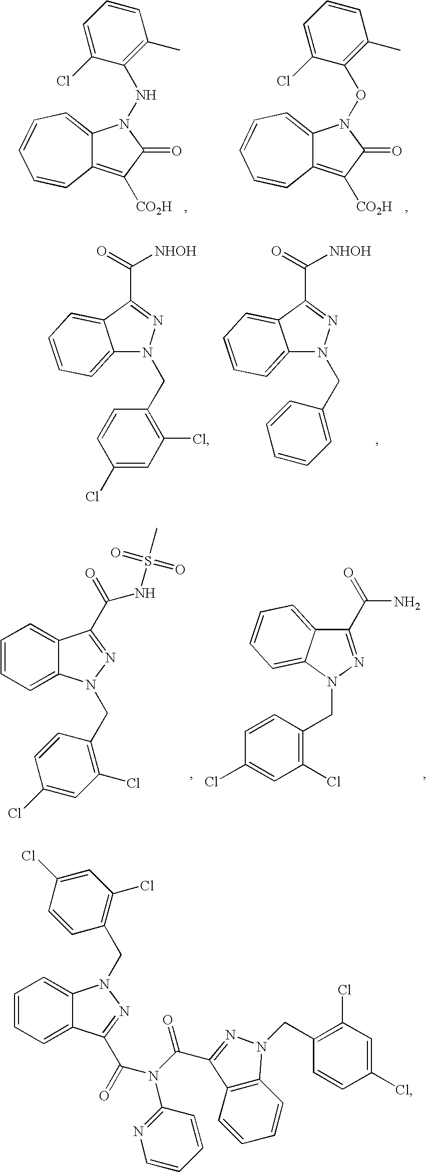 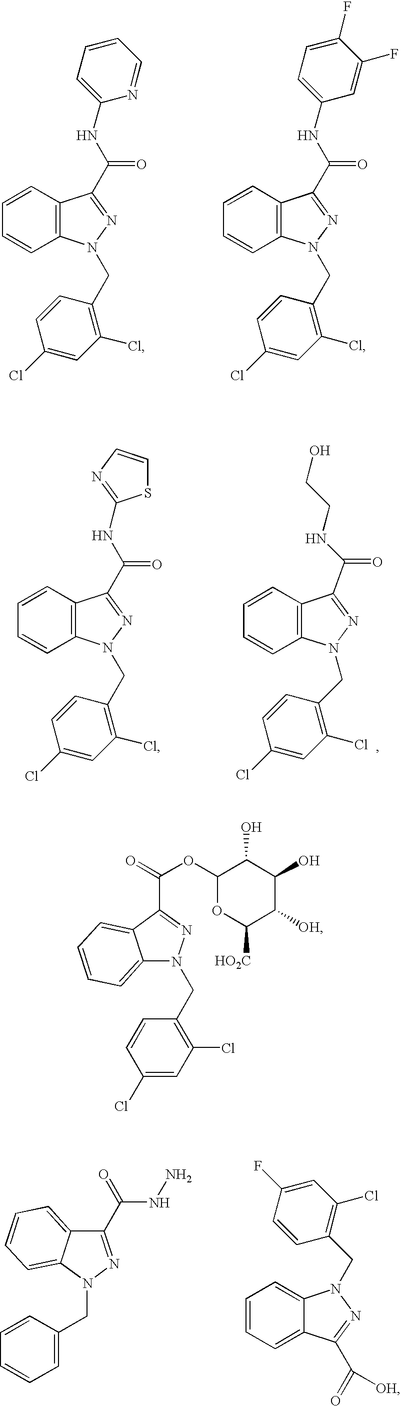 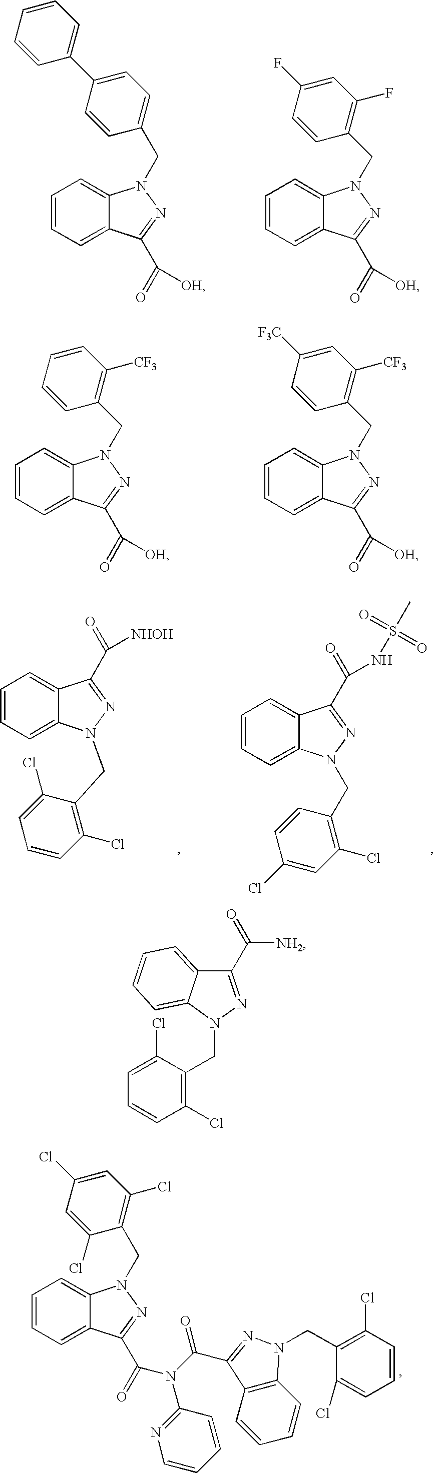 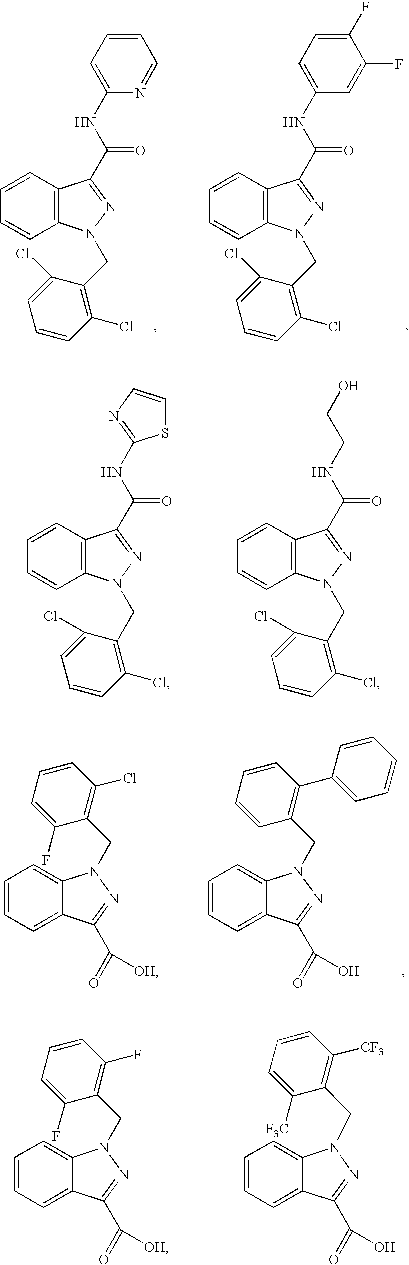 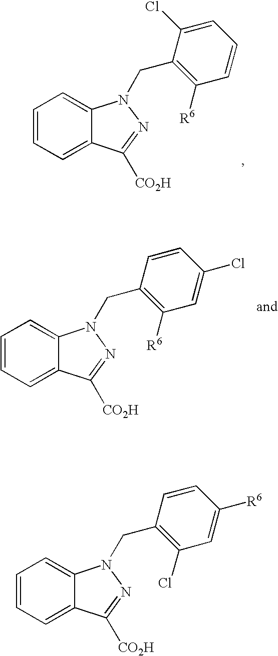
   wherein R⁶ is selected from the group consisting of H, F, Br, CN, CF₃, CH₃ CHMe₂, —C≡C—CH₃, CONHMe;

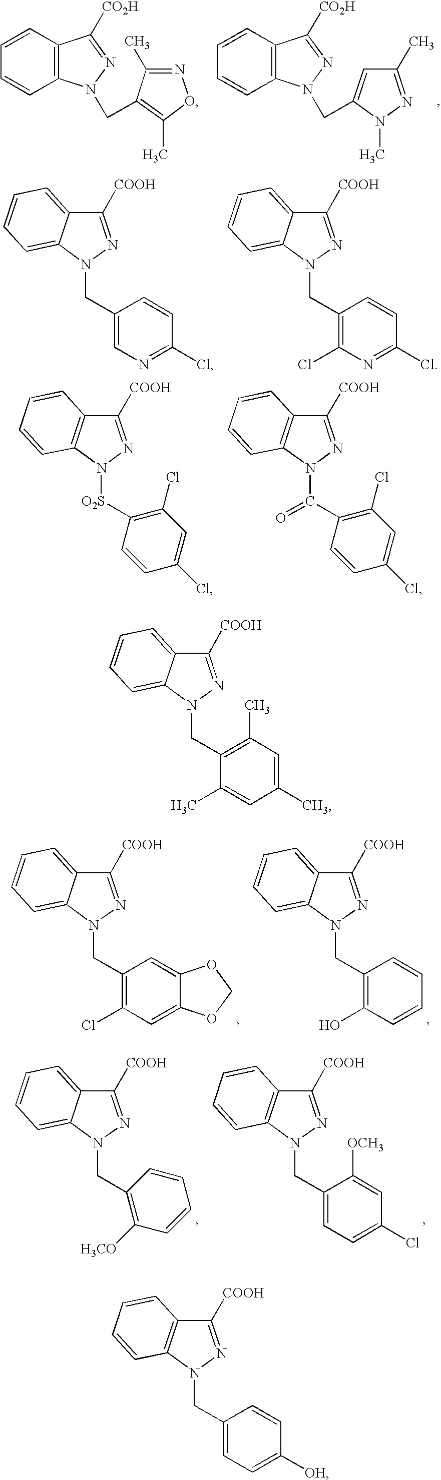

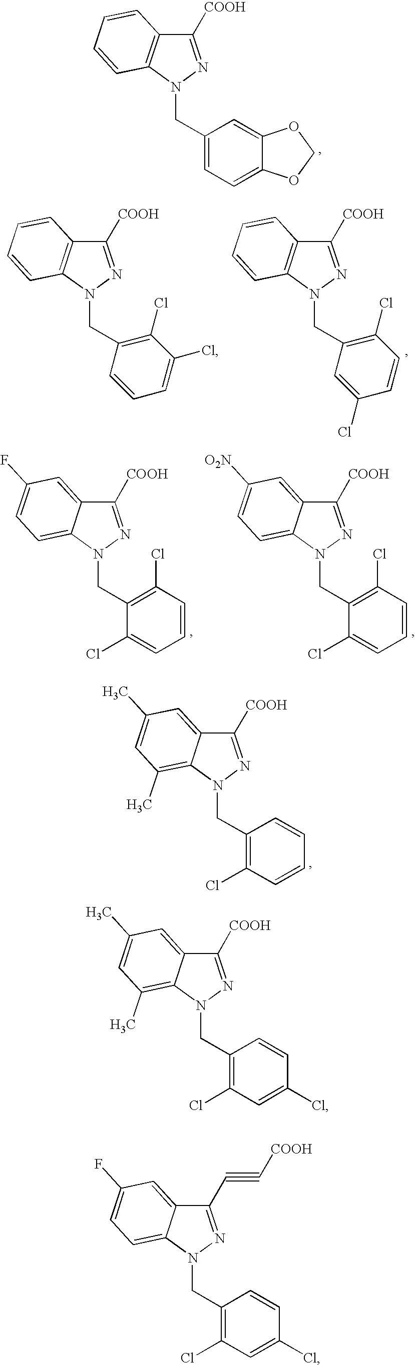

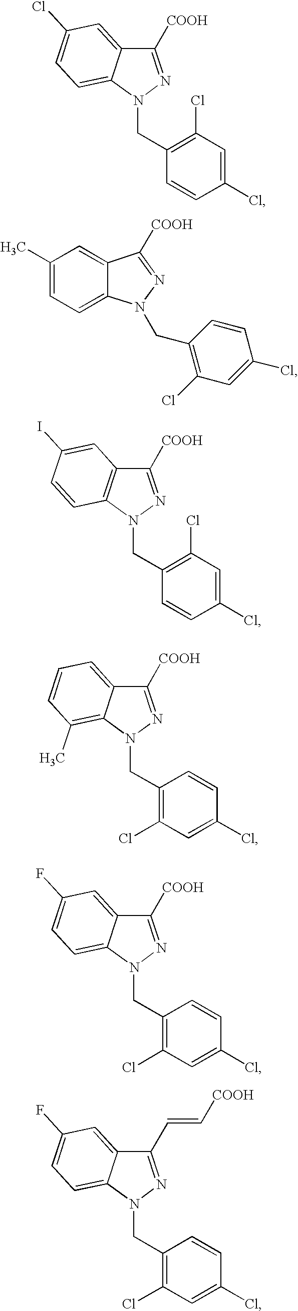

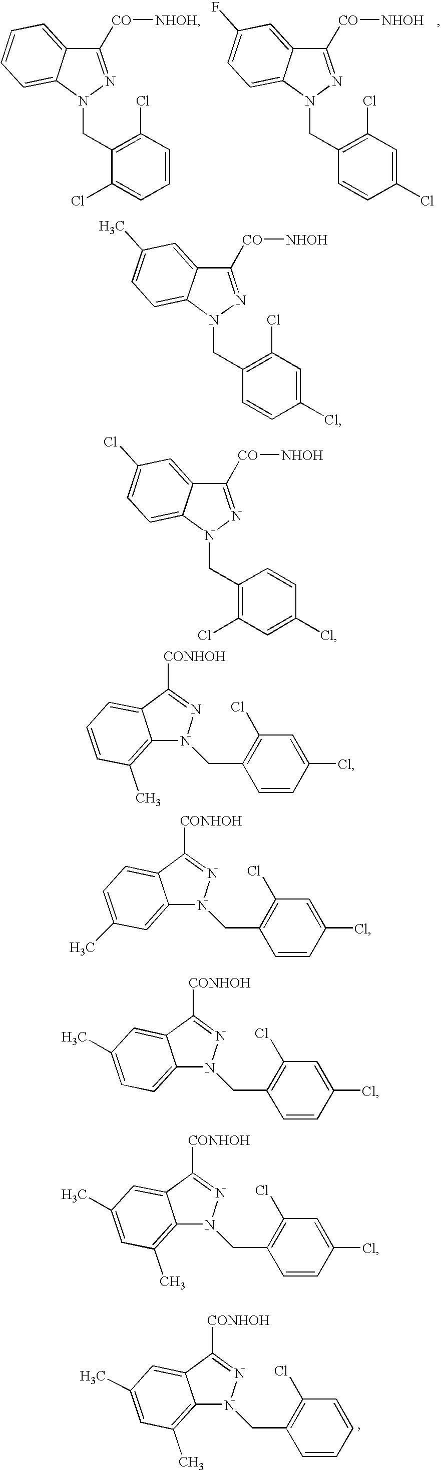

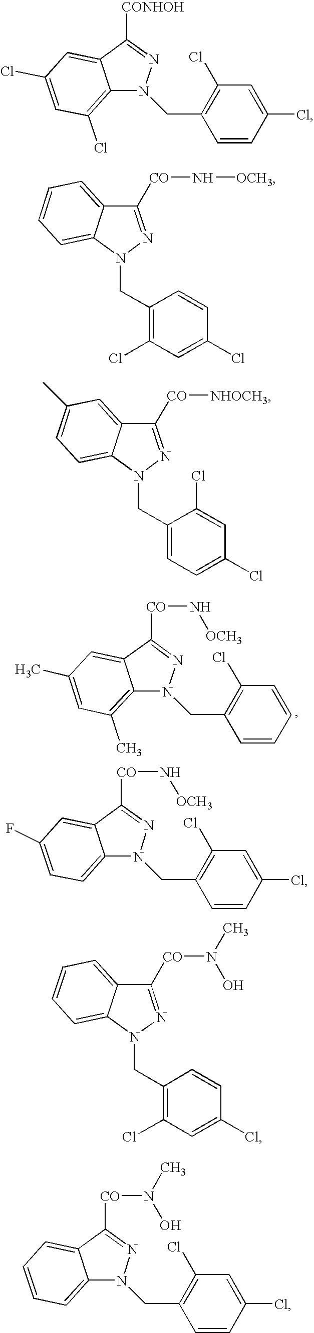

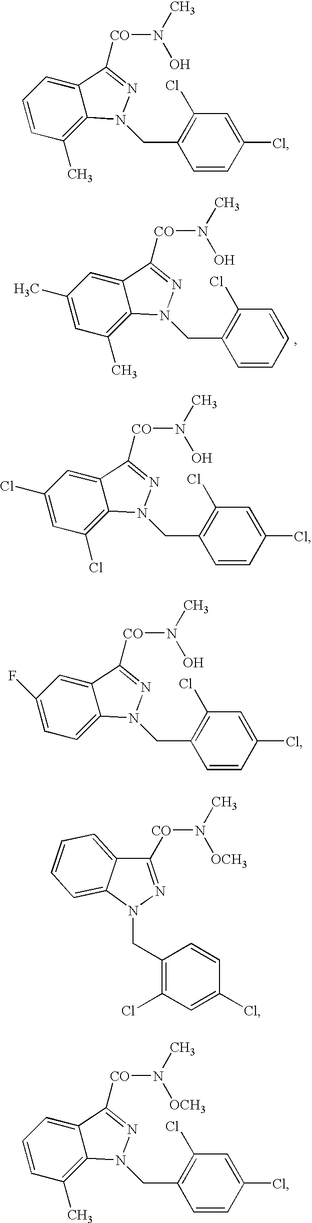

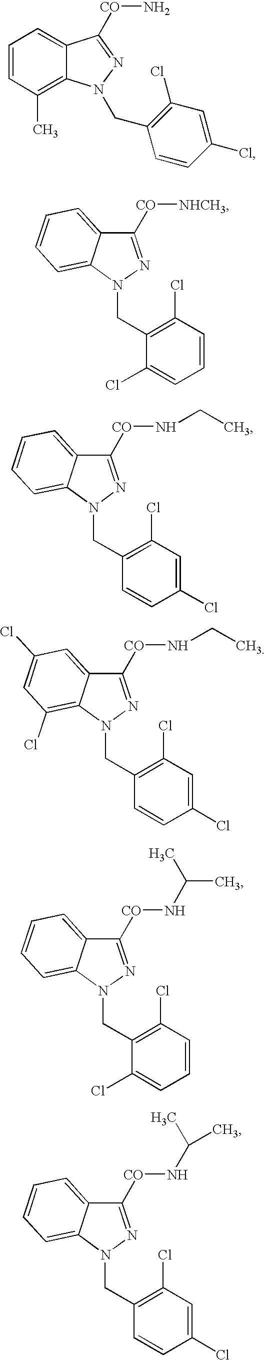

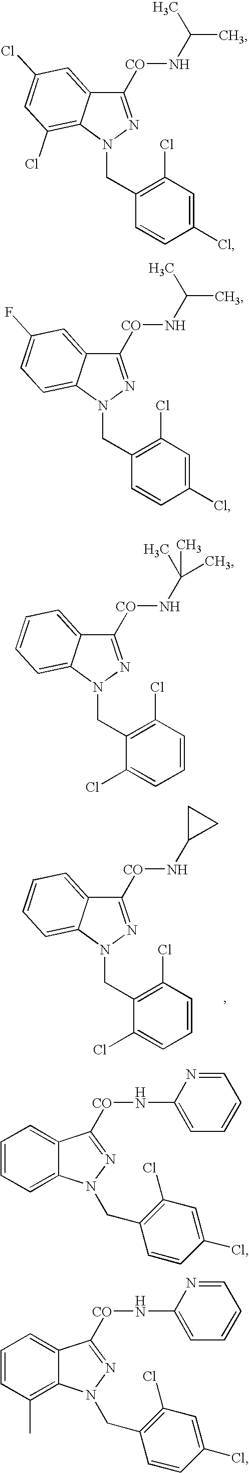

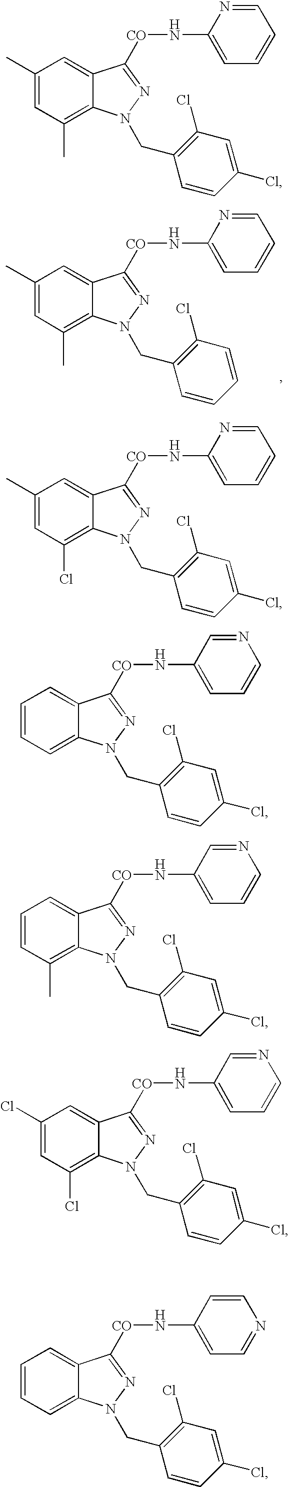

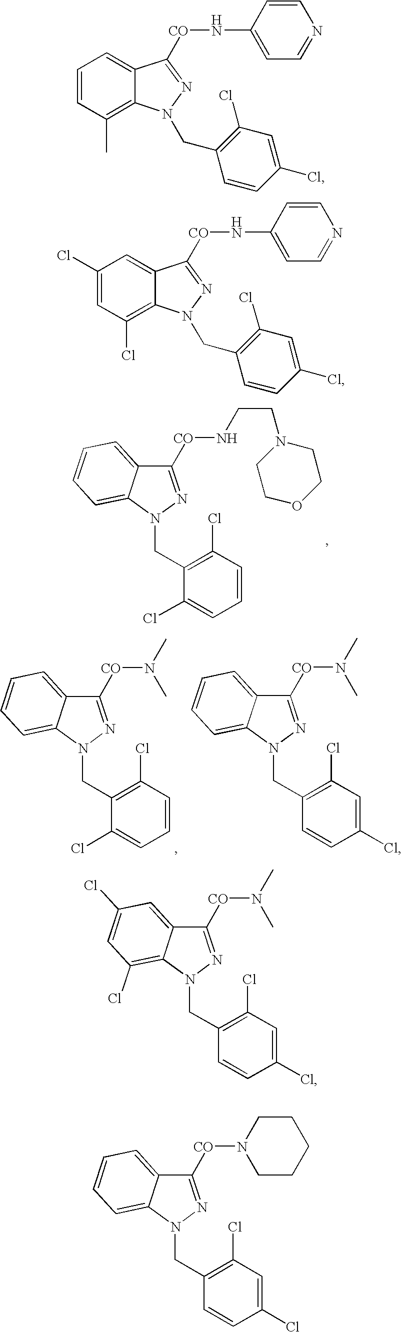

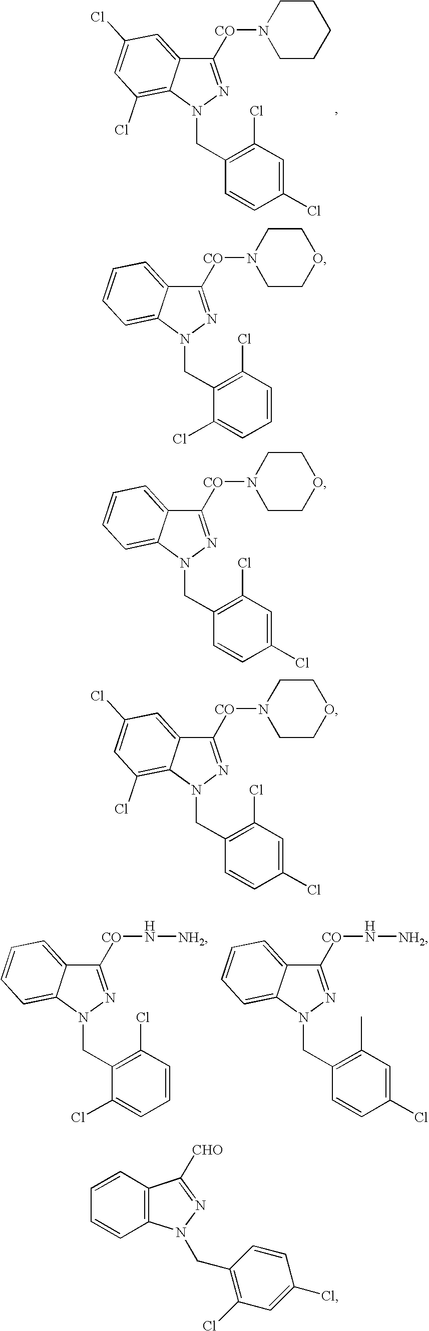

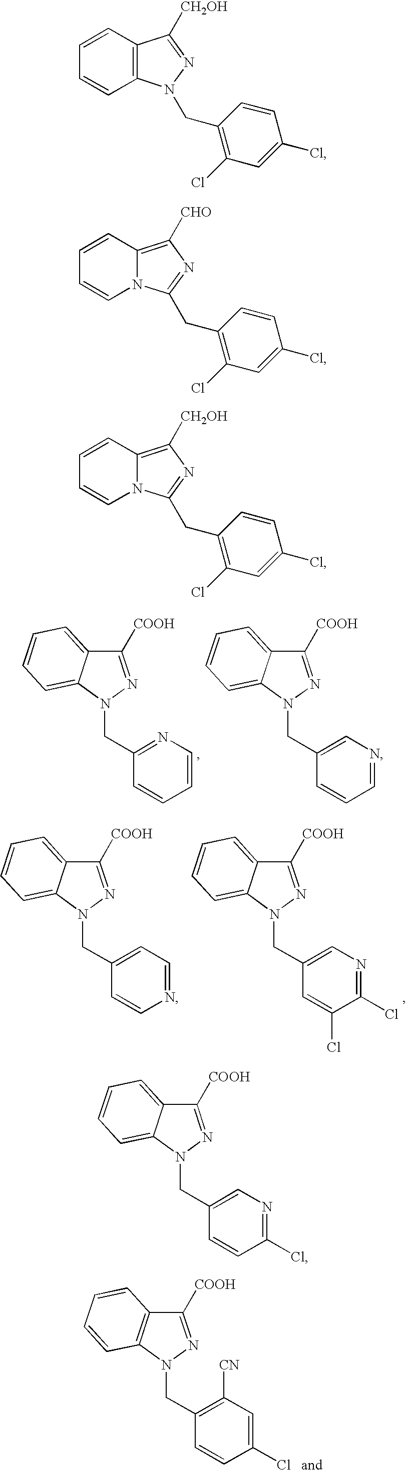

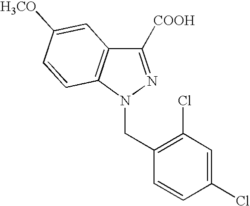

In another embodiment the compounds of the present invention include all of the compounds of the examples.

Monocyclic Embodiments

In another embodiment, compounds of the present invention have the formulae: 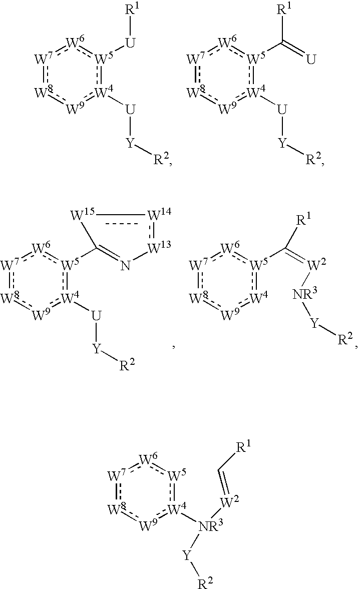
wherein the variables are as defined herein.

In another embodiment, compounds of the present invention have the formulae: 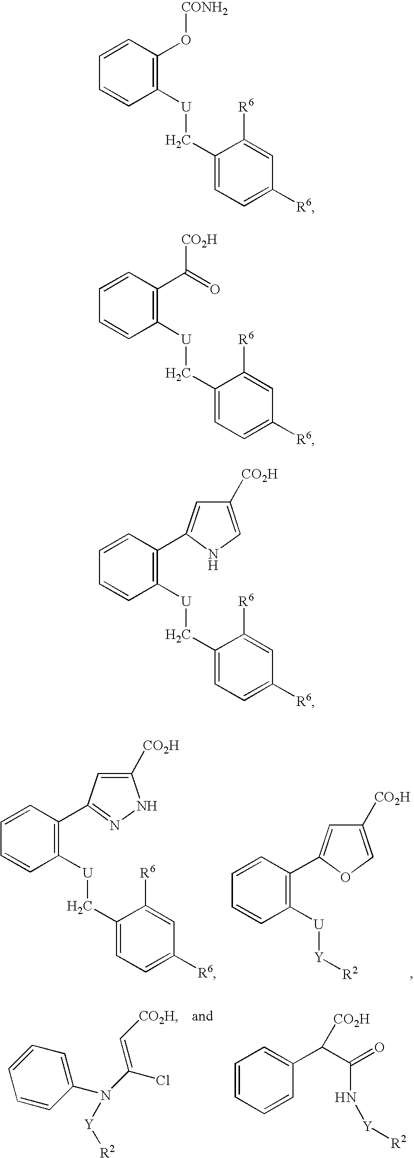
wherein the variables are as defined herein.

In another embodiment, compounds of the present invention have the formulae: 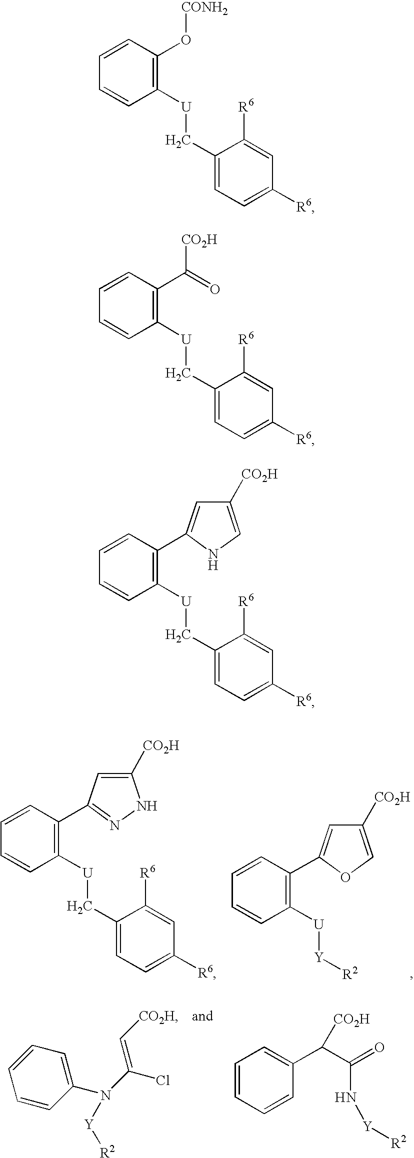
wherein the variables are as defined herein.
Acid Bioisosteres

In one aspect, R¹ is a bioisostere of CO₂H, CONH₂, CONHNH₂, or a derivative thereof selected from a cyclic 4, 5, or 6 membered hetero cycle, arene or heteroerene. In one embodiment, a squaric acid or a derivative thereof is a cyclic 4 membered arene based bioisostere of CO₂H, CONH₂, CONHNH₂, or a derivative thereof. In one embodiment, the squaric acid derivative can have a formula:

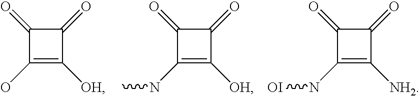

In another embodiment, the bioisostere of CO₂H, CONH₂, CONHNH₂, or a derivative thereof contains a hydroxyl substituted 5 or 6 membered arene or a heteroerene. In another embodiment, the bioisostere of CO₂H, CONH₂, CONHNH₂, or a derivative thereof contains the substituted 5 or 6 membered (C₁-C₈) heterocycle, arene or a heteroerene a moiety or formula

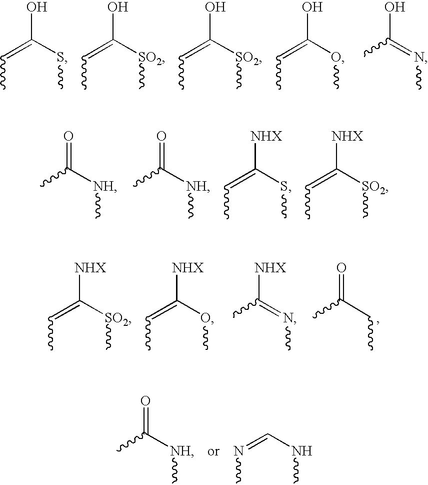

In one embodiment, the bioisostere of CO₂H, CONH₂, CONHNH₂, or a derivative thereof contains a moiety of formula 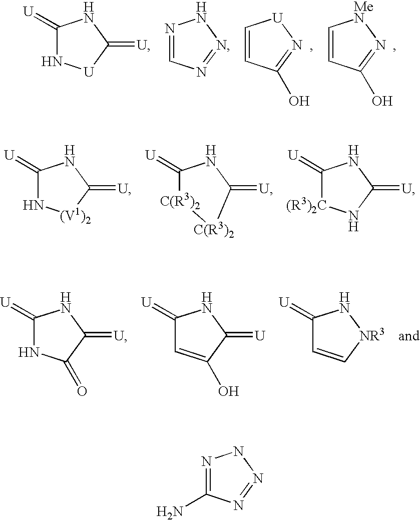
wherein the variables are as defined herein.

In one embodiment, the bioisostere of CO₂H, CONH₂, CONHNH₂, or a derivative thereof have a formula selected from the group consisting of: 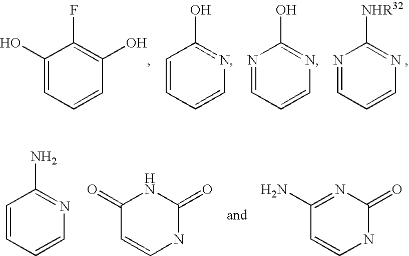
wherein the variables are as defined herein.

In one embodiment, the present invention provides carboxylic acid bioisosteres: COR³, COCOR³, COCHR³COR³, COC(R³)₂COR³, COCHR³CO₂R³, COC(R³)₂CO₂R³, COCHR³COR⁴, COC(R³)₂COR⁴, COCHR³COCOR³, COC(R³)₂COCOR³, COCHR³COCO₂R³, COC(R³)₂COCO₂R³, COCHR³COCOR⁴, COC(R³)₂COCOR⁴, and CF₃. In a related embodiment, the present invention provides carboxylic acid bioisosteres: COCF₃, COCOCH₂R³, and COCOCH₃.

Bioisosteres of carboxylic acid and derivatives, and indazole useful for the compounds of the present invention can be adapted for example from the references Lipinski et al., Annual Reports in Medicinal Chemistry-21, 1986, pages 283-91; Marfat, U.S. Pat. No. 6,391,872; Straub et al., Bioorg. Med. Chem. Lett., 2001, 11:781-4, Fenton, et al., U.S. Pat. No. 6,762,199; Gaster, et al., U.S. Pat. No. 5,705,498; Nicolaou, I. et al., J. Med. Chem., 2004; 47(10); 2706-9; and Hazeldine et al., J. Med. Chem., 2002; 45: 3130-7.

Dimers

In one aspect the present invention provides a multimeric-compound containing two or more lonidamine analog moieties. In one embodiment, the lonidamine analogs in the multimeric compound are both joined covalently by R⁹ substituents. In one embodiment, the lonidamine analogs in the multimeric compound are both joined covalently by R¹ substituents. In one embodiment, the lonidamine analogs in the multimeric compound are both joined covalently by V¹ substituents. In one embodiment, one of the lonidamine analogs in the multimeric compound is joined by one of R⁹, R¹, or V¹ substituent and the other lonidamine analogs in the multimeric compound is joined by one of R⁹, R¹, or V¹ substituent. The multimeric-compound as provided according to the present invention can have a higher affinity to a target organ, and/or target cells and show fewer side-effects upon administration.

Prodrugs

In one aspect, the present invention provides prodrugs of lonidamine analogs of formula (I). As used herein, a “prodrug” is a compound that, after administration, is metabolized or otherwise converted to an active or more active form with respect to at least one property. To produce a prodrug, a pharmaceutically active lonidamine analog (or a suitable precursor thereof) is modified chemically such that the modified form is less active or inactive, at least with respect to one biological property, relative to the pharmaceutically active compound, but the chemical modification is effectively reversible under certain biological conditions such that a pharmaceutically active form of the compound is generated by metabolic or other biological processes. A lonidamine analog prodrug may have, relative to the drug, altered metabolic stability or transport characteristics, fewer side effects or lower toxicity, or improved flavor, for example (see the reference Nogrady, 1985, Medicinal Chemistry A Biochemical Approach, Oxford University Press, New York, pages 388-392). Prodrugs can also be prepared using compounds that are not drugs.

In one aspect, the present invention provides prodrugs of lonidamine analogs of formula (I) wherein when R¹ represents COOR³, and R³ represents a group of the formula (CR¹⁵R¹⁶)ₘNR¹⁷R¹⁸ wherein each R¹⁵ and R¹⁶ is independently H, (C₁-C₈) alkyl, (C₁-C₈) heteroalkyl, (C₃-C₈) cycloalkyl, or (C₁-C₈) heterocyclyl or optionally, if both present on the same substituent, may be joined together to form a three- to eight-membered (C₃-C₈) cycloalkyl or (C₁-C₈) heterocyclyl ring system. Each R¹⁷ and R¹⁸ is (C₁-C₈) alkyl, heteroalkyl, (C₃-C₈) cycloalkyl, or (C₁-C₈) heterocyclyl or optionally, if both present on the same substituent, may be joined together to form a three- to eight-membered cycloalkyl or (C₁-C₈) heterocyclyl ring system.

A number of other groups of embodiments are preferred and are set forth below.

In a first group of embodiments, R¹ is preferably a COOR³ moiety.

In a first group of embodiments, R¹ is preferably a COOR³ moiety.

R³ can be (C₁-C₈) alkyl or (C₁-C₆) alkoxy, or a three- to eight-membered cycloalkyl or heterocyclyl ring system. For example, R³ can be (C₁-C₆) alkoxymethyl, such as methoxymethyl; (C₁-C₆) alkanoyloxymethyl esters such as pivaloyloxymethyl; phthalidyl esters; (C₃-C₈) cycloalkoxycarbonyloxy(C₁-C₆) alkyl such as 1-cyclohexyloxycarbonyloxyethyl; 1,3-dioxolen-2-onylmethyl esters, such as 5-methyl-1,3-dioxolen-2-on-ylmethyl; and (C₁-C₆) alkoxycarbonyloxyethyl such as 1-methoxycarbonyloxyethyl.

The subscript m is preferably 2 and each R¹⁵ and R¹⁶ is preferably independently selected from the group, H, CH₃, and a member in which R¹⁵ and R¹⁶ are joined together to form a cyclopropyl, cyclobutyl, cyclopentyl, cyclohexyl, 1,1-dioxo-hexahydro-1Δ⁶-thiopyran-4-yl or tetrahydropyran-4-yl group.

The prodrugs of the invention provide for the release of a drug lonidamine and its analogs. An illustrative example discussed below illustrated how a prodrug of the invention can be designed to exhibit increased aqueous solubility and extended pharmacokinetics in vivo.

In an embodiment of the invention, the prodrug moiety comprises a tertiary amine having a pKa near the physiological pH of 7.5. Any amines having a pKa within 1 unit of 7.5 are suitable alternatives amines for this purpose. The amine may be provided by the amine of a morpholino group. This pKa range of 6.5 to 8.5 allows for significant concentrations of the basic neutral amine to be present in the mildly alkaline small intestine. The basic, neutral form of the amine prodrug is lipophilic and is absorbed through the wall of the small intestine into the blood. Following absorption into the bloodstream, the prodrug moiety is cleaved by esterases which are naturally present in the serum to release lonidamine or the lonidamine analog. More strongly basic amines, such as a trialkyl derivatives with no heteroatom substitutions, will be nearly completely protonated under physiological conditions and will not be as efficiently adsorbed as shown.

In one aspect of the invention, the serum half live of the prodrug of the lonidamine and lonidamine analogs of the present invention is increased in vivo (compared to the parental form) by the presence of R¹⁵ and R¹⁶ groups. The R¹⁵ and R¹⁶ groups in the prodrug, as shown in the structure above, can independently be selected to modulate the rate of cleavage of the prodrug moiety from lonidamine. Increasing the amount of steric hindrance proximal to the ester carbonyl of lonidamine decreases the rate of cleavage of the prodrug moiety. Slowing the rate of cleavage of the prodrug moiety has the effect of increasing serum half life. Hydrogen groups facilitate cleavage of the prodrug moiety and alkyl groups hinder it. The larger and more branched the alkyl group, the more cleavage is hindered and the more serum half life is increased. Similarly, the closer the non-hydrogen substitution is to the lonidamine carbonyl, the more cleavage of the prodrug moiety is hindered and the more serum half life of the prodrug form is increased.

In a preferred embodiment, linkage of the tertiary amine to the lonidamine is stable enough so that the serum half life of the prodrug is from about 8 to about 24 hours.

In another aspect of the invention, R⁴ and R⁵ may be joined together to form a cyclic group further comprising heteroatoms. This aspect of the invention further improves upon the aqueous solubility of the compounds of the invention.

In one aspect, the present invention provides a prodrug D-Z-M of lonidamine or a lonidamine analog, said prodrug comprising, lonidamine or an analog, D; joined by a cleavable linker Z; to a moiety M. Prodrugs with this structure may be referred to as “linker prodrugs.” In some embodiments, the prodrug has a higher Vₘₐₓ for a transporter expressed in plasma membranes of cells than D alone. In some embodiments, the cells are epithelial cells lining a human colon, or small intestine, a prostate, or the like. In one embodiment, the transporter is expressed in the plasma membranes of epithelial cell lining in the human gut.

In another embodiment, the transporter is expressed in the plasma membranes of epithelial cell lining in the prostate. In one embodiment, the transporter is expressed in human kidney, brain, lung, liver and/or heart. In one embodiment, the moiety M is selected from the group consisting of an amino acid, a dipeptide, a tripeptide, a bile acid, and their derivatives. In one embodiment, the transporter is selected from the group consisting of ATBO, CAT-1, FATP4, MCT1, MCT4, NADC1, NADC2, OCTN2, PEPT1, PGT, RFC, SAT-1, SAT-6, SMVT, SUT2 and SVCT1 (for a description of these transporters see, e.g., Gallop et al., WO02100347). In one embodiment, the transporter is PEPT2, which is expressed in human kidney, brain, lung, liver, and heart. In one embodiment, the transport system is carrier mediated. In a related embodiment, the transport system is receptor mediated.

In one embodiment, the prodrug compound exhibits selective uptake by a subject's prostate as compared to another organ, such as the testis, heart, kidney, brain, lung, and/or liver. In a related embodiment, the prodrug is selectively taken up by subject's prostate compared to other organs. In another embodiment, the prodrug compound exhibits selective uptake by prostate epithelial cells as compared to other epithelial cells of, for example, the testis, heart, kidney, brain, lung, and/or liver. In a related embodiment, the prodrug is selectively taken up by prostate epithelial cell as compared to other epithelial cells.

In one embodiment, the M moiety is an androgen, an androgen analog, or a functional androgen analog that exhibits selective uptake by a subject's prostate as compared to another organ such as, for example, the testis, heart, kidney, brain, lung, and/or liver. In a related embodiment, the prodrug D is selectively taken up by subject's prostate. In another embodiment, the M moiety is an androgen, an androgen analog, or a functional androgen analog that exhibits selective uptake by prostate epithelial cells as compared to epithelial cells such as, for example, of the testis, heart, kidney, brain, lung, and/or liver. In a related embodiment, the prodrug is selectively taken up by prostate epithelial cells as compared to other epithelial cells.

In one embodiment, the present invention provides a prodrug of lonidamine or a lonidamine analog comprising a lonidamine- or a lonidamine analog-peptide conjugate, the peptide comprising an amino acid sequence having a cleavage site specific for an enzyme having a proteolytic activity of prostate specific antigen and wherein the peptide is linked to lonidamine or the lonidamine analog to inhibit the therapeutic activity of lonidamine or the lonidamine analog, and wherein lonidamine or the lonidamine analog is cleaved from the peptide upon proteolysis by an enzyme having a proteolytic activity of prostate specific antigen (PSA).

In one embodiment, the present invention provides a prodrug of lonidamine or a lonidamine analog comprising a lonidamine- or a lonidamine analog-peptide conjugate, the peptide comprising an amino acid sequence having a cleavage site specific for an enzyme having a proteolytic activity of prostate specific antigen and wherein the peptide is linked to lonidamine or the lonidamine analog to inhibit the therapeutic activity of lonidamine or the lonidamine analog, and wherein lonidamine or the lonidamine analog is cleaved from the peptide upon proteolysis by an enzyme having a proteolytic activity of prostate specific antigen (PSA).

In one embodiment, the present invention provides a prodrug of lonidamine or a lonidamine analog comprising a lonidamine- or a lonidamine analog-peptide conjugate, the peptide comprising an amino acid sequence having a cleavage site specific for an enzyme having a proteolytic activity of prostate specific antigen, wherein the peptide is 20 or fewer amino acids in length, wherein the sequence comprises the amino acids
G₅-G₄-G₃-G₂-G₁,
wherein G₅ is from 0 to 16 amino acids; G₄ is serine, isoleucine, or lysine; G₃ is serine or lysine; G₂ is leucine or lysine; and G₁ is glutamine, asparagine or tyrosine, and wherein the peptide is linked to lonidamine or the lonidamine analog to inhibit the therapeutic activity of the lonidamine or the lonidamine analog, and wherein lonidamine or the lonidamine analog is cleaved from the peptide upon proteolysis by an enzyme having a proteolytic activity of prostate specific antigen (PSA).

In one embodiment, the present invention provides a prodrug of lonidamine or an analog comprising a cephalosporin moiety, a dihydronicotinamide moiety, a triglyceride, a long chain fatty acid, or a long chain fatty alcohol.

In one embodiment, the present invention provides a prodrug of lonidamine or a lonidamine analog, wherein the moiety M is a vitamin or a vitamin precursor. In one embodiment, the present invention provides a prodrug of lonidamine or a lonidamine analog, wherein the moiety M is vitamin-D, a vitamin-D analog, or a vitamin-D precursor. In one embodiment, the present invention provides a prodrug of lonidamine or a lonidamine analog, wherein the moiety M is vitamin-E, a vitamin-E analog, or a vitamin-E precursor. In a related embodiment, the moiety M is α-tocopherol. In another related embodiment, the moiety M is an α-tocopherol-PEG conjugate. In another related embodiment, the moiety M is an α-tocopherol-α,ω-dicarboxylic acid-PEG conjugate. In another related embodiment, the moiety M is an α-tocopherol-succinic acid-PEG conjugate. Various α-tocopherol based conjugates employed in the present invention can be adapted from those described in the U.S. Patent Application No. US2005/0142189, to Lambert et al.

In one embodiment, the present invention provides a prodrug of lonidamine or a lonidamine analog, wherein the moiety M is a hormone or a hormone precursor.

In one embodiment, the present invention provides a prodrug of lonidamine or a lonidamine analog, wherein the moiety M is a hormone or a hormone precursor.

In one embodiment, the present invention provides a prodrug of lonidamine or a lonidamine analog wherein the prodrug is enzymatically modified to yield lonidamine or the lonidamine analog, wherein the enzyme is carboxypeptidase, aminohydrolase, or glycosidase. In one embodiment, the prodrug contains an Aryl-O—CO—N< moiety which is cleaved by a carboxypeptidase to yield lonidamine or a lonidamine analog from the prodrug.

Moiety M and linker Z that can be employed in a D-Z-M prodrug of the present invention is provided for example, in the reference Silverman, Jan. 15, 1992, Organic Chemistry of Drug Design and Drug Action, Academic Press; 1st edition.

Other M moieties including but not limited to a bile acid, an amino acid, and a peptide, and linker Z moieties that can be used in the compounds of the invention are described in the following US Patent Application Nos. 2004/0161424, 2003/0158254, 2003/0158089, and 2003/0017964; and PCT Publication Nos. WO 04/053192, WO 04/052844, WO 04/052841, WO 04/052360, WO 04/041203, WO 04/033655, WO 03/104184, WO 03/099338, WO 03/080588, WO 03/077902, WO 03/065982, WO 03/020214, WO 02/100392, WO 02/100347, WO 02/100344, WO 02/100172, WO 02/44324, WO 02/42414, WO 02/32376, WO 02/28883, WO 02/28882, WO 02/28881, and WO 02/28411. In a related embodiment, the moiety can be a targeting peptide, to target lonidamine or a lonidamine analog to a specific cell type. See, e.g., U.S. patent publication No. 2002/0147138.

In another aspect, the present invention provides a prodrug D-Z-M of lonidamine or a lonidamine analog, said prodrug comprising lonidamine or an analog, D, joined by a cleavable peptide linker Z, to a stabilizing moiety M. The peptide linker can be any cleavable peptide linker. In some embodiments, the linker is cleavable by an endogenous enzyme. In some embodiments, the linker is a tripeptide, P1-P2-P3, comprising natural or synthetic amino acids.

In some embodiments, P1 is Leucine, Sarcosine, Tyrosine, Phenylalanine, p-Cl-Phenylalanine, p-Nitrophenylalanine, Valine, Norleucine, Norvaline, Phenylglycine, Tryptophan, tetrahydroisoquinoline-3-carboxylic acid, 3-Pyridylalanine, Alanine, Glycine, or 2-Thienylalanine. In some embodiments, P2 can be Alanine, Leucine, Tyrosine, Glycine, Serine, 3-Pyridylalanine, or 2-Thienylalanine. In some embodiments, P3 can be Leucine, Phenylalanine, Isoleucine, Alanine, Glycine, Tyrosine, 2-Naphthylalanine, or Serine.

In some embodiments, the peptide linker can be one of the following: Leu-Ala-Leu, Tyr-Ala-Leu, Met-Ala-Leu, Tyr-Ala-Ile, Phe-Gly-Leu, Met-Gly-Leu, Met-Gly-Ile, Phe-Gly-Ile, Met-Gly-Phe, Leu-Ala-Gly, Nle-Ala-Leu, Phe-Gly-Phe, and Leu-Tyr-Leu. See also U.S. Patent Publication No. 2003/0181359.

In some embodiments, moiety M is a stabilizing moiety that protects the prodrug from cleavage in circulating blood when it is administered to the patient and allows the prodrug to reach the vicinity of the target cell relatively intact. The stabilizing group typically protects the prodrug from cleavage in blood and blood serum. In some embodiments, the stabilizing group is useful in the prodrug when it serves to protect the prodrug from degradation, i.e., inactivation, when tested by storage of the prodrug compound in human blood at 37° C. for 2 hours and results in less than 20%, particularly less than 2%, inactivation of the prodrug by the enzymes present in the human blood under the given assay conditions.

The stabilizing group can be, for example, an amino acid or an amino acid that is either (i) a non-genetically-encoded amino acid having four or more carbons or (ii) aspartic acid or glutamic acid attached to the N-terminus of the oligopeptide at the beta-carboxyl group of aspartic acid or the gamma-carboxyl group of glutamic acid. For example, dicarboxylic (or a higher order carboxylic) acid or a pharmaceutically acceptable salt thereof may be used as a stabilizing group. In other embodiments, the stabilizing group is not an amino acid.

In another aspect, linker prodrugs of the following formulae are provided: 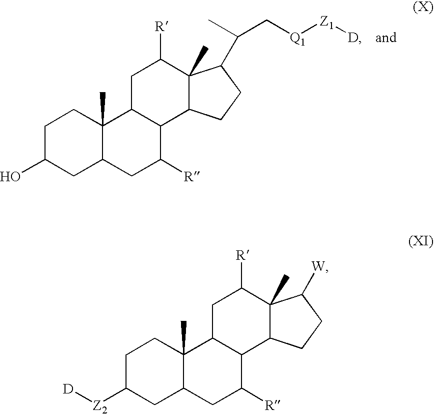
wherein D is a lonidamine analog of formula (I); Q₁ is O or CH₂; Z₁ and Z₂ are cleavable linkers; R′ is alpha-OH or hydrogen; R″ is alpha-OH, beta-OH or hydrogen; W is —CH(CH₃)W₁, wherein W₁ is a substituted alkyl group containing a moiety which is negatively charged at physiological pH, said moiety is selected from the group consisting of CO₂H, SO₃H, SO₂H, —P(O)(OR)(OH), —OP(O)(OR)(OH), and OSO₃H wherein R is C₁-C₆ alkyl, (C₁-C₈) heteroalkyl, (C₃-C₈) cycloalkyl, (C₁-C₈) heterocyclyl, aryl, or heteroaryl; and an individual isomer, a racemic or non-racemic-mixture of isomers, a bioisostere, a pharmacophore, a pharmaceutically acceptable salt, a solvate or a hydrate thereof.

In another aspect, compounds of the following formulae, and enantiomers and diastereomers thereof, are provided: 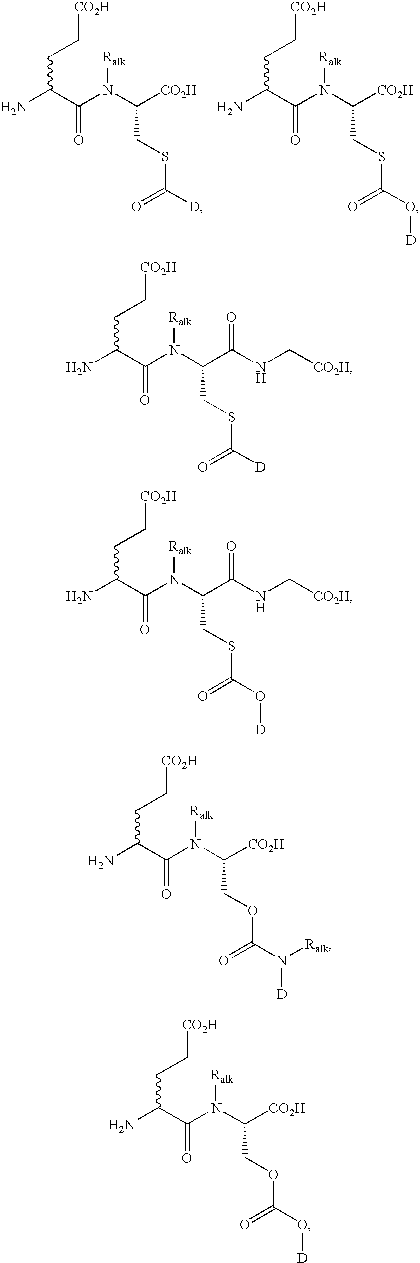 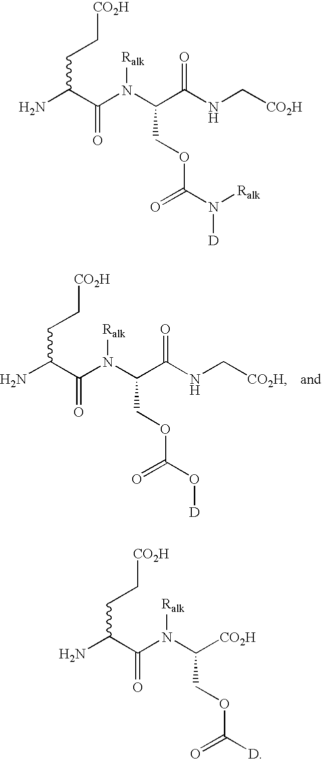
wherein Rₐₗₖ is alkyl (e.g., C₁-C₆ alkyl); and D is lonidamine or a lonidamine analog, and an individual isomer, a racemic or non-racemic mixture of isomers, a bioisostere, a pharmacophore, a pharmaceutically acceptable salt, a solvate or a hydrate thereof. In one embodiment, Rₐₗₖ is lower alkyl. In one embodiment, when D is covalently attached to a heteroatom in the formula above, then D is a lonidamine analog of formula (I), as defined above.

Various polyethylene glycol (PEG) moieties and methods for forming prodrugs with them that can be used in or to make compounds of the invention are described in U.S. Pat. Nos. 6,608,076; 6,395,266; 6,194,580; 6,153,655; 6,127,355; 6,111,107; 5,965,566; 5,880,131; 5,840,900; 6,011,042 and 5,681,567.

Various protecting groups and methods for forming prodrugs with them that can be used in or to make compounds of the invention can be adapted from the references Testa et al., Hydrolysis in Drug and Prodrug Metabolism, June 2003, Wiley-VCH, Zurich, 419-534 and Beaumont et al., Curr. Drug Metab. 2003, 4:461-85.

In another embodiment, the term “cleavable linker”, such as, e.g., Z, refers to a linker which has a short half life in vivo. The breakdown of the linker Z in a compound D-Z-M (supra) releases or generates lonidamine or a lonidamine analog. In one embodiment, the cleavable linker has a half life of less than ten hours. In one embodiment, the cleavable linker has a half life of less than an hour. In one embodiment, the half life of the cleavable linker is between one and fifteen minutes. In one embodiment, the cleavable linker has at least one connection with the structure: C*—C(═X*)X*—C* wherein C* is a substituted or unsubstituted methylene group, and X* is S or O. In one embodiment, the cleavable linker has at least one C*—C(═O)O—C* connection. In one embodiment, the cleavable linker has at least one C*—C(═O)S—C* connection. In one embodiment, the cleavable linker has at least one —C(═O)N*—C*—SO₂—N*-connection, wherein N* is —NH— or C₁-C₆ alkylamino. In one embodiment, the cleavable linker is hydrolyzed by an esterase enzyme.

In one embodiment, the linker is a self-immolating linker, such as that disclosed in U.S. patent publication 2002/0147138, to Firestone; PCT Appl. No. US05/08161 and PCT Pub. No. 2004/087075. In another embodiment, the linker is a substrate for enzymes. See generally Rooseboom et al., 2004, Pharmacol. Rev. 56:53-102.

Synthesis of Lonidamine Analogs

Lonidamine analogs of the invention can be prepared using by known synthetic methods in combination with the teaching herein. Synthesis of lonidamine is described in U.S. Pat. No. 3,895,026. Synthesis of certain lonidamine analogs, including tolnidamine (TND), has also been described (see, e.g., Corsi et al., 1976, “1-Halobenzyl-1H-Indazole-3-Carboxylic Acids. A New Class of Antispermatogenic Agents”, Journal of Medicinal Chemistry 19:778-83; Cheng et al., 2001, “Two new male contraceptives exert their effects by depleting germ cells prematurely from the testis” Biol Reprod. 65:449-61; Silvestrini, 1981, “Basic and Applied Research in the Study of Indazole Carboxylic Acids” Chemotherapy 27:9-20; Lobl et al., 1981, “Effects of Lonidamine (AF 1890) and its analogues on follicle-stimulating hormone, luteinizing hormone, testosterone and rat androgen binding protein concentrations in the rat and rhesus monkey” Chemotherapy 27:61-76; and U.S. Pat. Nos. 3,895,026 and 6,001,865.

Synthetic methodology applicable to other lonidamine analogs is generally described in U.S. Pat. No. 6,146,658, PCT app. No. PCT/US05/19350 (filed Jun. 2, 2005); and U.S. provisional application Nos. 60/576,968 (filed Jun. 20, 2004) and No. 60/588,694 (filed Jul. 15, 2004), as is administration of polymorphic forms, enantiomeric forms, tautomeric forms, solvates, hydrates, and the like. In one embodiment, the present invention provides novel prodrugs of compounds having formulas (I). Other exemplary prodrug forms of lonidamine and analogs thereof are described in copending PCT App. No. PCT/US2005/024434 (filed Jul. 8, 2005) entitled “Tertiary amine prodrugs of lonidamine and analogs,” and U.S. provisional application Nos. 60/586,934 (filed Jul. 8, 2004) and 60/624,505 (filed Nov. 1, 2004). Other exemplary lonidamine analogs are described in copending PCT application No. PCT/US2005/026929 (filed Jul. 29, 2005), U.S. provisional application No. 60/592,677 (filed Jul. 29, 2004); No. 60,599,664, (filed Aug. 5, 2004); and No. 60/651,671 (filed Feb. 9, 2005) all entitled “Multicyclic Lonidamine Analogs.” Each of the aforementioned applications is incorporated herein by reference. Methods for making lonidamine analogs wherein A-B ring is 2-chloroindole is can be adapted from the reference Andreani et al., Arch. Pharm., Weinheim, 1984, 317: 847-51.

Methods of synthesizing compounds of the present inventions are generally described in Schemes I-XX below. In one embodiment of the invention, methods for making lonidamine analogs of formula (I) are provided as shown in scheme 1 below:

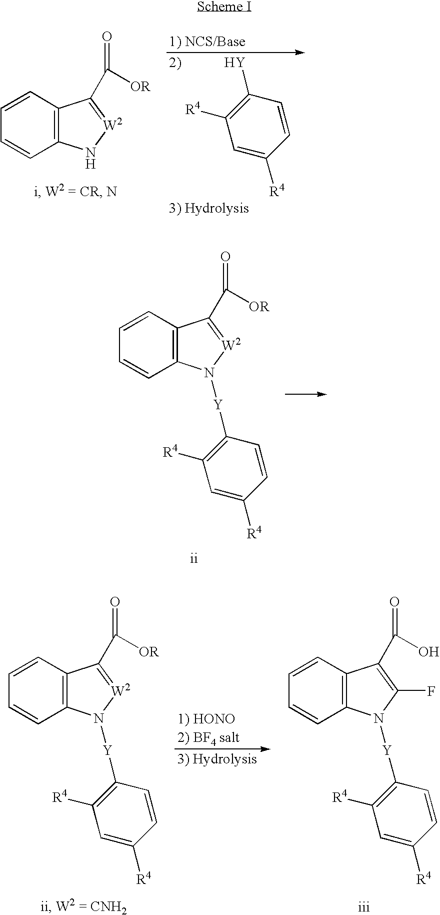

In another embodiment of the invention, methods for making lonidamine analogs of formula (I) are provided as shown in scheme II below:

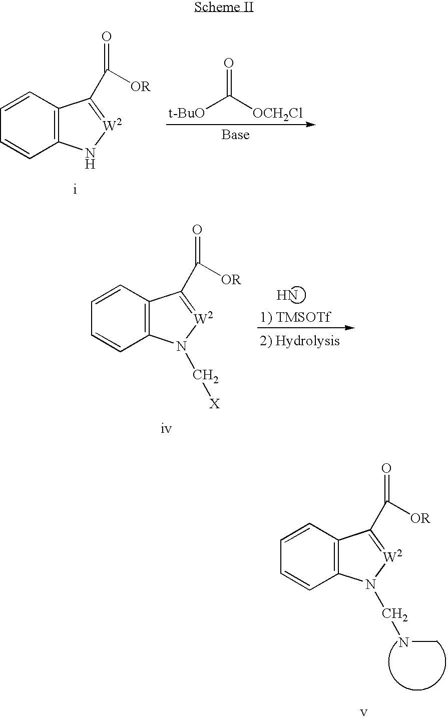

In another embodiment of the invention, methods for making lonidamine analogs of formula (I) are provided as shown in scheme III below:

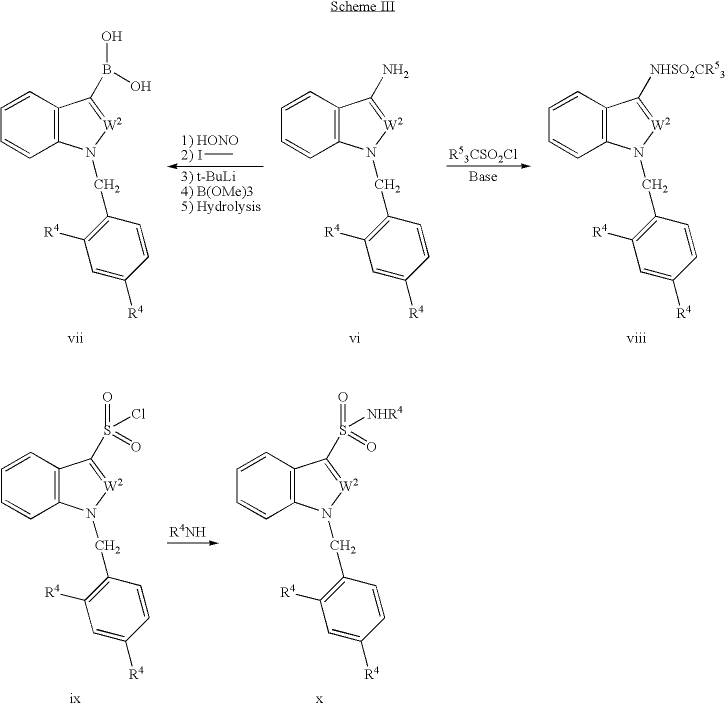

In another embodiment of the invention, methods for making lonidamine analogs of formula (I) are provided that relate generally to the methodology described in Bermudez et al., J. Med. Chem. 1990, 33:1924, Palacios et al., Tetrahedron 1995, 51(12):3683-3690 and Okuda et al., J. Org. Chem. 1991, 56, 6024, as shown in schemes IV-IX below:

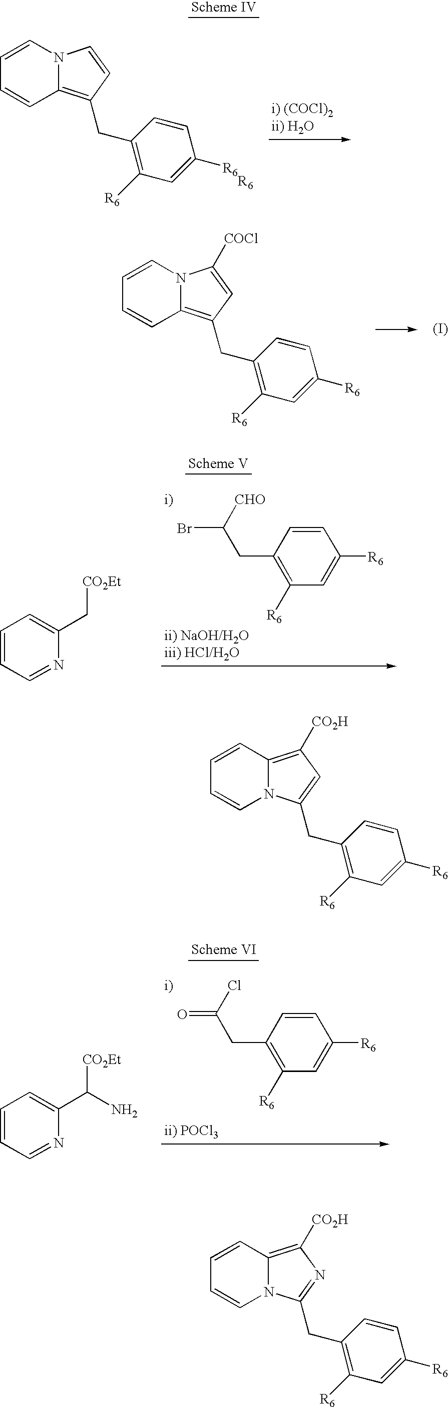

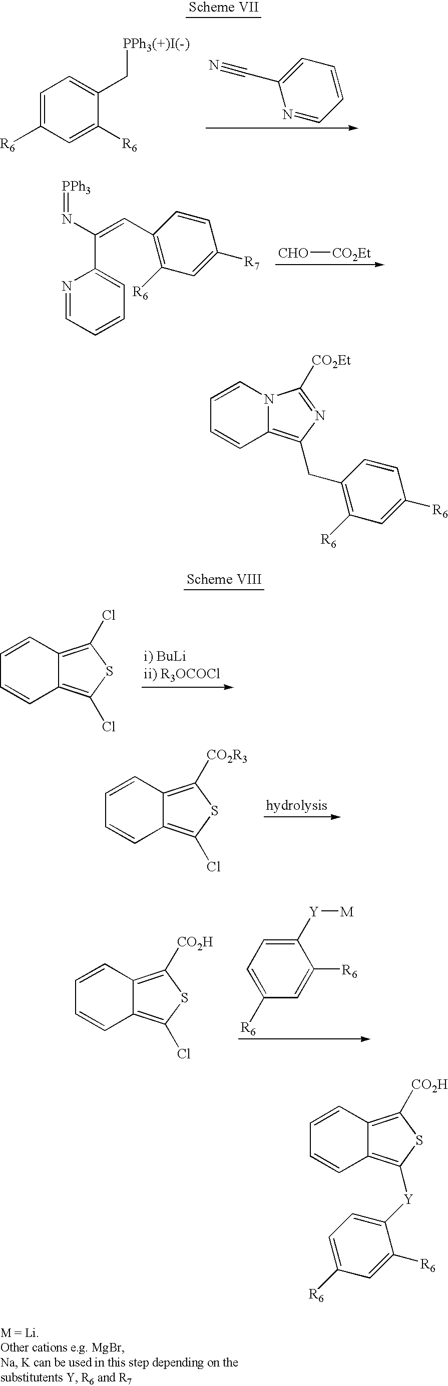

In another embodiment of the invention, methods for making lonidamine analogs of formula (I) are provided that relate generally to the methodology described in Tapia et al., J. Med. Chem. 1990, 33:1924, Palacios et al., Syn. Lett. 2002, 8: 1547-1549 as shown in schemes IX below:

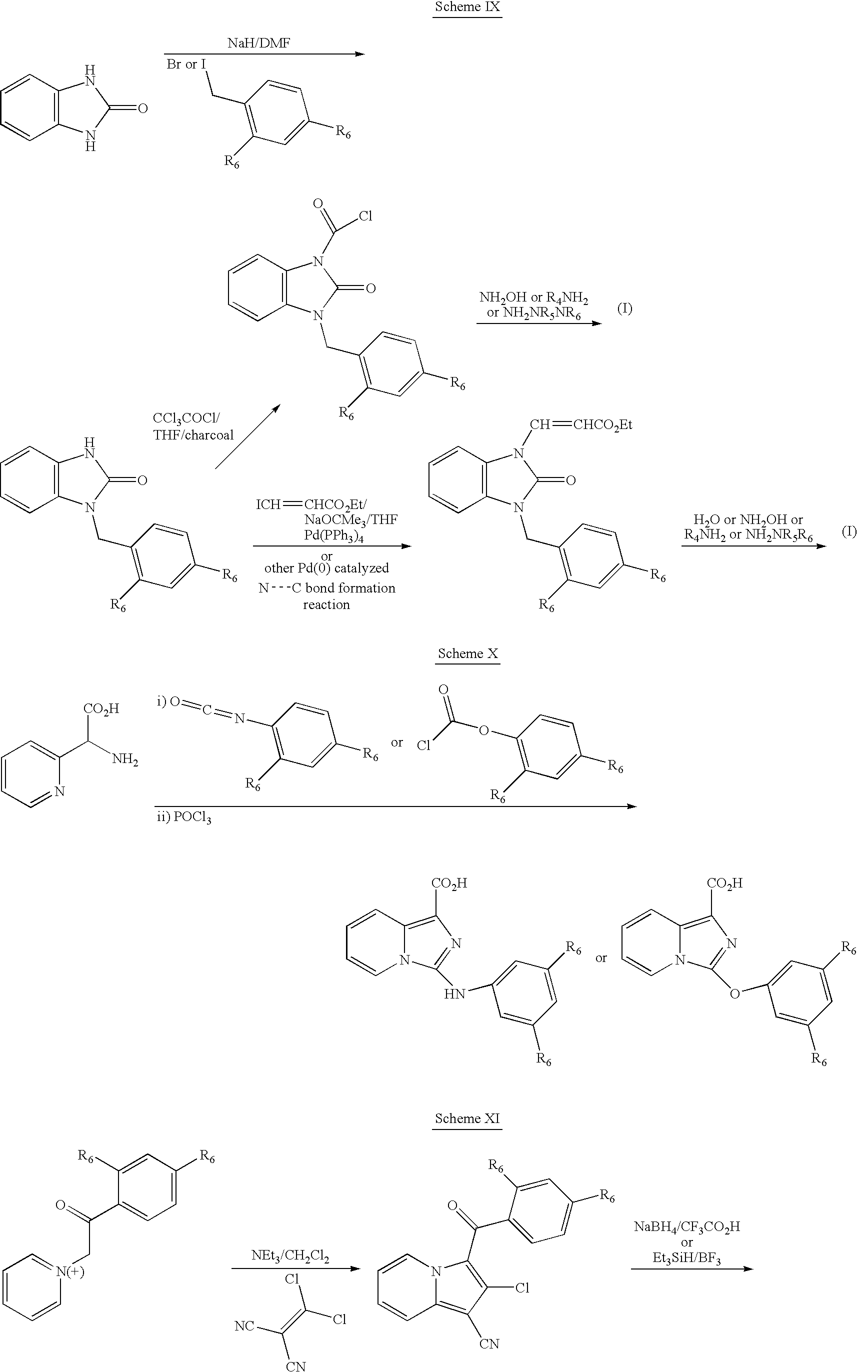

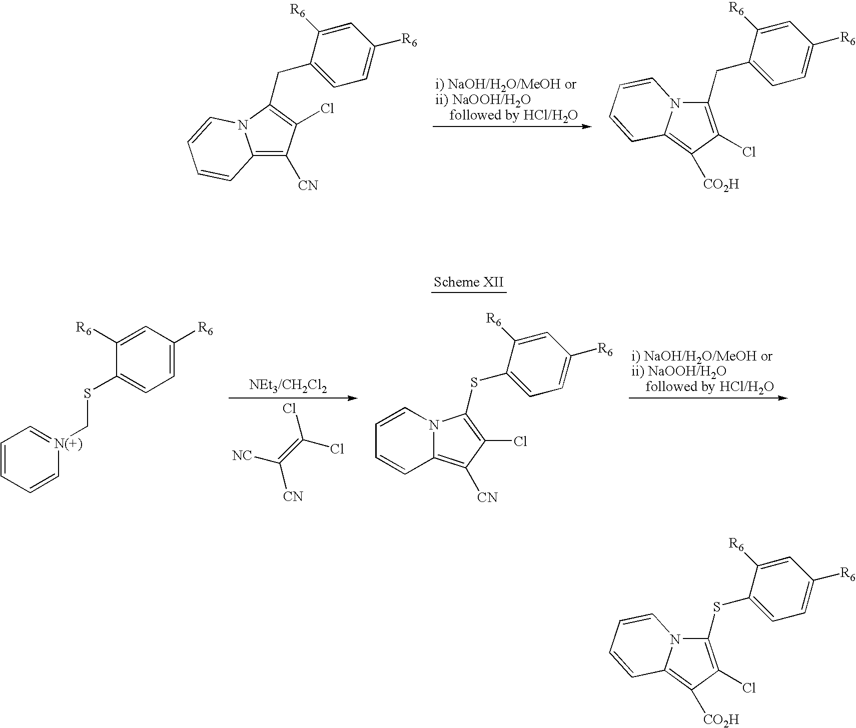

In another embodiment of the invention, methods for making lonidamine analogs of formula (I) are provided that relate generally to the methodology described in Tapia et al., Tetrahedron Lett. 2002, 8: 1547-1549 as shown in scheme XIII below:

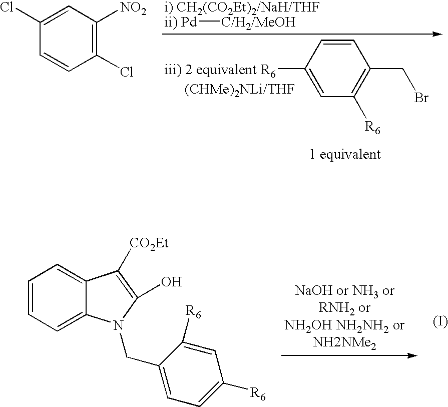

In another embodiment of the invention, methods for making lonidamine analogs of formula (I) are provided as shown in scheme XIV below:

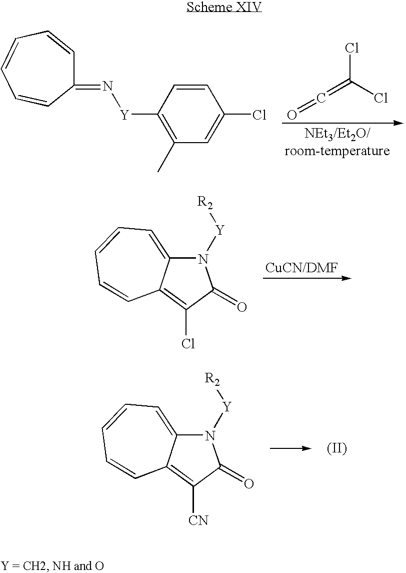

In another embodiment of the invention, methods for making lonidamine analogs of formula (I) are provided as shown in scheme XIV below:

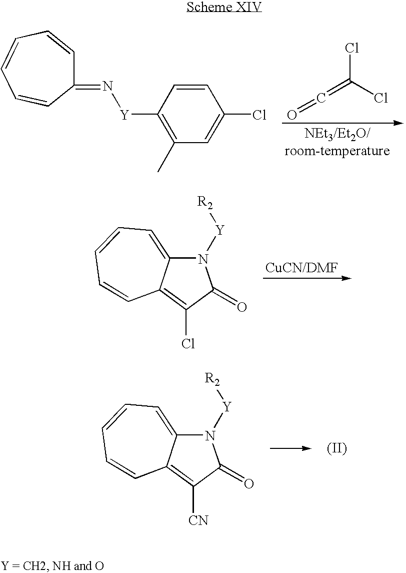

In another embodiment of the invention, methods of making lonidamine analogs of formula (I) are provided as shown below:

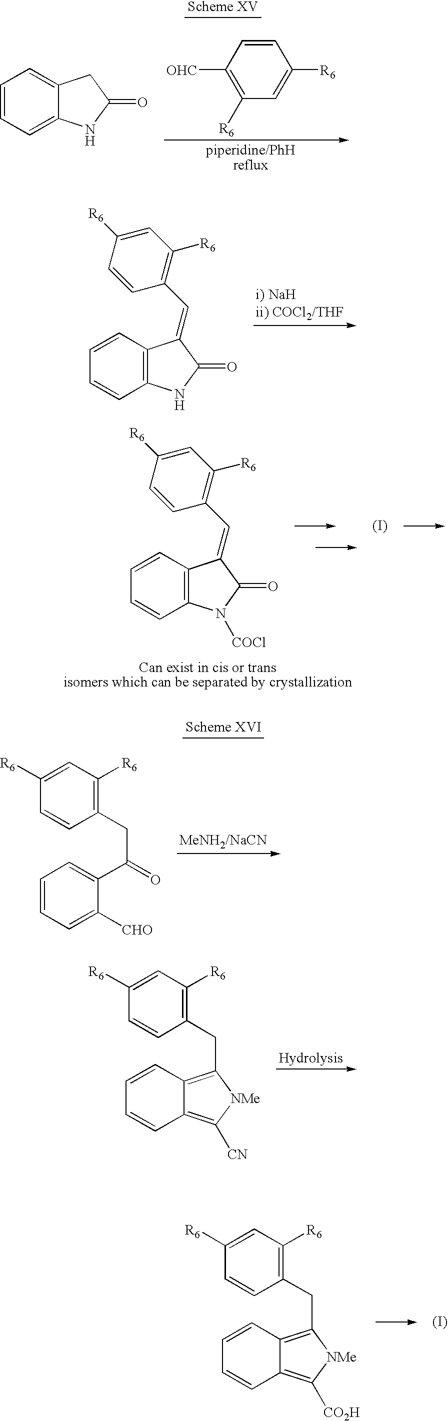

Carboxyl compound of formula (II) 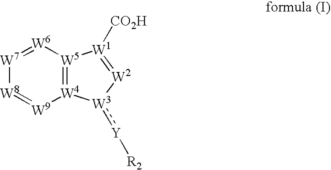
of the present invention can be synthesized as shown in several embodiments in schemes I-XVI above. The conversion of these carboxyl compounds and other precursors to compounds of the present invention having formula I wherein R¹ is CH═CH—CO₂H are provided hereafter.

Acrylic acid analogs of the present invention are synthesized from suitable carboxyl precursors as shown below in Scheme XVII:

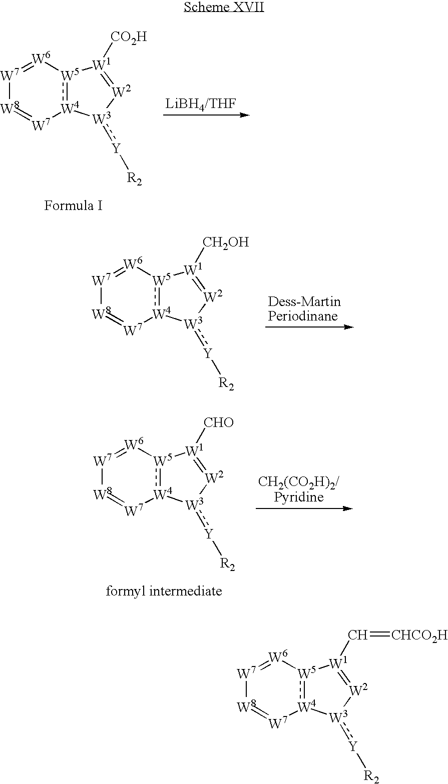

In another embodiment, acrylic acid analogs of the present invention are synthesized from suitable formyl precursors as shown below:

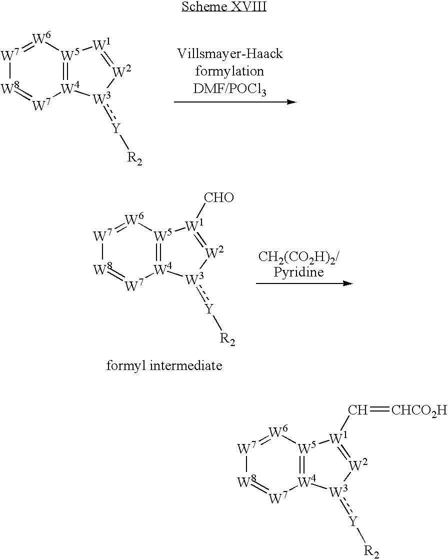

In another embodiment the formyl intermediate synthesized in scheme XVII is converted to an acrylic acid analog by employing a Witting Horner or a related carbon carbon double-bond forming reaction as shown in Scheme XIX:

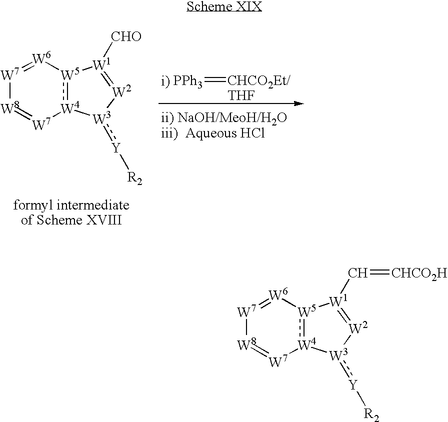

In another embodiment, a cyclopropano compound of formula (I) is synthesized by reacting an acrylate ester analog of formula (I) with substituted or unsubstituted carbene as shown below in Scheme XX. Diazomethane (CH₂N₂) or alkylsubstituted diazomethane (RₐₗₖCHN₂ wherein Rₐₗₖ is a substituted or unsubstituted C₁-C₄ alkyl group) is inserted into an acrylate ester or an acrylate ester analog double bond to yield a cyclopropano analog following rhodium or copper catalyzed insertion reactions. Simmons Smith reaction is used to insert a methylene group into an acrylate ester or an acrylate ester analog. Scheme XX

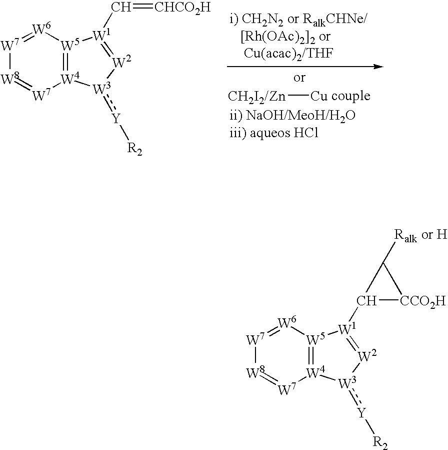

In another embodiment, propionic acid analogs of formula (I) are synthesized by reduction of final products obtained in Schemes I-XX. The reduction is performed employing Pd-charcoal, PtO₂-charcoal, Ra—Ni, Wilkinson's catalyst, L¹-liquid ammonia depending on the nature of substituents present in the starting compounds.

In one embodiment, compounds of formula I wherein one of W⁶-W⁹ is a carbon atom substituted with a C₁-C₄ alkyl, (C₁-C₄) heteroalkyl, halogen, hydroxy, (C₁-C₄) alkoxy, amino, cyano, nitro, C₁-C₄ alkylamino, and C₁-C₄ dialkylamino group are synthesized by employing methods described above and further employing starting materials which is suitably substituted. In another embodiment, employing as starting material: 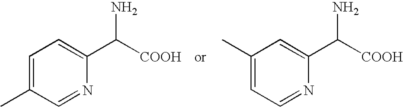
in scheme VI yields a compound of formula I.

Method of synthesis of compounds of the present invention wherein at least one of W⁶-W⁹ is a heteroatom is provided in the following section. A compound of the present invention wherein W¹-W⁹ define a pyrazolopyridine ring can be prepared by adapting synthetic procedures described by the references Lavecchia et al., Tetrahedron Lett., 2004, 45:2389-92; Straub et al., Bioorg. Med. Chem., 2001, 10:1711-7; and Straub et al., Bioorg. Med. Chem. Lett., 2001, 11:781-4. A compound of the present invention wherein W¹-W⁹ define a pyrrolo[2,3-d]-pyrimidine or a -pyrazolo[3,4-d]-pyrimidine ring can be prepared by adapting synthetic procedure described by the reference Kelley et al., J. Med. Chem. 1996, 38:3884-8. A compound of the present invention wherein W¹-W⁹ define a pyrrolo[1,2-c]pyrimidine can be prepared by adapting synthetic procedure described by the reference Minguez et al., J. Org. Chem. 1999, 64, 7788-801. A compound of the present invention wherein W¹-W⁹ define a pyrrolo[2,3-d]pyrimidin-2,4-dione and more particularly a 6-chloropyrrolo[2,3-d]pyrimidin-2,4-dione can be prepared by adapting synthetic procedure described by the reference Edstrom et al., Tetrahedron Lett., 1996, 37(6):759-62. A compound of the present invention wherein W¹-W⁹ define a 2-chloroindole can be prepared by adapting synthetic procedure described by the reference Engqvist et al. Eur. J. Org. Chem. 2004, 2589-92. A compound of the present invention wherein W¹-W¹³ define a thieno[2,3-b]indole moiety and more particularly a rotationally restricted lonidamine analog thieno[2,3-b]indole-2-carboxylate and a thieno[2,3-b]indole-2-carboxamide moiety can be prepared by adapting synthetic procedure described by the reference Engqvist et al., Eur. J. Org. Chem. 2004, 2589-92. A compound of the present invention wherein W¹-W⁹ define a Pyrazolo[3,4-b]pyridine moiety can be prepared by adapting synthetic procedure described by the reference Misra et al. Bioorg. Med. Chem. Lett., 2003, 13 2405-8. A compound of the present invention wherein W¹-W¹³ define a rotationally restricted lonidamine analog containing a pyridazinoindole moiety can be prepared by adapting synthetic procedure described by the reference Guven et al., Tetrahedron 1993, 49(48):11145-54. A compound of the present invention wherein W¹-W¹³ define a rotationally restricted lonidamine analog containing a triazolobenzimidazole moiety can be prepared by adapting synthetic procedure described by the reference Reddy et al., Indian J. Chem., 1992, 31B: 191-2. A compound of the present invention wherein W¹-W⁹ define a pyrano[2,3-c]pyrazoles and pyrano[2,3-c]pyrazole-6(1-H)-one moiety can be prepared by adapting synthetic procedure described by the reference Ueda et al., Chem. Pharm. Bull., 1981, 129(12):3522-8. A compound of the present invention wherein W¹-W⁹ define a pyrazolo[3,4-d]pyridazine moiety can be prepared by adapting synthetic procedure described by the reference Kaji et al., Chem. Pharm. Bull., 1984, 32(11):4437-46. A compound of the present invention wherein W¹-W⁹ define a pyrazolo[4,3-e][1,2,4]triazene moiety can be prepared by adapting synthetic procedure described by the reference Rykowski et al., Heterocycles, 2000, 53(10): 2175-81. A compound of the present invention wherein W¹-W⁹ define an imidazo[1,5-b]triazine[1,2,4] moiety can be prepared by adapting synthetic procedure described by the reference Guerret et al., Bull. Chem. Soc. France, 1974, (7-8):1453-4.

Syntheses of ester prodrugs of the invention may start with the free carboxylic acid of a lonidamine analog. The free acid is activated for ester formation in an aprotic solvent and then reacted with a free alcohol group in the presence of an inert base, such as triethylamine, to affect ester formation, producing the prodrug. Activating conditions for the carboxylic acid include forming the acid chloride using oxalyl chloride or thionyl chloride in an aprotic solvent, optionally with a catalytic amount of dimethyl formamide, followed by evaporation. Examples of aprotic solvents, include, but are not limited to methylene chloride, tetrahydrofuran, and the like. Alternatively, activations can be performed in situ by using reagents such as BOP (benzotriazol-1-yloxytris(dimethylamino) phosphonium hexafluorolphosphate) and the like (see Nagy et al., Proc. Natl. Acad. Sci. 90: 6373-6376, 1993) followed by reaction with the free alcohol. Isolation of the ester products can be affected by extraction with an organic solvent, such as ethyl acetate or methylene chloride, against a mildly acidic aqueous solution; followed by base treatment of the acidic aqueous phase so as to render it basic; followed by extraction with an organic solvent, for example ethyl acetate or methylene chloride; evaporation of the organic solvent layer; and recrystallization from a solvent, such as ethanol, which has been acidified with an acid, such as HCl or acetic acid. Alternatively, the crude reaction can be passed over an ion exchange column bearing sulfonic acid groups in the protonated form, washed with deionized water, and eluted with aqueous ammonia; followed by evaporation.

Suitable starting materials are reported in the art (see e.g. Kirshchke et al., Tet. Lett., 4281-4284 (1986); Corisii et al., J. Med. Chem. 778-783 (1976). Other starting materials are commercially available. Non-commercially available starting materials can be synthesized in via standard literature procedures. Such procedures can be identified via literature search tools such as SciFinder from the American Chemical Society or Beilstein, available from MDL Software.

In certain embodiments, the lonidamine analog is provided in the form of a pharmaceutically acceptable salt. Pharmaceutically acceptable salts include addition salts with acids, as well as the salts with bases. In one embodiment, suitable acids for the formation of acid addition salts are, for example, mineral acids, such as hydrochloric, hydrobromic, sulphuric or phosphoric acid, or organic acids, such as organic sulphonic acids, for example, benzenesulphonic, 4-toluenesulphonic or methanesulphonic acid, and organic carboxylic acids, such as acetic, lactic, palmitic, stearic, malic, maleic, fumaric, tartaric, ascorbic or citric acid. Acid salts of the tertiary amine moiety confer increased aqueous solubility. In one embodiment the salts are citric acid salts.

In another embodiment, suitable bases for the formulation of base addition salts of lonidamine and lonidamine analogs are a primary amine, a secondary amine, a tertiary amine, an amino acid, or a naturally occurring α-amino acid. Examples of aminoacids include but are limited to glycine, lysine, and arginine. In one embodiment, the cation employed in the base addition salt of lonidamine or a lonidamine analog is sodium, potassium, ammonium, or calcium. In one embodiment, base addition salts of lonidamine and lonidamine analogs are formed employing lysine, glycine, or arginine as a base. In one embodiment, one equivalent of an amine (wherein amine is as described above) is mixed with one equivalent of lonidamine or a lonidamine analog in water. The mixture is shaken or sonicated to yield a homogenous solution of the base addition salt of lonidamine or a lonidamine analog in water. In another embodiment, one equivalent lonidamine or a lonidamine analog is mixed in water with one equivalent of a metal hydroxide, oxide, bicarbonate, or carbonate wherein the metal comprises sodium, potassium, or calcium resulting in the formation of the metal salt of lonidamine or the analog. In one embodiment, the base addition salt of lonidamine and arginine is not administered intravenously to rats. In another embodiment, the base addition salt of lonidamine and glycine is not administered intravenously to normal dogs. In one embodiment, when in a base addition salt one component is lonidamine, the base is other than arginine or glycine.

The compounds of the invention are lonidamine analogs, including prodrug forms of the analogs. Certain prodrugs of the invention should exhibit, relative to lonidamine, increased aqueous solubility and extended pharmacokinetics in vivo.

In an embodiment of the invention, the prodrug moiety comprises a tertiary amine having a pKa near the physiological pH of 7.5. Any amines having a pKa within 1 unit of 7.5 are suitable alternatives amines for this purpose. The amine may be provided by the amine of a morpholino group. This pKa range of 6.5 to 8.5 allows for significant concentrations of the basic neutral amine to be present in the mildly alkaline small intestine. The basic, neutral form of the amine prodrug is lipophilic and is absorbed through the wall of the small intestine into the blood. Following absorption into the bloodstream, the prodrug moiety is cleaved by esterases that are naturally present in the serum to release the active agent lonidamine or the lonidamine analog. More strongly basic amines, such as trialkyl derivatives with no heteroatom substitutions, will be nearly completely protonated under physiological conditions and will not be as efficiently absorbed.

In one aspect of the invention, the serum half live of the lonidamine analogs of the present invention are increased in vivo compared to lonidamine.

In one embodiment, the lonidamine analog is stable enough so that the serum half life of the compound is from about 8 to about 24 hours.

Uses of Lonidamine Analogs

The lonidamine analogs described herein are suitable for any use contemplated for lonidamine, and in particular may be used for any as prophylactic, therapeutic and contraceptive agents. Exemplary pharmaceutical uses are described below. Other uses of the analogs of the invention include control of rodents.

Pharmaceutical Compositions

For use as a prophylactic, therapeutic or contraceptive agent, a lonidamine analog disclosed herein (including pharmaceutically acceptable salts, solvates, hydrates, and prodrugs) is usually formulated as a pharmaceutical composition comprising the analog and a pharmaceutically-acceptable carrier. The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical preparations for oral use can be obtained through combining active compounds with solid excipient and, optionally, other compounds. Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. For topical or nasal administration, penetrants appropriate to the particular barrier to be permeated are used in the formulation. Such penetrants are generally known in the art.

Further details on techniques for formulation and administration may be found in the latest edition of Remington's Pharmaceutical Sciences (Maack Publishing Co., Easton, Pa.); GOODMAN AND GILMAN'S: THE PHARMACOLOGICAL BASIS OF THERAPEUTICS 10^{TH} EDITION 2001 by Louis Sanford Goodman et al., McGraw-Hill Professional; PHARMACEUTICAL DOSAGE FORMS AND DRUG DELIVERY SYSTEMS 7^{th} Edition Howard C. Ansel, et al., 2004, Lippincott Williams & Wilkins Publishers; PHARMACEUTICAL CALCULATIONS 11^{th} Edition, 2001, by Mitchell J. Stoklosa et al., Lippincott Williams & Wilkins; PHYSICAL PHARMACY: PHYSICAL CHEMICAL PRINCIPLES IN THE PHARMACEUTICAL SCIENCES 4^{th} Edition by Pilar Bustamante, et al., 1993, Lea & Febiger.

Dosages and Administration

A variety of routes, dosage schedules, and dosage forms are appropriate for administration of pharmaceutical compositions of the invention. Appropriate dosage schedules and modes of administration will be apparent to the ordinarily skilled practitioner upon reading the present disclosure and/or can be determined using routine pharmacological methods and/or methods described herein.

The dose, schedule and duration of administration of the analog will depend on a variety of factors. The primary factor, of course, is the choice of a specific analog. Other important factors include the age, weight and health of the subject, the severity of symptoms, if any, the subject's medical history, co-treatments, goal (e.g., prophylaxis or the prevention of relapse), preferred mode of administration of the drug, the formulation used, patient response to the drug, and the like.

For example, an analog can be administered at a dose in the range of about 0.1 mg to about 100 mg of the analog per kg of body weight of the patient to be treated per day, optionally with more than one dosage unit being administered per day, and typically with the daily dose being administered on multiple consecutive days. In one embodiment, an analog is administered in a dose in the range of about 0.1 mg to about 5 mg per kg of body weight of the patient to be treated per day. In another embodiment, an analog is administered in a dose in the range of about 0.2 mg to about 1 mg per kg of body weight of the patient to be treated. In certain other embodiments, an analog is administered in a dose of about 25 to 250 mg. In another embodiment, a dose is about 25 to about 150 mg.

Guidance concerning administration is provided by prior experience using the analog for a different indication (e.g., lonidamine administered to treat cancer is administered in 150 mg or 300 mg doses three times a day for a period of about a month) and from new studies in humans (e.g., lonidamine administered to treat BPH has been administered in 150 mg doses once a day for a period of about a month) and other mammals. Cell culture studies are frequently used in the art to optimize dosages, and the assays disclosed herein can be used in determining such doses (e.g., to determine the dose that induces significant apoptosis in prostate epithelial cells but not in other cells, such as, for example, liver cells). In addition, appropriate dosages of the analogs of the invention can be estimated by comparison to lonidamine in terms of (a) bioavailability and (b) biological activity. Biological activity can be determined using assays such as, but not limited to, those described hereinbelow. Preferred lonidamine analog are from 1- to 1000-fold as effective than lonidamine in a bioassay (e.g., as an anti-spermatogenic agent).

For illustration, a therapeutically or prophylactically effective dose of an analog can be administered daily or once every other day or once a week to the patient. Controlled and sustained release formulations of the analogs may be used. Generally, multiple administrations of the analog are employed. For optimum treatment benefit, the administration of the prophylactically effective dose may be continued for multiple days, such as for at least five consecutive days, and often for at least a week and often for several weeks or more. In one embodiment, the analog is administered once (qday), twice (bid), three times (tid), or four times (qid) a day or once every other day (qod) or once a week (qweek), and treatment is continued for a period ranging from three days to two weeks or longer.

Use of Pharmaceutical Compositions

Benign Prostatic Hyperplasia (BPH)

The invention provides a method for treatment or prophylaxis of benign prostatic hyperplasia (BPH) by administering a therapeutically effective or prophylactically effective amount of a compound described herein. The use of lonidamine for treatment or prophylaxis of BPH has been described [see, e.g., U.S. patent application Ser. No. 10/759,337 published as US 20040167196; also see the reference Ditonno et al., 2005, Rev. Urol. 7(suppl 7):S27-33] which also provides exemplary dosage regimens and schedules for treatment of BPH.

In certain embodiments, a compound of one or more of the following Groups as described hereinabove is administered for the prevention or treatment of BPH: Group 2, or any of Groups 3-60, with the proviso that compounds of any one or more of Groups B-J, K1-K6, L1, L2, M1-M6, N1-N3, O, P1 and P2 are excluded.

Treatment of Cancer

In another aspect, the invention provides a method for treatment of cancer by administering a therapeutically effective amount of a compound described herein. The use of lonidamine for treatment of cancer has been described. Cancers that can be treated using analogs of the invention include leukemia, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteosarcoma, Ewing's sarcoma, veticulum cell sarcoma, myeloma, giant cell tumor, small-cell lung tumor, islet cell tumor, primary brain tumor, acute and chronic lymphocytic and granulocytic tumors, hairy-cell tumor, adenoma, hyperplasia, medullary carcinoma, pheochromocytoma, mucosal neurons, intestinal ganglloneuromas, hyperplastic corneal nerve tumor, marfanoid habitus tumor, Wilms tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, and epidermoid carcinomas. Analogs disclosed herein may be administered alone or in combination with other anti-cancer agents and other drugs (see PCT publication WO2004/064734 for a description of combination therapies using lonidamine). Other anticancer agents that can be used in combination with the analogs of the invention include busulfan, improsulfan, piposulfan, benzodepa, carboquone, 2-deoxy-D-glucose, meturedepa, uredepa, altretamine, imatinib, triethylenemelaamine, triethylenephosphoramide, triethylenethiophosphoramide, trimethylolomelamine, chlorambucil, chlomaphazine, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, mycophenolic acid, nogalaamycin, olivomycin, peplomycin, plicamycin, porfiromycin, puromycin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, 5-fluorouracil, tegafur, L-asparaginase, pulmozyme, aceglatone, aldophosphamide glycoside, aminolevulinic acid, amsacrine, bestrabucil, bisantrene, carboplatin, defofamide, demecolcine, diaziquone, elformithine, elliptinium acetate, etoglucid, flutamide, gallium nitrate, hydroxyurea, interferon-alpha, interferon-beta, interferon-gamma, interleukin-2, lentinan, mitoguazone, mitoxantrone, mopidamol, nitracrine, pentostatin, phenamet, pirarubicin, podophyllinic acid, 2-ethylhydrazide, procarbazine, razoxane, sizofuran, spirogermanium, paclitaxel, tamoxifen, teniposide, tenuazonic acid, triaziquone, 2,2′,2″-trichlorotriethylamine, urethan, vinblastine and vincristine.

In one embodiment a compound of one or more of the following Groups as described hereinabove is administered for treatment of cancer: Group 2, or any of Groups 3-60, optionally with the proviso that compounds of any one or more of Groups B-J, K1-K6, L1, L2, M1-M6, N1-N3, O, P1 and P2 are excluded.

Prevention of Epithelial Cancer

In another aspect, the invention provides a method for the prevention of cancer by administering a prophylactically effective amount of a compound described hereinabove. In one embodiment, the cancer is prostate cancer. In another embodiment the cancer is breast cancer. In other embodiments the cancer is an epithelial cell cancer. Candidates for prophylasis using the compounds of the invention are individuals at increased risk (compared to the general population) for developing cancer,

Indicators of increased risk for developing prostate cancer can include (1) abnormal results from a digital rectal examination or prostate imaging, (2) elevated prostate specific antigen (PSA) levels such as greater than about 2 ng/ml (e.g., greater than about 2 ng/ml but less than about 8 ng/ml), (3) rising PSA, (4) expression of prostate cancer-susceptibility markers (see e.g., WO9514772, WO9845436; WO9837418, WO987093; WO9403599; WO9839446, WO9845435 and U.S. Pat. No. 5,665,874; U.S. Pat. No. 6,902,892); (5) genetic predisposition to developing prostate cancer; and (6) familial history of prostate cancer. In addition, age is a risk factor for developing prostate cancer, with more than 75% percent of prostate cancer diagnosed in men ages 65 or older.

Indicators of increased risk for developing breast or other epithelial cancers can include (1) abnormal physical examination results (e.g., abnormal breast examination results) or abnormal results from an X-ray, ultrasonographic or other procedure, (2) detection of epithelial cancer-susceptibility markers [e.g., CA-125 (epithelial cancer), HER² (breast cancer), Topoisomerase II alpha (ovarian epithelial cancer), Werner helicase interacting protein (ovarian epithelial cancer), HEXIM1 (ovarian epithelial cancer), FLJ20267 (ovarian epithelial cancer), Deadbox protein-5 (ovarian epithelial cancer), Kinesin-like 6 (ovarian epithelial cancer), p53 (ovarian epithelial cancer) and NY-ESO-1 (ovarian epithelial cancer)]; (3) genetic predisposition to developing epithelial cancer (for example, polymorphic BRCA1, BRCA2, p53, PTEN, ATM, NBS1 or LKB1 loci associated with increased susceptibility to epithelial breast cancer; e.g., Dumitrescu et al., 2005, J. Cell. Mol. Med. 9:208-21; or (4) family history of epithelial cancer.

Candidates for administration of lonidamine analogs for the prevention of cancer are individuals not diagnosed or under treatment for cancer (e.g., lung, breast, prostate, brain, ovarian, epithelial cell or other cancer) and, in the case of men not under treatment for BPH. In some embodiments the subject has not previously been treated for BPH or cancer.

The use of lonidamine for the prevention of cancer has been described [see U.S. provisional application No. 60/587,017 and PCT application PCT/US05/24423, entitled “Prevention of Cancer” filed Jul. 8, 2004].

In certain embodiments of the invention, a compound of one or more of the following Groups as described hereinabove is administered for the prevention of cancer: Group 2, or any of Groups 3-60, optionally with the proviso that compounds of any one or more of Groups B-J, K1-K6, L1, L2, M1-M6, N1-N3, O, P1 and P2 are excluded.

Prostatic Intraegithelial Neoplasia (PIN)

Prostatic Intraepithelial Neoplasia (PIN) is characterized by abnormal cellular proliferation within the prostatic ducts, ductules and acini. Treatment of PIN using lonidamine is disclosed in commonly assigned copending patent application PCT PCT/US05/24423 entitled “Prevention of Cancer” filed Jul. 8, 2005. PIN can be characterized as high grade (HGPIN) or low grade (LGPIN). HGPIN is associated with the progressive development of abnormalities in the normal prostatic epithelium, leading to a cancerous condition. See, e.g., Bostwick, 1992, J. Cell Biochem. Suppl. 16H: 10-9. Patients diagnosed as having HGPIN have an increased likelihood of developing prostate cancer within 10 years.

The invention provides a method for treating an patient diagnosed with HGPIN by administering a therapeutic amount of a lonidamine analogs disclosed hereinabove. The invention also provides a method for treating an patient diagnosed with LGPIN by administering a therapeutic amount of a lonidamine analogs disclosed hereinabove. PIN is usually diagnosed by needle biopsy, but can be diagnosed by any method known to the skilled artisan and accepted in the medical community.

In certain embodiments of the invention, a compound of Formula I, optionally with the proviso that compounds of any one or more of Groups B-J are excluded, or one or more of the following Groups as described hereinabove is administered for treatment of PIN: Group 2, or any of Groups 3-60, optionally with the proviso that compounds of any one or more of Groups B-J, K1-K6, L1, L2, M1-M6, N1-N3, O, P1 and P2 are excluded.

In certain embodiments the patient is not also under treatment for BPH or cancer. In certain embodiments the patient has not previously been treated for BPH or cancer.

Macular Degeneration

Compounds of the invention find use for the treatment or prevention of macular degeneration. Macular degeneration (e.g., Age-related Macular Degeneration, AMD) is a degeneration of the cells of the macula, resulting in a loss of function of the portion of the eye responsible for central vision, blurred vision and ultimately blindness. The early stages of the disease are associated with reduced nutrient flow, including oxygen, to the retina and diseased and healthy retinal pigment epithelial cells (RPE). In response to the reduced nutrient flow and hypoxia to RPE and the retina, new blood vessels grow from the deeper choroidal layer up into the RPE layer and in between the RPE and the retina, a process known as choroidal neovascularization (CNV). Leakage from the new vessels damages the retina, leading to visual distortions. New vessels may also grow up into the retina, creating blind spots. In addition, hypoxia inducible factor (e.g., HIF-1alpha) can be over-expressed subadjacent to the retina, which can stimulate growth of new blood vessel. The use of lonidamine to treat macular degeneration is disclosed in commonly assigned copending U.S. provisional application No. 60/639,055 and PCT application filed on 22 Dec. 2005 (Attorney Docket No. 021305-004710PC). Administration of compounds of the invention that inhibit angiogenesis and/or HIF-1alpha expression may be used in macular degeneration therapy.

In certain embodiments of the invention, a compound of Formula I, optionally with the proviso that compounds of any one or more of Groups B-J are excluded, or one or more of the following Groups as described hereinabove is administered for treatment of macular degeneration: Group 1, Group 2, or any of Groups 3-60, optionally with the proviso that compounds of any one or more of Groups B-J, K1-K6, L1, L2, M1-M6, N1-N3, O, P1 and P2 are excluded.

In certain embodiments the patient is not also under treatment for BPH or cancer. In certain embodiments the patient has not previously been treated for BPH or cancer.

Antiangiogenesis

In another aspect, the invention provides a method for inhibition of angiogenesis-related endothelial cell functions by administering a therapeutically or prophylactically effective amount of a compound described herein. Lonidamine has been reported to inhibition of angiogenesis-related endothelial cell functions. See commonly assigned copending U.S. provisional application No. 60/639,055. Also see Del Bufalo et al., 2004, “Lonidamine causes inhibition of angiogenesis-related endothelial cell functions.” Neoplasia 6:513-22.

In certain embodiments of the invention, a compound of Formula I, optionally with the proviso that compounds of any one or more of Groups B-J are excluded, or one or more of the following Groups as described hereinabove is administered to inhibit angiogenesis in a tissue of a subject: Group 1, Group 2, or any of Groups 3-60, optionally with the proviso that compounds of any one or more of Groups B-J, K1-K6, L1, L2, M1-M6, N1-N3, O, P1 and P2 are excluded.

In certain embodiments the patient is not also under treatment for BPH or cancer. In certain embodiments the patient has not previously been treated for BPH or cancer.

Antispermatogenesis

Lonidamine was initially developed as a male contraceptive based on its antispermatogenic activity (see, e.g., Cheng et al., 2001, Biol. Reprod. 65:449-61 and U.S. Pat. No. 6,001,865). Compounds of the invention with similar activity find use as antispermatogenics (e.g., contraceptives or antifertility agents) in mammals, such as rodents, humans and nonhuman primates. Lonidamine and certain lonidamine analogs have been reported to have antispermatogenic activity (see Corsi et al., 1976, “1-Halobenzyl-1H-Indazole-3-Carboxylic Acids. A New Class of Antispermatogenic Agents,” J. Med. Chem. 19:778-83; Silvestrini, 1981, “Basic and Applied Research in the Study of Indazole Carboxylic Acids,” Chemotherapy 27:9-20; Lobl et al., 1981, “Effects of Lonidamine (AF 1890) and its analogues on follicle-stimulating hormone, luteinizing hormone, testosterone and rat androgen binding protein concentrations in the rat and rhesus monkey,” Chemotherapy 27:61-76; U.S. Pat. No. 6,001,865 entitled “3-Substituted 1-Benzyl-1H-Indazole Derivatives As Antifertility Agents”; Cheng et al., 2001, “Two new male contraceptives exert their effects by depleting germ cells prematurely from the testis,” Biol. Reprod. 65:449-61 Burroughs et al., 2004, “Identification of tissue, cellular, and molecular targets for the new non-hormonal male contraceptive Gamendazole© compared to Lonidamine”, Abstract of poster presentation, Future of Male Contraception, September 29-October 2, Seattle, Wash. (see also, www.futureofinalecontraception.com), and Georg et al., 2004, “Discovery of Gamendazole©: Design, Synthesis, and in vivo Evaluation of an Effective Orally Bioavailable Non-hormonal Male Contraceptive Agent” Abstract of oral presentation, Future of Male Contraception, September 29-October 2, Seattle, Wash. (see also, www.futureofinalecontraception.com) and the lonidamine analogs described herein have similar activities. Accordingly, the compounds described herein may find use as contraceptives.

In certain embodiments of the invention, a compound of Formula I, with the proviso that compounds of Groups A and B are excluded and optionally with the proviso that compounds of any one or more of Groups C-J are excluded, or one or more of the following Groups as described hereinabove is administered to inhibit spermatogenesis in a tissue of a subject: Group 1, Group 2, or any of Groups 3-60, optionally with the proviso that compounds of any one or more of Groups B-J, K1-K6, L1, L2, M1-M6, N1-N3, O, P1 and P2 are excluded.

In certain embodiments, when used in humans, the subject is not under treatment for BPH or cancer. In certain embodiments the subject has not previously been treated for BPH or cancer.

In a related use, compounds of the invention can be used to control fertility in animals (e.g., rodents).

Energolytic Activity

It has been suggested that lonidamine's anticancer properties result at least in part from a lonidamine-mediated disruption of the mitochondrial membrane, resulting in reduced activity of mitochondria-bound hexokinase and interference with ATP production by the glycolytic pathway and oxidative phosphorylation. See, Floridi et al., 1981, “Effect of lonidamine on the energy metabolism of Ehrlich ascites tumor cells” Cancer Res. 41:4661-6; Fanciulli et al., 1996, “Effect of the antitumor drug lonidamine on glucose metabolism of adriamycin-sensitive and -resistant human breast cancer cells” Oncology Research 3:111-120, and references numbered 15-22 therein; and Gatto, 2002, “Recent studies on lonidamine, the lead compound of the antispermatogenic indazol-carboxylic acids” Contraception 65:277-78. The lonidamine analogs described herein may be administered to reduce activity of mitochondria-bound hexokinase and/or interfere with ATP production by the glycolytic pathway and oxidative phosphorylation in a cell. Accordingly, these compounds may be used to treat any condition for which such reduction in ATP production is desirable in a cell or tissue.

In certain embodiments of the invention, a compound of Formula I, with the proviso that compounds of Groups A and B are excluded and optionally with the proviso that compounds of any one or more of Groups C-J are excluded, or one or more of the following Groups as described hereinabove is administered to inhibit angiogenesis in a tissue of a subject: Group 1, Group 2, or any of Groups 3-60, optionally with the proviso that compounds of any one or more of Groups B-J, K1-K6, L1, L2, M1-M6, N1-N3, O, P 1 and P2 are excluded.

In certain embodiments the patient is not also under treatment for BPH or cancer. In certain embodiments the patient has not previously been treated for BPH or cancer.

Further, the lonidamine analogs of the invention can be administered in treatment methods described in the following U.S. patent applications: U.S. patent application Ser. No. 10/759,337 (filed Jan. 16, 2004); U.S. provisional application nos. 60/592,883, entitled “Methods and Agents for Treatment of Benign Prostatic Hypertrophy” (filed Jul. 29, 2004) and 60/661,067 (filed Mar. 11, 2005); U.S. provisional application No. 60/587,017 (filed Jul. 8, 2004) and related PCT application PCT/US05/24423 (filed Jul. 8, 2005), entitled “Prevention of Cancer” each of which is incorporated herein by reference.

Biological Activities of Lonidamine Analogs.

In various embodiments, a pharmaceutical composition of the invention may be any compound described herein. In various embodiments, a pharmaceutical composition of the invention may comprise a compound of Formula I, or compounds of any of Groups 1-60, as described above. In certain embodiments, compounds of one, more than one, or all of Groups A-J, K1-K6, L1, L2, M1-M6, N1-N3, O, P1 and P2 are excluded. Lonidamine analogs best suited for use as pharmaceutical agents are those with biological activity and low toxicity (low therapeutic index). As is usual in the pharmaceutical arts, not every structural analog of a compound is pharmacologically active. Active forms can be identified by routine screening of analogs for the activity of the parent compound. A variety of assays and tests can be used to assess pharmacological activity of analogs of the invention, including in vitro assays, such as those described below and elsewhere herein, in vivo assays of prostate function (including citrate production and ATP production) in humans, non-human primates and other mammals, in vivo assays of prostate size in humans, non-human primates and other mammals, and/or clinical studies. The activity of a lonidamine analog of interest in any of the assays described below can be compared with that of lonidamine to provide guidance concerning dosage schedules for the compound, and other information.

Antiproliferation Assays

In certain embodiments the compounds of the invention have antiproliferative activity (i.e., addition of the compound interfere with or reduce the rate or extent of proliferation of mammalian cells in vitro, ex vivo, or in vivo). Numerous cell proliferation assays are known in the art. Suitable assays include the antiproliferation assays described in Examples 39-41, below. In some embodiments, a compound is used that has the same or greater antiproliferative activity than does lonidamine. In an aspect, the invention provides a method for inhibiting proliferation of a mammalian cell by contacting the cell with an compound of the invention. The compound and cell can be contacted in vivo or in vitro. In one embodiment the cell is cultured. In one embodiment the cell exhibits abnormal or unregulated growth in vivo (e.g., a malignant or benign tumor cell). In one embodiment the cell is an epithelial cell or epidermal cell (e.g., a skin cell of a subject with a proliferative skin disease such as psoriasis or contact dermatitis).

Apoptosis Assay in Cell Lines.

As shown in Example 3 of patent publication US 20040167196, lonidamine induces apoptosis in cell lines derived from human prostate cells. The induction of apoptosis is significantly greater in LNCaP cells (ATCC NO. CLR-1740), a prostate-derived cell line that is citrate-producing, than in PC3 cells (ATCC NO. CLR-1435), a prostate-derived cell line that is citrate-oxidizing, consistent with the susceptibility of the citrate-producing prostate cells to metabolic inhibitors such as lonidamine. In some methods of the invention, a lonidamine analog has similar apoptosis-inducing activity.

Also see Example 42, infra, for an apoptosis assay for characterizing analogs.

Apoptosis Assay in Primary Cell Cultures.

As shown in Example 3 of patent publication US 20040167196, lonidamine induces apoptosis in primary cultures of human prostate epithelial cells. The induction of apoptosis is significantly greater in primary cultures of prostate epithelial cells than in primary cultures of human prostate stromal cells, consistent with the susceptibility of citrate-producing prostate cells to metabolic inhibitors such as lonidamine. In some methods of the invention, a lonidamine analog has similar apoptosis-inducing activity is selected. In some embodiments of the invention, a lonidamine analog that induces apoptosis in primary cultures of prostate epithelial cells to a significantly greater degree than in primary cultures of human prostate stromal cells is used. In some embodiments of the invention, the lonidamine analog does not significantly induce apoptosis in stromal cells. In some embodiments of the invention, induction of apoptosis by the lonidamine analog is at least 2-fold greater in epithelial cells than in stromal cells (and sometimes at least 4-fold greater, sometimes at 10-fold greater, and sometimes at least 20-fold greater) when assayed at the concentration of analog at which the difference in the level of apoptosis in the two cell lines is greatest (provided that the concentration of analog used in the assay is not greater than 1 mM).

HIF-1-Alpha Expression Assays.

Example 2 of patent publication US 20040167196 suggests that lonidamine reduced HIF-1-alpha expression/accumulation (measured in the nuclear fraction) in cells cultured under conditions of hypoxia by almost 2-fold at 200 micromolar and by more than 5 fold (i.e., more than 10-fold) at higher lonidamine concentrations. Thus, in some embodiments of the invention, an energolytic agent reduces HIF-1-alpha expression (prevents HIF-1-alpha accumulation) in LNCaP cells cultured under hypoxic conditions by at least about 2-fold, at least about 5-fold or at least about 10-fold compared to culture in the absence of lonidamine.

Hexokinase Activity.

As discussed above, and without intending to be bound to any specific mechanism, the effects of lonidamine on the prostate may be mediated, at least in part, by its effects on mitochondria and mitochondrial hexokinase activity in secretory epithelial cells. Accordingly, some lonidamine analogs useful in the methods of the present invention have hexokinase inhibitory activity as great or greater than that of lonidamine. Assays for hexokinase activity are known in the art. See Fanciulli et al., 1996, Oncology Research 3:111-120; Floridi et al., 1981, Cancer Res. 41:4661-6.

Antispermatogenic Activity.

Likewise, it is believed that the antispermatogenic activity of lonidamine results, at least in part, from energolytic effects in germ cells. Some lonidamine analogs useful in the present invention have antispermatogenic activity as great, or greater, than that of lonidamine. Assays for antispermatogenic activity are known in the art. See, e Contraception.g., Grima et al., 2001, Biol Reprod. 64:1500-8; Lohiya et al., 1991, 43:485-96.

In one embodiment, the present invention provides a lonidamine analog for therapeutic or prophylactic use (e.g., therapy or prophylaxis of BPH or cancer) as an antispermatogenic agent wherein said lonidamine analog is 1-1000 fold more effective than lonidamine as a male contraceptive or an anti-spermatogenic agent.

In one embodiment, the present invention provides a lonidamine analog containing an acrylic acid moiety for therapeutic or prophylactic use (e.g., therapy or prophylaxis of BPH or cancer) or as an antispermatogenic agent wherein said lonidamine analog is 1-1000 fold more effective than lonidamine as a male contraceptive or an anti-spermatogenic agent.

In Vivo Measurements of Prostate Function.

The effect of a compound on prostate function, and, in particular, on respiration, can be assessed by monitoring prostate tissue metabolism following administration of the compound. Some lonidamine analogs useful in the present invention will detectably reduce ATP, citrate, and/or lactate production by the prostate in animals (including humans, non-human primates and other mammals). ATP, citrate, and/or lactate levels can be monitored directly and/or indirectly in vivo using techniques of magnetic resonance spectroscopy (MRS) or other methods. See, for example, Narayan and Kurhanewicz, 1992, Prostate Suppl. 4:43-50; Kurhanewicz et al., 1991, Magnetic Resonance in Medicine 22:404-13 and Thomas et al., 1990, J Magnetic Resonance 87:610-19, for MRS assays that can be applied for this purpose.

In Vivo Measurements of Prostate Size.

The effect of a compound on prostate size can be assessed following administration of the compound using standard methods (for example, ultrasonography or digital rectal examination, for humans, and ultrasonography and/or comparison of organ weight in animals). Assays can be conducted in humans or, more usually, in healthy non-human animals or in monkey, dog, rat, or other animal models of BPH (see, Jeyaraj et al., 2000, J Androl. 21:833-41; Lee et al., 1998, Neurourol Urodyn. 17:55-69 and Mariotti et al., 1982, J Urol. 127:795-7). Some lonidamine analogs useful in the present invention will detectably reduce prostate size in such assays and animal models.

Examples 44 and 45, infra, describe assays in mice of the effect of an analog on the prostate. Example 49, infra, describes assays in rats of the effect of an analog on the prostate.

### EXAMPLES

### Example 1

To a solution of 1-(2,4-dichlorobenzyl)-indazole carbonylchloride (see U.S. Pat. No. 3,895,026) in EtOAc was added a solution of aqueous NH₂OH. The organic solution was washed with water, volatiles removed in vacuo, and the residue crystallized from acetic acid to yield compound 1.

Compound 2 was made according to the method described above for compound 1 by reacting 1-benzylindazole carbonylchloride with aqueous NH₂OH. Compound 2 was purified from the crude reaction mixture by crystallization from acetic acid.

### Example 2

To a solution of 1-(2,4-dichlorobenzyl)-indazole carbonylchloride (see U.S. Pat. No. 3,895,026) in EtOAc was added excess aminoethanol (about 10 eq) and stirred for 5 min at rt. The organic portion was washed with water, dilute aqueous HCl, and concentrated in a rotary evaporator to yield a residue which was separated by column chromatography on silica gel using 10-100% EtOAc/hexane as a solvent to yield compound 10.

Compounds 16 and 17 were synthesized in the same way as provided for compound 10 using 1-(2,4-dichlorobenzyl)-indazole carbonylchloride and 1-benzylindazole carbonylchloride, respectively, and substituting hydrazine hydrate for aqueous NH₂OH.

### Example 3

To a solution of 2-aminopyridine (20 mmol) in pyridine (20 mL) was added 1-(2,4-dichlorobenzyl)-indazole carbonylchloride (20 mmol, see U.S. Pat. No. 3,895,026) and stirred for 3 h at rt and concentrated in a rotary evaporator to yield a residue which was separated by column chromatography on silica gel using 10-100% EtOAc/hexane as a solvent to yield compounds 6 and 7.

Compounds 8 and 9 were synthesized employing the procedure as provided for compound 7 and substituting 2-aminopyridine with 3,4-difluoroaniline and 2-amino thiazole, respectively.

### Example 4

To a solution of MeSO₂NH₂ (20 mmol) in pyridine (20 mL) was added 1-(2,4-dichlorobenzyl)-indazole carbonylchloride (20 mmol, see U.S. Pat. No. 3,895,026) and stirred for 16 h at rt and concentrated in a rotary evaporator to yield a residue which was separated by column chromatography on silica gel using 10-90% EtOAc/hexane as a solvent to yield compound 4.

### Example 5

To a solution of glucuronic acid (5 g) in DMF (20 mL) was added diazabicyloundecane (DBU, 1.1 equivalent) and stirred at rt for 15 min followed by the addition of allyl bromide (1.2 equivalent). The reaction mixture was stirred for 16 h, and concentrated in a rotary evaporator to yield a residue which was separated by column chromatography on silica gel employing 50-100% acetone in toluene to yield allyl glucuronidate: 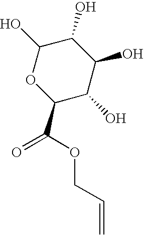
which was employed in the next reaction as follows.

To a solution of allyl glucuronate (2 g) and 1-(2,4-dichlorobenzyl)-indazolecarboxylic acid (2 equivalent) in THF (120 mL) was added triphenylphosphine (2 equivalent), diisopropyl azodicarboxylate (2 equivalent) and stirred for 1 h at rt and volatiles removed in a rotary evaporator. The residue was separated by column chromatography using silica gel and employing 20-70% acetone/toluene as eluent to yield: 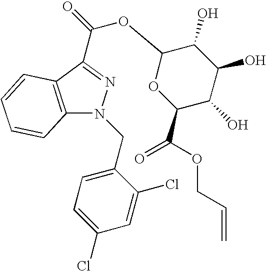
1.8 g of which was deprotected using Pd(PPh₃)₄ (0.1 equivalent) and pyrrolidine (0.56 equivalent) in THF (20 mL) to yield after column chromatographic separation on silica gel employing 0-30% water/MeCN as solvent to yield compound 15 in a 1:1.25 ratio of the α and β isomers.

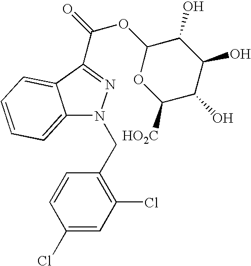

The compounds 18-22 were synthesized as described in the reference Corsi et al., (supra, see scheme above). In general, to a solution of indazole-3-carboxylic acid (10 mmol) and sodium hydroxide (20 mL 10% NaOH aq.) was added a benzylic chloride (R²—CH₂—Cl, 2 equivalent) wherein R² is 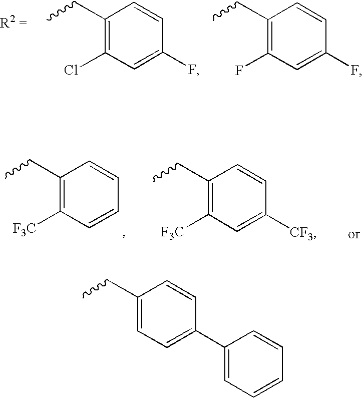
and stirred for 12 h at 70° C. The reaction mixture was then cooled to room-temperature and a white solid obtained was filtered, acidified with HCl (1N), and recrystallized from AcOH to yield pure 18-22 as white solids.

### Example 6

### Compound 24

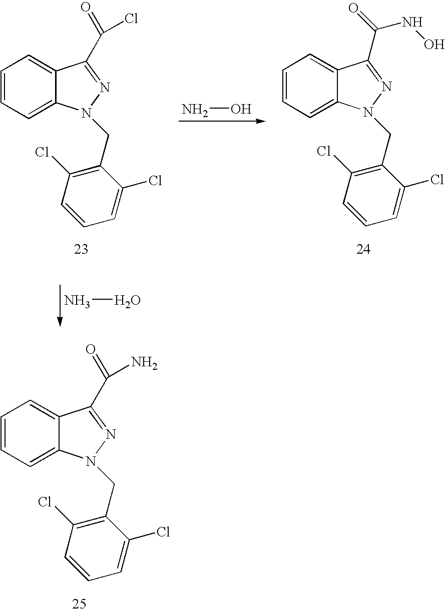 Preparation of Compound 24

Hydroxylamine (0.2 mL, 50% in water) was added to a solution of compound 23 (150 mg) in 2 mL EtOAc at RT. The mixture was stirred at RT for 10 min, and filtered. The solid was washed with water, 2-propanol, and ether to get 95 mg of a white solid 24.

### Example 7

### Compound 25

Preparation of Compound 25

NH₃.H₂O (0.2 mL, 28% in water) was added to a solution of compound 23 (150 mg) in 8 mL EtOAc at RT. The mixture was stirred at RT for 20 min. The organic phase was washed with 10% NaHCO₃, and brine, dried over Na₂SO₄, and concentrated under reduced pressure to give white solid 25.

### Example 8

### Compound 26

Typical Procedure for the Preparation of Substituted indazole 3-carboxylic acids. Preparation of 1-(4-chloro-2-methylbenzyl)-indazole 3-carboxylic acid, Compound 26

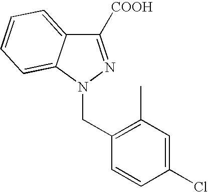

1H-indazole 3-carboxylic acid (1 g), K₂CO₃ (5 g) and 4-chloro-2-methylbenzyl chlorides (3.6 g) were suspended in dimethyl acetamide (DMA, 20 mL DMA), the mixture was then heated to 70° C. for 5 hrs. After the reaction was over, the reaction mixture was cooled to RT and 100 mL water was added and then the mixture was stirred for 2 hr at room temperature. Filtration gave the crude product 1-(4-chloro-2-methylbenzyl)-indazole 3-carboxylic acid 4-chloro-2-methylbenzyl ester. The crude ester was dissolved in methanol (50 mL), KOH (2 gram) was added into the reaction mixture and the reaction mixture was stirred for 8 hrs at 70° C. After the reaction was over, the reaction mixture was acidified with HCl to pH of about 1. Filtration gave the crude product. Recrystallization of the crude product in AcOH gave the pure product 26 as a white solid.

Acids 27, 28, 29, 30, 31, 32, 33, 34, 35 and 36 were prepared in a similar way as described above.

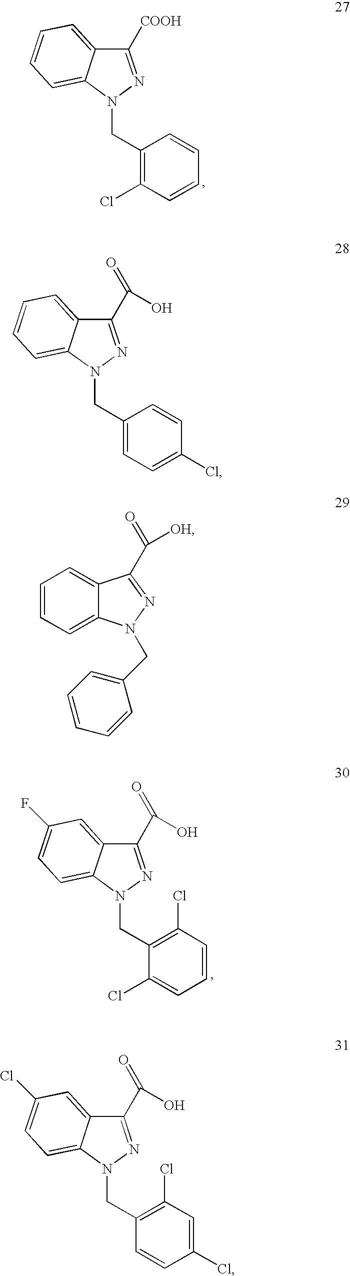

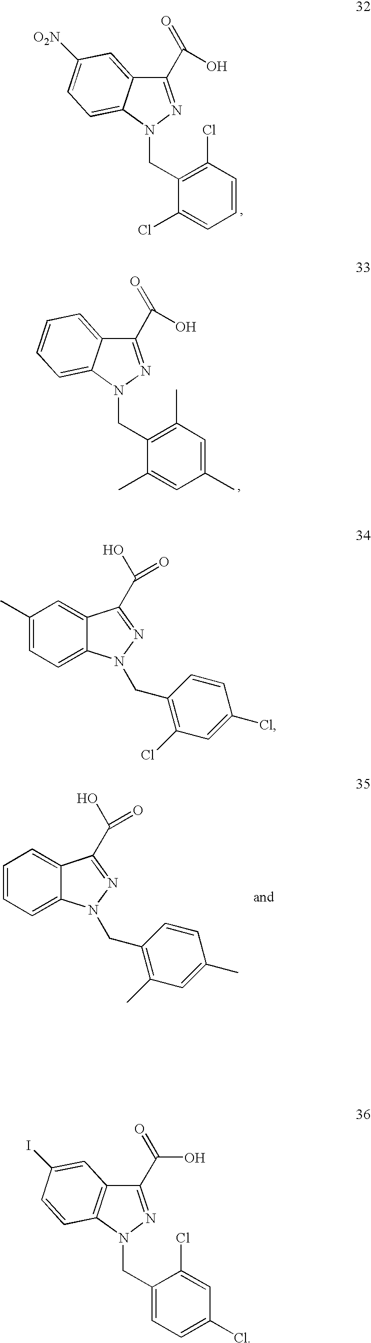

### Example 9

### Compound 37

Typical Procedure for Preparation of Amide Derivatives: Preparation of Compound 37.

1-(2,6-Dichlorobenzyl)-indazole-3-carboxyl chloride (0.1 g) was dissolved in DCM (5 mL). Methyl amine (1N in THF, 4 mL)) was then added into the reaction mixture at room temperature and the reaction was stirred over in 5 min. The mixture was then purified by flash chromatography (EtOAc/Hexane (0% to 100%)). Pure product 37 was obtained as a white solid.

Amides 38, 39, 40, 41, 42, 43, 44, 45, 46, 47, 48, 49, 50, 51, 52, 53, 54, 55, 56 and 57 were prepared in a similar way starting from the corresponding acyl chlorides and amines.

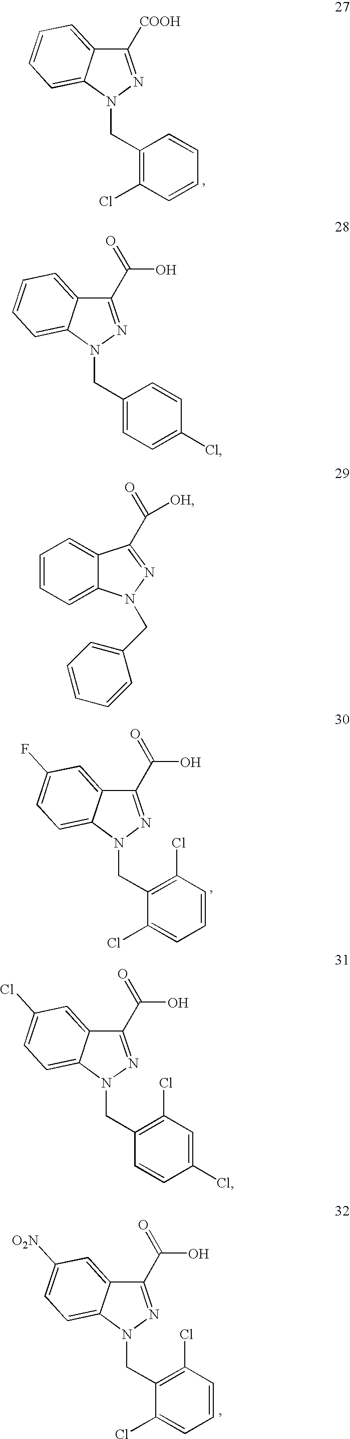

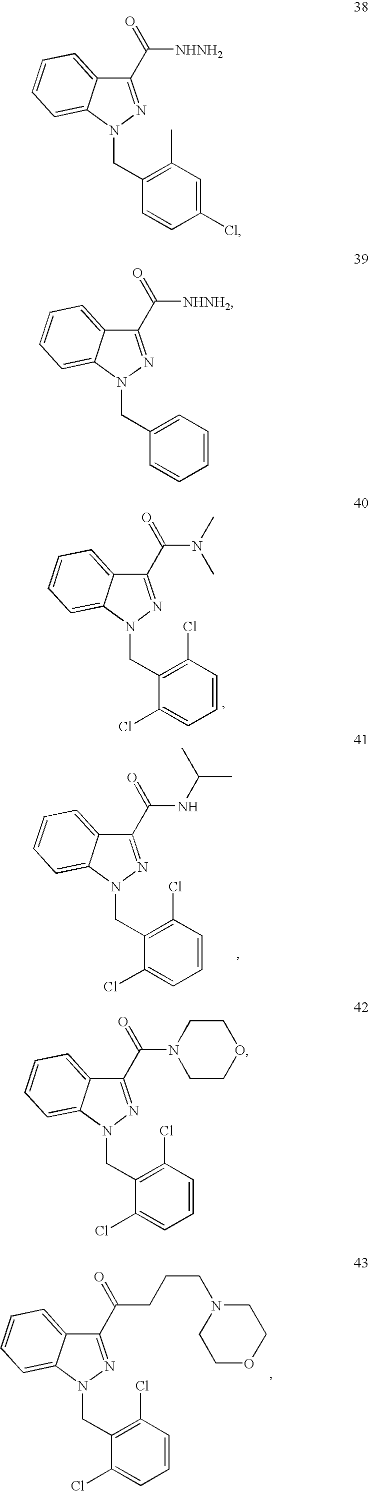

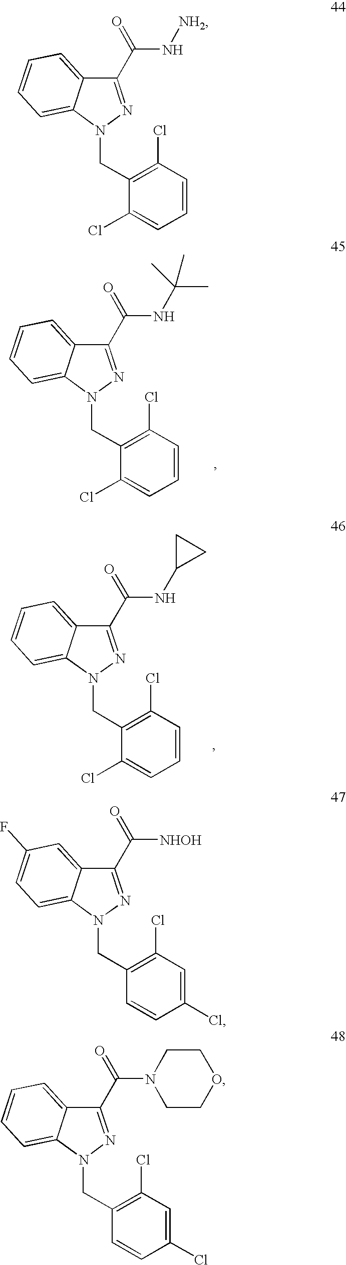

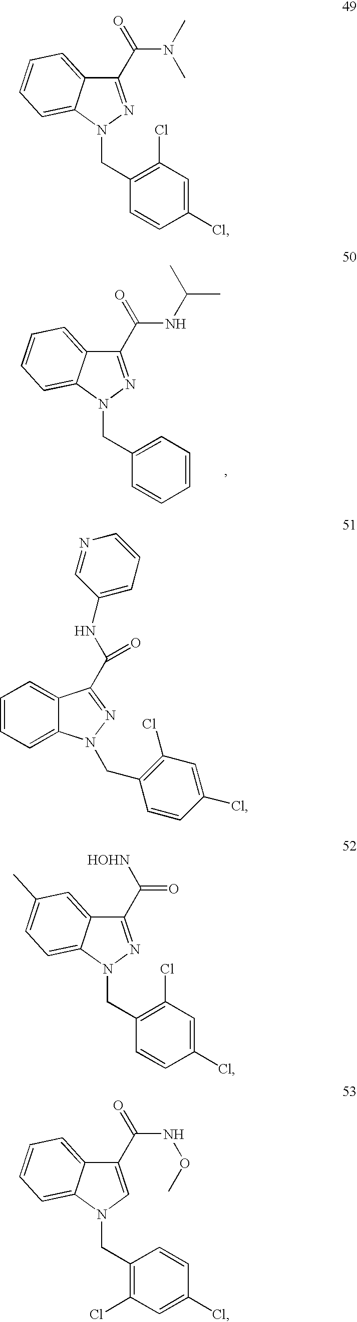

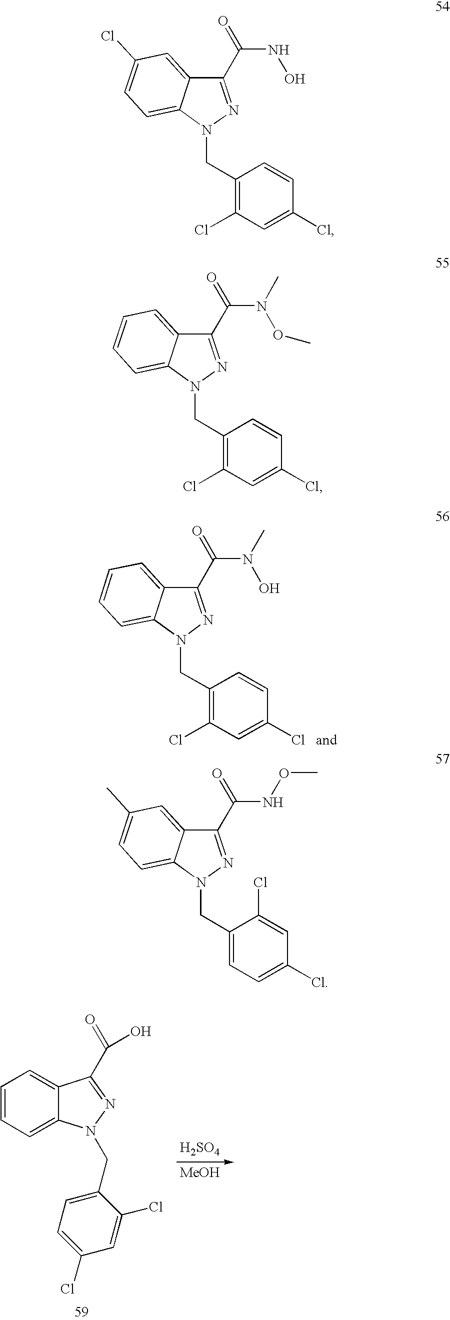

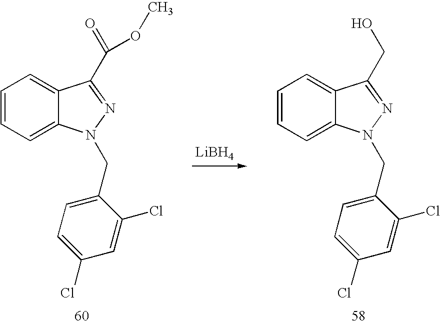

### Example 10

### Compound 58

Preparation of Compound 58

96% H₂SO₄ (40 mL) was added dropwise to a mixture of 59 (10 g) in MeOH (350 mL) over 30 min. The solution was refluxed for 6 hrs. Water (500 mL) was added and the mixture was extracted with EtOAc (3×200 mL). The organic phase was washed with 10% NaHCO₃, brine, dried over Na₂SO₄, and concentrated under reduced pressure to give 9.04 g of compound 60.

A solution of LiBH₄ (40 mL, 2.0 M) was added dropwise to a solution of compound 60 (6.7 g) in THF (80 mL) over 1 hr. After the mixture was stirred at rt overnight, water (200 mL) was added and the mixture was extracted with EtOAc (3×100 mL). The organic phase was washed with 10% HCl, 10% NaHCO₃, brine, dried over Na₂SO₄, and concentrated under reduced pressure. The residue was crystallized from MeOH to yield 4.2 g of compound 58.

### Example 11

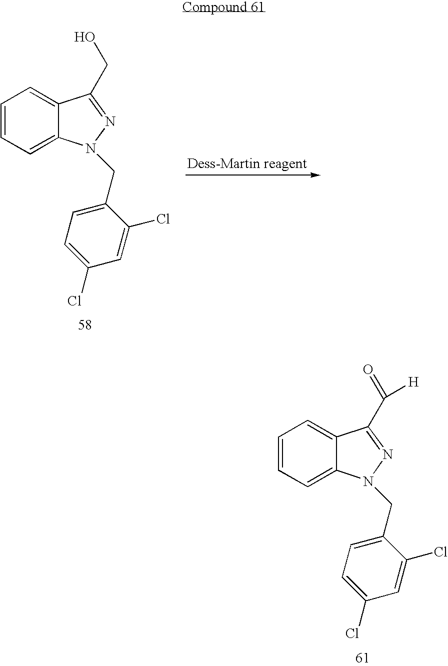 Preparation of Compound 61

Dess-Martin regent (0.3 M, 8 mL) was added to a solution of compound 58 (620 mg) in DCM (8 mL) at RT. After the solution was stirred for 1 hr at RT, DCM (10 mL) was added. NaOH (1 M) was added until the pH was about 7 and then the mixture was stirred for 5 min. The organic phase was washed with water, dried over Na₂SO₄, and concentrated under reduced pressure. Chromatography (Hex:AcOEt=100:25(V/V)) of the residue on silica gel afforded 610 mg of compound 61.

### Example 12

Preparation of Compound 64

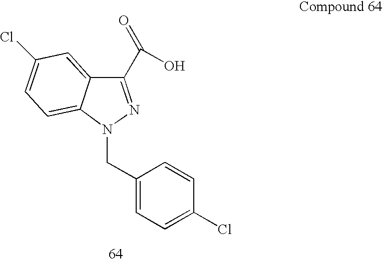

6-chloro-ethyl-1H-indazole-3-carboxylate (1.29 g, 5.74 mmol) was dissolved in dry DMF (10 ml) with potassium carbonate (1.59 g, 11.5 mmol). 4-Chlorobenzyl chloride (1.59 g, 6.89 mmol) was added and the reaction stirred at RT for 16 h. The reaction was poured into ethyl acetate (150 ml) and washed with water (2×100 ml) and sat. NaCl (1×100 ml). The organic phase was dried (MgSO4) and evaporated to give a tan solid. This solid was adsorbed onto silica gel and chromatographed with a gradient of hexane to hexane/ethyl acetate 7:3. The correct fractions were collected and reduced to give 1.18 g of a tan solid. ¹H NMR was consistent with the desired product.

The above ester (1.17 g) and sodium hydroxide (0.60 g) in ethanol (20 ml) and water (10 ml) were heated at 70° C. for 2 h. The reaction was diluted with water (150 ml), acidified to pH 2 (3M HCl), and the solid collected by filtration. After drying on the high vacuum, 719 mg of a pale yellow solid was obtained (compound 64). ¹H NMR was consistent with the desired product.

### Example 13

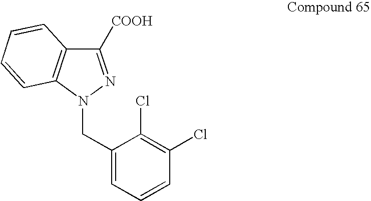 Preparation of Compound 65

### Preparation of Ethyl indazole-3-carboxylate (66)

Indazole-3-carboxylic acid (1.0 eq 92.5 mmol 15.0 g) is slurried in 300 mL EtOH. Thionyl chloride (3.0 eq 277.5 mmol 20.26 mL) is added via addition funnel over 45 min to the stirred slurry. The reaction mixture is heated to 80° C. under a water cooled reflux condenser and left refluxing overnight. The reaction mixture was cooled to room temperature and the solvent was carefully rotavaped off. 200 mL EtOH was added and rotary evaporated to remove all excess thionyl chloride. The reaction mixture was dissolved in 700 mL EtOAc, washed 3×100 mL with K₂CO₃ (aq) solution. The organic layer was washed 2×75 mL with a NaCl(aq) saturated solution. The organic layer was dried with magnesium sulfate, filtered with a medium frit and rotary evaporated to dryness. The crude product was recrystallized from boiling MeOH to yield 66.

Ethyl-1H-indazole-3-carboxylate 66 (1.60 g, 8.41 mmol) and 2,3-dichlorobenzyl alcohol (1.79 g, 10.1 mmol) were dissolved in dry THF under an argon atmosphere. Diisopropylazodicarboxylate (1.96 ml, 10.1 mmol) was added, followed by tri-n-butyl phosphine (2.49 ml, 10.1 mmol). The reaction was stirred at RT for 18 h, then evaporated to dryness. The residue was chromatographed on silica gel with a gradient of hexane to hexane/ethyl acetate 7:3. 1.14 g of the correct product was isolated as clear oil which later soldified. The ¹H NMR was consistent with the desired product.

The above compound (1.12 g, 3.12 mmol) and sodium hydroxide (0.90 g, 22.5 mmol) in ethanol (20 ml) and water (10 ml) were heated at 70° C. for 1.5 h. The reaction was diluted with water (150 ml), acidified to pH 2 (3M HCl), and extracted with 3×75 ml ethyl acetate. The organic phase was dried (MgSO₄) and evaporated to give 989 mg of a white solid compound 65. The ¹H NMR was consistent with the desired product.

### Example 14

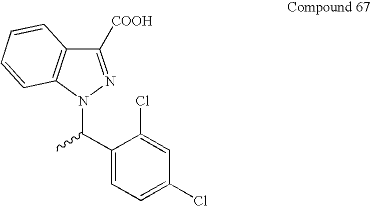 Preparation of Compound 67

A solution of 2,4-dichloro-alpha-methylbenzyl alcohol (4.00 g) in dry THF (5 ml) was added dropwise to thionyl chloride. The reaction was fitted with reflux condenser and heated at 80° C. for 2.5 h. The reaction mixture was then stripped on a rotory evaporator, 50 ml of toluene was added, and the mixture was stripped again. The resulting yellow oil was used without further purification.

Ethyl-1H-indazole-3-carboxylate 66 (1.01 g, 5.31 mmol) was dissolved in dry DMF (12 ml) with potassium carbonate (1.83 g, 13.3 mmol). The above yellow oil (1.33 g, 6.37 mmol) was added and the reaction stirred at RT for 16 h. and then heated at 60° C. for an additional 6.5 h. The reaction mixture was poured into ethyl acetate (150 ml) and washed with water (2×100 ml) and sat. NaCl (1×100 ml). The organic phase was dried (MgSO₄) and evaporated to give an orange oil. This material was chromatographed on silica gel with a gradient of hexane to hexane/ethyl acetate 7:3. The correct fractions were collected and reduced to give a 1.15 g of a clear oil. The ¹H NMR was consistent with the desired product.

The above product (1.14 g,) and sodium hydroxide (0.93 g) in ethanol (20 ml) and water (10 ml) were heated at 70° C. for 2 h. The reaction was diluted with water (150 ml), acidified to pH 2 (3M HCl) and extracted with 3×75 ml ethyl acetate. The organic phase was dried (MgSO₄) and evaporated to give 989 mg of a white solid compound 67. The ¹H NMR was consistent with the desired product.

### Example 15

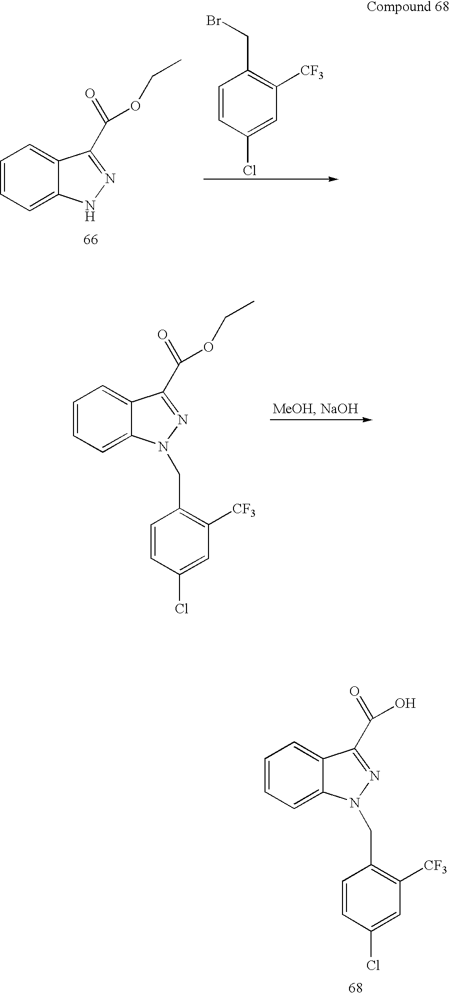 Preparation of Compound 68

To ethyl indazole-3-carboxylate 66 (1 eq, 1000 mg, 5.25 mmol) in a round-bottom flask, added N,N-dimethylformamide (50 mL), 4-chloro-2-(trifluoromethyl)benzyl bromide (1.2 eq, 1725.4 mg, 6.309 mmol) and potassium carbonate (1 eq, 725.6 mg, 5.25 mmol). The flask was heated in an oil bath to 70° C. for 2 hours. Water was added to the mixture, which became cloudy and resulted in a precipitate. The reaction mixture was filtered and the product was used directly in the next step.

To the starting material in a round-bottom flash, added methanol (50 mL) and NaOH 1M (50 mL). Heat the flash to 60 degree and leave stirring under reflux overnight. TLC showed the reaction complete. Added more water, acidified by 1% HCl, then use Ethyl acetate to extract 4 times. Dried over Sodium sulfate and rotavap-off the solvent. Recrystallized the product by adding Ethyl acetate (200 mL), then heated it up to dissolve all solid, then cooled down and added Hexane (50 mL), it started to crystallize. Filtered and washed with Hexane, then dried in high-vac to yield compound 68.

### Example 16

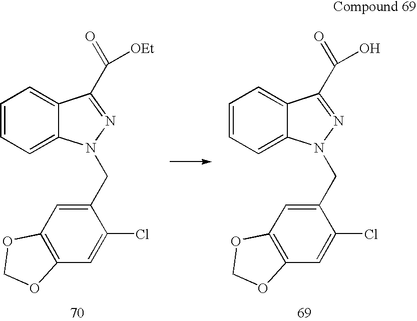 Typical Procedure for the Preparation of 69: Ethyl indazole-3-carboxylate Coupling with Substituted Methyl Chlorides:

Ethyl indazole-3-carboxylate (66) (1.0 eq 1.10 mmol 209 mg) is stirred in 4.0 mL anhydrous DMF. To this is added 6-chloropiperonyl chloride (1.3 eq 1.43 mmol 293 mg). Then K₂CO₃ (3.0 eq 3.30 mmol 456 mg) is added as a solid. This mixture was allowed to stir at room temp overnight. TLC done with mini work-up by sample into water extract with EtOAc, Hex 7:3 EtOAc. Two new spots form as a result of coupling to the nitrogen at the 1 position or at the 2 position of the indazole ring. The less polar of the two spots is the desired compound with the coupling occurring at the 1 position nitrogen. Reaction diluted into 60 mL of EtOAc. Wash 3×30 mL H₂O. Dry organic layer with magnesium sulfate. Filter medium frit, rotavap to dryness. Load sample onto Isco companion using a 40 gram disposable normal phase column. Run 0-40% EtOAc in Hexanes over 20 minutes.

Substituted 1-N-(6-Chloropiperonyl)-ethyl-indazole-3-carboxylate (70) (1.0 eq 0.25 mmol 90 mg) is dissolved in 4.0 mL EtOH and 2.0 mL H2O. Added NaOH pellets (3.0 eq 0.75 mmol 30 mg). Heat to 40 C. 3 hours check TLC by direct spotting on silica plate. Hex 1:1 EtOAc. Cool to room temperature. Dilute with 60 mL H₂O. Wash aqueous layer 1×40 mL EtOAc. Using 1M HCl bring aqueous layer to pH 4-5. Extract 3×70 mL EtOAc. Dry organic layers with magnesium sulfate, medium frit filter, rotavap to obtain product 69.

### Example 17

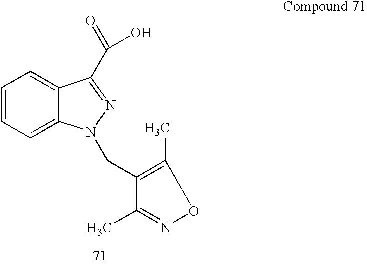 Preparation of Compound 71:

Obtained in the same manner as 69 by reaction of indazole-3-ethyl ester (66) (1.0 eq 1.10 mmol 210 mg) with 4-(chloromethyl)-3,5-dimethylisoxazole (1.3 eq 1.43 mmol 0.179 mL) and following same saponification procedure.

### Example 18

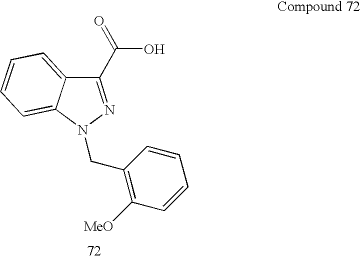 Preparation of Compound 72:

Obtained in the same manner as 69 by reaction of indazole-3-ethyl ester (66) (1.0 eq 1.13 mmol 214 mg) with 2-methoxybenzyl chloride (1.3 eq 1.47 mmol 0.149 mL) and following same saponification procedure.

### Example 19

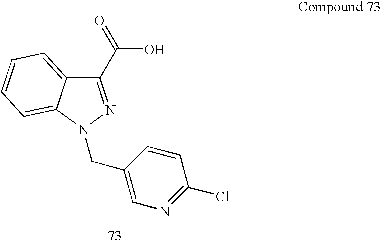 Preparation of Compound 73:

Obtained in the same manner as 69 by reaction of indazole-3-ethyl ester (66) (1.0 eq 1.08 mmol 208 mg) with 2-chloro-5-(chloromethyl) pyridine (1.3 eq 1.40 mmol 0.227 mL) and following same saponification procedure.

### Example 20

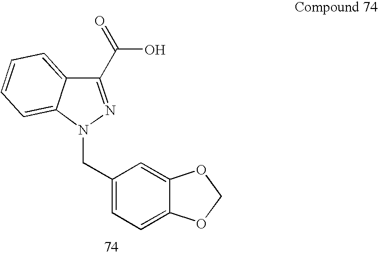 Preparation of Compound 74:

Obtained in the same manner as 69 by reaction of indazole-3-ethyl ester (66) (1.0 eq 1.05 mmol 200 mg) with 3,4-methylenedioxybenzyl chloride (1.3 eq 1.37 mmol 374 mg) and following same saponification procedure.

### Example 21

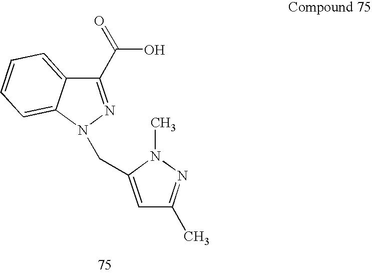 Preparation of Compound 75:

Obtained in the same manner as 69 by reaction of indazole-3-ethyl ester (66) (1.0 eq 1.05 mmol 200 mg) with 5-(chloromethyl)-1,3-dimethyl 1H-pyrazole (1.3 eq 1.37 mmol 198 mg) and following same saponification procedure.

### Example 22

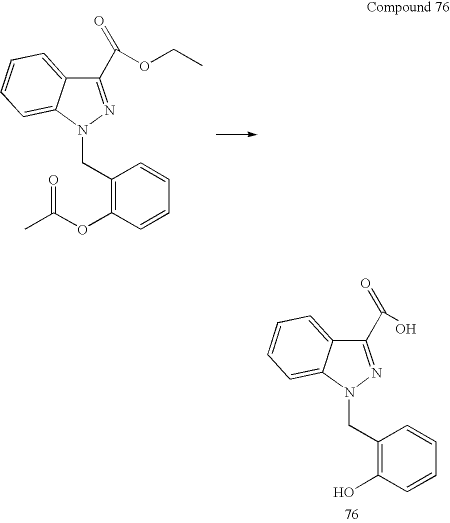 Preparation of Compound 76:

Obtained in the same manner as 69 by reaction of Indazole-3-ethyl ester (66) (1.0 eq 1.10 mmol 209 mg) with 2-(chloromethyl)-phenyl acetate (1.3 eq 1.43 mmol 264 mg) in DMF with K₂CO₃.

Saponification of the above ester (1.0 eq 0.18 mmol 60 mg) in 3.0 mL EtOH and 1.5 mL of H₂O with NaOH (3.0 eq 0.54 mmol 22 mg) at 40° C. 3 hours, check TLC by direct spotting on silica plate. Hex 1:1 EtOAc. Cool to room temperature. Dilute with 60 mL H2O. Wash aqueous layer 1×40 mL EtOAc. Using 1M HCl bring aqueous layer to pH 4-5. Extract 3×70 mL EtOAc. Dry organic layers with magnesium sulfate, medium flit filter, rotavap to obtain product 76. ¹H NMR confirmed removal of acetate and formation of alcohol.

### Example 23

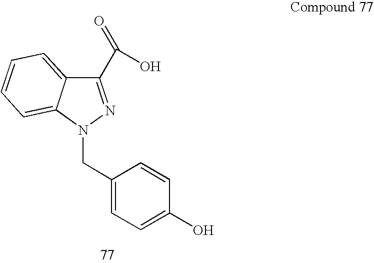 Preparation of Compound 77:

Obtained in the same manner as 76 by reaction of indazole-3-ethyl ester (66)(1.0 eq 1.08 mmol 206 mg) with 4-(chloromethyl)-phenyl acetate (1.3 eq 1.40 mmol 216 mg) in DMF with K₂CO₃ and following same saponification procedure.

### Example 24

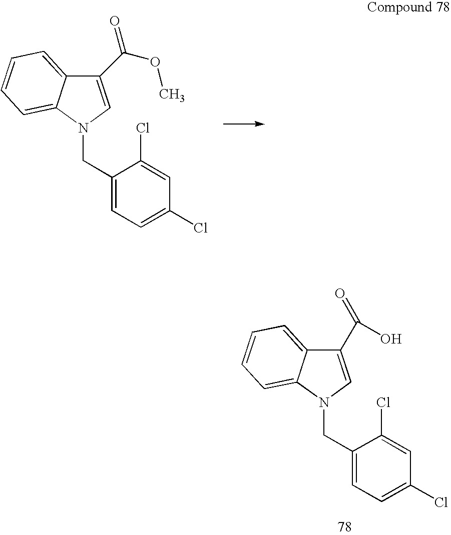 Preparation of Compound 78: Methyl Indole-3-carboxylate Coupling with Substituted Methyl Chlorides:

Methyl indole-3-carboxylate (1.0 eq 10.05 mmol 1.76 g) is stirred in 30.0 mL anhydrous DMF. To this is added 2,4-dichlorobenzyl chloride (1.3 eq 13.06 mmol 1.81 mL). Then K₂CO₃ (3.0 eq 30.15 mmol 4.17 g) was added as a solid. This mixture was allowed to stir at room temp overnight. TLC done with mini work-up by sample into water extract with EtOAc, Hex 7:3 EtOAc. Reaction diluted into 300 mL of EtOAc. Wash 3×100 mL H₂O. Dry organic layer with magnesium sulfate. Filter medium frit, rotavap to dryness. Load sample onto Isco companion using a 40 gram disposable normal phase column. Run 0-40% EtOAc in Hexanes over 20 minutes. Saponification of substituted methyl indole-3-carboxylate to carboxylic acid:

Substituted 1-N-(2,4-dichlorobenzyl)-methyl-indole-3-carboxylate (1.0 eq 0.25 mmol 90 mg) is dissolved in 4.0 mL EtOH and 2.0 mL H₂O. Added NaOH pellets (3.0 eq 0.75 mmol 30 mg). Heat to 40° C. 3 hours check TLC by direct spotting on silica plate. Hex 1:1 EtOAc. Cool to room temperature. Dilute with 60 mL H₂O. Wash aqueous layer 1×40 mL EtOAc. Using 1M HCl bring aqueous layer to pH 4-5. Extract 3×70 mL EtOAc. Dry organic layers with magnesium sulfate, medium frit filter, rotavap to obtain product 78.

### Example 25

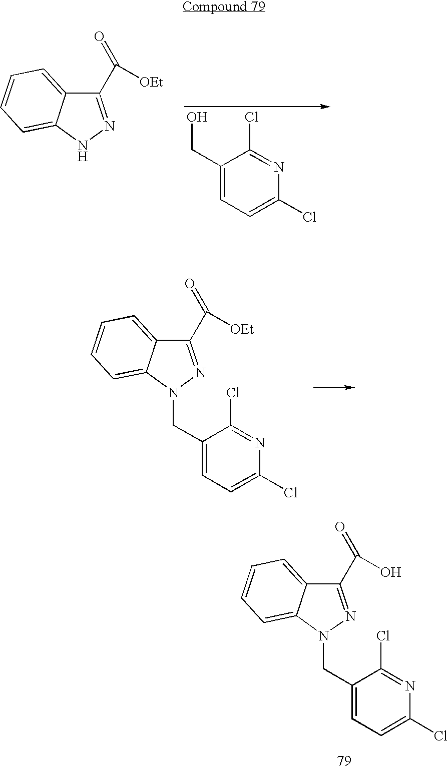 Preparation of Compound 79: Ethyl Indazole-3-carboxylate Coupling with 2,6-dichloropyridine-3-methyl Alcohol Via Mitsunobu.

2,6-dichloropyridine-3-carboxylic acid (1.0 eq 5.19 mmol 996 mg) is slowly added to a stirred solution of Lithium Aluminum Hydride (1.2 eq 6.23 mmol 237 mg) in 50 mL of anhydrous THF at −10° C. 1 hr TLC mini work-up consisting of dilution of sample in H2O and extraction with EtOAc. Hex 1:1 EtOAc, reaction complete. Warm reaction to room temp. Carefully add H₂O until foaming ceases. Extract by further diluting in 80 mL H2O and extracting with 3×150 mL EtOAc. Dry organic layer with magnesium sulfate and filter medium frit. Rotavap to dryness.

Ethyl indazole-3-carboxylate (1.0 eq 3.17 mmol 603 mg) is stirred in 12 mL anhydrous THF. 2,6-dichloropyridine-3-methyl alcohol (1.0 eq 3.17 mmol 503 mg) is added to stirred solution. DIAD (1.0 eq 3.17 mmol 0.615 mL) followed by slow addition of tri-n-butyl phosphine (1.0 eq 3.17 mmol 0.783 mL). Left at room temperature overnight. Dilute in 70 mL H₂O and extract with 3×100 mL EtOAc. Dry organic layer with magnesium sulfate and filter medium frit. Rotavap to dryness. Load sample onto Isco companion using a 40 gram disposable normal phase column. Run 0-40% EtOAc in Hexanes over 20 minutes.

The above ester (1.0 eq 0.41 mmol 145 mg) in 6.0 mL EtOH and 3.0 mL H₂O was saponified with NaOH (3.0 eq 1.23 mmol 49 mg) at 40° C. After 3 hours, the reaction mixture was cooled to room temperature and diluted with 100 mL H₂O. The aqueous layer was washed 1×80 mL with EtOAc, acidified to pH 4-5 using 1M HCl and extracted 3×150 mL of EtOAc. The organic layers were dried with magnesium sulfate, filtered with a medium frit filter and rotary evaporated to obtain product 79.

### Example 26

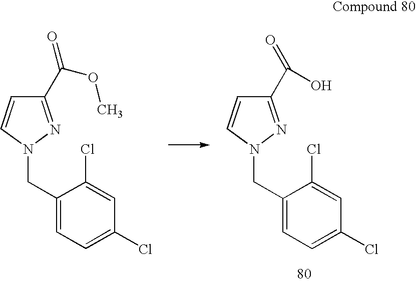 Procedure for the Preparation of Compound 80.

Methyl 1H-pyrazole-3-carboxylate (427 mg, 3.38 mmol) was dissolved in dry DMF (8 ml) with potassium carbonate (934 mg, 6.76 mmol). 2,4-Dichlorobenzyl chloride (564 uL, 4.06 mmol) was added and the reaction mixture was stirred for 2 h at RT. The reaction mixture was then poured into ethyl acetate (150 ml), and washed with water (2×75 ml) and saturated NaCl (1×75 ml). The organic phase was dried (MgSO₄) and evaporated to give a pale yellow oil. This oil was chromatographed on silica gel with a gradient of hexane to hexane/ethyl acetate 1:1, and 385 mg of the product was isolated as a colorless oil.

The above ester (1.0 eq 1.33 mmol 380 mg) in 8.0 mL EtOH and 4.0 mL H₂O was saponified by adding NaOH (3.0 eq 3.99 mmol 160 mg) at 40° C. After 3 hours, the reaction mixture was cooled to room temperature and diluted with 200 mL H₂O. The aqueous layer was washed 1×100 mL with EtOAc, acidified to pH 4.5 using 1M HCl, and extracted 3×250 mL with EtOAc. The organic layers were dried with magnesium sulfate, filtered with a medium frit filter and rotary evaporated to obtain product 80.

### Example 27

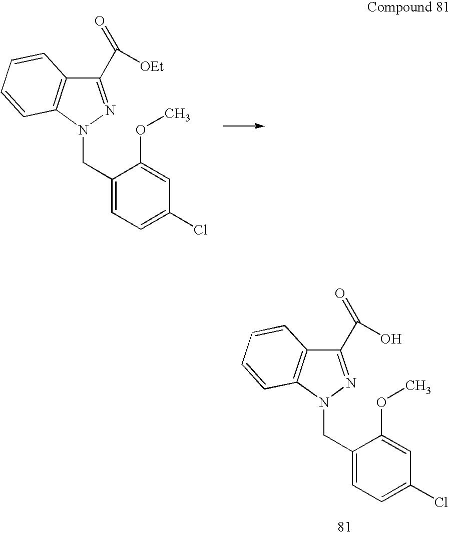 Procedure for Compound 81

Ethyl 1H-Indazole-3-carboxylate (150 mg, 0.79 mmol) and 4-chloro-2-methoxybenzyl alcohol (136 mg, 0.79 mmol) were dissolved in dry THF under an argon atmosphere. Diisopropylazodicarboxylate (153 uL, 0.79 mmol) was added, followed by tri-n-butyl phosphine (195 uL, 0.79 mmol). The reaction was stirred at RT for 21 hours, then stripped of solvent to give a pale yellow oil. This oil was chromatographed on silica gel with a gradient of hexane to hexane/ethyl acetate 7:13, and 104 mg of product was isolated as a colorless oil.

The above ester (1.0 eq 0.28 mmol 95 mg) was saponified in 6.0 mL EtOH/H₂O (2:1) by adding NaOH (3.0 eq 0.84 mmol 34 mg) and stirring at 40° C. for 3 hours. The reaction mixture was cooled to room temperature, diluted with 100 mL H₂O. The aqueous layer was washed 1×50 mL with EtOAc, acidified to pH 4.5 using 1M HCl, extracted 3×125 mL with EtOAc and the organic layers were dried with magnesium sulfate, filtered with a medium frit filter and rotory evaporated to obtain product 81.

### Example 28

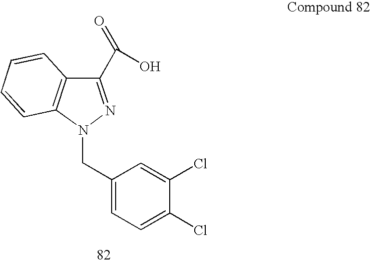 Preparation of Compound 82:

Obtained in the same manner as 69 by reaction of indazole-3-ethyl ester (23) (1.0 eq 8.04 mmol 1.53 g) with 3,4-dichlorobenzyl chloride (1.3 eq 10.46 mmol 1.45 mL) and followed by the same saponification procedure. 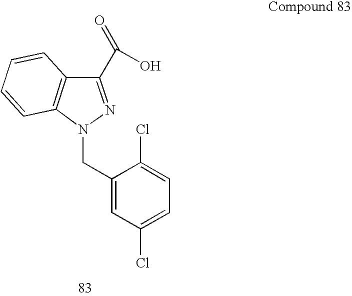
Preparation of Compound 83:

Obtained in the same manner as 69 by reaction of indazole-3-ethyl ester (23) (1.0 eq 8.20 mmol 1.56 g) with 2,5-Dichlorobenzyl bromide (1.3 eq 10.66 mmol 2.56 g) and followed by the same saponification procedure. 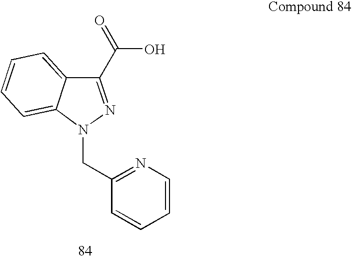
Preparation of Compound 84:

Obtained in the same manner as 79 by reaction of indazole-3-ethyl ester (23) (1.0 eq 2.68 mmol 510 mg) with 2-pyridylcarbinol (1.0 eq 2.68 mmol 258 uL) via Mitsunobu followed by the same saponification procedure.

### Example 31

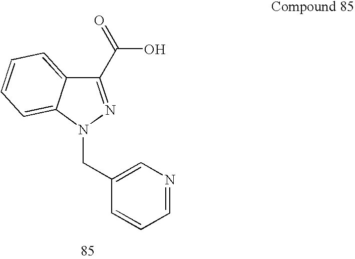 Preparation of Compound 85:

Obtained in the same manner as 79 by reaction of indazole-3-ethyl ester (23) (1.0 eq 2.70 mmol 514 mg) with 3-pyridylcarbinol (1.0 eq 2.70 mmol 260 uL) via Mitsunobu followed by the same saponification procedure.

### Example 32

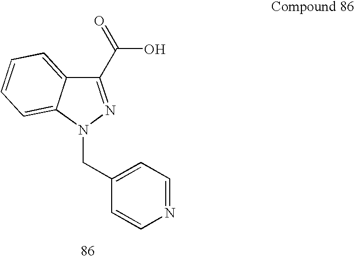 Preparation of Compound 86:

Obtained in the same manner as 79 by reaction of indazole-3-ethyl ester (23) (1.0 eq 2.71 mmol 516 mg) with 4-pyridylcarbinol (1.0 eq 2.71 mmol 296 μL) via Mitsunobu followed by the same saponification procedure.

### Example 33

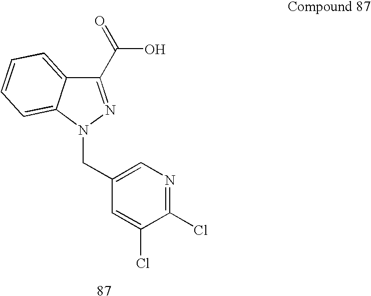 Preparation of Compound 87:

Obtained in the same manner as 79 by reaction of Indazole-3-ethyl ester (23) (1.0 eq 2.73 mmol 519 mg) with 5,6-Dichloro-3-pyridinemethanol (1.0 eq 2.73 mmol 486 mg) via Mitsunobu followed by the same saponification procedure.

### Example 34

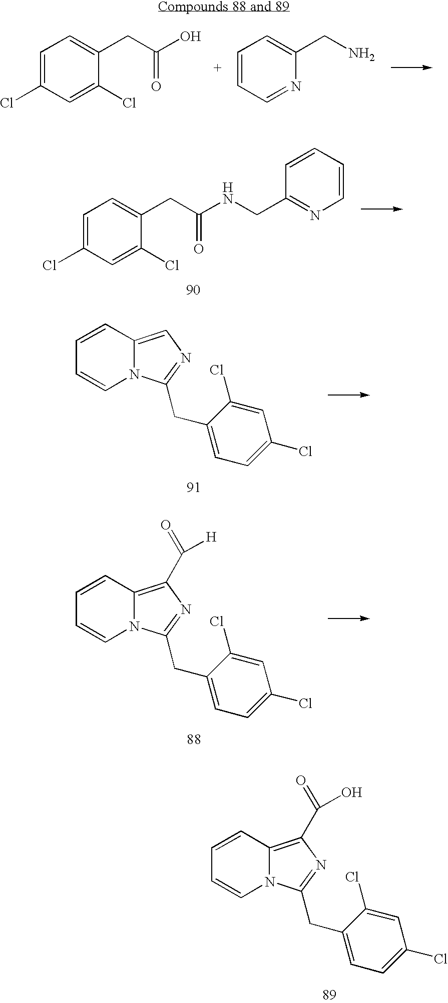 Preparation of Compounds 88 and 89

To a solution of 2,4-dichlorophenylacetic acid (1 gm, 4.88 mmol) and 2-(aminomethyl)pyridine (503 μl, 4.88 mmol) in DMF (20 ml) was added HBTU (2.04 gm, 5.37 mmol) at C, followed by adding DIEA (1.7 ml, 9.76 mmol). The reaction mixture was warmed up to room temperature and stirred over night. The reaction mixture was diluted with ethyl acetate and washed with NaHCO₃ twice. The organic layer was dried with MgSO₄, filtered and concentrated. The residue was purified with silica gel chromatography (acetone in toluene from 0 to 100%) to give white solid product 90.

Compound 90 (0.86 gm, 2.91 mmol) was dissolved in THF (20 ml) and TEA (2 ml) and the resulting solution was chilled was cooled to −15° C. A solution PCl₃ (1.6 ml, 2 M in DCM) in THF (3 ml) was added slowly. After 30 minutes, TLC showed a complete reaction. Reaction mixture was poured into brine and extracted with ethyl acetate twice. The combined extracts were dried with MgSO₄, filtered and concentrated. The residue was purified with silica gel flash chromatography column (ethyl acetate in hexane 0 to 60%). Pure product 91 was obtained.

To a solution of compound 71 (50 mg, 0.18 mmol) in dry DMF (3 ml) was added Vilsmeier reagent (POCl₃ (33 μl, 0.36 mmol) in DMF (0.5 ml)) and the mixture was stirred at room temperature for one hour. The reaction solution was treated with cold water and made basic with solid Na₂CO₃ and extracted with ethyl acetate four times. The combined organic layers were dried with MgSO₄, filtered and concentrated. The residue was purified with silica gel column (ethyl acetate in hexane from 0 to 60%) to give light yellow solid product 88.

To a suspension of 88 (70 mg, 0.23 mmol) in EtOH (3 ml) was added a suspension of AgNO₃ (80 mg, 0.46 mmol) and NaOH (37 mg, 0.92 mmol) in water (3 ml). The mixture was sonicated for half hour and then vigorously stirred at room temperature for two days. After acidified with HCl, the mixture was extracted with DCM four times. The combined extracts were dried and the solvent was removed The residue was purified with flash silica gel column (MeOH in DCM, 0 to 30%) to produce white solid 89.

### Example 35

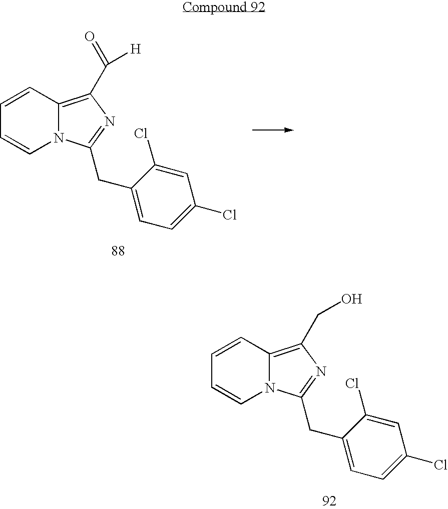 Preparation of Compounds 92

To a solution of 88 (22 mg, 0.072 mmol) in THF (2 ml) was added lithium borohydride (72 μl, 0.144 mmol, 2 M in DCM) at room temperature. The resulting solution was stirred for overnight. Solvent was removed and the residue was purified with silica gel chromatography column (MeOH in DCM from 0 to 20%) to give product 92.

### Example 36

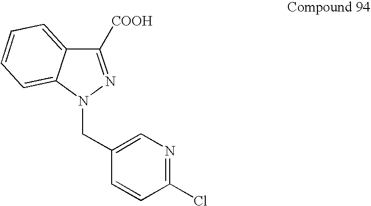 Preparation of Compound 94:

Obtained in the same manner as 79 by reaction of Indazole-3-ethyl ester (23) (1.0 eq 2.73 mmol 519 mg) with 4-chloro-3-pyridylcarbinol (1.0 eq 2.73 mmol 486 mg) via Mitsunobu followed by the same saponification procedure.

### Example 37

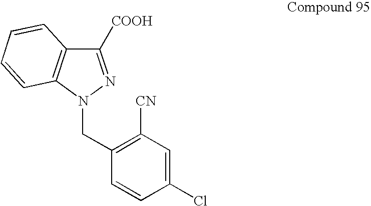 Preparation of Compound 95:

### 4-chloro-2-cyanobenzyl bromide

5-chloro-2-methylbenzonitrile (1.00 g), N-bromosuccinimide (2.82 g), and benzoyl peroxide (80 mg) were suspended in carbon tetrachloride (25 ml) and heated at 80° C. for 80 minutes. The reaction mixture was stripped of solvent, dissolved in 10 ml CH₂Cl₂, filtered and evaporated to give a yellow oil. This was chromatographed on silica gel using a gradient of Hexane/0-15% ethyl acetate to give 640 mg of 4-chloro-2-cyanobenzyl bromide as a white solid. ¹H NMR was consistent with the desired product.

t-Butyl Indazole-3-carboxylate (497 mg), 4-chloro-2-cyanobenzyl bromide (630 mg) and potassium carbonate (630 mg) were stirred in DMF (6 ml) for 18 hours at room temperature. The reaction mixture was then added to ethyl acetate (80 ml) and washed with water (2×50 ml) and brine (1×50 ml). The organic phase was dried over MgSO₄ and evaporated to give a yellow oil. This was chromatographed on silica gel using a gradient of Hexane/0-25% ethyl acetate to give 264 mg of a white solid. ¹H NMR was consistent with the desired product.

The above compound (260 mg) was dissolved in CH₂Cl₂ (7 ml) and trifluoroacetic acid (1.5 ml) added. The reaction mixture was stirred at room temperature for 2.25 hours, then evaporated to dryness. After standing on the high vacuum, compound 95 was obtained as a white solid. ¹H NMR was consistent with the desired product.

### Example 38

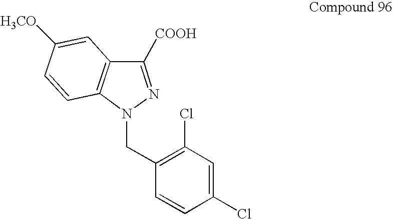 Preparation of Compound 96:

Compound 96 was prepared in a similar way as compound 26, using 5-methoxyl-1H-indazole 3-carboxylic acid and 2,4-dichlorobenzyl chloride as starting materials

### Example 39

### Antiproliferation Assay

To determine the effect of lonidamine and analogs thereof on cell proliferation, the antiproliferative activity of these compounds was tested in a multi-well Alamar Blue based assay (at 2 h and 3 days). Cell growth in the presence and absence of the test compound (compounds 1-22) was compared, as measured by a fluorescence plate reader at excitation 550 nm and emission 590 nm (see Biosource International Inc., Tech Application Notes, Use of Alamar Blue in the measurement of Cell Viability and Toxicity, Determining IC₅₀). H460 cells (ATCC HTB-177 (NCI-H40), 4,000 cells/well/200 μl) and LNCap cells (ATCC CRL-1740, 6,000 cells/well/200 μl) were seeded in a 96 well plate in RPMI medium (Invitrogen Corporation, Carlsbad, Calif.). After 24 hours, these plates were divided into 3 groups—Control group, 2 h treatment group and 3 day treatment group. A test compound was added to each plate in the treatment groups (2 h and 3 day) at a concentration as tabulated in Table 1 (in 50 ml of medium). In the 2 h treatment group, after 2 h the cells were rinsed to remove the test compound and incubated for 3 days, followed by staining with AlamarBlue. The cells in the 3 day treatment group were incubated for 3 days, followed by staining with AlamarBlue. In the Control group, AlamarBlue was added to the plate at (i) day 0 and (ii) day 3 and measured to establish the control reading. In all the groups, the capacity of the cells to proliferate was measured 6 hours after addition of AlamarBlue by a fluorescence plate reader at excitation 550 nm and emission 590 nm and the 50% growth inhibitory concentration (GI₅₀ (also referred to IC₅₀ herein)) of lonidamine and lonidamine analogs was calculated. The results of the assay are tabulated in Table 3.

TABLE 3
GI₅₀ (μM) of lonidamine and lonidamine
analogs in proliferation assay

| | |
|---|---|
| H460 cell | LNCaP cell |

| Compound No. | 2 hr | 3 days | 3 days |
|---|---|---|---|
| 1 | 50 | 15.8 | 40 |
| 2 | 400 | 31.6 | — |
| 3 | 398 | 250 | 180 |
| 4 | 281 | 158 | 178 |
| 5 | 40 | 20 | 20 |
| 6 | >400 | >400 | — |
| 7 | >400 | 20 | — |
| 8 | >400 | 316 | — |
| 9 | >400 | >400 | — |
| 10 | 78 | 50 | — |
| 11 | — | 199.5 | — |
| 12 | 251 | >400 | 125 |
| 13 | >400 | 200 | 200 |
| 14 | | >400 | >640 |
| 15 | >400 | 316 | |
| 18 | >400 | >400 | |
| 19 | >400 | 200 | |
| 20 | >400 | >400 | |
| 21 | >400 | 398 | |
| 22 | >400 | 158 | |

wherein compounds 1-15 have the following structure

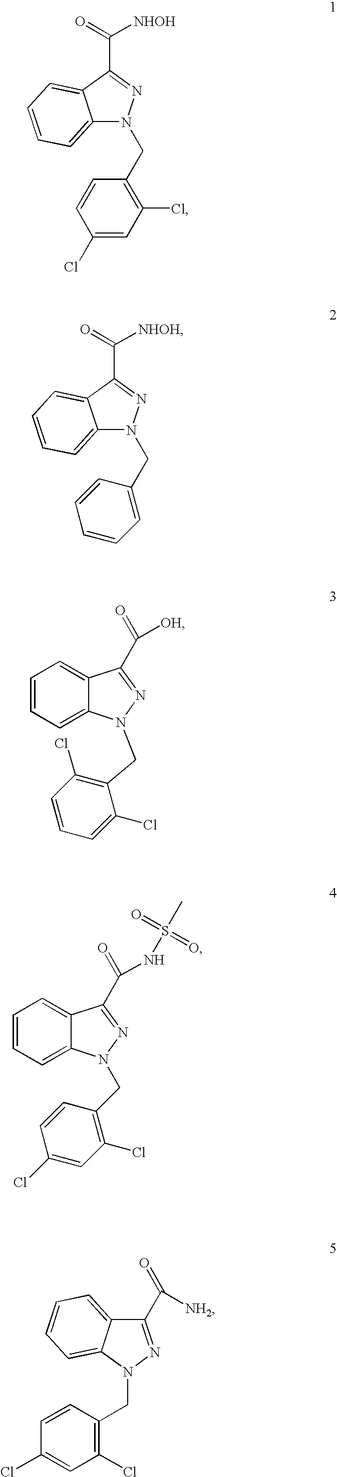

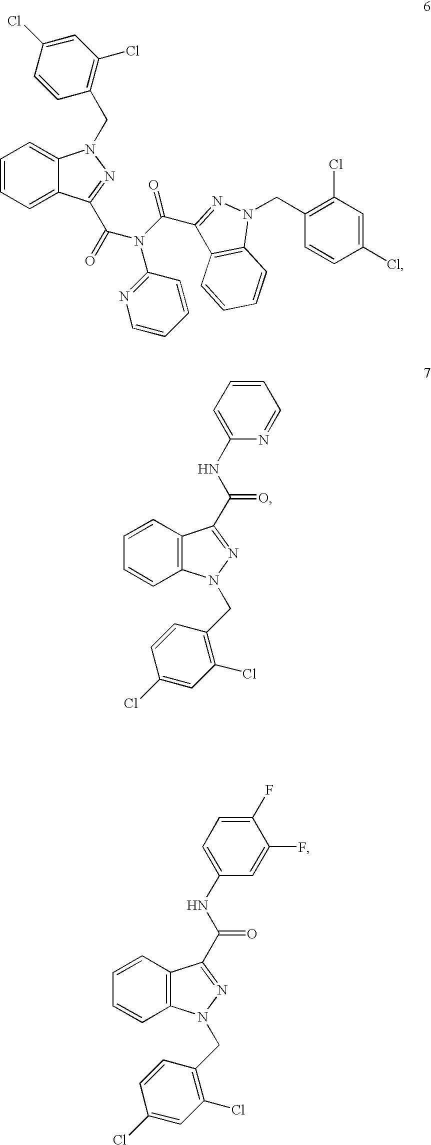

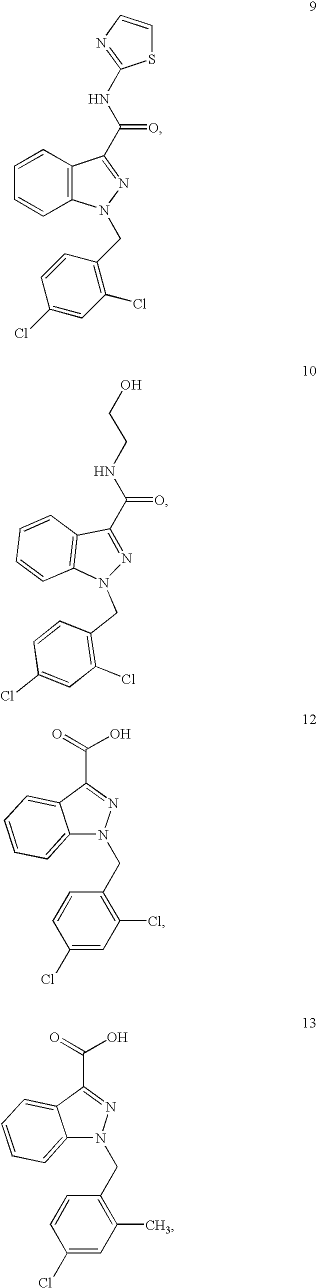

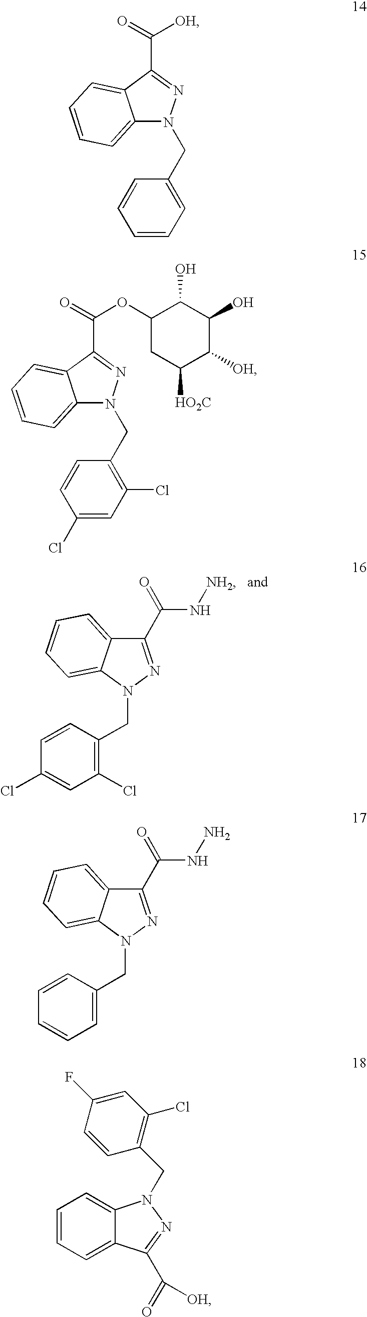

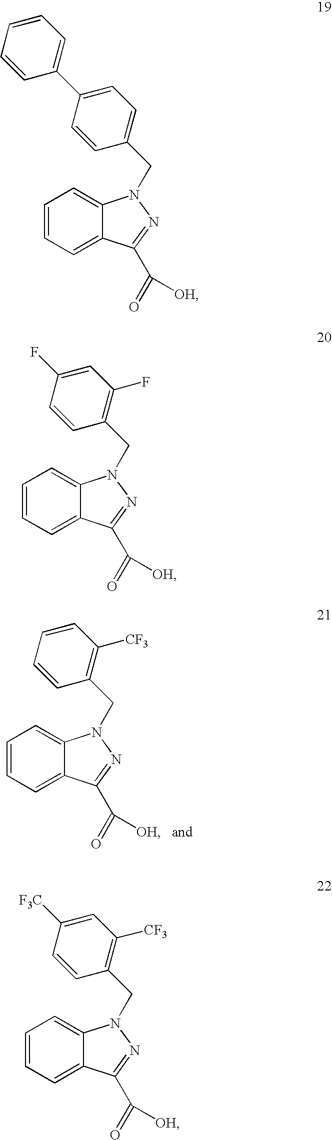

### Example 40

### Antiproliferation Assay

The effect of lonidamine and analogs thereof on cell proliferation in PWR-1E cells (ATCC CRL-11611) was determined in an antiproliferative assay using PWR-1E cells (5000 cells/well) in Keratinocyte SFM medium (Gibco Products, Invitrogen Corporation, Carlsbad, Ca.) according to the procedure detailed in Example 39 above. The results of the assay are tabulated in Table 4.

TABLE 4
IC₅₀ (μM) of lonidamine and lonidamine
analogs in proliferation assay

| Compound No. | PWR-1E cell |
|---|---|
| 1 | 4 |
| 2 | 18 |
| 3 | 25 |
| 4 | 18 |
| 5 | 4 |
| 11 | 27 |
| 12 | 20 |
| 13 | 25 |
| 14 | >400 |

### Example 41

### Antiproliferation Assay

To determine the effect of lonidamine and analogs thereof on cell proliferation, the antiproliferative activity of these compounds was tested in a multi-well Alamar Blue based assay (at 3 days). Cell growth in the presence and absence of the test compound was compared, as measured by a fluorescence plate reader at excitation 550 nm and emission 590 nm (see Biosource International Inc., Tech Application Notes, Use of Alamar Blue in the measurement of Cell Viability and Toxicity, Determining IC₅₀). The following cell lines were tested: PWR-1E cells ((ATCC CRL-11611), 3500 cells/well/200 μl in Keratinocyte SFM medium (Gibco Products, Invitrogen Corporation, Carlsbad, Calif.)); DU-145 cells ((ATCC HTB-81), 4000 cells/well/200 μl in MEM Eagles medium (ATCC, Manassas, Va.)); PC3 cells ((ATCC CRL-1435), 4000 cells/well/200 μl in Modified HAM's F12 medium (ATCC, Manassas, Va.)); PNt2 cells ((Sigma-Aldrich, 95012613-1 VL), 4000 cells/well/200 μl in RPMI medium (Gibco Products, Invitrogen Corporation, Carlsbad, Calif.), BPH-1 ((DSMZ ACC-143), 4000 cells/well/200 μl in RPMI medium (Gibco Products, Invitrogen Corporation, Carlsbad, Ca.), and NCI-H460 cells ((ATCC HTB-177), 4000 cells ((ATCC HTB-177), 4000 cells/well/200 μl in RPMI medium (Gibco Products, Invitrogen Corporation, Carlsbad, Calif.)). The cells were seeded in a 96 well plate in a medium as specified above. After 24 hours, these plates were divided into 2 groups—Control group and 3 day treatment group. A test compound was added to each plate in the treatment groups at concentrations of 300, 100, 30, 10, 3, 1 and 0.3 μl M as tabulated in Table 1 (in 2 μl 1 of 100% DMSO, final DMSO concentration 1% in all wells). For NCI-H460 cells, test compounds were added to each plate in the treatment groups at concentrations of 600, 300, 100, 30, 10, 3, and 1 μM (in 2 μl of 100% DMSO, final DMSO concentration 1% in all wells). The cells in the 3 day treatment group were incubated for 3 days, followed by staining with AlamarBlue. In the Control group, AlamarBlue was added to the plate at (i) day 0 and (ii) day 3 and measured to establish the control reading. In all the groups except those tested in NCI-H460 cells, the capacity of the cells to proliferate was measured 6 hours after addition of AlamarBlue by a fluorescence plate reader at excitation 550 nm and emission 590 nm and the 50% growth inhibitory concentration (GI₅₀ (also referred to IC₅₀ herein)) of lonidamine and lonidamine analogs was calculated. For the NCI-H460 cells, the capacity of the cells to proliferate was measured 5 hours after addition of AlamarBlue using the same fluorescence plate reader as stated above. The results of the assay are tabulated in Table 5.

TABLE 5
IC₅₀ (μM) of lonidamine and lonidamine analogs in proliferation assay

| | | | | | | NCI- |
|---|---|---|---|---|---|---|
| | PWR1e cell | Du-145 cell | BPH-1 cell | PNT2c cell | PC-3 cell | H460 cell |
| | line IC50 | line IC50 | line IC50 | line IC50 | line IC50 | line IC50 |
| Compound | (μM) | (μM) | (μM) | (μM) | (μM) | (μM) |
| 12 | 31 | 214.5 | 230 | 197.5 | 195 | |
| 14 | >400 | >300 | >300 | >300 | >300 | |
| 13 | 24 | | | | | |
| 3 | 69 | 224 | 215 | 257 | 191 | |
| 1 | 5.3 | 11 | 17.5 | 17 | 15 | |
| 2 | 18 | | | | | |
| 6 | >10 | | | | | |
| 8 | >3 | | | | | |
| 9 | >100 | | | | | |
| 10 | 14 | | | | | |
| 5 | 5.5 | | | | | |
| 25 | >100 | | | | | |
| 15 | 80 | | | | | |
| 18 | 100 | | | | | |
| 20 | 100 | | | | | |
| 21 | >100 | | | | | |
| 22 | 25 | | | | | |
| 27 | 30 | | | | | |
| 28 | 75 | >100 | | >100 | >100 | |
| 37 | 36 | | | | | |
| 40 | 20 | | | | | |
| 41 | 9.7 | | | | | |
| 42 | 22 | | | | | |
| 43 | 45 | | | | | |
| 44 | >10 | | | | | |
| 30 | 22 | | | | | |
| 46 | >30 | | | | | |
| 47 | 5.6 | | | | | |
| 48 | >10 | | | | | |
| 49 | >100 | | | | | |
| 50 | 23 | | | | | |
| 31 | 9.5 | | | | | |
| 32 | >10 | | | | | |
| 33 | 52 | | | | | |
| 51 | | | | | | |
| 52 | 0.34 | | | | | |
| 53 | | | | | | |
| 54 | 1.3 | | | | | |
| 56 | 2.6 | 3.25 | 1.65 | 1.25 | 4 | |
| 58 | | | | | | |
| 61 | 1.5 | | | | | |
| 34 | 18 | 88.5 | 91.5 | 101 | 70 | |
| 62 | 0.9 | | | | | |
| 35 | >300 | | | | | |
| 69 | 145 | | | | | |
| 76 | 288 | | | | | |
| 71 | >300 | | | | | |
| 72 | 245 | | | | | |
| 73 | >300 | | | | | |
| 57 | 39 | | | | | |
| 63 | 102 | | | | | |
| 80 | >100 | | | | | 520 |
| 81 | 70 | | | | | 182 |
| 77 | >100 | | | | | |
| 74 | >100 | | | | | 290 |
| 75 | >300 | | | | | >180 |
| 79 | 245 | | | | | >180 |
| 78 | 22 | | | | | 89 |
| 82 | 36 | | | | | 128 |
| 83 | 38 | | | | | 126 |
| 65 | 29 | | | | | 165 |
| 84 | | | | | | >600 |
| 85 | | | | | | >600 |
| 86 | | | | | | >600 |
| 87 | | | | | | 323 |
| 94 | | | | | | >600 |
| 88 | 12 | | | | | 48 |
| 89 | 10 | | | | | 41 |
| 92 | 11 | | | | | 43 |
| 95 | 85 | | | | | 275 |

### Example 42

### BrdU-TUNEL Assay

The effect of compound 1 (as described in Example 1 above) on apoptosis was determined as follows. PWR-1E cells (2×10⁵ cells/ml/well) were seeded in a 24 well plate. After 24 h compound 1 was added at various concentrations as tabulated in Table 6. The culture media were removed after 24 h, the cells were rinsed with PBS buffer (200 μL) and incubated (5 min, 37° C.) with a solution of Guava Viacount CDR in PBS (1:3 v/v). Media (750 μL) containing at least 5% FBS was added to each well, the cells released by repeated pipeting, centrifuged, and the supernatant aspirated. The cells were resuspended in PBS buffer (150 μL) and fixed by incubating (60 min, 4° C.) with 4% paraformaldehyde in PBS. The cells were centrifuged, and the supernatant removed to a final volume of 15 mL. The cell pellets were resuspended, followed by dropwise addition of 200 μl of ice-cold ethanol (70%), and the cells incubated at −20° C. at least for 2 hr. The cells were centrifuged, the supernatant removed, washed, and incubated with the DNA labeling mix (37° C., 60 min). The cells were washed, incubated (30 min) with anti-BrdU staining mix, washed again and analyzed on a Guava PCA-96 system (Guava Technologies, 25801 Industrial Boulevard, Hayward Calif. 94545-2991, USA).

The effect of compound 1 on apoptosis of LNCaP cells was determined using the same protocol as described in Example 38.

TABLE 6

| Compound 1 (μM) | % apoptotic cells | % Non-apoptotic cells |
|---|---|---|
| 0 | 12 | 88 |
| 3.1 | 10 | 90 |
| 6.2 | 12 | 88 |
| 12.5 | 22 | 78 |
| 25 | 52 | 48 |

As tabulated in Table 6, Compound 1 induces apoptosis in PWR1E cells.

### Example 43

### Cell Cycle Analysis

The effect of compound 1 (as described in Example 1 above) on the cell cycle was determined as follows. LNCaP cells (2×10⁵ cells/ml/well) were seeded in a 24 well plate. After 24 h, compound 1 was added at various concentrations as tabulated in Table 7. The culture media were removed after 24 h, the cells were trypsinized and centrifuged. The cell pellets were resuspended in 10011 PBS buffer, after which 300 μl of ice-cold ethanol (96%) added dropwise, and the cells were incubated at 4° C. for at least 24 hr. The cells were centrifuged and the supernatant was discarded. The cell cycle staining reagent (Guava Technologies, Hayward, Calif., USA, 200 μl) was added to each well. The cells were shielded from light and incubated at room temperature for 30 min. The samples were analyzed (Guava PCA-96 instrument, Cytosoft software, Guava Technologies, 25801 Industrial Boulevard, Hayward Calif. 94545-2991, USA) as tabulated below.

TABLE 7

| Compound 1 (μM) | % G0/G^{a} | % S^{b} | % G2^{c}/M^{d} |
|---|---|---|---|
| 0 | 52 | 13 | 32 |
| 2.5 | 59 | 11 | 27 |
| 7.4 | 56 | 15 | 26 |
| 22.2 | 57 | 11 | 25 |
| 66.7 | 51 | 19 | 24 |
| 200 | 41 | 18 | 17 |

^{a}= G0/G1 (Gap 1), phase when cells prepare for cell division cycle
^{b}= S phase, DNA synthesis or replication phase
^{c}= G2 (Gap 2), phase when cells prepare for mitosis
^{d}= M phase, mitosis i.e. cell division phase.

Compound 1 does not have a measurable effect on the cell cycle.

### Example 44

### Mouse Studies

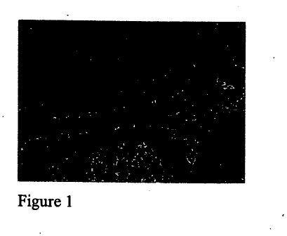

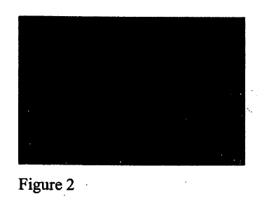

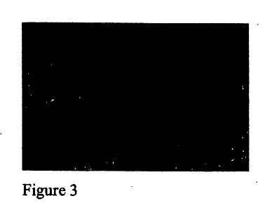

The effect of Compound 1 on the mouse prostate was determined as follows. Compound 1 was orally administered daily for 5 days to male, C57Bl/6J mice, (n=5, 6-8 weeks old) 1 at 2, 5, and 20 mg/kg (as a 1% carboxymethylcellulose formulation). The control mice received an equal amount of the vehicle (carboxymethylcellulose). On day 6 the mice were sacrificed and the entire prostate and the individual lobes (e.g., the dorsal lobe and the ventral lobe) were weighed to measure absolute weights. Relative weights of prostate and individual lobes were calculated by dividing the absolute weight by the total weight of the mouse. Relative weights of the entire prostate, the dorsal prostate, and the ventral prostate were calculated by dividing the absolute weight by the total weight of the mouse. Both the absolute entire prostate and relative entire prostate weights reduced in the 5 and 20 mg/kg groups compared to the control group. The histomorphology of the prostate was also analyzed and compared to that of the control or untreated prostate as illustrated in FIGS. 1-3, showing upon administration of Compound 1. The results show a dose-dependent disorganization of the epithelial cells in animals receiving Compound 1.

### Example 45

### Mouse Studies

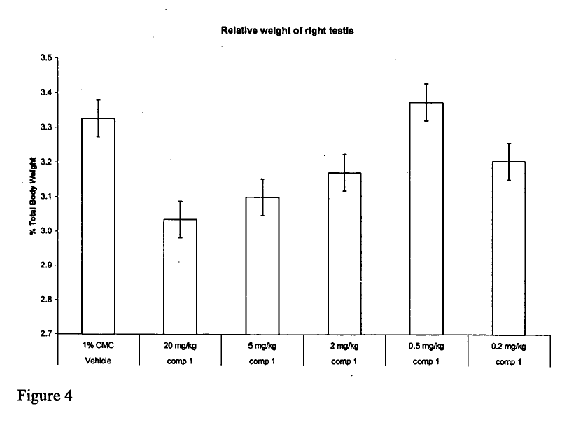

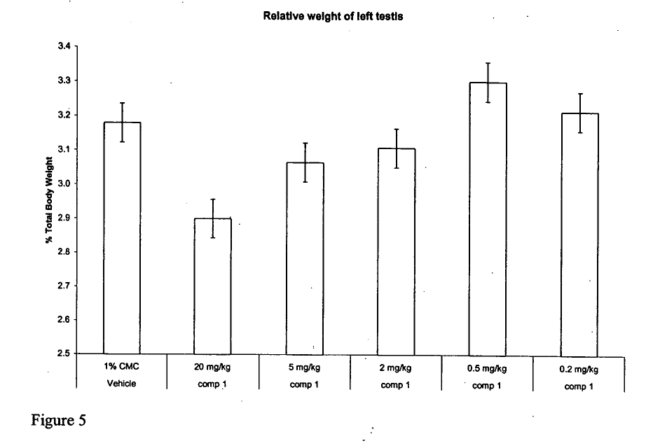

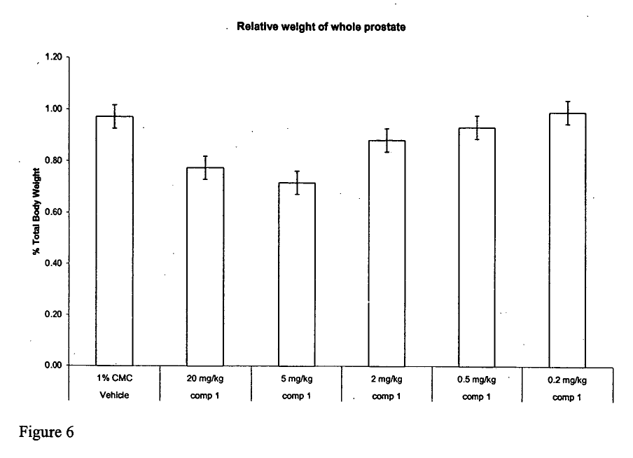

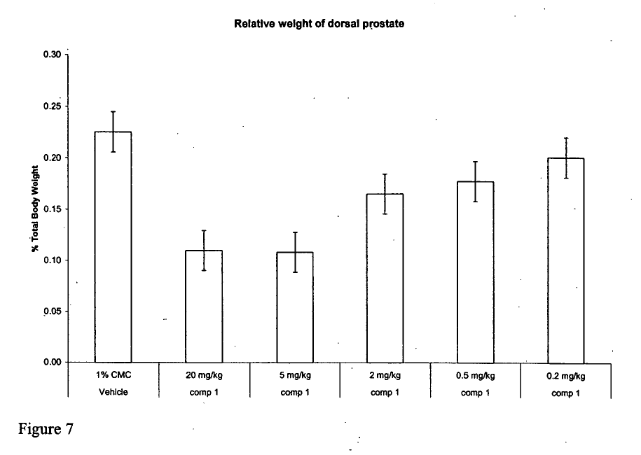

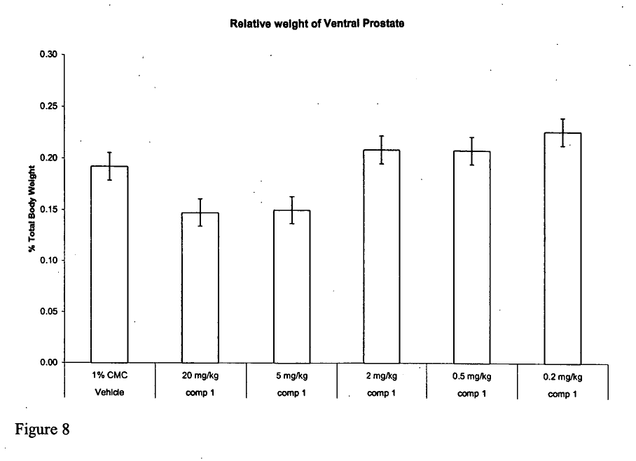

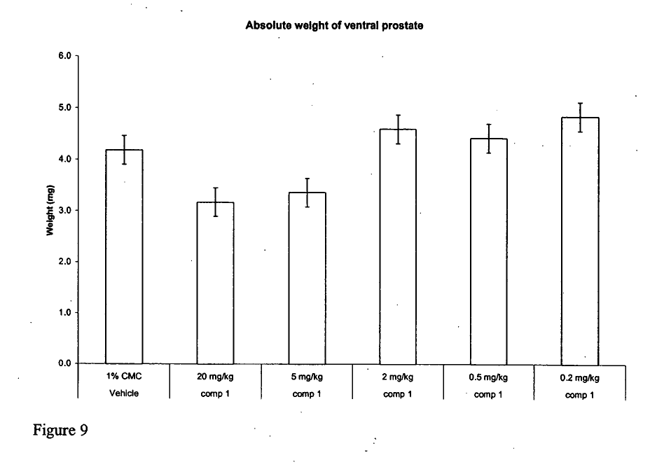

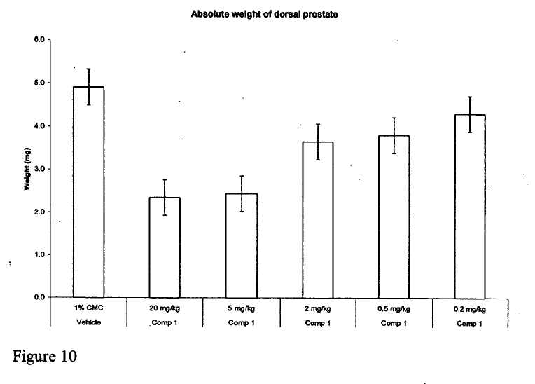

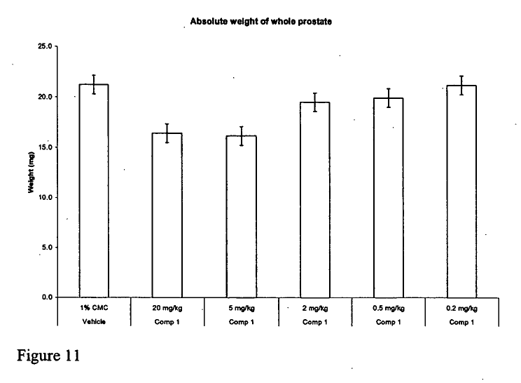

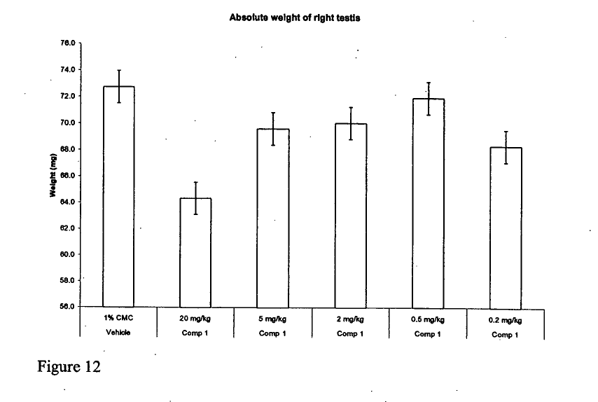

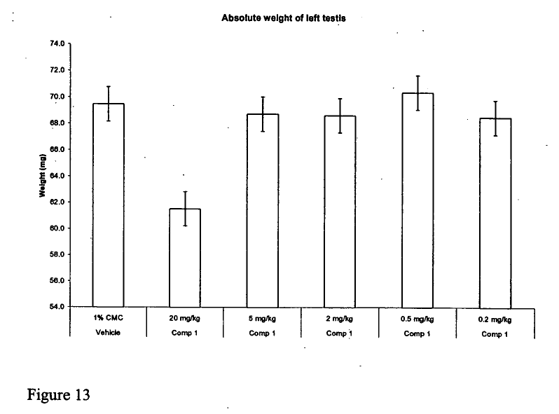

The effect of Compound 1 on the mouse prostate was determined as follows. Compound 1 was orally administered daily for 10 days to male, C57Bl/6J mice, (n=8, 6-8 weeks old) at 0.2, 0.5, 2, 5, and 20 mg/kg as a 1% carboxymethylcellulose formulation for 10 days. The control mice received an equal amount of the vehicle (carboxymethylcellulose). On day 11 the mice were sacrificed and the left and right testis, the entire prostate and the individual prostatic lobes (e.g., the dorsal lobe and the ventral lobe) were weighed to measure absolute weights. Relative weights of entire prostate and individual lobes were calculated by dividing the corresponding absolute weight by the total weight of the mouse. Relative weights of the entire prostate, the dorsal prostate, and the ventral prostate were calculated by dividing the absolute weight by the total weight of the mouse. Relative weights of the left and right testis were calculated by dividing the corresponding absolute weights by the total weights of the mouse. The results are tabulated in FIGS. 4-13 and show upon administration of Compound 1 a dose dependent reduction in prostate weight.

### Example 46

### Mouse Studies

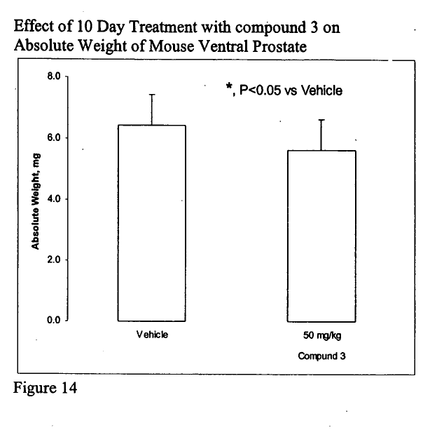

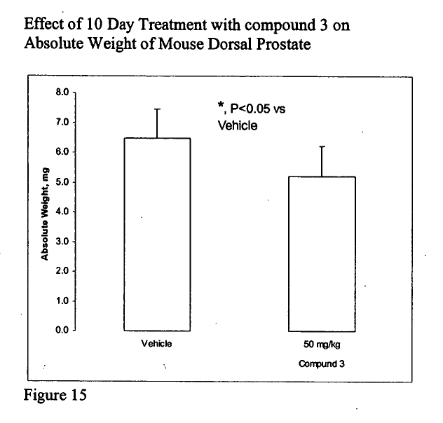

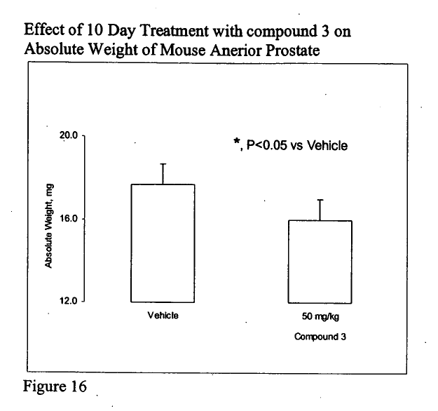

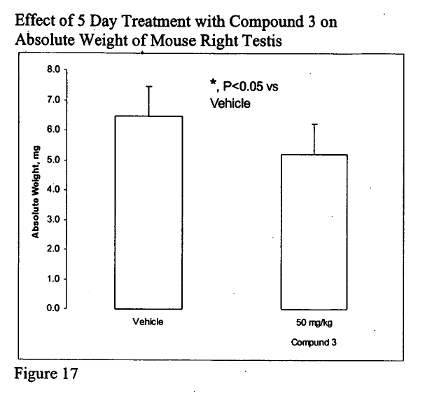

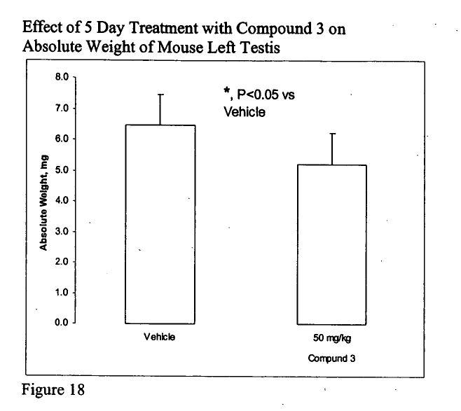

The 10 day effect of Compound 3 on the mouse prostate and its 5 day effect on mouse testis were determined as in Example 45 by using 10 mice per experiment group and the results are illustrated graphically in FIGS. 14-18.

### Example 47

### Mouse Studies

The tolerance of mice to compound 1 was determined by treating CD-1 mice daily with a single oral dose of compound 1 at 100, 200, and 500 mg/kg for 5 days. The mice were observed for a total of eight days and then euthanized. The toxicological end-points in this study were standard clinical observations such as changes in movement, breathing, food consumption, mortality and decreased body weight. The results of the study indicated a tolerated dose of Compound 1 in mice of 500 mg/kg/day upon oral dosing for 5 days and a 10 fold higher therapeutic index in mice compared to lonidamine when used for prostate weight reduction.

### Example 48

### In Vivo Viability and Proliferation of Mouse Prostate Cells

Prostate cells harvested from mice treated with 20 mg/kg of Compound 1 were assayed by the TUNEL assay (e.g., see Example 38). The prostate cells were more apoptotic as determined by the TUNEL assay and showed greater cell cycle inhibition as determined by immunohistochemistry of the S phase related proliferating cell nuclear antigen (PCNA assay) with respect to vehicle.

### Example 49

### 5 Day Systemic Administration of Test Compounds in Male SH Rats

The effect of test compounds (lonidamine and lonidamine analogs) on the SH rat prostate was determined as follows. The test compound was orally administered daily for 5 days to male, SH rat, (n=6, 14 weeks old, Charles River Labs) at 50 mg/ml concentrations (as a 0.5% carboxymethylcellulose formulation). The control rats received an equal amount of the vehicle (10 mg/ml). Blood samples were collected 2 hours after administration on day 6, for pharmokinetic analysis of the compounds. On day 6 the rats were sacrificed and the entire prostate and the individual lobes (e.g., the ventral, dorso-lateral, and anterior lobes) were removed and weighed to measure absolute weights. Relative weights of prostate and individual lobes were calculated by dividing the absolute weight by the total body weight of the mouse. Relative weights of the entire prostate, the dorsal prostate, and the ventral prostate were calculated by dividing the absolute weight by the total weight of the mouse. The results are tabulated in Table 8. The histomorphology of the prostate was also analyzed and compared to that of the control or untreated prostate. The results show a compound-dependent (related) reduction of the weight of the prostate and the testes in animals receiving the test compounds.

TABLE 8

| | | Prostate shrinkage | Testis shrinkage |
|---|---|---|---|
| Test Compound | PK (μg/ml) | (%) | (%) |
| Vehicle | | 0 | 0 |
| 12 | 153 | 17 | 42 |
| 3 | 243 | −2.9 | 0.4 |
| 1 | | −4.8 | −2.5 |
| 97 | 0 | 5.5 | 0.9 |
| 5 | 12 | −8.1 | −3.5 |
| 28 | 513 | −6.2 | 2.7 |
| 48 | | 3.3 | 4.3 |
| 31 | 210 | −4.3 | −1.8 |
| 51 | 0.45 | 8.7 | 2.2 |
| 53 | 1 | 6.9 | 9.2 |
| 54 | 120 | 6.8 | 0.3 |
| 58 | 0 | 12.9 | 26.5 |
| 34 | 498 | 16.9 | 33 |
| 62 | 0 | 9.8 | 2.2 |
| 63 | | 5.3 | 1.5 |
| 64 | 614 | −0.4 | −1.3 |
| 36 | | 0.4 | −2.4 |
| 78 | | 10.1 | 5.7 |
| 82 | | 5 | −1.1 |
| 83 | | 31.7 | 48 |
| 65 | | 7.5 | 2.2 |
| 67 | | 18.2 | 34.9 |
| 68 | | 10.4 | 11.8 |

Although the present invention has been described in detail with reference to specific embodiments, those of skill in the art will recognize that modifications and improvements are within the scope and spirit of the invention, as set forth in the claims which follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

1. (canceled)

2. (canceled)

3. (canceled)

4. (canceled)

5. (canceled)

6. (canceled)

7. (canceled)

8. The compound of formula IIIB, 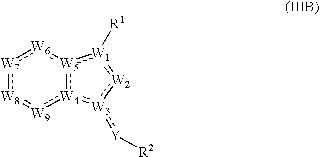
wherein
R¹ is selected from the group consisting of COOR³, COR⁴, CONR³COR³, CH═CHCO₂R³, B(OR³)₂, SO₂R⁴, NHSO₂CR⁵₃, NHSO₂CR³₃, CONHSO₂CR³₃, NHSO₂Ar, C(═NCN)NH₂, COCOR⁴CON(R³)N═CR³R⁷, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl; and L¹-V⁵ wherein L¹ is selected from the group consisting of —C≡C—, —C(V¹)═C(V³)—, —C(V¹V²)C(V³V⁴)—, 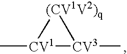
—NHCO— and —NHNH— wherein each V¹, V², V³, and V⁴ is independently selected from the group consisting of hydrogen, (C₁-C₄) alkyl or (C₁-C₈) heteroalkyl, halogen, hydroxy, (C₁-C₆) alkoxy, cyano, nitro, amino, (C₁-C₄) alkylamino and (C₁-C₄) dialkylamino or V¹ and V³ together form a (C₃-C₈) cycloalkyl, a (C₁-C₈) heterocycloalkyl, a (C₃-C₈) cycloalkenyl, an aryl or a heteroaryl ring; with the proviso that if one of V¹ and V² is hydroxyl, amino, (C₁-C₄) alkylamino or (C₁-C₄) dialkylamino, then the other is hydrogen or alkyl; and if one of V³ and V⁴ is hydroxyl, amino, (C₁-C₄) alkylamino, and (C₁-C₄) dialkylamino, then the other is hydrogen or alkyl; q is 1-6; V⁵ is selected from the group consisting of COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂Ar and C(═NCN)NH₂; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar or C(═NCN)NH₂; and when L¹ is —NHNH— then V⁵ is COOR³, COR⁴, COCOR⁴, B(OR³)₂, SO₂R⁴, or C(═NCN)NH₂;
R² is an aryl or heteroaryl group, optionally substituted with from one to three R⁶ substituents that are independently selected from the group consisting of H, halogen, C₁-C₈alkyl, C₁-C₈ heteroalkyl, aryl, heteroaryl, NR³COR³, hydroxy, alkoxy and CO₂R³;
R³ is H, (C₁-C₈) alkyl, (C₁-C₈) heteroalkyl, (C₃-C₈) cycloalkyl, (C₁-C₈) heterocyclyl, aryl or heteroaryl;
each R⁴ is a member independently selected from the group consisting of NR³R⁷, NR³OR⁷, NR⁷NR³R⁷ and NR³CN;
R⁵ is H, OH or halogen;
R⁷ is H, (C₁-C₈) alkyl, (C₁-C₈) heteroalkyl, (C₃-C₈) cycloalkyl, (C₁-C₈) heterocyclyl, aryl or heteroaryl;
R³ and R⁷ together are (C₁-C₈) heteroalkyl or heteroaryl;
Ar is aryl or heteroaryl;
each W¹, W³, W⁴ or W⁵ is independently N or C;
W² is a member selected from the group consisting of N, CR⁵, CO, O, NR⁷ and S;
each W⁶, W⁷, W⁸ or W⁹ is independently N or CV⁶ wherein V⁶ is selected from the group consisting of hydrogen, (C₁-C₄) alkyl, (C₁-C₈) heteroalkyl, halogen, hydroxy, (C₁-C₆) alkoxy, amino, cyano, nitro, oxo, U¹—R³, U¹—COR³, (C₁-C₄) alkylamino and (C₁-C₄) dialkylamino;
Y is CHR⁸, CR⁸₂, NR⁸, S or O;
R⁸ is H, (C₁-C₈) alkyl or (C₁-C₈) heteroalkyl; [symbol]
represents a single, double or normalized bond; and
pharmaceutically acceptable salts, solvates, hydrates, and prodrugs thereof;
provided that the compound does not have a formula selected from the group consisting of: 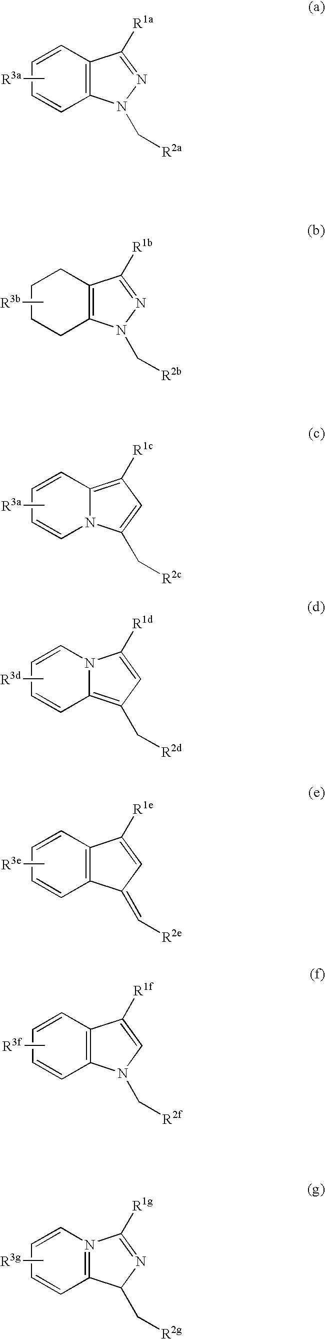 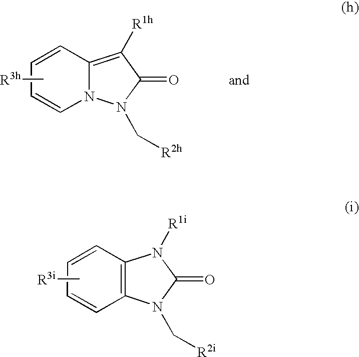
R^{1a} R^{1b} R^{1c} R^{3a}- N R^{3b}- N R^{3a} (a) R<a (b) R^{2b} (c) R^{2c} (a) R^{2a} b R^{1d} R^{1e} R^{1f} ^{3d}_N R^{3e} R^{3f} -(d) R^{2d} R^{2e} (f) R^{2f} R¹⁹ R^{1h} R¹ⁱ R³gs- R^{3h} O R³ⁱ NO (g) R^{2g} (h) k R^{2h} (i) CR²ⁱ
wherein
in formula (a):
(i) R^{1a} is selected from the group consisting of CONHNH₂, CONHN(CH₃)₂, and —CH═CHCO₂H:
R^{2a} is a group having the formula: 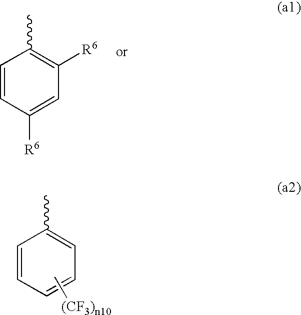
R⁶ or CF3)nio (al) (a2)
wherein each R⁶ independently is a halogen, and n10 is 1 or 2; and
R^{3a} is hydrogen;
(ii) R^{1a} is CO₂H;
R^{2a} is selected from the group consisting of 4-chlorophenyl, 3-chlorophenyl, 2-chlorophenyl, 4-fluorophenyl, 4-bromophenyl, 4-iodophenyl, 3-trifluoromethylphenyl, 4-cyanophenyl, 4-phenylsulfonyl-phenyl, 3,4-dichlorophenyl, 2,4-dichlorophenyl, 2,6-dichlorophenyl, 2,4-dibromophenyl, 2,4,5-trichlorophenyl, 4-chlorophenyl, 4-methylphenyl, 3-methylphenyl, 2-methylphenyl, 4-chlorophenyl, 3-benzoylphenyl, 4-methylsulfonylphenyl, 4-chloronaphthylmethyl, 2,4-dimethylphenyl and 2-methyl-4-chlorophenyl; and
R^{3a} is hydrogen;
(iii) R^{1a} is CO₂H
R^{2a} is 4-chlorophenyl; and
R^{3a} is chloro, OH, methyl, or OMe,
(iv) R^{1a} is selected from the group consisting of CO₂Me, CO₂Et, —CO-glyceryl, COCH₃, CONH₂, CH₂CO₂H, CH₂CH₂CO₂H and 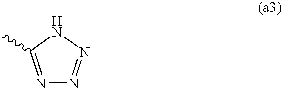
R^{2a} is 4-chlorophenyl, and
R^{3a} is H;
(v) R^{1a} is CO₂H,
R^{2a} is 2,4-dichlorophenyl,
R^{3a} is selected from the group consisting of —(OCH3)ₙ₁₀ wherein ₙ₁₀ is 1 or 2, chloro, bromo, fluoro, CO₂H, and CH₂CO₂H;
(vi) R^{1a} is —O—PO₃H, —O—SO₃H, —O—CH₂CO₂H, O—CH(CO₂H)₂, NHCH(CO₂H)₂, CH₂CH(NH₂)CO₂H, CONHCH(CO₂H)₂, and CONH(CH₂)ₙ₁₁-cyclopropyl wherein n11 is 0 or 1,
R^{2a} is 2,4-dichlorophenyl,
R^{3a} is H; and
(vii) R^{1a} is selected from the group consisting of —COCH₃, —SH, -tetrahydrofurfuryl,
—CH₂CO₂H, —CH₂CH₂CO₂H, —H, —CH₃, —CH₂OH, —NH₂, —CN, -tetrazin-2-yl, O—(CH₂)₁₋₂CO₂H, O—CH₂CO₂C₁-C₄alkyl, —O—PO₃H, —O—SO₃H, O—CH(CO₂H)₂, NHCH(CO₂H)₂ and CH₂CHNH₂CO₂H;
R^{2a} is selected from the group consisting of phenyl, 2-chlorophenyl, 2-methylphenyl, 3-fluorophenyl, 3-chlorophenyl, 3-bromophenyl, 3-methylphenyl, trifluoromethylphenyl, 3-benzoyl, 4-halophenyl, 4-methylsulfonylphenyl, 4-methylphenyl, 4-cyanophenyl, 4-phenylsulfonylphenyl, 4-methoxyphenyl, 4-chloronapth-1-yl, 2,3-dimethylphenyl, 2,4-dihalophenyl, 2,4-dimethylphenyl, 2,6-dichlorophenyl, 2,6-dimethylphenyl, 3,4-dichlorophenyl, bis-trifluoromethylphenyl, 4-chloro-2-methylphenyl, 5-chloro-2-methoxyphenyl, 2,4,5-trichlorophenyl, 2,6-dimethyl-3-dimethylsulfamoylphenyl, 4-imidazoyl;
R^{3a} is selected from the group consisting of H, 2-dimethylaminoethyl, 5-amino, chloro, bromo, 5-hydroxy, 5-methyl, methoxy, dimethoxy, fluoro, CO₂H, CH₂CO₂H, 5-nitro, 5-acetamido and 7-chloro;
(viii) compounds having the formulae: 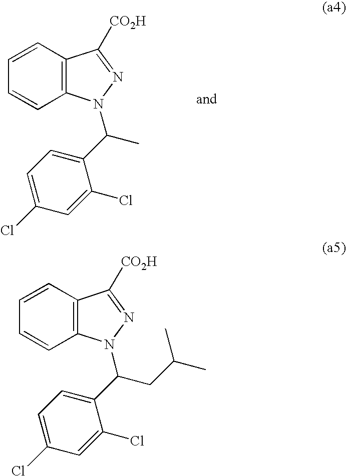
(ix) compounds having the formula: 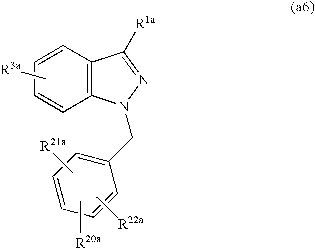
wherein R^{1a} is COOH, CONH₂, COO CH₂CH₂OH, COOCH₂CHOHCH₂OH, or COOCH(CH₂OH)₂;
R^{22a} is H or halo,
R^{20a} is halo, Me, methoxy, trifluoromethyl, CONH₂, or methanesulfonyl, and
R^{21a} is H, Me, halo, or a group forming with the benzene ring to which it is attached a naphthyl ring; and
R^{3a} is H, Me, methoxy and halogen;
in formula (b):
R^{1b} is CO₂H;
R^{2b} is phenyl;
R^{3b} is H;
in formula (c):
(i) R^{1c} is CH₂CONH₂:
R^{2c} is phenyl, 2-phenyl-phenyl, 2-benzyl-phenyl, 3-chlorophenyl, 3-trifluoromethylphenyl, 4-phenyl-phenyl, naphthyl, 3,5-di-t-butylphenyl, benzyl, 2-thienyl, 3-(thien-2-yl)thienyl, cyclohexylmethyl, 3-methoxyphenyl, 3-nitrophenyl, cyclopentylmethyl, cycloheptylmethyl, pentyl, 4-heptyl, 1-adamantyl, trans-4-pentyl-cyclohexyl, 2-phenylethenyl, 2-phenylethyl,
R^{3c} is selected from the group consisting of H, methyl, ethyl, t-butyl, cyclopropyl, —O(CH₂CH₂CH₂)₁₋₄CO₂H, —OCH₂-tetraazo-2-yl and —SCH₃:
(ii) compounds having the formula: 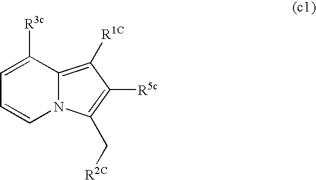
when R^{1c} is COCONH₂; R^{5c} and R^{2c} are defined below; and
R^{3c} is benzyl, then compounds i-xxv, xxvii, xxix, xxxvii, and xxxix are excluded;
R^{3c} is Me, then compound xxvi is excluded;
R^{3c} is H, then compounds i-xxix, xxxviii, and xxxix are excluded;
R^{3c} is —CH₂—CO₂Me, then compounds i, ii, iv, vi, viii-xxiii, xxiv, xxx-xxxviii, and xxxix are excluded;
R^{3c} is —CH₂—CO₂Et, then compounds iii, v, and vii are excluded; and
R^{3c} is —CH₂—CO₂H, then compounds i-xxix, xxx-xxxvii, and xxxix are excluded;

| Comp | R^{5c} | R^{2c} |
|---|---|---|
| I | Et | Ph |
| ii | Et | o-Ph-C₆H₄ |
| iii | Et | m-Cl—C₆H₄ |
| iv | Et | m-CF₃—C₆H₄ |
| V | Et | 1-naphthyl |
| vi | cycloPr | o-Ph-C₆H4 |
| vii | Me | Ph |
| viii | Et | p-Ph-C₆H₄ |
| ix | Et | cyclohexyl |
| X | Et | cyclopentyl |
| xi | Et | cycloheptyl |
| xii | Et | n-Bu |
| xiii | Et | Pent-4-yl |
| xiv | Et | 2-naphthyl |
| xv | Et | 3,5-(t-Bu)2-C₆H₃ |
| xvi | Et | Bn |
| xvii | Et | o-Bn-C₆H₄ |
| xviii | Et | 2-thienyl |
| xix | Et | 3-(thienyl-2-yl)thienyl-2-yl |
| xx | Et | m-MeO—C₆H₄ |
| xxi | Et | o-NO₂—C₆H₄ |
| xxii | Et | trans-4-(m-n-pentyl)cyclohexyl |
| xxiii | Me | 1-adamantyl |
| xxiv | Me | o-Ph-C₆H₄ |
| xxv | cycloPr | Ph |
| xxvi | Et | p-n-Bu-C₆H₄ |
| xxvii | Me | cyclohexyl |
| xxviii | cycloPr | cyclopentyl |
| xxix | Me | cyclopentyl |
| xxx | cycloPr | cyclohexyl |
| xxxi | iPr | o-Ph-C₆H₄ |
| xxxii | tBu | o-Ph-C₆H₄ |
| xxxiii | cyclopentyl | o-Ph-C₆H₄ |
| xxxiv | Et | m-Ph-C₆H₄ |
| xxxv | Et | cinnamyl |
| xxxvi | Et | phenethyl |
| xxxvii | cycloPr 1-naphthyl | |
| xxxviii | OMe | o-Ph-C₆H₄ |
| xxxix | SMe | o-Ph-C₆H₄ |
| xl | Me | Ph |
| xli | Me | cyclohexyl |

(iii) compounds having the following structure 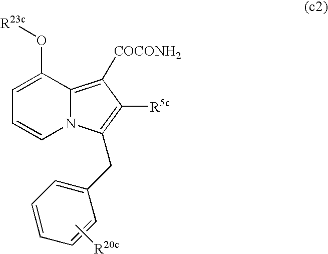
(a) wherein R^{23c} is CH₂CN or tetrazolyl,
R^{5c} is ethyl
R^{20c} is 3-chloro; and
(b) R^{23c} is CH₂-tetrazolyl, CH₂-2-pyridyl, CH₂-4-pyridyl, CH₂-2-quinolinyl, —(CH₂)₃—CO₂H, —(CH₂)₂—CO₂H,
R^{5c} is ethyl;
R^{20c} is 2-phenyl; and
(c) R^{23c} is OCH₂CO₂H,
R^{5c} is ethyl and
R^{20c} is H;
(d) R^{23c} is Me or H, and
R^{5c} is ethyl when R^{20c} is hydrogen,
R^{5c} is cyclopropyl when R^{20c} is 2-phenyl, and
R^{5c} is ethyl when R^{20c} is 2-phenyl;
(e) wherein R^{23c} is —(CH₂)₃—CO₂Et or —(CH₂)₃—CO₂H, and
R^{5c} is ethyl when R^{20c} is hydrogen,
R^{5c} is cyclopropyl when R^{20c} is 2-phenyl, and
R^{5c} is ethyl when R^{20c} is 2-phenyl;
(f) wherein R^{23c} is —(CH₂)₂—CO₂Et, —(CH₂)₂—CO₂H, —CH₂—CO₂Et or —CH₂—CO₂H, R^{5c} is ethyl and R^{20c} is 2-phenyl;
(iv) compounds having the following structure 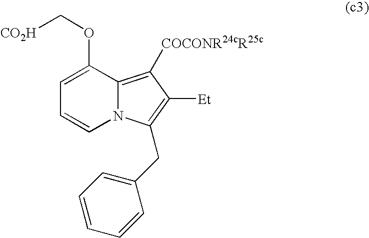
wherein R^{24c} is H or Me and R^{25c} is Me;
in formula (d):
R^{1d} is CH₂CONH₂;
R^{2d} is selected from the group consisting of phenyl, 2-phenyl-phenyl, 2-benzyl-phenyl, 3-chlorophenyl, 3-trifluoromethylphenyl, 4-phenyl-phenyl, naphthyl, 3,5-di-t-butylphenyl, benzyl, 2-thienyl, 3-(thien-2-yl)thienyl, cyclohexylmethyl, 3-methoxyphenyl, 3-nitrophenyl, cyclopentylmethyl, cycloheptylmethyl, pentyl, 4-heptyl, 1-adamantyl, trans-4-pentyl-cyclohexyl, 2-phenylethenyl and 2-phenylethyl;
R^{3d} is selected from the group consisting of H, methyl, ethyl, t-butyl, cyclopropyl, —O(CH₂CH₂CH₂)₁₋₄CO₂H, —OCH₂-tetraazo-2-yl and —SCH₃;
in formula (e):
(i) R^{1e} is CH₂CONH₂,
R^{2e} is selected from the group consisting of phenyl, 2-phenyl-phenyl, 2-benzyl-phenyl, 3-chlorophenyl, 3-trifluoromethylphenyl, 4-phenyl-phenyl, naphthyl, 3,5-di-t-butylphenyl, benzyl, 2-thienyl, 3-(thien-2-yl)thienyl, cyclohexylmethyl, 3-methoxyphenyl, 3-nitrophenyl, cyclopentylmethyl, cycloheptylmethyl, pentyl, 4-heptyl, 1-adamantyl, trans-4-pentyl-cyclohexyl, 2-phenylethenyl and 2-phenylethyl;
R^{3e} is selected from the group consisting of H, methyl, ethyl, t-butyl, cyclopropyl, —O(CH₂CH₂CH₂)₁₋₄CO₂H, —OCH₂-tetraazo-2-yl and —SCH₃;
in formula (f):
R^{1f} is CO₂H;
R^{2f} is selected from the group consisting of phenyl, 2-chlorophenyl, 3-chlorophenyl, 4-chlorophenyl, 2,4-dichlorophenyl, 2,5-dichlorophenyl, 2,6-dichlorophenyl, 3,4-dichlorophenyl and 3,5-dichlorophenyl;
R^{3f} is H;
in formula (g):
R^{1g} is CO₂Et;
R^{2g} is phenyl;
R^{3g} is H;
in formula (h):
R^{1h} is CO₂Et or C(═NH)OEt;
R^{2h} is phenyl;
R^{3h} is H, 5-methyl or 7-methyl;
in formula (i):
R¹ⁱ is CONHCH₂CH₂Cl or CONHCH₂CH₂-piperazin-4-yl;
R²ⁱ is benzyl; and
R³ⁱ is H.

9. The compound of claim 8 of formula (IIID): 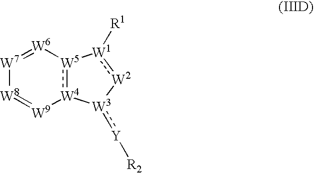
wherein
R¹ is selected from the group consisting of COOR³, COR⁴, CONR³COR³, CH═CHCO₂R³, B(OR³)₂, SO₂R⁴, NHSO₂CR⁵₃, NHSO₂CR³₃, CONHSO₂CR³₃, NHSO₂Ar, C(═NCN)NH₂, COCOR⁴ and L¹-V⁵ wherein L¹ is selected from the group consisting of —C≡C—, —C(V¹)═C(V³)—, —C(V¹V²)C(V³V⁴)—, 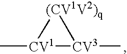
—NHCO— and —NHNH— wherein each V¹, V², V³, and V⁴ is independently selected from the group consisting of hydrogen, (C₁-C₄) alkyl or (C₁-C₈) heteroalkyl, halogen, hydroxy, (C₁-C₆) alkoxy, cyano, nitro, amino, (C₁-C₄) alkylamino and (C₁-C₄) dialkylamino or V¹ and V³ together form a (C₃-C₈) cycloalkyl, a (C₁-C₈) heterocycloalkyl, a (C₃-C₈) cycloalkenyl, an aryl or a heteroaryl ring; with the proviso that if one of V¹ and V² is hydroxyl, amino, (C₁-C₄) alkylamino or (C₁-C₄) dialkylamino, then the other is hydrogen or alkyl; and if one of V³ and V⁴ is hydroxyl, amino, (C₁-C₄) alkylamino, and (C₁-C₄) dialkylamino, then the other is hydrogen or alkyl; q is 1-6; V⁵ is selected from the group consisting of COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂Ar and C(═NCN)NH₂; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar or C(═NCN)NH₂; and when L¹ is —NHNH— then V⁵ is COOR³, COR⁴, COCOR⁴, B(OR³)₂, SO₂R⁴, or C(═NCN)NH₂;
R² is an aryl or heteroaryl group, optionally substituted with from one to three R⁶ substituents that are independently selected from the group consisting of H, halogen, C₁-C₈alkyl, C₁-C₈ heteroalkyl, aryl, heteroaryl, NR³COR³, hydroxy, alkoxy and CO₂R³;
R³ is H, (C₁-C₈) alkyl or (C₁-C₈) heteroalkyl, (C₃-C₈) cycloalkyl or (C₁-C₈) heterocyclyl, or aryl or heteroaryl;
each R⁴ is a member independently selected from the group consisting of NR³R, NR³OR⁷, NR⁷NR³R⁷ and NR³CN;
R⁵ is H, OH or halogen;
R⁷ is H, (C₁-C₈) alkyl or (C₁-C₈) heteroalkyl, (C₃-C₈) cycloalkyl or (C₁-C₈) heterocyclyl, or aryl or heteroaryl;
R³ and R⁷ together are (C₁-C₈) heteroalkyl or heteroaryl;
Ar is aryl or heteroaryl;
each W¹, W³, W⁴ or W⁵ is independently N or C;
W² is a member selected from the group consisting of N, CR⁵, CO, O, NR⁷ and S;
each W⁶, W⁷, W⁸ or W⁹ is independently N or CV⁶ wherein V⁶ is selected from the group consisting of hydrogen, (C₁-C₄) alkyl or (C₁-C₈) heteroalkyl, halogen, hydroxy, (C₁-C₆) alkoxy, amino, cyano, nitro, (C₁-C₄) alkylamino and (C₁-C₄) dialkylamino;
Y is CHR⁸, CR⁸₂, NR⁸, S or O;
R⁸ is H, (C₁-C₈) alkyl or (C₁-C₈) heteroalkyl group; [symbol]
represents a single, double or normalized bond; and
pharmaceutically acceptable salts, solvates, hydrates, and prodrugs thereof.

10. The compound of claim 9 wherein the A-B ring system is selected from the group consisting of: 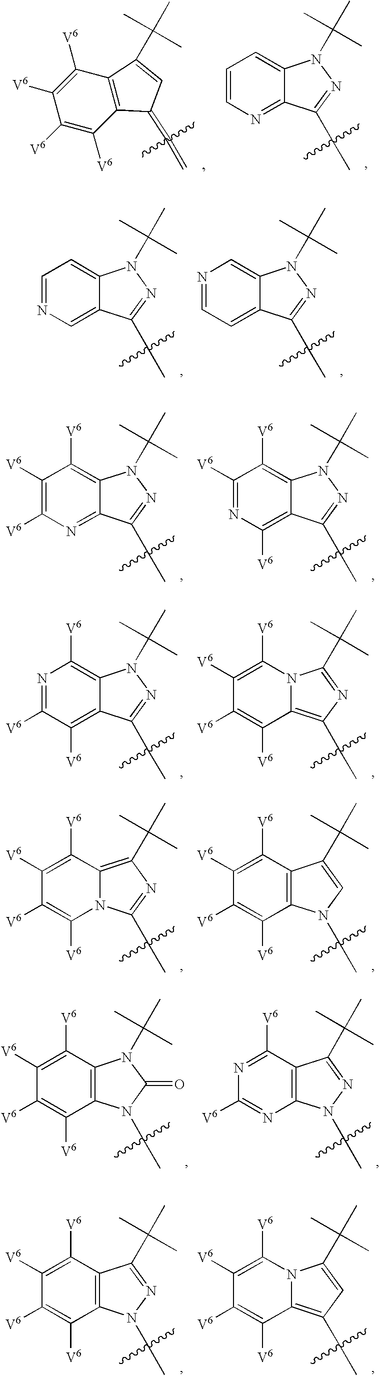 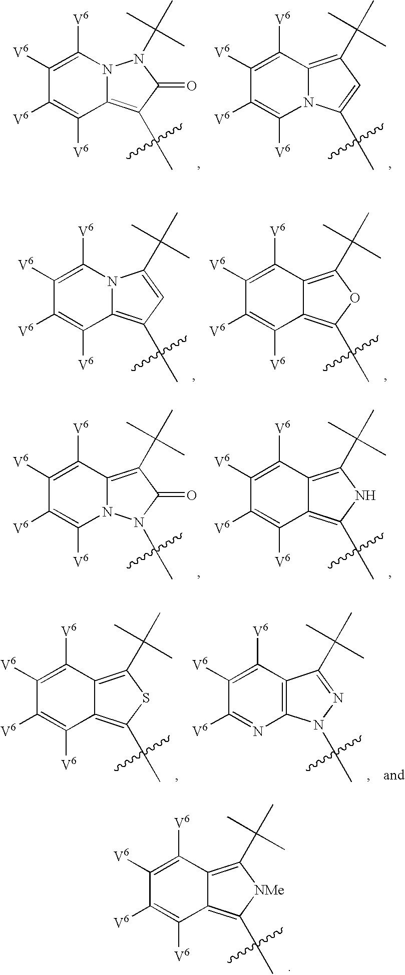
wherein the solid line indicates the point of attachment to R¹ and the wavy line indicates the point of attachment to Y and V⁶ is defined as above.

11. The compounds of any one of claim 8 wherein the A-B ring system has the structure 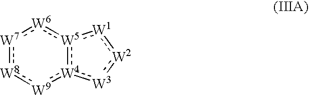
wherein W¹-W⁵ is defined as follows in Table 1A:
TABLE 1A

| Ring B | W¹ | W² | W³ | W⁴ | W⁵ |
|---|---|---|---|---|---|
| 1 | C | N | N | C | C |
| 2 | N | N | C | C | C |
| 3 | N | C═O | N | C | C |
| 4 | N | SO₂ | N | C | C |
| 5 | N | SO | N | C | C |
| 6 | N | C═O | C | C | N |
| 7 | N | SO₂ | C | C | N |
| 8 | N | SO | C | C | N |
| 9 | C | C═O | N | N | C |
| 10 | C | SO₂ | N | N | C |
| 11 | C | SO | N | N | C |
| 12 | C | N | C | N | C |
| 13 | C | N | C | C | N |
| 14 | C | CR⁵ | C | N | C |
| 15 | C | CR⁵ | C | C | N |
| 16 | C | O | C | C | C |
| 17 | C | S | C | C | C |
| 18 | C | SO | C | C | C |
| 19 | C | SO₂ | C | C | C |
| 20 | C | NR⁷ | C | C | C |
| 21 | C | CR⁵ | C | C | C |

and for each ring B 1-21 as defined above, W⁶-W⁹ is defined as follows in Table 1B:
TABLE 1B

| Ring A | W⁶ | W⁷ | W⁸ | W⁹ |
|---|---|---|---|---|
| 1 | CV⁶ | CV⁶ | CV⁶ | CV⁶ |
| 2 | CV⁶ | CV⁶ | CV⁶ | N |
| 3 | CV⁶ | CV⁶ | N | CV⁶ |
| 4 | CV⁶ | N | CV⁶ | CV⁶ |
| 5 | N | CV⁶ | CV⁶ | CV⁶ |
| 6 | CV⁶ | CV⁶ | N | N |
| 7 | CV⁶ | N | N | CV⁶ |
| 8 | N | N | CV⁶ | CV⁶ |
| 9 | CV⁶ | N | CV⁶ | N |
| 10 | N | CV⁶ | N | CV⁶ |
| 11 | N | CV⁶ | CV⁶ | N |
| 12 | N | N | N | CV⁶ |
| 13 | N | N | CV⁶ | N |
| 14 | N | CV⁶ | N | N |
| 15 | CV⁶ | N | N | N |

12. The compounds of any one of claim 8 wherein the A-B ring system has the structure 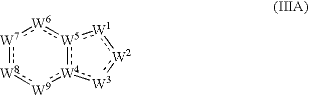
wherein W¹-W⁵ is defined as follows in Table 1A:
TABLE 1A

| Ring B | W¹ | W² | W³ | W⁴ | W⁵ |
|---|---|---|---|---|---|
| 1 | C | N | N | C | C |
| 2 | N | N | C | C | C |
| 3 | N | C═O | N | C | C |
| 4 | N | SO₂ | N | C | C |
| 5 | N | SO | N | C | C |
| 6 | N | C═O | C | C | N |
| 7 | N | SO₂ | C | C | N |
| 8 | N | SO | C | C | N |
| 9 | C | C═O | N | N | C |
| 10 | C | SO₂ | N | N | C |
| 11 | C | SO | N | N | C |
| 12 | C | N | C | N | C |
| 13 | C | N | C | C | N |
| 14 | C | CR⁵ | C | N | C |
| 15 | C | CR⁵ | C | C | N |
| 16 | C | O | C | C | C |
| 17 | C | S | C | C | C |
| 18 | C | SO | C | C | C |
| 19 | C | SO₂ | C | C | C |
| 20 | C | NR⁷ | C | C | C |
| 21 | C | CR⁵ | C | C | C |

and for each ring B 1-21 as defined above, W¹-W⁹ is defined as follows in Table 1C:
TABLE 1C

| | | |
|---|---|---|
| 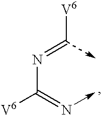 | 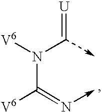 | 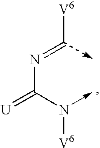 |
| 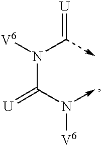 | 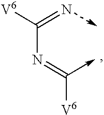 | 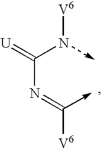 |
| 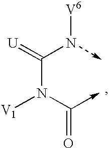 | 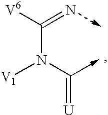 | 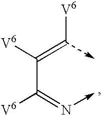 |
| 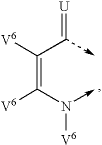 | 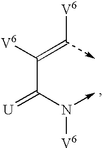 | 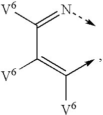 |
| 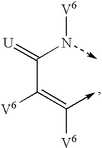 | 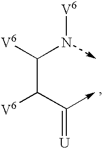 | 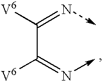 |
| 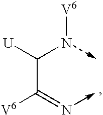 | 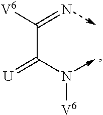 | 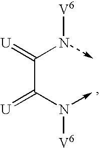 |
| 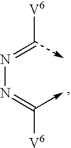 | 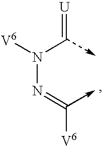 | 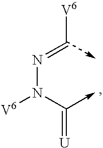 |
| 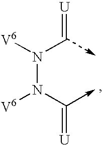 | 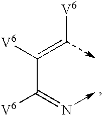 | 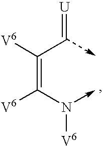 |
| 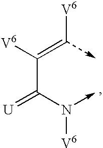 | 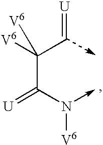 | 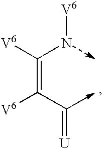 |
| 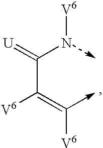 | 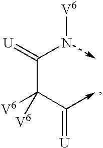 | 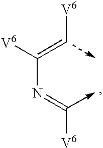 |
| 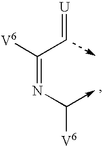 | 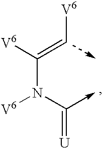 | 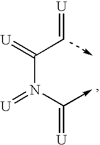 |
| 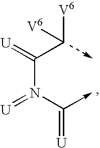 | 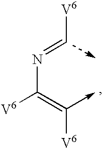 | 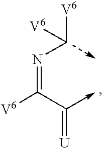 |
| 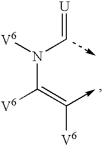 | 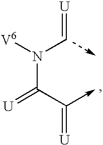 | 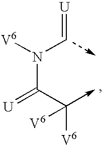 |
| 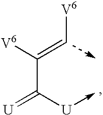 | 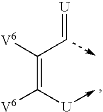 | 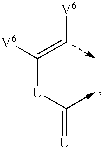 |
| | |
|---|---|
| 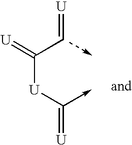 | 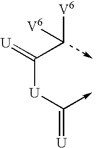 |

wherein → indicates a single bond to W⁴ and ---------→ indicates a single bond to W⁵ and V⁶ and U are as defined above.

13. The compound of claim 9 wherein R¹ is selected from the group consisting of:
CONHNH₂, CONH₂, CONHNMe₂, CONMe₂

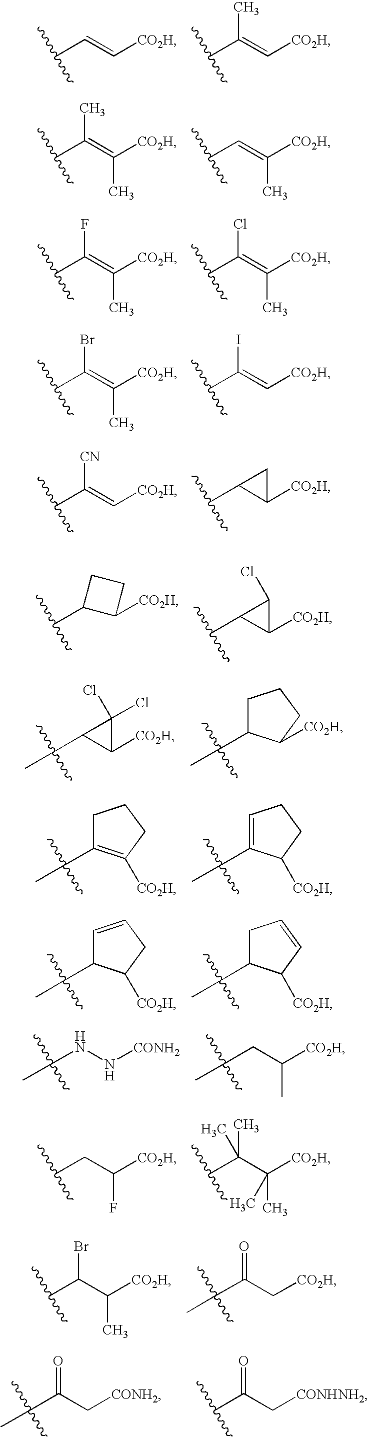

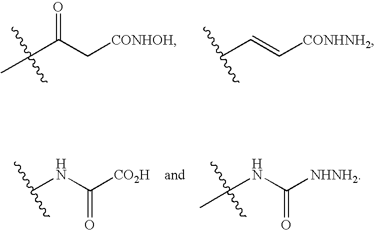

14. The compound of claim 9 wherein, R¹ is a COOR³ or L¹-CO₂R³, L¹; R³ is H or (CH₂)_{q}NR⁹R¹⁰; each R⁹ and R¹⁰ is (C₁-C₈) alkyl, or optionally, if both are present on the same substituent, joined together to form a three- to eight-membered heterocyclyl ring system; and the subscript q is an integer of from 1 to 4.

15. The compound of claim 13 wherein R² is selected from the group consisting of pyrroyl, pyrazoyl, imidazoyl, pyridinyl, dihydropyridinyl, pyrazinyl, pyridazinyl, pyrimidinyl and phenyl, optionally substituted with from one to two substituents selected from the group consisting of halo and (C₁-C₈) alkyl.

16. The compound of claim 9 wherein R² is selected from the group consisting of 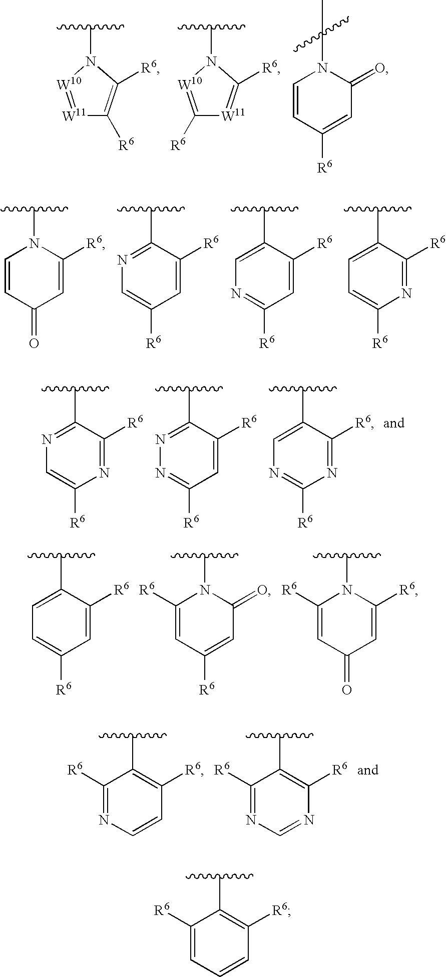
wherein each W¹⁰ or W¹¹ is independently selected from the group consisting of N, C, and CH; R⁹ is halo or (C₁-C₈) alkyl; and the wavy line indicates the point of attachment to the rest of the molecule.

17. The compound of claim 9 wherein R⁶ is F, Cl, Br, CN, CF₃, CH₃, CHMe₂, —C≡CH—C≡C—CH₃, or CONHMe; and each R³, R⁷, and R⁸ are independently selected from the group consisting of: H, —CH₃, —CH₂CH₃,

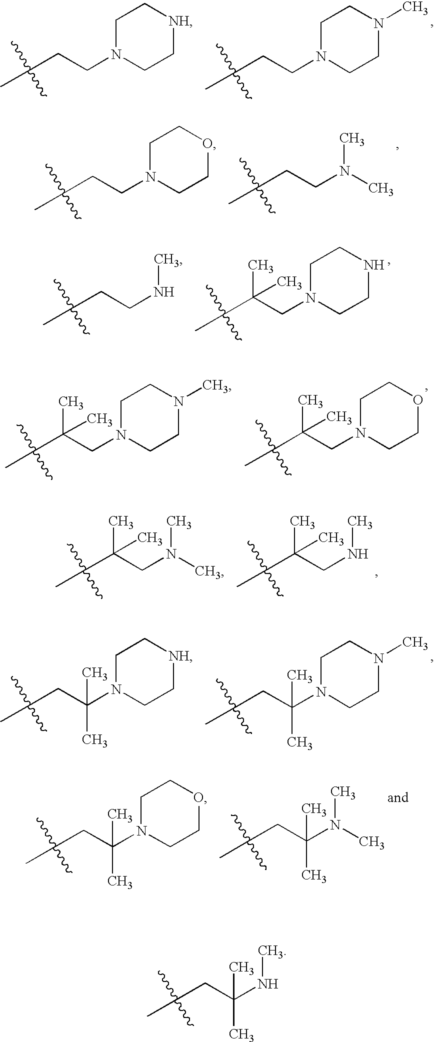

18. The compound of claim 8 of formula: 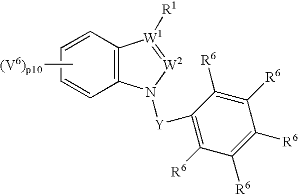
wherein R¹ is selected from the group consisting of CO₂R³, COR⁴, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH— V⁵, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl;
L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₄) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then a V¹ attached to the same atom is hydrogen or alkyl;
each R³ is a member independently selected from the group consisting of H, (C₁-C₈) alkyl, (C₁-C₈) heteroalkyl, (C₃-C₈) cycloalkyl, (C₁-C₈) heterocyclyl, aryl and heteroaryl; each R⁴ is selected from the group consisting of NR³R⁷, NR³OR⁷, NR⁷NR³R⁷ and NR³CN;
R⁵ is H, OH or halogen;
each R⁶ is a member independently selected from the group consisting of H, halogen, C₁-C₈alkyl, C₁-C₈ heteroalkyl, aryl, heteroaryl, NR³COR³, hydroxy, alkoxy and CO₂R³;
R⁷ is selected from the group consisting of H, (C₁-C₈) alkyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;
each V⁵ is a member independently selected from the group consisting of COOR³, COR⁴, CONR³COR³, COCOR⁴, B(OR³)₂, SO₂R⁴, NHSO₂CR⁵₃, NHSO₂R³₃, CONHSO₂CR³₃ and C(═NCN)NH₂;
each V⁶ is independently a member selected from the group consisting of hydrogen, halo, oxo, cyano, nitro, (C₁-C₈) alkyl, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl, O—R³, S—R³, R⁴, NR³—COR³, NR³—CONR³R⁷, NR³—CSNR³R⁷, NR³—C(═NR³)NR³R⁷, NR³—CO₂R³, NR³—SO₂R³, COR³, CO₂R³, CSNR³R⁷, C(═NR³)NR³R⁷, CONR³COR³, CONR³C(═NR³)R³, SO₂R³, SOR³, SO₃R³¹, SO₂NR³R⁷, PO(OR³)₂, PS(OR³)₂ and PO(NR³R⁷)₂, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;
the subscript p10 is an integer of from 0 to 4;
W¹ independently C or N;
W² is N, CR⁵ or CO;
Y is CHR⁸, CR⁸₂, NR⁸, S or O; and
R⁸ is H, (C₁-C₈) alkyl or (C₁-C₈) heteroalkyl.

19. The compound of claim 8 of formula: 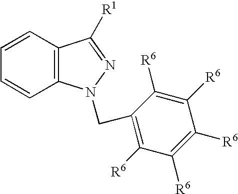
wherein R¹ is selected from the group consisting of: CO₂R³, COR⁴ and CONHSO₂CR³₃;
each R³ is a member independently selected from the group consisting of H, (C₁C₈) alkyl, aryl, (C₁-C₈) heteroalkyl and (C₁-C₈) heterocyclyl;
each R⁴ is a member independently selected from the group consisting of NR³R⁷, NR³OR⁷ and NR⁷NR³R⁷;
R⁶ is independently selected from the group consisting of H hydrogen, F, Cl, Br, OH, OCH₃, OCF₃, CN, CF₃, CH₂F, CHF₂, CH₃, CHMe₂, —C≡CH, —C≡C—CH₃, and CONHMe and
R⁷ is selected from the group consisting of H, (C₁-C₈) alkyl, (C₁-C₈) heteroalkyl, aryl and (C₁-C₈) heterocyclyl

20. The compound of claim 8 selected from the group consisting of formulae (V-A), (V-B), (V-C), (V-D), (V-E), (V-F), (V-G), (V-H), (V-I) and (V-J): 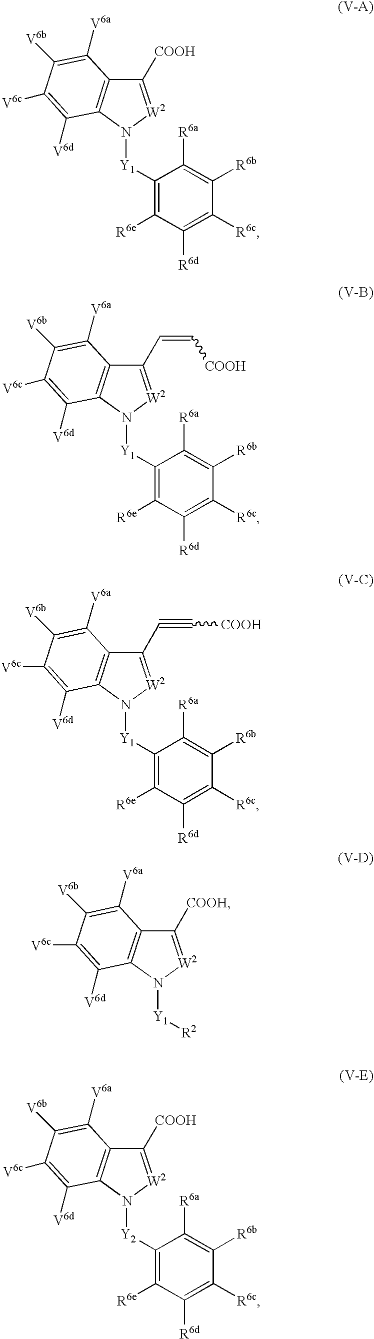 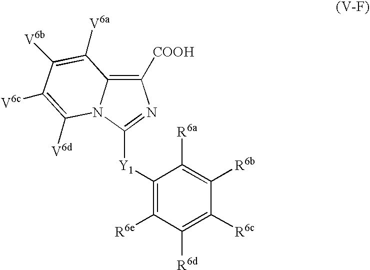 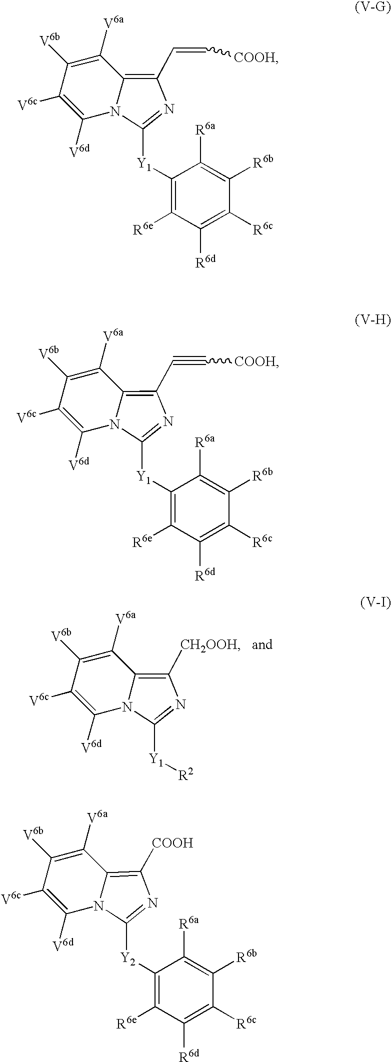
wherein
each V^{6a}, V^{6b}, V^{6c} and V^{6d} are independently a member selected from the group consisting of hydrogen, halogen, C₁-C₈ alkyl, C₁-C₈ heteroalkyl, aryl, heteroaryl, hydroxyl, amino, alkylamino, dialkylamino, nitro, cyano, C₂-C₆ alkenyl, C₂-C₆ alkynyl, C₁-C₆ alkoxy, and CO₂R³;
each R^{6a}, R^{6b}, R^{6c}, R^{6d} and R^{6e} are independently a member selected from the group consisting of H, halogen, C₁-C₈alkyl, C₁-C₈ heteroalkyl, aryl, heteroaryl, NR³COR³, hydroxy, alkoxy and CO₂R³, or R^{6c} and R^{6d} may be taken together to form a dioxomethylene bridge;
R² is a defined above;
W² is N, CH or COH;
Y₁ is C(R⁸)₂ wherein R⁸ is hydrogen, alkyl, heteroalkyl, aryl or heteroaryl;
Y₂ is CO or SO₂;
and pharmaceutically acceptable salts thereof.

21. The compound of claim 8 selected from the group consisting of formulae (VI-A), (VI-B), (VI-C), (VI-D), (VI-E) and (VI-F): 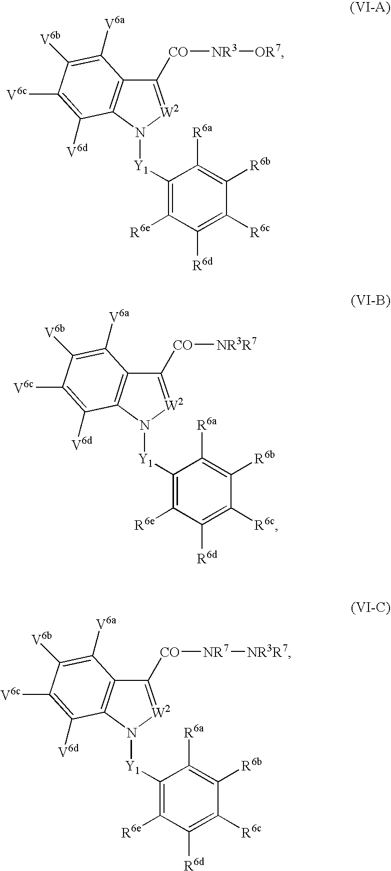 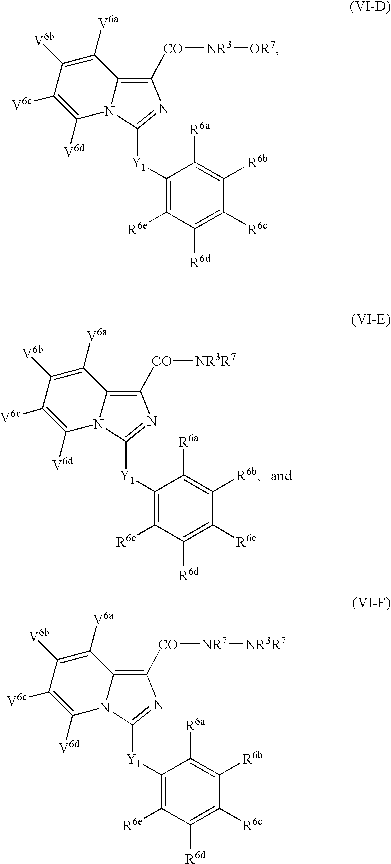
wherein
each V^{6a}, V^{6b}, V^{6c} and V^{6d} are independently a member selected from the group consisting of hydrogen, halogen, C₁-C₈ alkyl, C₁-C₈ heteroalkyl, aryl, heteroaryl, hydroxyl, amino, alkylamino, dialkylamino, nitro, cyano, C₂-C₆ alkenyl, C₂-C₆ alkynyl, C₁-C₆ alkoxy, and CO₂R³,
each R^{6a}, R^{6b}, R^{6c}, R^{6d} and R^{6e} are independently a member selected from the group consisting of H, halogen, C₁-C₈ alkyl, C₁-C₈ heteroalkyl, aryl, heteroaryl, NR³COR³, hydroxy, alkoxy and CO₂R³ or R^{6c} and R^{6d} may be taken together to form a dioxomethylene bridge;
W² is N, CH or COH;
Y₁ is C(R⁸)₂ wherein R⁸ is hydrogen, alkyl heteroalkyl, aryl or heteroaryl;
each R³ and R⁷ is a member independently selected from the group consisting of hydrogen, C₁-C₈ alkyl, C₁-C₈ heteroalkyl, C₃-C₈ cycloalkyl, C₃-C₈ heterocyclyl, aryl, heteroaryl; R³ and R⁷ taken together form a C₃-C₈ heterocyclyl or heteroaryl ring;
and pharmaceutically acceptable salts thereof.

22. The compound of claim 8 selected from the group consisting of formulae (VII-A) and (VII-B): 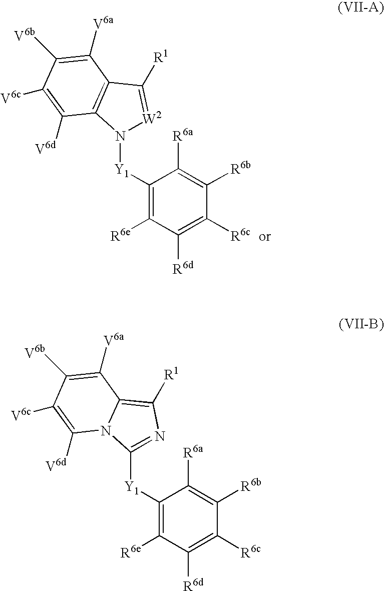
wherein
R¹ is selected from CHO, CR³R⁷OR⁷, CONR³SO₂R⁷, SO₂NR³R⁷, and tetrazole;
each V^{6a}, V^{6b}, V^{6c} and V^{6d} are independently a member selected from the group consisting of hydrogen, halogen, C₁-C₈ alkyl, C₁-C₈ heteroalkyl, aryl, heteroaryl, hydroxyl, amino, alkylamino, dialkylamino, nitro, cyano, C₂-C₆ alkenyl, C₂-C₆ alkynyl, C₁-C₆ alkoxy, and CO₂R³;
each R^{6a}, R^{6b}, R^{6c}, R^{6d} and R^{6e} are independently a member selected from the group consisting of H, halogen, C₁-C₈ alkyl, C₁-C₈ heteroalkyl, aryl, heteroaryl, NR³COR³, hydroxy, and alkoxy or R^{6c} and R^{6d} may be taken together to form a dioxomethylene bridge;
each R³ and R⁷ is a member independently selected from the group consisting of hydrogen, C₁-C₈ alkyl, C₁-C₈ heteroalkyl, C₃-C₈ cycloalkyl, C₃-C₈ heterocyclyl, aryl, heteroaryl;
W² is N, CH or COH;
Y, is C(R⁸)₂ wherein R⁸ is hydrogen, alkyl heteroalkyl, aryl or heteroaryl;
and pharmaceutically acceptable salts thereof.

23. A method for prophylaxis or treatment of benign prostatic hypertrophy (BPH) comprising administering an effective amount of a compound of formula (I) to a human subject in need of such treatment: 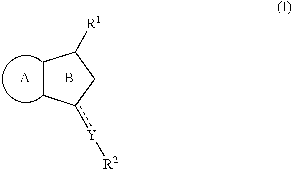
wherein A-B is a 7,5, 6,5 or a 5,5 cyclic ring system, optionally substituted with from one to five V⁶ substituents, each independently selected from the group consisting of hydrogen, amino, halo, oxo, nitro, (C₁-C₈) alkyl, (C₁-C₆) alkoxy, nitro, acetamido, L¹-CO₂H, L¹-dialkylamino, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₃), NR³SOR³, N—(SOR³)₂, NR³—PU₂R³, N—(PU₂R³)₂, NR³—P(═U)(UR³)R³, CUR³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷))₂, SO₂R³, SOR³, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, cyano, nitrileoxide, and —NO, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; R¹ is selected from the group consisting of CO₂R³, COR⁴, COCOR³, CONR³COR³, CH═CHCO₂R³, B(OR³)₂, SO₂R⁴, NHSO₂CR⁵₃, NHSO₂CR³₃, CONHSO₂CR³₃, C(═NCN)NH₂, —NHCO—V⁵, —NHNH—V⁵, COCOR⁴, CON(R³)N═CR³R⁷, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl; or may be taken together with a V⁶ attached to adjacent or within two atoms to form a (C₃-C₈) cycloalkyl, a (C₁-C₈) heterocycloalkyl, a (C₃-C₈) cycloalkenyl, an aryl or a heteroaryl ring;
L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₆) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl;
R² is an aryl or heteroaryl group, optionally substituted with from one to five R⁶ substituents independently selected from the group consisting of halo, nitro, cyano, nitrileoxide, —NO, R³, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₃R³, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, nitrileoxide, and —NO;
each R³ is a member independently selected from the group consisting of H, (C₁-C₈) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl and heteroaryl;
each R⁴ is a member independently selected from the group consisting of NR³R⁷, NR³OR⁷, NR⁷NR³R⁷ or NR³CN;
R⁵ is H, OH or halogen;
R⁷ is selected from the group consisting of H, (C₁-C₈) alkyl, (C₂-C₆) alkenyl, (C₂-C₈) alkynyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;
R⁸ is H, halo, nitro, cyano, nitrileoxide, —NO, R³, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, or 2R⁸ taken together form a (C₃-C₈) cycloalkyl, (C₃-C₈) heterocyclyl or heteroaryl ring;
R³¹ is aryl or heteroaryl;
each V⁵ is a member independently selected from the group consisting of COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂R³, CONHSO₂R³, and C(═NCN)NH₂;
Y is CR⁸₂, CR⁸, NR⁸, S or O;
U is O, S, NR³, NCOR³, or NCONR³R⁷;
U¹ is O or S; [symbol]
represents a single or double bond.

24. The method of claim 23 comprising administering an effective amount of a compound of formula IIIB to the subject 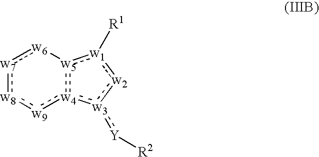
wherein
R¹ is selected from the group consisting of COOR³, COR⁴, CONR³COR³, CH═CHCO₂R³, B(OR³)₂, SO₂R⁴, NHSO₂CR⁵₃, NHSO₂CR³₃, CONHSO₂CR³₃, NHSO₂Ar, C(═NCN)NH₂, COCOR⁴CON(R³)N═CR³R⁷, -L¹CO₂R³, —CN, -tetrazin-2-yl, —O-L¹CO₂R³—O—PO₃H, —O—SO₃H, O-L¹(CO₂H)₂, —NHL¹(CO₂H)₂, COHNL¹(CO₂H)₂ and CONHL¹-(C₃-C₈) cycloalkyl; and L¹-V⁵ wherein L¹ is selected from the group consisting of —C—, —C(V¹)═C(V³)—, —C(V¹V²)C(V³V⁴)—, 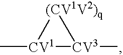
—NHCO— and —NHNH— wherein each V¹, V 2, V³, and V⁴ is independently selected from the group consisting of hydrogen, (C₁-C₄) alkyl or (C₁-C₈) heteroalkyl, halogen, hydroxy, (C₁-C₆) alkoxy, cyano, nitro, amino, (C₁-C₄) alkylamino and (C₁-C₄) dialkylamino or V¹ and V³ together form a (C₃-C₈) cycloalkyl, a (C₁-C₈) heterocycloalkyl, a (C₃-C₈) cycloalkenyl, an aryl or a heteroaryl ring; with the proviso that if one of V¹ and V² is hydroxyl, amino, (C₁-C₄) alkylamino or (C₁-C₄) dialkylamino, then the other is hydrogen or alkyl; and if one of V³ and V⁴ is hydroxyl, amino, (C₁-C₄) alkylamino, and (C₁-C₄) dialkylamino, then the other is hydrogen or alkyl; q is 1-6; V⁵ is selected from the group consisting of COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂Ar and C(═NCN)NH₂; with the proviso that in NHSO₂CR⁵₃, R⁵ is not OH; when L¹ is —NHCO— then V⁵ is COR⁴, NHSO₂CR⁵₃, NHSO₂CR³₃, NHSO₂Ar or C(═NCN)NH₂; and when L¹ is —NHNH— then V⁵ is COOR³, COR⁴, COCOR⁴, B(OR³)₂, SO₂R⁴, or C(═NCN)NH₂;
R² is an aryl or heteroaryl group, optionally substituted with from one to three R⁶ substituents that are independently selected from the group consisting of H, halogen, C₁-C₈alkyl, C₁-C₈ heteroalkyl, aryl, heteroaryl, NR³COR³, hydroxy, alkoxy and CO₂R³;
R³ is H, (C₁-C₈) alkyl, (C₁-C₈) heteroalkyl, (C₃-C₈) cycloalkyl, (C₁-C₈) heterocyclyl, aryl or heteroaryl;
each R⁴ is a member independently selected from the group consisting of NR³R⁷, NR³OR⁷, NR⁷NR³R⁷ and NR³CN;
R⁵ is H, OH or halogen;
R⁷ is H, (C₁-C₈) alkyl, (C₁-C₈) heteroalkyl, (C₃-C₈) cycloalkyl, (C₁-C₈) heterocyclyl, aryl or heteroaryl;
R³ and R⁷ together are (C₁-C₈) heteroalkyl or heteroaryl;
Ar is aryl or heteroaryl;
each W¹, W³, W⁴ or W⁵ is independently N or C;
W² is a member selected from the group consisting of N, CR⁵, CO, O, NR⁷ and S;
each W⁶, W⁷, W⁸ or W⁹ is independently N or CV⁶ wherein V⁶ is selected from the group consisting of hydrogen, (C₁-C₄) alkyl, (C₁-C₈) heteroalkyl, halogen, hydroxy, (C₁-C₆) alkoxy, amino, cyano, nitro, oxo, U¹—R³, U¹—COR³, (C₁-C₄) alkylamino and (C₁-C₄) dialkylamino;
Y is CHR⁸, CR⁸, NR⁸, S or O;
R⁸ is H, (C₁-C₈) alkyl or (C₁-C₈) heteroalkyl; [symbol]
represents a single, double or normalized bond.

25.-45. (canceled)

46. A method for treating cancer, said method comprising administering to a mammal a therapeutically effective amount of a compound of formula (I) to a human subject in need of such treatment: 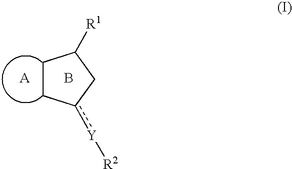
wherein A-B is a 7,5, 6,5 or a 5,5 cyclic ring system, optionally substituted with from one to five V⁶ substituents, each independently selected from the group consisting of hydrogen, amino, halo, oxo, nitro, (C₁-C₈) alkyl, (C₁-C₆) alkoxy, nitro, acetamido, L¹-CO₂H, L¹-dialkylamino, (C₁-C₈) heteroalkyl, (C₂-C₈) alkenyl, (C₂-C₈) alkynyl, (C₃-C₈) cycloalkyl, (C₁-C₈) heterocyclyl, aryl, heteroaryl; U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CUR³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₂R³, SOR³, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, cyano, nitrileoxide, and —NO, or any two V⁶ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring;
R¹ is selected from the group consisting of CO₂R³, COR⁴, COCOR³, CONR³COR³, CH═CHCO₂R³, B(OR³)₂ SO₂R⁴, NHSO₂CR⁵₃, NHSO₂CR³₃, CONHSO₂CR³₃, C(═NCN)NH₂, —NHCO— V⁵, —NHNH— V⁵, COCOR⁴, CON(R³)N═CR³R⁷, L¹-V⁵, -L¹CO₂R³, —CN, -tetrazin-2-yl, —O-L¹CO₂R³, —O—PO₃H, —O—SO₃H, O-L¹(CO₂H)₂, —NHL¹(CO₂H)₂, COHNL¹(CO₂H)₂ and CONHL¹-(C₃-C₈) cycloalkyl; or may be taken together with a V⁶ attached to adjacent or within two atoms to form a (C₃-C₈) cycloalkyl, a (C₁-C₈) heterocycloalkyl, a (C₃-C₈) cycloalkenyl, an aryl or a heteroaryl ring;
L¹ is selected from the group consisting of (C₁-C₈) alkylene, (C₂-C₈) alkenyl, (C₂-C₈) alkynyl, and (C₃-C₈) cycloalkylene, optionally substituted with from one to fourteen V¹ wherein each V¹ is independently selected from the group consisting of (C₁-C₄) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl, heteroaryl, halogen, hydroxy, (C₁-C₆) alkoxy, cyano, nitro, amino, —NO, (C₁-C₄) alkylamino and (C₁-C₄) dialkylamino, or any two V¹ attached to the same or adjacent atoms may be taken together with the atoms with which they are attached to form a (C₃-C₈) cycloalkyl, a (C₁-C₈) heterocycloalkyl, a (C₃-C₈) cycloalkenyl, an aryl or a heteroaryl ring; with the proviso that if one of V¹ is hydroxyl, amino, (C₁-C₄) alkylamino or (C₁-C₄) dialkylamino, then an V¹ attached to the same atom is hydrogen or alkyl;
R² is an aryl or heteroaryl group, optionally substituted with from one to five R⁶ substituents independently selected from the group consisting of halo, nitro, cyano, nitrileoxide, —NO, R³, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R⁷), PU(NR³R⁷)₂, PU(NR³COR³)₂, PU(NR³CU₂R³)₂, PU(NR³CUNR³R⁷)₂, NR³(NR³)₂, nitrileoxide, and —NO;
each R³ is a member independently selected from the group consisting of H, (C₁-C₈) alkyl, (C₁-C₈) heteroalkyl, (C₂-C₆) alkenyl, (C₂-C₈) alkynyl, (C₃-C₈) cycloalkyl, (C₁-C₈) heterocyclyl, aryl and heteroaryl;
each R⁴ is a member independently selected from the group consisting of NR³R⁷, NR³OR⁷, NR⁷NR³R⁷ or NR³CN;
R⁵ is H, OH or halogen;
R⁷ is selected from the group consisting of H, (C₁-C₈) alkyl, (C₂-C₆) alkenyl, (C₂-C₈) alkynyl, (C₁-C₈) heteroalkyl, (C₃-C₈) cycloalkyl, (C₁-C₈) heterocyclyl, aryl, heteroaryl; or R³ and R⁷ are taken together form a (C₁-C₈) heterocyclyl or heteroaryl ring;
R⁸ is H, halo, nitro, cyano, nitrileoxide, —NO, R³, U¹—R³, U¹—COR³, U¹—CUNR³R⁷, U¹—CU₂R³, R⁴, NR³OR³, NR³—CUR³, N—(CUR³)₂, NR³—CUNR³R⁷, N—(CUNR³R⁷)₂, NR³—CU₂R³, N—(CU₂R³)₂, NR³—SO₂R³, N—(SO₂R³)₂, NR³—SOR³, N—(SOR³)₂, NR³—PU₂R³, N—(PU₂R³)₂, NR³—P(═U)(UR³)R³, CU₂R³, CUNR³R⁷, CUNR³CUR³, CUN(CUR³)₂, CUNR³CU₂R³, CUN(CU₂R³)₂, CUNR³CUNR³R⁷, CUN(CUNR³R⁷)₂, SO₃R³¹, SO₂NR³R⁷, SO₂NR³CUR³, SO₂N(CUR³)₂, SO₂NR³CU₂R³, SO₂N(CU₂R³)₂, SO₂NR³CUNR³R⁷, SO₂N(CUNR³R⁷)₂, PU(UR³)₂, PU(UR³)(NR³R), PU(NR³R⁷)₂, PU(NR³COR³)₂, PU(NR³CU₂R³)₂, PU(NR³CUNR³R⁷)₂, NR³(NR³)₂, or 2R⁸ taken together form a (C₃-C₈) cycloalkyl, (C₃-C₈) heterocyclyl or heteroaryl ring;
R³¹ is aryl or heteroaryl;
each V⁵ is a member independently selected from the group consisting of COOR³, COR⁴, CONR³COR³, COCOR⁴, B(OR³)₂, SO₂R⁴, NHSO₂CR⁵₃, NHSO₂CR³₃, CONHSO₂CR³₃, NHSO₂R³, CONHSO₂R³, and C(═NCN)NH₂;
Y is CR⁸₂, CR⁸, NR⁸, S or O;
U is O, S, NR³, NCOR³, or NCONR³R⁷;
U¹ is O or S; [symbol]
represents a single or double bond.

47. The method of claim 46 for treating cancer further comprising administering a therapeutically effective amount of one or more additional chemotherapeutic agents.
